SEQUENCE LISTING (72 sequences)

SEQ ID NO: 1 - protein, 71 aa, Cardiovirus B
  REGION 1..71: note = Cardiovirus Theiler's-like virus
MACKHGYPLMCPLCTALDKTSDGLFTLLFDNEWYPTDLLTVDLEDEVFYPDDPHMEWTDLPLIQDIEMEPQ

SEQ ID NO: 2 - protein, 67 aa, Cardiovirus A
  REGION 1..67: note = Cardiovirus Encephalomyocarditis virus
MATTMEQETCAHSLTFEECPKCSALQYRNGFYLLKYDEEWYPEELLTDGEDDVFDPELDMEVVFELQ

SEQ ID NO: 3 - protein, 127 aa, Enterovirus C
NYHLATQDDLQNAVNVMWSRDLLVTESRAQGTDSIARCNCNAGVYYCESRRKYYPVSFVGPTFQYMEANNYYPARYQSHMLIGHGFASPGDCGGILRCHHGVIGIITAGGEGLVAFSDIRDLYAYEE

SEQ ID NO: 4 - protein, 219 aa, Erbovirus A
  REGION 1..219: note = Erbovirus Equine rhinitis B virus 1
MVTMAGNMICNVFAGLATEICSPKQGPLLDNELPLPLELAEFPNKDNNCWVAALSHYYTLCDVTNHVTKVTPTTSGIRYYLTAWQSILQTDLFNGYYPAAFAVETGLCHGPFPMQQHGYVRNATSHPYNFCLCSEPVPGEDYWHAVVKVDLSRTEARVDKWLCIDDDRMYLSGPPTRVKLASSYKIPTWIESLAQFCLQLHPVQHRRTLANSLRNEQCR

SEQ ID NO: 5 - protein, 67 aa, Cardiovirus A
  REGION 1..67: note = Cardiovirus Encephalomyocarditis virus
MATTMEQEICAHSMTFEECPKCSALQYRNGFYLLKYDEEWYPEESLTDGEDDVFDPDLDMEVVFETQ

SEQ ID NO: 6 - protein, 71 aa, Cardiovirus B
MACKHGYPFLCPLCTAIDTTHDGSFTLLIDNEWYPTDLLTVDLDDDVFHPDDSVMEWTDLPLIQDVVMEPQ

SEQ ID NO: 7-11 - skipped (5 entries)

SEQ ID NO: 12 - protein, 76 aa, Cardiovirus B
MACKHGYPDVCPICTAVDATPGFEYLLMADGEWYPTDLLCVDLDDDVFWPSDTSNQSQTMDWTDVPLIRDIVMEPQ

SEQ ID NO: 13 - protein, 1273 aa, synthetic construct
  REGION 1..1273: note = SARS-CoV-2 Spike
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 14 - DNA, 3822 bp, synthetic construct
  misc_feature 1..3822: note = SARS-CoV-2 Spike
  source 1..3822: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 15 - skipped

SEQ ID NO: 16 - DNA, 201 bp, synthetic construct
  misc_feature 1..201: note = L protein from EMCV
  source 1..201: mol_type = other DNA
atggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacag

SEQ ID NO: 17 - protein, 22 aa, synthetic construct
  REGION 1..22: note = 2A site
GSGATNFSLLKQAGDVEENPGP

SEQ ID NO: 18 - DNA, 66 bp, synthetic construct
  misc_feature 1..66: note = 2A site
  source 1..66: mol_type = other DNA
ggaagcggagctactaacttcagcctgctgaagcaggctggagatgtggaggagaaccctggacct

SEQ ID NO: 19 - DNA, 669 bp, synthetic construct
  misc_feature 1..669: note = SARS-CoV-2 M protein
  source 1..669: mol_type = other DNA
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 20 - protein, 419 aa, synthetic construct
  REGION 1..419: note = SARS-CoV-2 N protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 21 - DNA, 1257 bp, synthetic construct
  misc_feature 1..1257: note = SARS-CoV-2 N protein
  source 1..1257: mol_type = other DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcc

SEQ ID NO: 22 - protein, 75 aa, synthetic construct
  REGION 1..75: note = SARS-CoV-2 E protein
MYSFVSEETGTLIVNSVLLFLAFVVFLLVTLAILTALRLCAYCCNIVNVSLVKPSFYVYSRVKNLNSSRVPDLLV

SEQ ID NO: 23 - DNA, 228 bp, synthetic construct
  misc_feature 1..228: note = SARS-CoV-2 E protein
  source 1..228: mol_type = other DNA
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 24 - DNA, 565 bp, synthetic construct
  misc_feature 1..565: note = IRES
  source 1..565: mol_type = other DNA
tcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataat

SEQ ID NO: 25 - protein, 311 aa, synthetic construct
  REGION 1..311: note = CoVEG2 polypeptide 1 (fusion of EMCV L protein, 2A site, M protein)
MATTMEQETCAHSLTFEECPKCSALQYRNGFYLLKYDEEWYPEELLTDGEDDVFDPELDMEVVFELQGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 26 - protein, 605 aa, synthetic construct
  REGION 1..605: note = CoVEG2 polypeptide 2 (fusion of EMCV L protein, 2A site, N protein, 2A site and E protein)
MATTMEQETCAHSLTFEECPKCSALQYRNGFYLLKYDEEWYPEELLTDGEDDVFDPELDMEVVFELQGSGATNFSLLKQAGDVEENPGPMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGSGATNFSLLKQAGDVEENPGPMYSFVSEETGTLIVNSVLLFLAFVVFLLVTLAILTALRLCAYCCNIVNVSLVKPSFYVYSRVKNLNSSRVPDLLV

SEQ ID NO: 27 - DNA, 380 bp, synthetic construct
  misc_feature 1..380: note = CMV enhancer
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 28 - DNA, 204 bp, synthetic construct
  misc_feature 1..204: note = CMV promoter
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 29 - DNA, 110 bp, synthetic construct
  misc_feature 1..110: note = Cloning site
  source 1..110: mol_type = other DNA
ggtttagtgaaccgtcagatccgctagcgctaccggactcagatctcgagctcaagcttcgaattctgcagtcgacggtaccgcgggcccgggatccaccggtcgccacg

SEQ ID NO: 30 - DNA, 8399 bp, synthetic construct
  misc_feature 1..8399: note = CoVEG2 insert sequence
  source 1..8399: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccacgatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagggaagcggagctactaacttcagcctgctgaagcaggctggagatgtggaggagaaccctggacctatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaacccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagggaagcggagctactaacttcagcctgctgaagcaggctggagatgtggaggagaaccctggacctatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggccggaagcggagctactaacttcagcctgctgaagcaggctggagatgtggaggagaaccctggacctatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 31 - DNA, 6308 bp, synthetic construct
  misc_feature 1..6308: note = CoVEG1 insert sequence
  source 1..6308: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccacgatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagggaagcggagctactaacttcagcctgctgaagcaggctggagatgtggaggagaaccctggacctatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagggaagcggagctactaacttcagcctgctgaagcaggctggagatgtggaggagaaccctggacctatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

SEQ ID NO: 32 - protein, 408 aa, synthetic construct
  REGION 1..408: note = CoVEG1 polypeptide (fusion of EMCV L protein, 2A site, M protein, 2A site and E protein)
MATTMEQETCAHSLTFEECPKCSALQYRNGFYLLKYDEEWYPEELLTDGEDDVFDPELDMEVVFELQGSGATNFSLLKQAGDVEENPGPMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQGSGATNFSLLKQAGDVEENPGPMYSFVSEETGTLIVNSVLLFLAFVVFLLVTLAILTALRLCAYCCNIVNVSLVKPSFYVYSRVKNLNSSRVPDLLV

SEQ ID NO: 33 - protein, 222 aa, synthetic construct
  REGION 1..222: note = SARS-CoV-2 M protein
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 34 - DNA, 661 bp, synthetic construct
  misc_feature 1..661: note = Viral packaging signal (CoVEG8)
  source 1..661: mol_type = other DNA
gtgtcaaagcgcgaggaactgttcaccggagttttgcccatcctggtcgagctggacggcgatgtgaacggccacaagttcagcgtttctggcgagggtgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcata

SEQ ID NO: 35 - DNA, 7705 bp, synthetic construct
  misc_feature 1..7705: note = CoVEG3 Expression Cassette
  source 1..7705: mol_type = other DNA
atggccgacagcaacggcacaatcaccgtggaagagctgaagaaactgctggaacagtggaacctggtcatcggcttcctgtttctgacctggatctgtctgctgcagttcgcttatgccaatcggaacagattcctgtacatcatcaagctgatcttcctgtggctgctgtggcctgtgaccctggcttgcttcgtgctggccgctgtgtaccggatcaactggatcacaggcggaatcgccatcgccatggcctgcctggtgggcctgatgtggctgagctacttcatcgcttctttcagactgttcgccagaacccggagcatgtggtccttcaaccccgagacaaacatcctgctgaacgtgcctctgcacggcaccatcctgacaagacctctgctcgagagcgagctggtgattggcgcagtgattctgagaggccatctgaggatcgccggacaccacctgggcagatgcgacatcaaggaccttccaaaggaaatcaccgttgccaccagccggaccctgtcctactacaaactgggcgccagccaaagagtggccggcgatagcggctttgccgcctacagcagataccgcatcggaaattacaagctcaacaccgaccacagcagctcttctgataacatcgccctgctggtgcagtaatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagggaagcggagctactaacttcagcctgctgaagcaggctggagatgtggaggagaaccctggacctatgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctagacgggccagaagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggacaaggtggaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacctaacccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagggaagcggagctactaacttcagcctgctgaagcaggctggagatgtggaggagaaccctggacctatgagcgacaacggccctcaaaaccagagaaatgcccctcggatcacatttggcggacctagcgacagcaccggcagcaaccagaatggagaaagaagcggcgccagatccaagcagcggagacctcagggactgcccaacaacaccgctagctggttcaccgccctgacccaacacggcaaggaagatctgaagttccccagaggccagggcgtgcctatcaacacaaactcttctcccgacgaccagatcggatactatagacgggccactcggagaattcggggcggcgacggaaaaatgaaggacctttctccaagatggtacttctactacctcggcacaggccctgaggccggcctgccttacggcgccaacaaggatggcatcatctgggtcgccaccgagggcgccctgaacacccctaaggaccacatcggcacaagaaaccccgctaacaacgccgcaatcgtgctgcagctgcctcagggcaccaccctgcccaagggcttctacgccgagggctctagaggtggctcccaggcttctagccgctcctccagccgcagcagaaacagcagcaggaacagcacccccggcagctcccggggcaccagccccgccagaatggccggaaatggcggcgatgccgccctggccctgctcctgctggacagactgaatcagctggaaagcaagatgagcggcaaaggacagcagcagcaaggccagaccgtgaccaagaaaagcgctgctgaagcctccaagaaacctagacaaaagcggaccgccacaaaggcctacaacgtgacccaagcctttggaagaagaggccccgagcagacacagggcaatttcggcgaccaggagctgatccggcagggaaccgactacaagcactggcctcagatcgcccagttcgcccctagcgccagcgccttcttcggcatgagcagaatcggcatggaagtgaccccttctggcacctggctgacctacaccggcgctatcaagctggacgataaggatcctaacttcaaggaccaagtgatcctgctgaacaagcatatcgacgcctataagacctttccacctacagagcctaagaaagataagaagaagaaagccgacgagacacaggccctgcctcagagacagaaaaagcagcagacagtgacactgctgccagccgctgacctggatgacttcagcaagcagctgcagcagagcatgtcttctgctgatagcacccaggccggaagcggagctactaacttcagcctgctgaagcaggctggagatgtggaggagaaccctggacctatgtattcttttgtgtccgaggaaaccggcacactgatcgttaatagcgtgctgctcttcctggccttcgtggtgttcctgctggtgaccctggctatcctgaccgccctgagactgtgtgcctactgctgcaacatcgtgaacgtgtctctggtcaagcctagcttctacgtgtacagccgggtgaagaacctgaacagcagcagagtgcccgacctgctggtgtga

SEQ ID NO: 36 - DNA, 5656 bp, synthetic construct
  misc_feature 1..5656: note = CoVEG4 expression cassette
  source 1..5656: mol_type = other DNA
atggccgacagcaacggcacaatcaccgtggaagagctgaagaaactgctggaacagtggaacctggtcatcggcttcctgtttctgacctggatctgtctgctgcagttcgcttatgccaatcggaacagattcctgtacatcatcaagctgatcttcctgtggctgctgtggcctgtgaccctggcttgcttcgtgctggccgctgtgtaccggatcaactggatcacaggcggaatcgccatcgccatggcctgcctggtgggcctgatgtggctgagctacttcatcgcttctttcagactgttcgccagaacccggagcatgtggtccttcaaccccgagacaaacatcctgctgaacgtgcctctgcacggcaccatcctgacaagacctctgctcgagagcgagctggtgattggcgcagtgattctgagaggccatctgaggatcgccggacaccacctgggcagatgcgacatcaaggaccttccaaaggaaatcaccgttgccaccagccggaccctgtcctactacaaactgggcgccagccaaagagtggccggcgatagcggctttgccgcctacagcagataccgcatcggaaattacaagctcaacaccgaccacagcagctcttctgataacatcgccctgctggtgcagcgaaaacggcgcggaagcggaggaagcggagctactaacttcagcctgctgaagcaggctggagatgtggaggagaaccctggacctatgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctagacgggccagaagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggacaaggtggaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacccgaaaacggcgcggaagcggaggaagcggagctactaacttcagcctgctgaagcaggctggagatgtggaggagaaccctggacctatgtattcttttgtgtccgaggaaaccggcacactgatcgttaatagcgtgctgctcttcctggccttcgtggtgttcctgctggtgaccctggctatcctgaccgccctgagactgtgtgcctactgctgcaacatcgtgaacgtgtctctggtcaagcctagcttctacgtgtacagccgggtgaagaacctgaacagcagcagagtgcccgacctgctggtgtaatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagtaa

SEQ ID NO: 37 - DNA, 7000 bp, synthetic construct
  misc_feature 1..7000: note = CoVEG5 Expression cassette
  source 1..7000: mol_type = other DNA
atggccgacagcaacggcacaatcaccgtggaagagctgaagaaactgctggaacagtggaacctggtcatcggcttcctgtttctgacctggatctgtctgctgcagttcgcttatgccaatcggaacagattcctgtacatcatcaagctgatcttcctgtggctgctgtggcctgtgaccctggcttgcttcgtgctggccgctgtgtaccggatcaactggatcacaggcggaatcgccatcgccatggcctgcctggtgggcctgatgtggctgagctacttcatcgcttctttcagactgttcgccagaacccggagcatgtggtccttcaaccccgagacaaacatcctgctgaacgtgcctctgcacggcaccatcctgacaagacctctgctcgagagcgagctggtgattggcgcagtgattctgagaggccatctgaggatcgccggacaccacctgggcagatgcgacatcaaggaccttccaaaggaaatcaccgttgccaccagccggaccctgtcctactacaaactgggcgccagccaaagagtggccggcgatagcggctttgccgcctacagcagataccgcatcggaaattacaagctcaacaccgaccacagcagctcttctgataacatcgccctgctggtgcagcgaaaacggcgcggaagcggaggaagcggagctactaacttcagcctgctgaagcaggctggagatgtggaggagaaccctggacctatgagcgacaacggccctcaaaaccagagaaatgcccctcggatcacatttggcggacctagcgacagcaccggcagcaaccagaatggagaaagaagcggcgccagatccaagcagcggagacctcagggactgcccaacaacaccgctagctggttcaccgccctgacccaacacggcaaggaagatctgaagttccccagaggccagggcgtgcctatcaacacaaactcttctcccgacgaccagatcggatactatagacgggccactcggagaattcggggcggcgacggaaaaatgaaggacctttctccaagatggtacttctactacctcggcacaggccctgaggccggcctgccttacggcgccaacaaggatggcatcatctgggtcgccaccgagggcgccctgaacacccctaaggaccacatcggcacaagaaaccccgctaacaacgccgcaatcgtgctgcagctgcctcagggcaccaccctgcccaagggcttctacgccgagggctctagaggtggctcccaggcttctagccgctcctccagccgcagcagaaacagcagcaggaacagcacccccggcagctcccggggcaccagccccgccagaatggccggaaatggcggcgatgccgccctggccctgctcctgctggacagactgaatcagctggaaagcaagatgagcggcaaaggacagcagcagcaaggccagaccgtgaccaagaaaagcgctgctgaagcctccaagaaacctagacaaaagcggaccgccacaaaggcctacaacgtgacccaagcctttggaagaagaggccccgagcagacacagggcaatttcggcgaccaggagctgatccggcagggaaccgactacaagcactggcctcagatcgcccagttcgcccctagcgccagcgccttcttcggcatgagcagaatcggcatggaagtgaccccttctggcacctggctgacctacaccggcgctatcaagctggacgataaggatcctaacttcaaggaccaagtgatcctgctgaacaagcatatcgacgcctataagacctttccacctacagagcctaagaaagataagaagaagaaagccgacgagacacaggccctgcctcagagacagaaaaagcagcagacagtgacactgctgccagccgctgacctggatgacttcagcaagcagctgcagcagagcatgtcttctgctgatagcacccaggcccgaaaacggcgcggaagcggaggaagcggagctactaacttcagcctgctgaagcaggctggagatgtggaggagaaccctggacctatgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctggcagcgccagcagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggaccctcccgaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacccgaaaacggcgcggaagcggaggaagcggagctactaacttcagcctgctgaagcaggctggagatgtggaggagaaccctggacctatgtattcttttgtgtccgaggaaaccggcacactgatcgttaatagcgtgctgctcttcctggccttcgtggtgttcctgctggtgaccctggctatcctgaccgccctgagactgtgtgcctactgctgcaacatcgtgaacgtgtctctggtcaagcctagcttctacgtgtacagccgggtgaagaacctgaacagcagcagagtgcccgacctgctggtgtaatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagtaa

SEQ ID NO: 38 - DNA, 7705 bp, synthetic construct
  misc_feature 1..7705: note = CoVEG6 expression cassette
  source 1..7705: mol_type = other DNA
atgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctagacgggccagaagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggacaaggtggaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacctaatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagggaagcggagctactaacttcagcctgctgaagcaggctggagatgtggaggagaaccctggacctatggccgacagcaacggcacaatcaccgtggaagagctgaagaaactgctggaacagtggaacctggtcatcggcttcctgtttctgacctggatctgtctgctgcagttcgcttatgccaatcggaacagattcctgtacatcatcaagctgatcttcctgtggctgctgtggcctgtgaccctggcttgcttcgtgctggccgctgtgtaccggatcaactggatcacaggcggaatcgccatcgccatggcctgcctggtgggcctgatgtggctgagctacttcatcgcttctttcagactgttcgccagaacccggagcatgtggtccttcaaccccgagacaaacatcctgctgaacgtgcctctgcacggcaccatcctgacaagacctctgctcgagagcgagctggtgattggcgcagtgattctgagaggccatctgaggatcgccggacaccacctgggcagatgcgacatcaaggaccttccaaaggaaatcaccgttgccaccagccggaccctgtcctactacaaactgggcgccagccaaagagtggccggcgatagcggctttgccgcctacagcagataccgcatcggaaattacaagctcaacaccgaccacagcagctcttctgataacatcgccctgctggtgcagtaacccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagggaagcggagctactaacttcagcctgctgaagcaggctggagatgtggaggagaaccctggacctatgagcgacaacggccctcaaaaccagagaaatgcccctcggatcacatttggcggacctagcgacagcaccggcagcaaccagaatggagaaagaagcggcgccagatccaagcagcggagacctcagggactgcccaacaacaccgctagctggttcaccgccctgacccaacacggcaaggaagatctgaagttccccagaggccagggcgtgcctatcaacacaaactcttctcccgacgaccagatcggatactatagacgggccactcggagaattcggggcggcgacggaaaaatgaaggacctttctccaagatggtacttctactacctcggcacaggccctgaggccggcctgccttacggcgccaacaaggatggcatcatctgggtcgccaccgagggcgccctgaacacccctaaggaccacatcggcacaagaaaccccgctaacaacgccgcaatcgtgctgcagctgcctcagggcaccaccctgcccaagggcttctacgccgagggctctagaggtggctcccaggcttctagccgctcctccagccgcagcagaaacagcagcaggaacagcacccccggcagctcccggggcaccagccccgccagaatggccggaaatggcggcgatgccgccctggccctgctcctgctggacagactgaatcagctggaaagcaagatgagcggcaaaggacagcagcagcaaggccagaccgtgaccaagaaaagcgctgctgaagcctccaagaaacctagacaaaagcggaccgccacaaaggcctacaacgtgacccaagcctttggaagaagaggccccgagcagacacagggcaatttcggcgaccaggagctgatccggcagggaaccgactacaagcactggcctcagatcgcccagttcgcccctagcgccagcgccttcttcggcatgagcagaatcggcatggaagtgaccccttctggcacctggctgacctacaccggcgctatcaagctggacgataaggatcctaacttcaaggaccaagtgatcctgctgaacaagcatatcgacgcctataagacctttccacctacagagcctaagaaagataagaagaagaaagccgacgagacacaggccctgcctcagagacagaaaaagcagcagacagtgacactgctgccagccgctgacctggatgacttcagcaagcagctgcagcagagcatgtcttctgctgatagcacccaggccggaagcggagctactaacttcagcctgctgaagcaggctggagatgtggaggagaaccctggacctatgtattcttttgtgtccgaggaaaccggcacactgatcgttaatagcgtgctgctcttcctggccttcgtggtgttcctgctggtgaccctggctatcctgaccgccctgagactgtgtgcctactgctgcaacatcgtgaacgtgtctctggtcaagcctagcttctacgtgtacagccgggtgaagaacctgaacagcagcagagtgcccgacctgctggtgtga

SEQ ID NO: 39 - DNA, 6976 bp, synthetic construct
  misc_feature 1..6976: note = CoVEG7 Expression cassette
  source 1..6976: mol_type = other DNA
atggccgacagcaacggcacaatcaccgtggaagagctgaagaaactgctggaacagtggaacctggtcatcggcttcctgtttctgacctggatctgtctgctgcagttcgcttatgccaatcggaacagattcctgtacatcatcaagctgatcttcctgtggctgctgtggcctgtgaccctggcttgcttcgtgctggccgctgtgtaccggatcaactggatcacaggcggaatcgccatcgccatggcctgcctggtgggcctgatgtggctgagctacttcatcgcttctttcagactgttcgccagaacccggagcatgtggtccttcaaccccgagacaaacatcctgctgaacgtgcctctgcacggcaccatcctgacaagacctctgctcgagagcgagctggtgattggcgcagtgattctgagaggccatctgaggatcgccggacaccacctgggcagatgcgacatcaaggaccttccaaaggaaatcaccgttgccaccagccggaccctgtcctactacaaactgggcgccagccaaagagtggccggcgatagcggctttgccgcctacagcagataccgcatcggaaattacaagctcaacaccgaccacagcagctcttctgataacatcgccctgctggtgcagcgaaaacggcgcggaagcggaggaagcggagctactaacttcagcctgctgaagcaggctggagatgtggaggagaaccctggacctatgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctagacgggccagaagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggacaaggtggaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacccgaaaacggcgcggaagcggaggaagcggagctactaacttcagcctgctgaagcaggctggagatgtggaggagaaccctggacctatgtattcttttgtgtccgaggaaaccggcacactgatcgttaatagcgtgctgctcttcctggccttcgtggtgttcctgctggtgaccctggctatcctgaccgccctgagactgtgtgcctactgctgcaacatcgtgaacgtgtctctggtcaagcctagcttctacgtgtacagccgggtgaagaacctgaacagcagcagagtgcccgacctgctggtgtaatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagggaagcggagctactaacttcagcctgctgaagcaggctggagatgtggaggagaaccctggacctatgagcgacaacggccctcaaaaccagagaaatgcccctcggatcacatttggcggacctagcgacagcaccggcagcaaccagaatggagaaagaagcggcgccagatccaagcagcggagacctcagggactgcccaacaacaccgctagctggttcaccgccctgacccaacacggcaaggaagatctgaagttccccagaggccagggcgtgcctatcaacacaaactcttctcccgacgaccagatcggatactatagacgggccactcggagaattcggggcggcgacggaaaaatgaaggacctttctccaagatggtacttctactacctcggcacaggccctgaggccggcctgccttacggcgccaacaaggatggcatcatctgggtcgccaccgagggcgccctgaacacccctaaggaccacatcggcacaagaaaccccgctaacaacgccgcaatcgtgctgcagctgcctcagggcaccaccctgcccaagggcttctacgccgagggctctagaggtggctcccaggcttctagccgctcctccagccgcagcagaaacagcagcaggaacagcacccccggcagctcccggggcaccagccccgccagaatggccggaaatggcggcgatgccgccctggccctgctcctgctggacagactgaatcagctggaaagcaagatgagcggcaaaggacagcagcagcaaggccagaccgtgaccaagaaaagcgctgctgaagcctccaagaaacctagacaaaagcggaccgccacaaaggcctacaacgtgacccaagcctttggaagaagaggccccgagcagacacagggcaatttcggcgaccaggagctgatccggcagggaaccgactacaagcactggcctcagatcgcccagttcgcccctagcgccagcgccttcttcggcatgagcagaatcggcatggaagtgaccccttctggcacctggctgacctacaccggcgctatcaagctggacgataaggatcctaacttcaaggaccaagtgatcctgctgaacaagcatatcgacgcctataagacctttccacctacagagcctaagaaagataagaagaagaaagccgacgagacacaggccctgcctcagagacagaaaaagcagcagacagtgacactgctgccagccgctgacctggatgacttcagcaagcagctgcagcagagcatgtcttctgctgatagcacccaggcc

SEQ ID NO: 40 - DNA, 7640 bp, synthetic construct
  misc_feature 1..7640: note = CoVEG8 expression cassette
  source 1..7640: mol_type = other DNA
atggccgacagcaacggcacaatcaccgtggaagagctgaagaaactgctggaacagtggaacctggtcatcggcttcctgtttctgacctggatctgtctgctgcagttcgcttatgccaatcggaacagattcctgtacatcatcaagctgatcttcctgtggctgctgtggcctgtgaccctggcttgcttcgtgctggccgctgtgtaccggatcaactggatcacaggcggaatcgccatcgccatggcctgcctggtgggcctgatgtggctgagctacttcatcgcttctttcagactgttcgccagaacccggagcatgtggtccttcaaccccgagacaaacatcctgctgaacgtgcctctgcacggcaccatcctgacaagacctctgctcgagagcgagctggtgattggcgcagtgattctgagaggccatctgaggatcgccggacaccacctgggcagatgcgacatcaaggaccttccaaaggaaatcaccgttgccaccagccggaccctgtcctactacaaactgggcgccagccaaagagtggccggcgatagcggctttgccgcctacagcagataccgcatcggaaattacaagctcaacaccgaccacagcagctcttctgataacatcgccctgctggtgcagcgaaaacggcgcggaagcggaggaagcggagctactaacttcagcctgctgaagcaggctggagatgtggaggagaaccctggacctatgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctagacgggccagaagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggacaaggtggaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacccgaaaacggcgcggaagcggaggaagcggagctactaacttcagcctgctgaagcaggctggagatgtggaggagaaccctggacctatgtattcttttgtgtccgaggaaaccggcacactgatcgttaatagcgtgctgctcttcctggccttcgtggtgttcctgctggtgaccctggctatcctgaccgccctgagactgtgtgcctactgctgcaacatcgtgaacgtgtctctggtcaagcctagcttctacgtgtacagccgggtgaagaacctgaacagcagcagagtgcccgacctgctggtgtaatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagggaagcggagctactaacttcagcctgctgaagcaggctggagatgtggaggagaaccctggacctatgagcgacaacggccctcaaaaccagagaaatgcccctcggatcacatttggcggacctagcgacagcaccggcagcaaccagaatggagaaagaagcggcgccagatccaagcagcggagacctcagggactgcccaacaacaccgctagctggttcaccgccctgacccaacacggcaaggaagatctgaagttccccagaggccagggcgtgcctatcaacacaaactcttctcccgacgaccagatcggatactatagacgggccactcggagaattcggggcggcgacggaaaaatgaaggacctttctccaagatggtacttctactacctcggcacaggccctgaggccggcctgccttacggcgccaacaaggatggcatcatctgggtcgccaccgagggcgccctgaacacccctaaggaccacatcggcacaagaaaccccgctaacaacgccgcaatcgtgctgcagctgcctcagggcaccaccctgcccaagggcttctacgccgagggctctagaggtggctcccaggcttctagccgctcctccagccgcagcagaaacagcagcaggaacagcacccccggcagctcccggggcaccagccccgccagaatggccggaaatggcggcgatgccgccctggccctgctcctgctggacagactgaatcagctggaaagcaagatgagcggcaaaggacagcagcagcaaggccagaccgtgaccaagaaaagcgctgctgaagcctccaagaaacctagacaaaagcggaccgccacaaaggcctacaacgtgacccaagcctttggaagaagaggccccgagcagacacagggcaatttcggcgaccaggagctgatccggcagggaaccgactacaagcactggcctcagatcgcccagttcgcccctagcgccagcgccttcttcggcatgagcagaatcggcatggaagtgaccccttctggcacctggctgacctacaccggcgctatcaagctggacgataaggatcctaacttcaaggaccaagtgatcctgctgaacaagcatatcgacgcctataagacctttccacctacagagcctaagaaagataagaagaagaaagccgacgagacacaggccctgcctcagagacagaaaaagcagcagacagtgacactgctgccagccgctgacctggatgacttcagcaagcagctgcagcagagcatgtcttctgctgatagcacccaggcctaagtgtcaaagcgcgaggaactgttcaccggagttttgcccatcctggtcgagctggacggcgatgtgaacggccacaagttcagcgtttctggcgagggtgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcata

SEQ ID NO: 41 - DNA, 7000 bp, synthetic construct; identical to SEQ ID NO: 37
  misc_feature 1..7000: note = CoVEG9 expression cassette
  source 1..7000: mol_type = other DNA

SEQ ID NO: 42 - DNA, 7661 bp, synthetic construct
  misc_feature 1..7661: note = CoVEG10 expression cassette
  source 1..7661: mol_type = other DNA
atggccgacagcaacggcacaatcaccgtggaagagctgaagaaactgctggaacagtggaacctggtcatcggcttcctgtttctgacctggatctgtctgctgcagttcgcttatgccaatcggaacagattcctgtacatcatcaagctgatcttcctgtggctgctgtggcctgtgaccctggcttgcttcgtgctggccgctgtgtaccggatcaactggatcacaggcggaatcgccatcgccatggcctgcctggtgggcctgatgtggctgagctacttcatcgcttctttcagactgttcgccagaacccggagcatgtggtccttcaaccccgagacaaacatcctgctgaacgtgcctctgcacggcaccatcctgacaagacctctgctcgagagcgagctggtgattggcgcagtgattctgagaggccatctgaggatcgccggacaccacctgggcagatgcgacatcaaggaccttccaaaggaaatcaccgttgccaccagccggaccctgtcctactacaaactgggcgccagccaaagagtggccggcgatagcggctttgccgcctacagcagataccgcatcggaaattacaagctcaacaccgaccacagcagctcttctgataacatcgccctgctggtgcagcgaaaacggcgcggaagcggaggaagcggagctactaacttcagcctgctgaagcaggctggagatgtggaggagaaccctggacctatgagcgacaacggccctcaaaaccagagaaatgcccctcggatcacatttggcggacctagcgacagcaccggcagcaaccagaatggagaaagaagcggcgccagatccaagcagcggagacctcagggactgcccaacaacaccgctagctggttcaccgccctgacccaacacggcaaggaagatctgaagttccccagaggccagggcgtgcctatcaacacaaactcttctcccgacgaccagatcggatactatagacgggccactcggagaattcggggcggcgacggaaaaatgaaggacctttctccaagatggtacttctactacctcggcacaggccctgaggccggcctgccttacggcgccaacaaggatggcatcatctgggtcgccaccgagggcgccctgaacacccctaaggaccacatcggcacaagaaaccccgctaacaacgccgcaatcgtgctgcagctgcctcagggcaccaccctgcccaagggcttctacgccgagggctctagaggtggctcccaggcttctagccgctcctccagccgcagcagaaacagcagcaggaacagcacccccggcagctcccggggcaccagccccgccagaatggccggaaatggcggcgatgccgccctggccctgctcctgctggacagactgaatcagctggaaagcaagatgagcggcaaaggacagcagcagcaaggccagaccgtgaccaagaaaagcgctgctgaagcctccaagaaacctagacaaaagcggaccgccacaaaggcctacaacgtgacccaagcctttggaagaagaggccccgagcagacacagggcaatttcggcgaccaggagctgatccggcagggaaccgactacaagcactggcctcagatcgcccagttcgcccctagcgccagcgccttcttcggcatgagcagaatcggcatggaagtgaccccttctggcacctggctgacctacaccggcgctatcaagctggacgataaggatcctaacttcaaggaccaagtgatcctgctgaacaagcatatcgacgcctataagacctttccacctacagagcctaagaaagataagaagaagaaagccgacgagacacaggccctgcctcagagacagaaaaagcagcagacagtgacactgctgccagccgctgacctggatgacttcagcaagcagctgcagcagagcatgtcttctgctgatagcacccaggcccgaaaacggcgcggaagcggaggaagcggagctactaacttcagcctgctgaagcaggctggagatgtggaggagaaccctggacctatgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctggcagcgccagcagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggaccctcccgaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacccgaaaacggcgcggaagcggaggaagcggagctactaacttcagcctgctgaagcaggctggagatgtggaggagaaccctggacctatgtattcttttgtgtccgaggaaaccggcacactgatcgttaatagcgtgctgctcttcctggccttcgtggtgttcctgctggtgaccctggctatcctgaccgccctgagactgtgtgcctactgctgcaacatcgtgaacgtgtctctggtcaagcctagcttctacgtgtacagccgggtgaagaacctgaacagcagcagagtgcccgacctgctggtgtaatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagtaagtgtcaaagcgcgaggaactgttcaccggagttttgcccatcctggtcgagctggacggcgatgtgaacggccacaagttcagcgtttctggcgagggtgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcata

SEQ ID NO: 43 - DNA, 7893 bp, synthetic construct
  misc_feature 1..7893: note = CoVEG11 expression cassette
  source 1..7893: mol_type = other DNA
aaaaaaaaaaattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggccgacagcaacggcacaatcaccgtggaagagctgaagaaactgctggaacagtggaacctggtcatcggcttcctgtttctgacctggatctgtctgctgcagttcgcttatgccaatcggaacagattcctgtacatcatcaagctgatcttcctgtggctgctgtggcctgtgaccctggcttgcttcgtgctggccgctgtgtaccggatcaactggatcacaggcggaatcgccatcgccatggcctgcctggtgggcctgatgtggctgagctacttcatcgcttctttcagactgttcgccagaacccggagcatgtggtccttcaaccccgagacaaacatcctgctgaacgtgcctctgcacggcaccatcctgacaagacctctgctcgagagcgagctggtgattggcgcagtgattctgagaggccatctgaggatcgccggacaccacctgggcagatgcgacatcaaggaccttccaaaggaaatcaccgttgccaccagccggaccctgtcctactacaaactgggcgccagccaaagagtggccggcgatagcggctttgccgcctacagcagataccgcatcggaaattacaagctcaacaccgaccacagcagctcttctgataacatcgccctgctggtgcagcgaaaacggcgcggaagcggaggaagcggagctactaacttcagcctgctgaagcaggctggagatgtggaggagaaccctggacctatgagcgacaacggccctcaaaaccagagaaatgcccctcggatcacatttggcggacctagcgacagcaccggcagcaaccagaatggagaaagaagcggcgccagatccaagcagcggagacctcagggactgcccaacaacaccgctagctggttcaccgccctgacccaacacggcaaggaagatctgaagttccccagaggccagggcgtgcctatcaacacaaactcttctcccgacgaccagatcggatactatagacgggccactcggagaattcggggcggcgacggaaaaatgaaggacctttctccaagatggtacttctactacctcggcacaggccctgaggccggcctgccttacggcgccaacaaggatggcatcatctgggtcgccaccgagggcgccctgaacacccctaaggaccacatcggcacaagaaaccccgctaacaacgccgcaatcgtgctgcagctgcctcagggcaccaccctgcccaagggcttctacgccgagggctctagaggtggctcccaggcttctagccgctcctccagccgcagcagaaacagcagcaggaacagcacccccggcagctcccggggcaccagccccgccagaatggccggaaatggcggcgatgccgccctggccctgctcctgctggacagactgaatcagctggaaagcaagatgagcggcaaaggacagcagcagcaaggccagaccgtgaccaagaaaagcgctgctgaagcctccaagaaacctagacaaaagcggaccgccacaaaggcctacaacgtgacccaagcctttggaagaagaggccccgagcagacacagggcaatttcggcgaccaggagctgatccggcagggaaccgactacaagcactggcctcagatcgcccagttcgcccctagcgccagcgccttcttcggcatgagcagaatcggcatggaagtgaccccttctggcacctggctgacctacaccggcgctatcaagctggacgataaggatcctaacttcaaggaccaagtgatcctgctgaacaagcatatcgacgcctataagacctttccacctacagagcctaagaaagataagaagaagaaagccgacgagacacaggccctgcctcagagacagaaaaagcagcagacagtgacactgctgccagccgctgacctggatgacttcagcaagcagctgcagcagagcatgtcttctgctgatagcacccaggcccgaaaacggcgcggaagcggaggaagcggagctactaacttcagcctgctgaagcaggctggagatgtggaggagaaccctggacctatgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctggcagcgccagcagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggaccctcccgaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacccgaaaacggcgcggaagcggaggaagcggagctactaacttcagcctgctgaagcaggctggagatgtggaggagaaccctggacctatgtattcttttgtgtccgaggaaaccggcacactgatcgttaatagcgtgctgctcttcctggccttcgtggtgttcctgctggtgaccctggctatcctgaccgccctgagactgtgtgcctactgctgcaacatcgtgaacgtgtctctggtcaagcctagcttctacgtgtacagccgggtgaagaacctgaacagcagcagagtgcccgacctgctggtgtaatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagtaagtgtttacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtgcgaaattgttgttgtt

SEQ ID NO: 44 - DNA, 7618 bp, synthetic construct
  misc_feature 1..7618: note = CoVEG12 expression cassette
  source 1..7618: mol_type = other DNA
atggccgacagcaacggcacaatcaccgtggaagagctgaagaaactgctggaacagtggaacctggtcatcggcttcctgtttctgacctggatctgtctgctgcagttcgcttatgccaatcggaacagattcctgtacatcatcaagctgatcttcctgtggctgctgtggcctgtgaccctggcttgcttcgtgctggccgctgtgtaccggatcaactggatcacaggcggaatcgccatcgccatggcctgcctggtgggcctgatgtggctgagctacttcatcgcttctttcagactgttcgccagaacccggagcatgtggtccttcaaccccgagacaaacatcctgctgaacgtgcctctgcacggcaccatcctgacaagacctctgctcgagagcgagctggtgattggcgcagtgattctgagaggccatctgaggatcgccggacaccacctgggcagatgcgacatcaaggaccttccaaaggaaatcaccgttgccaccagccggaccctgtcctactacaaactgggcgccagccaaagagtggccggcgatagcggctttgccgcctacagcagataccgcatcggaaattacaagctcaacaccgaccacagcagctcttctgataacatcgccctgctggtgcagcgaaaacggcgcggaagcggaggaagcggagctactaacttcagcctgctgaagcaggctggagatgtggaggagaaccctggacctatgagcgacaacggccctcaaaaccagagaaatgcccctcggatcacatttggcggacctagcgacagcaccggcagcaaccagaatggagaaagaagcggcgccagatccaagcagcggagacctcagggactgcccaacaacaccgctagctggttcaccgccctgacccaacacggcaaggaagatctgaagttccccagaggccagggcgtgcctatcaacacaaactcttctcccgacgaccagatcggatactatagacgggccactcggagaattcggggcggcgacggaaaaatgaaggacctttctccaagatggtacttctactacctcggcacaggccctgaggccggcctgccttacggcgccaacaaggatggcatcatctgggtcgccaccgagggcgccctgaacacccctaaggaccacatcggcacaagaaaccccgctaacaacgccgcaatcgtgctgcagctgcctcagggcaccaccctgcccaagggcttctacgccgagggctctagaggtggctcccaggcttctagccgctcctccagccgcagcagaaacagcagcaggaacagcacccccggcagctcccggggcaccagccccgccagaatggccggaaatggcggcgatgccgccctggccctgctcctgctggacagactgaatcagctggaaagcaagatgagcggcaaaggacagcagcagcaaggccagaccgtgaccaagaaaagcgctgctgaagcctccaagaaacctagacaaaagcggaccgccacaaaggcctacaacgtgacccaagcctttggaagaagaggccccgagcagacacagggcaatttcggcgaccaggagctgatccggcagggaaccgactacaagcactggcctcagatcgcccagttcgcccctagcgccagcgccttcttcggcatgagcagaatcggcatggaagtgaccccttctggcacctggctgacctacaccggcgctatcaagctggacgataaggatcctaacttcaaggaccaagtgatcctgctgaacaagcatatcgacgcctataagacctttccacctacagagcctaagaaagataagaagaagaaagccgacgagacacaggccctgcctcagagacagaaaaagcagcagacagtgacactgctgccagccgctgacctggatgacttcagcaagcagctgcagcagagcatgtcttctgctgatagcacccaggcccgaaaacggcgcggaagcggaggaagcggagctactaacttcagcctgctgaagcaggctggagatgtggaggagaaccctggacctatgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctagacgggccagaagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggacaaggtggaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacccgaaaacggcgcggaagcggaggaagcggagctactaacttcagcctgctgaagcaggctggagatgtggaggagaaccctggacctatgtattcttttgtgtccgaggaaaccggcacactgatcgttaatagcgtgctgctcttcctggccttcgtggtgttcctgctggtgaccctggctatcctgaccgccctgagactgtgtgcctactgctgcaacatcgtgaacgtgtctctggtcaagcctagcttctacgtgtacagccgggtgaagaacctgaacagcagcagagtgcccgacctgctggtgtaatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagtaagtgtttacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtgcgaaattgttgttgtt

SEQ ID NO: 45 - DNA, 7882 bp, synthetic construct
  misc_feature 1..7882: note = CoVEG13 expression cassette
  source 1..7882: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaaatggccgacagcaacggcacaatcaccgtggaagagctgaagaaactgctggaacagtggaacctggtcatcggcttcctgtttctgacctggatctgtctgctgcagttcgcttatgccaatcggaacagattcctgtacatcatcaagctgatcttcctgtggctgctgtggcctgtgaccctggcttgcttcgtgctggccgctgtgtaccggatcaactggatcacaggcggaatcgccatcgccatggcctgcctggtgggcctgatgtggctgagctacttcatcgcttctttcagactgttcgccagaacccggagcatgtggtccttcaaccccgagacaaacatcctgctgaacgtgcctctgcacggcaccatcctgacaagacctctgctcgagagcgagctggtgattggcgcagtgattctgagaggccatctgaggatcgccggacaccacctgggcagatgcgacatcaaggaccttccaaaggaaatcaccgttgccaccagccggaccctgtcctactacaaactgggcgccagccaaagagtggccggcgatagcggctttgccgcctacagcagataccgcatcggaaattacaagctcaacaccgaccacagcagctcttctgataacatcgccctgctggtgcagcgaaaacggcgcggaagcggaggaagcggagctactaacttcagcctgctgaagcaggctggagatgtggaggagaaccctggacctatgagcgacaacggccctcaaaaccagagaaatgcccctcggatcacatttggcggacctagcgacagcaccggcagcaaccagaatggagaaagaagcggcgccagatccaagcagcggagacctcagggactgcccaacaacaccgctagctggttcaccgccctgacccaacacggcaaggaagatctgaagttccccagaggccagggcgtgcctatcaacacaaactcttctcccgacgaccagatcggatactatagacgggccactcggagaattcggggcggcgacggaaaaatgaaggacctttctccaagatggtacttctactacctcggcacaggccctgaggccggcctgccttacggcgccaacaaggatggcatcatctgggtcgccaccgagggcgccctgaacacccctaaggaccacatcggcacaagaaaccccgctaacaacgccgcaatcgtgctgcagctgcctcagggcaccaccctgcccaagggcttctacgccgagggctctagaggtggctcccaggcttctagccgctcctccagccgcagcagaaacagcagcaggaacagcacccccggcagctcccggggcaccagccccgccagaatggccggaaatggcggcgatgccgccctggccctgctcctgctggacagactgaatcagctggaaagcaagatgagcggcaaaggacagcagcagcaaggccagaccgtgaccaagaaaagcgctgctgaagcctccaagaaacctagacaaaagcggaccgccacaaaggcctacaacgtgacccaagcctttggaagaagaggccccgagcagacacagggcaatttcggcgaccaggagctgatccggcagggaaccgactacaagcactggcctcagatcgcccagttcgcccctagcgccagcgccttcttcggcatgagcagaatcggcatggaagtgaccccttctggcacctggctgacctacaccggcgctatcaagctggacgataaggatcctaacttcaaggaccaagtgatcctgctgaacaagcatatcgacgcctataagacctttccacctacagagcctaagaaagataagaagaagaaagccgacgagacacaggccctgcctcagagacagaaaaagcagcagacagtgacactgctgccagccgctgacctggatgacttcagcaagcagctgcagcagagcatgtcttctgctgatagcacccaggcccgaaaacggcgcggaagcggaggaagcggagctactaacttcagcctgctgaagcaggctggagatgtggaggagaaccctggacctatgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctagacgggccagaagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggacaaggtggaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacccgaaaacggcgcggaagcggaggaagcggagctactaacttcagcctgctgaagcaggctggagatgtggaggagaaccctggacctatgtattcttttgtgtccgaggaaaccggcacactgatcgttaatagcgtgctgctcttcctggccttcgtggtgttcctgctggtgaccctggctatcctgaccgccctgagactgtgtgcctactgctgcaacatcgtgaacgtgtctctggtcaagcctagcttctacgtgtacagccgggtgaagaacctgaacagcagcagagtgcccgacctgctggtgtaatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagtaagtgtttacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtgcgaaattgttgttgtt

SEQ ID NO: 46 - DNA, 8773 bp, synthetic construct
  misc_feature 1..8773: note = CoVEG14 expression cassette
  source 1..8773: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaaatggccgacagcaacggcacaatcaccgtggaagagctgaagaaactgctggaacagtggaacctggtcatcggcttcctgtttctgacctggatctgtctgctgcagttcgcttatgccaatcggaacagattcctgtacatcatcaagctgatcttcctgtggctgctgtggcctgtgaccctggcttgcttcgtgctggccgctgtgtaccggatcaactggatcacaggcggaatcgccatcgccatggcctgcctggtgggcctgatgtggctgagctacttcatcgcttctttcagactgttcgccagaacccggagcatgtggtccttcaaccccgagacaaacatcctgctgaacgtgcctctgcacggcaccatcctgacaagacctctgctcgagagcgagctggtgattggcgcagtgattctgagaggccatctgaggatcgccggacaccacctgggcagatgcgacatcaaggaccttccaaaggaaatcaccgttgccaccagccggaccctgtcctactacaaactgggcgccagccaaagagtggccggcgatagcggctttgccgcctacagcagataccgcatcggaaattacaagctcaacaccgaccacagcagctcttctgataacatcgccctgctggtgcagcgaaaacggcgcggaagcggaggaagcggagctactaacttcagcctgctgaagcaggctggagatgtggaggagaaccctggacctatgagcgacaacggccctcaaaaccagagaaatgcccctcggatcacatttggcggacctagcgacagcaccggcagcaaccagaatggagaaagaagcggcgccagatccaagcagcggagacctcagggactgcccaacaacaccgctagctggttcaccgccctgacccaacacggcaaggaagatctgaagttccccagaggccagggcgtgcctatcaacacaaactcttctcccgacgaccagatcggatactatagacgggccactcggagaattcggggcggcgacggaaaaatgaaggacctttctccaagatggtacttctactacctcggcacaggccctgaggccggcctgccttacggcgccaacaaggatggcatcatctgggtcgccaccgagggcgccctgaacacccctaaggaccacatcggcacaagaaaccccgctaacaacgccgcaatcgtgctgcagctgcctcagggcaccaccctgcccaagggcttctacgccgagggctctagaggtggctcccaggcttctagccgctcctccagccgcagcagaaacagcagcaggaacagcacccccggcagctcccggggcaccagccccgccagaatggccggaaatggcggcgatgccgccctggccctgctcctgctggacagactgaatcagctggaaagcaagatgagcggcaaaggacagcagcagcaaggccagaccgtgaccaagaaaagcgctgctgaagcctccaagaaacctagacaaaagcggaccgccacaaaggcctacaacgtgacccaagcctttggaagaagaggccccgagcagacacagggcaatttcggcgaccaggagctgatccggcagggaaccgactacaagcactggcctcagatcgcccagttcgcccctagcgccagcgccttcttcggcatgagcagaatcggcatggaagtgaccccttctggcacctggctgacctacaccggcgctatcaagctggacgataaggatcctaacttcaaggaccaagtgatcctgctgaacaagcatatcgacgcctataagacctttccacctacagagcctaagaaagataagaagaagaaagccgacgagacacaggccctgcctcagagacagaaaaagcagcagacagtgacactgctgccagccgctgacctggatgacttcagcaagcagctgcagcagagcatgtcttctgctgatagcacccaggcccgaaaacggcgcggaagcggaggaagcggagctactaacttcagcctgctgaagcaggctggagatgtggaggagaaccctggacctatgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctagacgggccagaagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggacaaggtggaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacccgaaaacggcgcggaagcggaggaagcggagctactaacttcagcctgctgaagcaggctggagatgtggaggagaaccctggacctatgtattcttttgtgtccgaggaaaccggcacactgatcgttaatagcgtgctgctcttcctggccttcgtggtgttcctgctggtgaccctggctatcctgaccgccctgagactgtgtgcctactgctgcaacatcgtgaacgtgtctctggtcaagcctagcttctacgtgtacagccgggtgaagaacctgaacagcagcagagtgcccgacctgctggtgtaatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagggaagcggagctactaacttcagcctgctgaagcaggctggagatgtggaggagaaccctggacctatggacctgttcatgagaatcttcaccatcggcaccgtgacactgaagcagggcgagatcaaggatgccacccctagcgacttcgtgagagccaccgccacaattcctatccaggctagcctgccttttggatggctgatcgtgggcgtcgccctgctcgccgtgttccagagcgcctctaagatcattacactgaagaaaagatggcagctggccctctccaaaggcgtgcacttcgtgtgtaatctgctgctgctttttgtgacagtgtacagccacctgctgctggttgctgctggcctggaagcccctttcctgtacctgtacgccctggtctacttcctgcagtctatcaacttcgtgcggatcatcatgcggctgtggctgtgctggaagtgcagaagcaagaacccactgctgtacgacgccaattacttcctgtgttggcacaccaactgctacgactactgcatcccctacaacagcgtgaccagcagcatcgtgatcacctctggcgacggaacaaccagccctatcagcgagcatgattaccagatcggcggatatacagagaagtgggagagcggcgtgaaggactgcgtggtgctgcacagctactttacctccgattactaccaactgtattctacccagctgagcaccgacaccggcgtggaacacgtgaccttcttcatctacaacaagatcgtggacgagcctgaggaacacgtgcagatccacactatcgacggcagctctggcgttgtgaaccctgtgatggaacccatctacgatgagcccaccacaacaacctccgtgcccctgtaagtgtttacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtgcgaaattgttgttgtt

SEQ ID NO: 47 - DNA, 7677 bp, synthetic construct
  misc_feature 1..7677: note = CoVEG15 expression cassette
  source 1..7677: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaaatggccgacagcaacggcacaatcaccgtggaagagctgaagaaactgctggaacagtggaacctggtcatcggcttcctgtttctgacctggatctgtctgctgcagttcgcttatgccaatcggaacagattcctgtacatcatcaagctgatcttcctgtggctgctgtggcctgtgaccctggcttgcttcgtgctggccgctgtgtaccggatcaactggatcacaggcggaatcgccatcgccatggcctgcctggtgggcctgatgtggctgagctacttcatcgcttctttcagactgttcgccagaacccggagcatgtggtccttcaaccccgagacaaacatcctgctgaacgtgcctctgcacggcaccatcctgacaagacctctgctcgagagcgagctggtgattggcgcagtgattctgagaggccatctgaggatcgccggacaccacctgggcagatgcgacatcaaggaccttccaaaggaaatcaccgttgccaccagccggaccctgtcctactacaaactgggcgccagccaaagagtggccggcgatagcggctttgccgcctacagcagataccgcatcggaaattacaagctcaacaccgaccacagcagctcttctgataacatcgccctgctggtgcagcgaaaacggcgcggaagcggaggaagcggagctactaacttcagcctgctgaagcaggctggagatgtggaggagaaccctggacctatgagcgacaacggccctcaaaaccagagaaatgcccctcggatcacatttggcggacctagcgacagcaccggcagcaaccagaatggagaaagaagcggcgccagatccaagcagcggagacctcagggactgcccaacaacaccgctagctggttcaccgccctgacccaacacggcaaggaagatctgaagttccccagaggccagggcgtgcctatcaacacaaactcttctcccgacgaccagatcggatactatagacgggccactcggagaattcggggcggcgacggaaaaatgaaggacctttctccaagatggtacttctactacctcggcacaggccctgaggccggcctgccttacggcgccaacaaggatggcatcatctgggtcgccaccgagggcgccctgaacacccctaaggaccacatcggcacaagaaaccccgctaacaacgccgcaatcgtgctgcagctgcctcagggcaccaccctgcccaagggcttctacgccgagggctctagaggtggctcccaggcttctagccgctcctccagccgcagcagaaacagcagcaggaacagcacccccggcagctcccggggcaccagccccgccagaatggccggaaatggcggcgatgccgccctggccctgctcctgctggacagactgaatcagctggaaagcaagatgagcggcaaaggacagcagcagcaaggccagaccgtgaccaagaaaagcgctgctgaagcctccaagaaacctagacaaaagcggaccgccacaaaggcctacaacgtgacccaagcctttggaagaagaggccccgagcagacacagggcaatttcggcgaccaggagctgatccggcagggaaccgactacaagcactggcctcagatcgcccagttcgcccctagcgccagcgccttcttcggcatgagcagaatcggcatggaagtgaccccttctggcacctggctgacctacaccggcgctatcaagctggacgataaggatcctaacttcaaggaccaagtgatcctgctgaacaagcatatcgacgcctataagacctttccacctacagagcctaagaaagataagaagaagaaagccgacgagacacaggccctgcctcagagacagaaaaagcagcagacagtgacactgctgccagccgctgacctggatgacttcagcaagcagctgcagcagagcatgtcttctgctgatagcacccaggcccgaaaacggcgcggaagcggaggaagcggagctactaacttcagcctgctgaagcaggctggagatgtggaggagaaccctggacctatgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctagacgggccagaagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggacaaggtggaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacccgaaaacggcgcggaagcggaggaagcggagctactaacttcagcctgctgaagcaggctggagatgtggaggagaaccctggacctatgtattcttttgtgtccgaggaaaccggcacactgatcgttaatagcgtgctgctcttcctggccttcgtggtgttcctgctggtgaccctggctatcctgaccgccctgagactgtgtgcctactgctgcaacatcgtgaacgtgtctctggtcaagcctagcttctacgtgtacagccgggtgaagaacctgaacagcagcagagtgcccgacctgctggtgtaatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagtgtttacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtgcgaaattgttgttgtt

SEQ ID NO: 48 - DNA, 8509 bp, synthetic construct
  misc_feature 1..8509: note = CoVEG16 expression cassette
  source 1..8509: mol_type = other DNA
atggccgacagcaacggcacaatcaccgtggaagagctgaagaaactgctggaacagtggaacctggtcatcggcttcctgtttctgacctggatctgtctgctgcagttcgcttatgccaatcggaacagattcctgtacatcatcaagctgatcttcctgtggctgctgtggcctgtgaccctggcttgcttcgtgctggccgctgtgtaccggatcaactggatcacaggcggaatcgccatcgccatggcctgcctggtgggcctgatgtggctgagctacttcatcgcttctttcagactgttcgccagaacccggagcatgtggtccttcaaccccgagacaaacatcctgctgaacgtgcctctgcacggcaccatcctgacaagacctctgctcgagagcgagctggtgattggcgcagtgattctgagaggccatctgaggatcgccggacaccacctgggcagatgcgacatcaaggaccttccaaaggaaatcaccgttgccaccagccggaccctgtcctactacaaactgggcgccagccaaagagtggccggcgatagcggctttgccgcctacagcagataccgcatcggaaattacaagctcaacaccgaccacagcagctcttctgataacatcgccctgctggtgcagcgaaaacggcgcggaagcggaggaagcggagctactaacttcagcctgctgaagcaggctggagatgtggaggagaaccctggacctatgagcgacaacggccctcaaaaccagagaaatgcccctcggatcacatttggcggacctagcgacagcaccggcagcaaccagaatggagaaagaagcggcgccagatccaagcagcggagacctcagggactgcccaacaacaccgctagctggttcaccgccctgacccaacacggcaaggaagatctgaagttccccagaggccagggcgtgcctatcaacacaaactcttctcccgacgaccagatcggatactatagacgggccactcggagaattcggggcggcgacggaaaaatgaaggacctttctccaagatggtacttctactacctcggcacaggccctgaggccggcctgccttacggcgccaacaaggatggcatcatctgggtcgccaccgagggcgccctgaacacccctaaggaccacatcggcacaagaaaccccgctaacaacgccgcaatcgtgctgcagctgcctcagggcaccaccctgcccaagggcttctacgccgagggctctagaggtggctcccaggcttctagccgctcctccagccgcagcagaaacagcagcaggaacagcacccccggcagctcccggggcaccagccccgccagaatggccggaaatggcggcgatgccgccctggccctgctcctgctggacagactgaatcagctggaaagcaagatgagcggcaaaggacagcagcagcaaggccagaccgtgaccaagaaaagcgctgctgaagcctccaagaaacctagacaaaagcggaccgccacaaaggcctacaacgtgacccaagcctttggaagaagaggccccgagcagacacagggcaatttcggcgaccaggagctgatccggcagggaaccgactacaagcactggcctcagatcgcccagttcgcccctagcgccagcgccttcttcggcatgagcagaatcggcatggaagtgaccccttctggcacctggctgacctacaccggcgctatcaagctggacgataaggatcctaacttcaaggaccaagtgatcctgctgaacaagcatatcgacgcctataagacctttccacctacagagcctaagaaagataagaagaagaaagccgacgagacacaggccctgcctcagagacagaaaaagcagcagacagtgacactgctgccagccgctgacctggatgacttcagcaagcagctgcagcagagcatgtcttctgctgatagcacccaggcccgaaaacggcgcggaagcggaggaagcggagctactaacttcagcctgctgaagcaggctggagatgtggaggagaaccctggacctatgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctggcagcgccagcagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggaccctcccgaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacccgaaaacggcgcggaagcggaggaagcggagctactaacttcagcctgctgaagcaggctggagatgtggaggagaaccctggacctatgtattcttttgtgtccgaggaaaccggcacactgatcgttaatagcgtgctgctcttcctggccttcgtggtgttcctgctggtgaccctggctatcctgaccgccctgagactgtgtgcctactgctgcaacatcgtgaacgtgtctctggtcaagcctagcttctacgtgtacagccgggtgaagaacctgaacagcagcagagtgcccgacctgctggtgtaatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagggaagcggagctactaacttcagcctgctgaagcaggctggagatgtggaggagaaccctggacctatggacctgttcatgagaatcttcaccatcggcaccgtgacactgaagcagggcgagatcaaggatgccacccctagcgacttcgtgagagccaccgccacaattcctatccaggctagcctgccttttggatggctgatcgtgggcgtcgccctgctcgccgtgttccagagcgcctctaagatcattacactgaagaaaagatggcagctggccctctccaaaggcgtgcacttcgtgtgtaatctgctgctgctttttgtgacagtgtacagccacctgctgctggttgctgctggcctggaagcccctttcctgtacctgtacgccctggtctacttcctgcagtctatcaacttcgtgcggatcatcatgcggctgtggctgtgctggaagtgcagaagcaagaacccactgctgtacgacgccaattacttcctgtgttggcacaccaactgctacgactactgcatcccctacaacagcgtgaccagcagcatcgtgatcacctctggcgacggaacaaccagccctatcagcgagcatgattaccagatcggcggatatacagagaagtgggagagcggcgtgaaggactgcgtggtgctgcacagctactttacctccgattactaccaactgtattctacccagctgagcaccgacaccggcgtggaacacgtgaccttcttcatctacaacaagatcgtggacgagcctgaggaacacgtgcagatccacactatcgacggcagctctggcgttgtgaaccctgtgatggaacccatctacgatgagcccaccacaacaacctccgtgcccctgtaagtgtttacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtgcgaaattgttgttgtt

SEQ ID NO: 49 - DNA, 8773 bp, synthetic construct
  misc_feature 1..8773: note = CoVEG17 expression cassette
  source 1..8773: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaaatggccgacagcaacggcacaatcaccgtggaagagctgaagaaactgctggaacagtggaacctggtcatcggcttcctgtttctgacctggatctgtctgctgcagttcgcttatgccaatcggaacagattcctgtacatcatcaagctgatcttcctgtggctgctgtggcctgtgaccctggcttgcttcgtgctggccgctgtgtaccggatcaactggatcacaggcggaatcgccatcgccatggcctgcctggtgggcctgatgtggctgagctacttcatcgcttctttcagactgttcgccagaacccggagcatgtggtccttcaaccccgagacaaacatcctgctgaacgtgcctctgcacggcaccatcctgacaagacctctgctcgagagcgagctggtgattggcgcagtgattctgagaggccatctgaggatcgccggacaccacctgggcagatgcgacatcaaggaccttccaaaggaaatcaccgttgccaccagccggaccctgtcctactacaaactgggcgccagccaaagagtggccggcgatagcggctttgccgcctacagcagataccgcatcggaaattacaagctcaacaccgaccacagcagctcttctgataacatcgccctgctggtgcagcgaaaacggcgcggaagcggaggaagcggagctactaacttcagcctgctgaagcaggctggagatgtggaggagaaccctggacctatgagcgacaacggccctcaaaaccagagaaatgcccctcggatcacatttggcggacctagcgacagcaccggcagcaaccagaatggagaaagaagcggcgccagatccaagcagcggagacctcagggactgcccaacaacaccgctagctggttcaccgccctgacccaacacggcaaggaagatctgaagttccccagaggccagggcgtgcctatcaacacaaactcttctcccgacgaccagatcggatactatagacgggccactcggagaattcggggcggcgacggaaaaatgaaggacctttctccaagatggtacttctactacctcggcacaggccctgaggccggcctgccttacggcgccaacaaggatggcatcatctgggtcgccaccgagggcgccctgaacacccctaaggaccacatcggcacaagaaaccccgctaacaacgccgcaatcgtgctgcagctgcctcagggcaccaccctgcccaagggcttctacgccgagggctctagaggtggctcccaggcttctagccgctcctccagccgcagcagaaacagcagcaggaacagcacccccggcagctcccggggcaccagccccgccagaatggccggaaatggcggcgatgccgccctggccctgctcctgctggacagactgaatcagctggaaagcaagatgagcggcaaaggacagcagcagcaaggccagaccgtgaccaagaaaagcgctgctgaagcctccaagaaacctagacaaaagcggaccgccacaaaggcctacaacgtgacccaagcctttggaagaagaggccccgagcagacacagggcaatttcggcgaccaggagctgatccggcagggaaccgactacaagcactggcctcagatcgcccagttcgcccctagcgccagcgccttcttcggcatgagcagaatcggcatggaagtgaccccttctggcacctggctgacctacaccggcgctatcaagctggacgataaggatcctaacttcaaggaccaagtgatcctgctgaacaagcatatcgacgcctataagacctttccacctacagagcctaagaaagataagaagaagaaagccgacgagacacaggccctgcctcagagacagaaaaagcagcagacagtgacactgctgccagccgctgacctggatgacttcagcaagcagctgcagcagagcatgtcttctgctgatagcacccaggcccgaaaacggcgcggaagcggaggaagcggagctactaacttcagcctgctgaagcaggctggagatgtggaggagaaccctggacctatgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctggcagcgccagcagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggaccctcccgaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacccgaaaacggcgcggaagcggaggaagcggagctactaacttcagcctgctgaagcaggctggagatgtggaggagaaccctggacctatgtattcttttgtgtccgaggaaaccggcacactgatcgttaatagcgtgctgctcttcctggccttcgtggtgttcctgctggtgaccctggctatcctgaccgccctgagactgtgtgcctactgctgcaacatcgtgaacgtgtctctggtcaagcctagcttctacgtgtacagccgggtgaagaacctgaacagcagcagagtgcccgacctgctggtgtaatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagggaagcggagctactaacttcagcctgctgaagcaggctggagatgtggaggagaaccctggacctatggacctgttcatgagaatcttcaccatcggcaccgtgacactgaagcagggcgagatcaaggatgccacccctagcgacttcgtgagagccaccgccacaattcctatccaggctagcctgccttttggatggctgatcgtgggcgtcgccctgctcgccgtgttccagagcgcctctaagatcattacactgaagaaaagatggcagctggccctctccaaaggcgtgcacttcgtgtgtaatctgctgctgctttttgtgacagtgtacagccacctgctgctggttgctgctggcctggaagcccctttcctgtacctgtacgccctggtctacttcctgcagtctatcaacttcgtgcggatcatcatgcggctgtggctgtgctggaagtgcagaagcaagaacccactgctgtacgacgccaattacttcctgtgttggcacaccaactgctacgactactgcatcccctacaacagcgtgaccagcagcatcgtgatcacctctggcgacggaacaaccagccctatcagcgagcatgattaccagatcggcggatatacagagaagtgggagagcggcgtgaaggactgcgtggtgctgcacagctactttacctccgattactaccaactgtattctacccagctgagcaccgacaccggcgtggaacacgtgaccttcttcatctacaacaagatcgtggacgagcctgaggaacacgtgcagatccacactatcgacggcagctctggcgttgtgaaccctgtgatggaacccatctacgatgagcccaccacaacaacctccgtgcccctgtaagtgtttacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtgcgaaattgttgttgtt

SEQ ID NO: 50 - DNA, 12 bp, synthetic construct
  misc_feature 1..12: note = Furin cleavage site
  source 1..12: mol_type = other DNA
cgaaaacggcgc

SEQ ID NO: 51 - protein, 1273 aa, synthetic construct
  REGION 1..1273: note = Mutant S protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 52 - DNA, 3819 bp, synthetic construct
  misc_feature 1..3819: note = Mutant S gene (CoVEG9)
  source 1..3819: mol_type = other DNA
atgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctggcagcgccagcagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggaccctcccgaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacc

SEQ ID NO: 53 - protein, 275 aa, synthetic construct
  REGION 1..275: note = ORF3 protein
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 54 - DNA, 828 bp, synthetic construct
  misc_feature 1..828: note = ORF3 gene (CoVEG14)
  source 1..828: mol_type = other DNA
atggacctgttcatgagaatcttcaccatcggcaccgtgacactgaagcagggcgagatcaaggatgccacccctagcgacttcgtgagagccaccgccacaattcctatccaggctagcctgccttttggatggctgatcgtgggcgtcgccctgctcgccgtgttccagagcgcctctaagatcattacactgaagaaaagatggcagctggccctctccaaaggcgtgcacttcgtgtgtaatctgctgctgctttttgtgacagtgtacagccacctgctgctggttgctgctggcctggaagcccctttcctgtacctgtacgccctggtctacttcctgcagtctatcaacttcgtgcggatcatcatgcggctgtggctgtgctggaagtgcagaagcaagaacccactgctgtacgacgccaattacttcctgtgttggcacaccaactgctacgactactgcatcccctacaacagcgtgaccagcagcatcgtgatcacctctggcgacggaacaaccagccctatcagcgagcatgattaccagatcggcggatatacagagaagtgggagagcggcgtgaaggactgcgtggtgctgcacagctactttacctccgattactaccaactgtattctacccagctgagcaccgacaccggcgtggaacacgtgaccttcttcatctacaacaagatcgtggacgagcctgaggaacacgtgcagatccacactatcgacggcagctctggcgttgtgaaccctgtgatggaacccatctacgatgagcccaccacaacaacctccgtgcccctgtaa

SEQ ID NO: 55 - DNA, 5651 bp, synthetic construct
  misc_feature 1..5651: note = WNV Minimal plasmid sequence
  source 1..5651: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccacatgggcggcaagacaggcatcgccgtgatgatcggcctgatcgcctccgtgggcgccgtgaccctgagcaacttccagggcaaggtgatgatgacagtgaacgccacagatgttaccgatgttatcacaatccctaccgccgctggaaagaacttgtgcatcgtacgggccatggatgtggggtacatgtgcgacgacaccatcacctacgagtgccctgtgctgagcgccggcaatgaccccgaggacatcgactgctggtgcaccaagtctgccgtttacgtgagatatggcaggtgtaccaaaaccagacacagcaggagatctcggagaagcctgaccgtgcaaacacacggcgagtccaccctggccaacaagaagggcgcatggatggacagcaccaaggccactcggtacctggtgaagaccgagagctggatcctgagaaaccctggatacgccctggtggccgccgtgattggctggatgctgggctctaacaccatgcagagagtggtgttcgtggtgctactgctcctagtggctcctgcttacagcttcaactgcctgggcatgtctaaccgggacttcctggaaggcgtgtccggcgctacatgggtggacctggtgctcgagggagatagctgcgtgaccatcatgtcaaaggacaagcccaccatcgacgtgaaaatgatgaacatggaagctgctaatctggccgaggtcagatcttactgctacctggccacagtgagtgatctgagcacaaaggccgcctgccccaccatgggcgaggcccacaacgataagcgggccgatcctgccttcgtgtgtagacagggcgtggtggaccggggatggggcaacggctgcgggctgttcggcaagggcagcatcgatacctgtgccaaattcgcctgtagcaccaaggccatcggccggaccattctgaaagaaaacatcaagtacgaggtggctatcttcgtgcatggccctaccaccgtcgagagccacggcaactactccacacaggtgggcgccacacaggccggccgattttctatcacacctgccgcccccagctatacactgaaactgggcgagtacggcgaagtgacagtggattgcgagcctagaagcggcatcgacactaacgcctactacgtgatgaccgtgggcacaaaaaccttcctggttcacagagagtggttcatggacctgaacctgccttggtccagcgccggcagcaccgtgtggcgcaatagagagacactgatggaattcgaggaacctcacgccaccaagcagagcgtgatcgccctcggtagccaggagggcgccctgcaccaggccctggctggcgccatccccgtggaattctctagcaacaccgtgaaactgaccagcggccacctgaagtgcagagtgaagatggaaaagctccaactgaagggaacaacttacggcgtctgcagcaaggctttcaagttcctgggcacccctgccgacaccggacacggaaccgtcgtgctggaactgcagtacaccggcacagacggcccatgtaaagtgcctatcagcagcgtggcctccctgaacgacctgacaccagtgggcagactggtgaccgttaatcctttcgtcagcgtggctactgccaatgccaaggtgctgatcgagctggaaccccccttcggcgactcttatatcgtggtgggaagaggagaacaacagatcaaccaccactggcacaagagcggttcgtctatcggaaaggcttttaccaccacactgaagggcgctcagcggctggccgccctgggcgacacagcctgggacttcggcagcgtgggcggagtatttacgtccgtcggcaaggccgtccatcaggtgtttggaggagcctttcggagcctgttcggaggcatgagctggatcacccagggcctgctgggcgcgctgctgctgtggatgggaattaacgccagagatagaagcatcgccctgacattcctggccgtgggcggcgtgctgctgtttctgtctgtgaacgtgcacgcgtaacccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagtaagcgaaattgttgttgttaacttgtttattgcagcttataatggttacaaataaagcaatagcatcacaaatttcacaaataaagcatttttttcactgcattctagttgtggtttgtccaaactcatcaatgtatcttaaggcgtcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 56 - DNA, 255 bp, synthetic construct
  misc_feature 1..255: note = Origin
  source 1..255: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataa

SEQ ID NO: 57 - DNA, 584 bp, synthetic construct
  misc_feature 1..584: note = CMV promoter and enhancer sequence
  source 1..584: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 58 - DNA, 54 bp, synthetic construct
  misc_feature 1..54: note = Signal sequence
  source 1..54: mol_type = other DNA
ggcggcaagacaggcatcgccgtgatgatcggcctgatcgcctccgtgggcgcc

SEQ ID NO: 59 - DNA, 501 bp, synthetic construct
  misc_feature 1..501: note = prM gene
  source 1..501: mol_type = other DNA
gtgaccctgagcaacttccagggcaaggtgatgatgacagtgaacgccacagatgttaccgatgttatcacaatccctaccgccgctggaaagaacttgtgcatcgtacgggccatggatgtggggtacatgtgcgacgacaccatcacctacgagtgccctgtgctgagcgccggcaatgaccccgaggacatcgactgctggtgcaccaagtctgccgtttacgtgagatatggcaggtgtaccaaaaccagacacagcaggagatctcggagaagcctgaccgtgcaaacacacggcgagtccaccctggccaacaagaagggcgcatggatggacagcaccaaggccactcggtacctggtgaagaccgagagctggatcctgagaaaccctggatacgccctggtggccgccgtgattggctggatgctgggctctaacaccatgcagagagtggtgttcgtggtgctactgctcctagtggctcctgcttacagc

SEQ ID NO: 60 - DNA, 1506 bp, synthetic construct
  misc_feature 1..1506: note = Envelope gene
  source 1..1506: mol_type = other DNA
ttcaactgcctgggcatgtctaaccgggacttcctggaaggcgtgtccggcgctacatgggtggacctggtgctcgagggagatagctgcgtgaccatcatgtcaaaggacaagcccaccatcgacgtgaaaatgatgaacatggaagctgctaatctggccgaggtcagatcttactgctacctggccacagtgagtgatctgagcacaaaggccgcctgccccaccatgggcgaggcccacaacgataagcgggccgatcctgccttcgtgtgtagacagggcgtggtggaccggggatggggcaacggctgcgggctgttcggcaagggcagcatcgatacctgtgccaaattcgcctgtagcaccaaggccatcggccggaccattctgaaagaaaacatcaagtacgaggtggctatcttcgtgcatggccctaccaccgtcgagagccacggcaactactccacacaggtgggcgccacacaggccggccgattttctatcacacctgccgcccccagctatacactgaaactgggcgagtacggcgaagtgacagtggattgcgagcctagaagcggcatcgacactaacgcctactacgtgatgaccgtgggcacaaaaaccttcctggttcacagagagtggttcatggacctgaacctgccttggtccagcgccggcagcaccgtgtggcgcaatagagagacactgatggaattcgaggaacctcacgccaccaagcagagcgtgatcgccctcggtagccaggagggcgccctgcaccaggccctggctggcgccatccccgtggaattctctagcaacaccgtgaaactgaccagcggccacctgaagtgcagagtgaagatggaaaagctccaactgaagggaacaacttacggcgtctgcagcaaggctttcaagttcctgggcacccctgccgacaccggacacggaaccgtcgtgctggaactgcagtacaccggcacagacggcccatgtaaagtgcctatcagcagcgtggcctccctgaacgacctgacaccagtgggcagactggtgaccgttaatcctttcgtcagcgtggctactgccaatgccaaggtgctgatcgagctggaaccccccttcggcgactcttatatcgtggtgggaagaggagaacaacagatcaaccaccactggcacaagagcggttcgtctatcggaaaggcttttaccaccacactgaagggcgctcagcggctggccgccctgggcgacacagcctgggacttcggcagcgtgggcggagtatttacgtccgtcggcaaggccgtccatcaggtgtttggaggagcctttcggagcctgttcggaggcatgagctggatcacccagggcctgctgggcgcgctgctgctgtggatgggaattaacgccagagatagaagcatcgccctgacattcctggccgtgggcggcgtgctgctgtttctgtctgtgaacgtgcacgcgtaa

SEQ ID NO: 61 - DNA, 563 bp, synthetic construct
  misc_feature 1..563: note = IRES encoding sequence
  source 1..563: mol_type = other DNA
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 62 - DNA, 204 bp, synthetic construct
  misc_feature 1..204: note = EMCV L protein encoding sequence
  source 1..204: mol_type = other DNA
atggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagtaa

SEQ ID NO: 63 - DNA, 122 bp, synthetic construct
  misc_feature 1..122: note = SV40 poly A encoding sequence
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 64 - DNA, 2203 bp, synthetic construct
  misc_feature 1..2203: note = Expression cassette of WNV standard plasmid
  source 1..2203: mol_type = other DNA
atgggcggcaagacaggcatcgccgtgatgatcggcctgatcgcctccgtgggcgccgtgaccctgagcaacttccagggcaaggtgatgatgacagtgaacgccacagatgttaccgatgttatcacaatccctaccgccgctggaaagaacttgtgcatcgtacgggccatggatgtggggtacatgtgcgacgacaccatcacctacgagtgccctgtgctgagcgccggcaatgaccccgaggacatcgactgctggtgcaccaagtctgccgtttacgtgagatatggcaggtgtaccaaaaccagacacagcaggagatctcggagaagcctgaccgtgcaaacacacggcgagtccaccctggccaacaagaagggcgcatggatggacagcaccaaggccactcggtacctggtgaagaccgagagctggatcctgagaaaccctggatacgccctggtggccgccgtgattggctggatgctgggctctaacaccatgcagagagtggtgttcgtggtgctactgctcctagtggctcctgcttacagcttcaactgcctgggcatgtctaaccgggacttcctggaaggcgtgtccggcgctacatgggtggacctggtgctcgagggagatagctgcgtgaccatcatgtcaaaggacaagcccaccatcgacgtgaaaatgatgaacatggaagctgctaatctggccgaggtcagatcttactgctacctggccacagtgagtgatctgagcacaaaggccgcctgccccaccatgggcgaggcccacaacgataagcgggccgatcctgccttcgtgtgtagacagggcgtggtggaccggggatggggcaacggctgcgggctgttcggcaagggcagcatcgatacctgtgccaaattcgcctgtagcaccaaggccatcggccggaccattctgaaagaaaacatcaagtacgaggtggctatcttcgtgcatggccctaccaccgtcgagagccacggcaactactccacacaggtgggcgccacacaggccggccgattttctatcacacctgccgcccccagctatacactgaaactgggcgagtacggcgaagtgacagtggattgcgagcctagaagcggcatcgacactaacgcctactacgtgatgaccgtgggcacaaaaaccttcctggttcacagagagtggttcatggacctgaacctgccttggtccagcgccggcagcaccgtgtggcgcaatagagagacactgatggaattcgaggaacctcacgccaccaagcagagcgtgatcgccctcggtagccaggagggcgccctgcaccaggccctggctggcgccatccccgtggaattctctagcaacaccgtgaaactgaccagcggccacctgaagtgcagagtgaagatggaaaagctccaactgaagggaacaacttacggcgtctgcagcaaggctttcaagttcctgggcacccctgccgacaccggacacggaaccgtcgtgctggaactgcagtacaccggcacagacggcccatgtaaagtgcctatcagcagcgtggcctccctgaacgacctgacaccagtgggcagactggtgaccgttaatcctttcgtcagcgtggctactgccaatgccaaggtgctgatcgagctggaaccccccttcggcgactcttatatcgtggtgggaagaggagaacaacagatcaaccaccactggcacaagagcggttcgtctatcggaaaggcttttaccaccacactgaagggcgctcagcggctggccgccctgggcgacacagcctgggacttcggcagcgtgggcggagtatttacgtccgtcggcaaggccgtccatcaggtgtttggaggagcctttcggagcctgttcggaggcatgagctggatcacccagggcctgctgggcgcgctgctgctgtggatgggaattaacgccagagatagaagcatcgccctgacattcctggccgtgggcggcgtgctgctgtttctgtctgtgaacgtgcacgcgtaagcgaaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 65 - DNA, 4591 bp, synthetic construct
  misc_feature 1..4591: note = Control S only plasmid expression cassette
  source 1..4591: mol_type = other DNA
atgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctggcagcgccagcagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggaccctcccgaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacctaatcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggaacaagagacttgcgcgcactctctcacttttgaggaatgcccaaaatgctctgctctacaataccgtaatggattttacctgctaaagtatgatgaagaatggtacccagaggagttattgactgatggagaggatgatgtctttgatcccgaattagacatggaagtcgttttcgagttacagtaa

SEQ ID NO: 66 - DNA, 669 bp, synthetic construct
  misc_feature 1..669: note = SARS CoV2 M gene
  source 1..669: mol_type = other DNA
atggccgacagcaacggcacaatcaccgtggaagagctgaagaaactgctggaacagtggaacctggtcatcggcttcctgtttctgacctggatctgtctgctgcagttcgcttatgccaatcggaacagattcctgtacatcatcaagctgatcttcctgtggctgctgtggcctgtgaccctggcttgcttcgtgctggccgctgtgtaccggatcaactggatcacaggcggaatcgccatcgccatggcctgcctggtgggcctgatgtggctgagctacttcatcgcttctttcagactgttcgccagaacccggagcatgtggtccttcaaccccgagacaaacatcctgctgaacgtgcctctgcacggcaccatcctgacaagacctctgctcgagagcgagctggtgattggcgcagtgattctgagaggccatctgaggatcgccggacaccacctgggcagatgcgacatcaaggaccttccaaaggaaatcaccgttgccaccagccggaccctgtcctactacaaactgggcgccagccaaagagtggccggcgatagcggctttgccgcctacagcagataccgcatcggaaattacaagctcaacaccgaccacagcagctcttctgataacatcgccctgctggtgcagtaa

SEQ ID NO: 67 - DNA, 565 bp, synthetic construct; identical to SEQ ID NO: 24
  misc_feature 1..565: note = IRES encoding sequence
  source 1..565: mol_type = other DNA

SEQ ID NO: 68 - DNA, 201 bp, synthetic construct; identical to SEQ ID NO: 16
  misc_feature 1..201: note = L peptide from ECMV encoding sequence
  source 1..201: mol_type = other DNA

SEQ ID NO: 69 - DNA, 66 bp, synthetic construct; identical to SEQ ID NO: 18
  misc_feature 1..66: note = P2A skipping site encoding sequence
  source 1..66: mol_type = other DNA

SEQ ID NO: 70 - DNA, 3822 bp, synthetic construct
  misc_feature 1..3822: note = SARS CoV2 Spike gene
  source 1..3822: mol_type = other DNA
atgttcgtgttcctggtgctgctgcctctggtcagctcccagtgtgtgaacctgaccaccagaacccagctgccacctgcttatacaaactccttcactcggggggtatactaccccgacaaggtgttcagatctagcgtgctgcattctacacaagacctgttcctgcccttcttcagcaacgtgacctggttccacgccatccacgtgtctggaaccaacggaaccaagagattcgacaaccccgtgctgcctttcaacgacggcgtgtacttcgccagcaccgagaagtccaacatcatcagaggatggattttcggcaccacactggacagcaaaacccagagcctgctgatcgtgaacaacgccaccaacgtggtgatcaaggtgtgcgagttccagttctgcaatgatcccttcctgggcgtgtactaccacaagaacaacaagtcttggatggaaagcgagttcagagtgtattccagcgccaacaattgcaccttcgagtacgtgagccaaccctttctgatggaccttgaaggcaagcagggcaacttcaaaaatctgcgagaatttgtgttcaagaacatcgacggatacttcaagatctactctaagcacacgccaatcaacctggtgagagatctgccccagggctttagcgctttggaacctctggtggacctgcctatcggaatcaacatcaccagatttcaaactctcctggccctgcacagatcttatctgacccctggggacagtagtagcggctggacagccggcgccgccgcctactacgtgggatacctgcagcctagaacattcctgctgaagtacaatgagaacggaacaatcacagacgccgtggactgcgccctggatcctttgagcgagacaaagtgcaccctgaagtcgttcaccgtcgaaaaaggcatctaccagaccagcaacttccgcgtgcagcctacggaatctatcgtgcggttccccaacatcaccaacctgtgccctttcggcgaggtgtttaacgctacaaggttcgccagcgtgtatgcctggaacagaaagagaatcagcaattgcgtggccgattatagcgttctgtacaacagcgcttccttcagcaccttcaagtgctacggcgtgtctccaaccaagctgaacgacctctgcttcaccaatgtctacgctgactctttcgtgattagaggcgatgaggttagacagatcgcacctggccagaccggcaaaatcgctgactacaactacaagctgcctgatgacttcacaggctgtgtcattgcctggaactcaaataacctggactctaaagtgggcggcaactacaactacctgtaccggctgttccggaagagcaatctgaaaccttttgagcgggacatctctacagagatctaccaggccggcagcacaccctgcaacggcgttgagggcttcaactgctacttccctctgcagagctacggctttcagccaacaaatggagtgggctaccagccgtacagagtggtggtgctgagcttcgaactgctgcatgccccagccacagtgtgtggacctaagaagtctaccaacctggtgaagaacaagtgcgtgaactttaactttaacggcctgaccggcacaggcgtgctgaccgaatccaacaaaaagttcctgcccttccaacagttcggcagagacatcgccgatacaaccgatgccgtgcgggacccccagaccttagaaatcctagatatcaccccgtgcagcttcggcggagtctctgttattactcctggcaccaacaccagcaaccaagtggctgttctgtaccaaggcgtgaactgcaccgaagtgcctgtggctatccacgccgatcagctgaccccaacctggcgggtgtatagcaccggctctaacgtgttccagacccgggctggctgcctgatcggcgccgaacacgtcaacaactcctatgaatgtgacatccccatcggggctggcatctgcgccagttaccagacacagacaaatagccctagacgggccagaagcgtggcctcccagagtatcattgcctacaccatgagcctgggcgccgagaacagcgtggcctattctaacaatagcatcgcaatccctaccaactttaccatctctgtgacaaccgagatcctgcctgtgagcatgaccaaaaccagcgtggactgcacgatgtacatctgtggcgacagcacagaatgcagtaatctgttgctgcagtacggcagcttttgcacccagttgaatagagccctgaccggaatcgccgtagagcaggacaaaaatacccaggaggtgttcgcccaggtgaaacagatctacaagacacctcccattaaggacttcggaggttttaacttcagccagatcctgcccgacccttccaagcctagcaaacgctccttcatcgaggacctgctcttcaacaaggtgacactggctgatgccggcttcatcaagcagtacggagattgtctgggagacatcgccgctagagatctgatctgcgcccaaaagttcaacggcctgaccgtgctgcctcctctgcttacagacgagatgatcgcccagtacaccagcgccctgctggctggcaccatcacaagcggctggaccttcggagccggagccgctctgcaaatcccctttgccatgcagatggcctaccggttcaacggcatcggcgtgacacagaatgtgctgtacgagaaccagaagctgatcgctaaccagtttaacagcgctatcggcaagatccaggactcgctgagtagcaccgcctctgccctgggcaagctgcaggacgtcgtgaaccagaacgcccaagccctgaacacactggtgaaacagctgagcagcaacttcggcgccatcagctctgtgctgaacgatatcctgagcagactggacaaggtggaagccgaggtccagatcgacagactgatcacaggaagactgcagagcctgcaaacgtacgtgacacagcagctgatccgggcagccgaaatccgggccagcgccaatctggccgctaccaagatgagcgagtgcgtgttaggccagagcaagcgggtggatttctgcggtaagggataccacctgatgagctttccccagagcgctcctcacggcgtggtgtttctgcacgtgacctacgttcctgcccaggaaaagaacttcaccaccgcccctgctatctgccacgatggcaaggcccacttccctagagagggcgttttcgtgtctaacggcacacactggtttgtgacccagagaaacttctacgagcctcagatcatcaccacagacaacacctttgtgagcggcaattgcgacgtggtgatcggaattgttaataataccgtgtacgaccctctgcagcctgagctcgacagcttcaaggaagagctggacaagtacttcaagaaccacacctccccagatgtggacctgggcgatatttcaggcatcaacgcctccgtcgtgaatatccagaaggagatcgaccggctcaacgaggtggccaagaaccttaacgagagcctgatcgacctgcaggaactgggcaaatatgagcagtacatcaagtggccttggtacatctggctgggctttatcgcaggcctgatcgctatcgtgatggtgaccattatgctgtgttgtatgaccagctgttgtagttgtctgaagggctgctgttcttgcggcagctgctgcaagttcgacgaagacgactcagagcccgtgctgaaaggcgtgaagctgcactacacctaa

SEQ ID NO: 71 - DNA, 1257 bp, synthetic construct
  misc_feature 1..1257: note = SARS CoV2 N gene
  source 1..1257: mol_type = other DNA
atgagcgacaacggccctcaaaaccagagaaatgcccctcggatcacatttggcggacctagcgacagcaccggcagcaaccagaatggagaaagaagcggcgccagatccaagcagcggagacctcagggactgcccaacaacaccgctagctggttcaccgccctgacccaacacggcaaggaagatctgaagttccccagaggccagggcgtgcctatcaacacaaactcttctcccgacgaccagatcggatactatagacgggccactcggagaattcggggcggcgacggaaaaatgaaggacctttctccaagatggtacttctactacctcggcacaggccctgaggccggcctgccttacggcgccaacaaggatggcatcatctgggtcgccaccgagggcgccctgaacacccctaaggaccacatcggcacaagaaaccccgctaacaacgccgcaatcgtgctgcagctgcctcagggcaccaccctgcccaagggcttctacgccgagggctctagaggtggctcccaggcttctagccgctcctccagccgcagcagaaacagcagcaggaacagcacccccggcagctcccggggcaccagccccgccagaatggccggaaatggcggcgatgccgccctggccctgctcctgctggacagactgaatcagctggaaagcaagatgagcggcaaaggacagcagcagcaaggccagaccgtgaccaagaaaagcgctgctgaagcctccaagaaacctagacaaaagcggaccgccacaaaggcctacaacgtgacccaagcctttggaagaagaggccccgagcagacacagggcaatttcggcgaccaggagctgatccggcagggaaccgactacaagcactggcctcagatcgcccagttcgcccctagcgccagcgccttcttcggcatgagcagaatcggcatggaagtgaccccttctggcacctggctgacctacaccggcgctatcaagctggacgataaggatcctaacttcaaggaccaagtgatcctgctgaacaagcatatcgacgcctataagacctttccacctacagagcctaagaaagataagaagaagaaagccgacgagacacaggccctgcctcagagacagaaaaagcagcagacagtgacactgctgccagccgctgacctggatgacttcagcaagcagctgcagcagagcatgtcttctgctgatagcacccaggcc

SEQ ID NO: 72 - DNA, 228 bp, synthetic construct
  misc_feature 1..228: note = SARS CoV2 Envelope gene
  source 1..228: mol_type = other DNA
atgtattcttttgtgtccgaggaaaccggcacactgatcgttaatagcgtgctgctcttcctggccttcgtggtgttcctgctggtgaccctggctatcctgaccgccctgagactgtgtgcctactgctgcaacatcgtgaacgtgtctctggtcaagcctagcttctacgtgtacagccgggtgaagaacctgaacagcagcagagtgcccgacctgctggtgtga

(19) United States
(12) Patent Application Publication
(10) Pub. No.: US 2024/0156948 A1
(43) Pub. Date: May 16, 2024

(54) VACCINE COMPOSITIONS AND METHODS OF USE THEREOF

(71) Applicant: Excepgen Inc., San Francisco, CA (US)

(72) Inventors: Barbara MERTINS, San Francisco, CA (US); Thomas FOLLIARD, San Francisco, CA (US); Imre MÄGER, San Francisco, CA (US)

(21) Appl. No.: 18/466,251

(22) Filed: Sep. 13, 2023

Related U.S. Application Data
(63) Continuation of application No. PCT/US2022/020774, filed on Mar. 17, 2022.
(60) Provisional application No. 63/162,496, filed on Mar. 17, 2021.

(51) Int. Cl. A61K 39/215; A61K 39/12; A61K 39/145; A61K 39/29; A61P 37/04; C07K 14/005

(52) U.S. Cl. CPC A61K 39/215; A61K 39/12; A61K 39/145; A61K 39/292; A61P 37/04; C07K 14/005; A61K 2039/5258

(57) ABSTRACT

The present disclosure provides compositions and methods for use in vaccines, comprising polynucleotides encoding one or more viral antigen proteins and an enhancer protein, wherein the enhancer protein is a picornavirus leader (L) or a functional variant thereof. The compositions and methods provided herein may improve the production of functional viral-like particles (VLP).

### CROSS-REFERENCE TO RELATED APPLICATIONS

This application is a continuation of International Patent Application No. PCT/US2022/020774, filed Mar. 17, 2022, which claims priority to U.S. provisional patent application No. 63/162,496, filed Mar. 17, 2021, the disclosures of each of which are incorporated by reference herein in their entirety.

### INCORPORATION OF THE SEQUENCE LISTING

The contents of the electronic sequence listing (EXCI_003_03US_SeqList_ST26.xml; Size: 254,290 bytes; and Date of Creation: Jan. 30, 2024) are herein incorporated by reference in their entirety.

### BACKGROUND

Viral infectious diseases, e.g., the flu, are extremely widespread, and often contagious. Viral diseases result in a wide variety of symptoms that vary in character and severity depending on the type of viral infection and other factors, including the person's age and overall health. Viral infections can be treated with varying degrees of success, depending on the type of virus and other factors. Sometimes, the treatment may involve just management of symptoms.

The most effective way to combat viral infections is through vaccination, which can induce a holistic cellular and/or humoral immune response that is protective against future infections. Vaccinations offer enormous public health and economic benefits by preventing the occurrence of, or minimizing, the severity of viral infections. Although vaccines are available to prevent more than 20 life-threatening diseases, including viral infections, the appearance of new infectious viruses, e.g., SARS CoV2, can necessitate rapid research and development of vaccines against new viral targets. However, the development of vaccines, even with the latest genome sequencing and other technological advancements, is still time-consuming and expensive.

Therefore, there is a need for methods and compositions that have the versatility to be quickly adapted for the development of vaccines targeting different viruses.

### BRIEF DESCRIPTION OF FIGURES

FIGS. 1A and 1B show the plasmid maps of CoVEG2 (FIG. 1A) and CoVEG1 (FIG. 1B). Also shown are the relative positions of cytomegalovirus (CMV) enhancer and promoter and Simian virus 40 Poly A (SV40PA).

FIG. 2 shows the expression of SARS-CoV-2 S protein, SARS-CoV-2 M protein, SARS-CoV-2 E protein and SARS-CoV-2 N protein from CoVEG2 in HEK293 cells.

FIG. 3 shows that SARS-CoV-2 S, N, M and E proteins expressed from CoVEG2 are able to assemble into VLPs and are secreted from cells. See Example 3. FIG. 3A shows results from an SDS PAGE experiment showing S, N, M, and E protein bands in the size exclusion chromatography void sample. FIG. 3B shows the chromatogram of the size exclusion VLP isolation run, whereas the void volume peak, which contains the VLPs, is indicated with an arrow. FIG. 3C shows results from a Western Blotting experiment showing S, N, M, and E protein bands in the size exclusion chromatography void sample. E: envelope protein; M: membrane protein; N: nucleocapsid protein; S: spike protein; NT: non-transfected.

FIG. 4 shows the study design to test the immunogenicity of CoVEG1 and CoVEG2 plasmids in mice.

FIG. 5A-5O show the plasmid maps of each of CoVEG 3-17, respectively. FIG. 5P shows the plasmid map of the control S only plasmid. CMV: cytomegalovirus; SV40PA: Simian virus 40 Poly A.

FIG. 6 shows a schematic depiction of the expression cassettes in each of CoVEG 3-17. Each of the plasmids CoVEG 3-17 vary in the genes that are encoded, the order of the genetic elements and the presence or absence of regulatory elements, e.g., the viral packaging signal. The boxes marked “M”, “S”, “N” and “E” indicate the genes encoding the membrane protein, spike protein, nucleocapsid protein and the envelope protein, respectively. The box marked “L” refers to the gene encoding the L enhancer protein. “S (Mut)” denotes the prefusion conformation-stabilized spike protein mutant in which an internal endogenous furin cleavage site has been mutated to comprise the following amino acid substitutions: R682G, R683S, R685S; and which further has the two amino acid substitutions, K986P and V987P.

FIG. 7 shows the images from anti-spike (S) protein antibody immunostaining of HEK293T cells transfected with each of the indicated plasmids or a control plasmid that expresses only the S protein without the other viral proteins (M, N or E). The images confirm the expression of the S protein in these cells.

FIG. 8 shows the results from the Western Blot analysis of a preparation of viral-like particles (VLPs) isolated from cell culture supernatants when transiently transfected with the indicated CoVEG constructs. “S” denotes transient transfection with a control plasmid that expresses only the S protein without the other viral proteins (M, N or E). Staining with anti-RBD antibody (left) and anti-N protein antibody (right) show that the tested plasmids are capable of expressing and secreting viral structural proteins as VLPs into the cell culture supernatants. The red arrow indicates the full length protein.

FIG. 9A shows the total signal values obtained from an ELISA analysis using 1:500 diluted serum samples from BALB/c mice injected with each of the indicated CoVEG plasmids after 56 days. FIG. 9B shows the endpoint titer values obtained from an ELISA analysis using serum samples from BALB/c mice injected with each of the indicated CoVEG plasmids after 56 days. Endpoint titer refers to the reciprocal maximal antibody dilution at which the ELISA signal (absorbance at 450 nm) is above 3 standard deviations of background signal.

FIG. 10 shows the percent (%) inhibition of the in vitro binding of the Spike protein receptor binding domain (RBD) to the ACE2 receptor by serum samples obtained from mice injected with each of the indicated plasmids using the commercial cPass™ neutralization assay (GenScript). The results show that the serum samples obtained from CoVEG5 and CoVEG8-injected mice have neutralizing antibodies. The positive and negative controls are the assay controls of the cPass neutralization assay kit and contain a known amount of SARS-CoV-2 neutralizing antibodies or a seronegative sample, respectively, and are used according to the manufacturer's protocol.

FIG. 11 shows the results from the Western Blot analysis of a preparation of viral-like particles (VLPs) isolated from cell culture supernatants when transiently transfected with the indicated CoVEG constructs. Staining with anti-RBD antibody (left) and anti-N protein antibody (right) show that the tested plasmids are capable of expressing and secreting viral structural proteins as VLPs into the cell culture supernatants with varying efficiencies.

FIG. 12 shows the results from the Western Blot analysis of a preparation of viral-like particles (VLPs) isolated from cell culture supernatants when transiently transfected with the indicated CoVEG constructs. Staining with anti-RBD antibody (left) and anti-N protein antibody (right) show that the tested plasmids are capable of expressing and secreting viral structural proteins as VLPs into the cell culture supernatants with varying efficiencies.

FIG. 13 shows the total signal values obtained from an ELISA analysis from BALB/c mice injected either intradermally (ID) or intramuscularly (IM) with each of the indicated CoVEG plasmids after 15 days. “S-only” denotes administration of the Spike protein alone.

FIG. 14A shows the map of a plasmid encoding West Nile virus proteins, preM protein and envelope protein, along with the enhancer protein (EMCV L1 protein). FIG. 14B shows the map of a control plasmid encoding just the West Nile virus proteins, preM protein and envelope protein CMV: cytomegalovirus; SV40PA: Simian virus 40 Poly A.

FIGS. 15A and 15B show the total signal values obtained from an ELISA analysis of VLP secretion in HEK293 cells. FIG. 15A shows ELISA analysis of VLP secretion performed with anti-spike (S protein) antibodies. FIG. 15B shows ELISA analysis of VLP secretion performed with anti-nucleocapsid (N protein) antibodies.

FIG. 16 shows transmission electron micrographs (TEM) of CoVEG10 and CoVEG20 protein expression. FIG. 16, left shows TEM of CoVEG10 which contains the L regulatory protein. FIG. 16, right shows TEM of CoVEG20 which lacks the L regulatory protein.

FIG. 17 shows images from anti-nucleocapsid (N) protein antibody immunostaining of HEK293T cells transfected with each of the indicated plasmids.

FIG. 18 shows the results from Western Blot analysis of a preparation of viral-like particles (VLPs) isolated from cell culture supernatants when transiently transfected with the indicated CoVEG constructs. Staining with anti-RBD antibody (left) and anti-N protein antibody (right) show that the tested plasmids are capable of expressing and secreting viral structural proteins as VLPs into the cell culture supernatants with varying efficiencies.

FIG. 19 shows the western blot results of co-immunoprecipitation experiments of the CoVEG 10 plasmid. (Left side) The receptor binding domain (RBD) pull-down signal of the N protein in the elution indicates the N protein was retained within the particles. (Right side) A co-IP was performed without the anti-RBD antibody demonstrating that the N protein did not bind the precipitation resin non-specifically.

FIG. 20 shows antibody binding titers from CoVEG 5 and 9-14 plasmids, as well as a spike (S) protein only containing plasmid, after intramuscular (IM) or intradermal (ID) injection in C57BL/6 mice.

FIG. 21 shows the ELISA analysis of the neutralizing antibodies from the samples shown in FIG. 20.

FIG. 22 shows the antibody binding titers from CoVEG 9, 10, and 18-20 plasmids, as well as a spike (S) protein only containing plasmid, after intramuscular (IM) or intradermal (ID) injection in C57BL/6 mice.

FIG. 23 shows the ELISA analysis of neutralizing antibodies produced in response to CoVEG 9, 10 and 20 plasmids intramuscular (IM) or intradermal (ID) injection in C57BL/6 mice.

FIGS. 24A and 24B show the results of T-cell analysis of CoVEG10 and CoVEG20. FIG. 24 A shows the results of T-cell analysis of CoVEG10. FIG. 24B shows the results of T-cell analysis of CoVEG20.

FIG. 25 shows ELISA analysis of VLPs that were purified from cell culture supernatants of HEK293T cells transfected with isolated and resuspended West-Nile Virus (WNV) constructs with the enhancer protein (WNV+EG, circles) and without the enhancer protein (WNC, squares). The specificity of the ELISA was tested against a plasmid containing GFP that does not give a signal in the specific ELISA assay. The ELISA analysis revealed that the isolation of VLPs by ultracentrifugation with the addition of the enhancer protein contained more active VLPs than in the absence of the enhancer protein. This was especially surprising because the total amount of produced protein was higher in the absence of the enhancer protein, further demonstrating that the addition of an enhancer proteins increased the quality of the expressed target protein.

FIGS. 26A and 26B show the time course analysis of cell culture supernatants obtained from HEK293T cells overexpressing the supernatant containing over expressed West-Nile Virus (WNV) constructs with the enhancer protein (WNV+L) and without the enhancer protein (WNV). FIG. 26A shows ELISA analysis demonstrating that the VLP concentration in the WNV+L (left) construct peaked at 72 h after transfection and gradually decreased thereafter. This was evidence of VLP secretion from healthy cells, as the expression profile followed expected production of VLP from transient transfections and on the related VLP particle half-life. In contrast the WNV construct in the absence of the enhancer protein showed a constant increase of VLP (right) in the supernatant that is more consistent with unspecific release of protein from cells during cell death rather than active secretion. FIG. 26 B confirmed ELISA analysis with cell images. WNV+L cell images (left) revealed very little to no cell death whereas the WNV cell images revealed visible signs of cell death starting at 72 hours after transfection (right, cell death indicated by black stars).

FIGS. 27A and 27B show the analysis of neutralizing antibodies of CoVEG9, the Spike construct with the enhancer protein (Spike+L) and the Spike construct without the enhancer protein (Spike) on days 42 and 70 after immunization of mice. The presence of neutralizing antibodies was detected using the commercial cPass™ SARS-CoV-2 Surrogate Virus Neutralization Test (sVNT) Kit (GenScript). The analysis showed that although all constructs lose some neutralization ability over time, the addition of the enhancer protein prolonged the presence of the desired neutralizing antibodies in the serum of tested animals (CoVEG9 and Spike+L). FIG. 27A shows the individual animals by group on days 42 and 70. FIG. 27B shows the median neutralizing antibody levels in each treatment group of the same data.

FIG. 28 shows immunofluorescence images from cells overexpressing either West-Nile Virus (WNV) constructs with the enhancer protein (WNV+L, left) or without the enhancer protein (WNV, right). As observed in other examples, the total amount of protein decreased when an enhancer protein was added, as indicated by the weaker Alexa Fluor 488 Fluor signal of the secondary antibody used in the immunostaining (WNV+L, left) compared to the WNV (right). However, the absence of the enhancer protein led to the formation of nuclei foci, consistent with the aggregation or misfolding of the expressed protein (right, arrows) indicating a lower quality of the expressed protein compared to the construct with the enhancer protein (WNV+L).

### SUMMARY

The disclosure provides compositions for use as a vaccine, comprising an expression cassette comprising a polynucleotide encoding a viral protein and a polynucleotide encoding an enhancer protein. In some embodiments, the enhancer protein is a picornavirus leader (L) protein or a functional variant thereof. In some embodiments, the amino acid sequence of the enhancer protein has at least 95% identity to SEQ ID NO: 1, or at least 95% identity to SEQ ID NO: 2. In some embodiments, the amino acid sequence of the enhancer protein is SEQ ID NO: 1, or SEQ ID NO: 2. In some embodiments, the polynucleotide encoding the enhancer protein is operatively linked to a polynucleotide encoding an internal ribosome entry site (IRES). In some embodiments, the polynucleotide encoding the IRES is SEQ ID NO: 24.

In some embodiments, the viral protein is a viral antigen. In some embodiments, the viral protein is derived from a virus selected from the group consisting of coronavirus, influenza virus, Hepatitis B virus, Human Papilloma virus (HPV), West Nile virus, and Human Immunodeficiency Virus (HIV) virus. In some embodiments, the viral protein is derived from a coronavirus. In some embodiments, the coronavirus is a betacoronavirus. In some embodiments, the betacoronavirus is severe acute respiratory syndrome (SARS) virus. In some embodiments, the SARS virus is a SARS-CoV-2 virus. In some embodiments, the betacoronavirus is Middle East respiratory syndrome (MERS) virus.

In some embodiments, the coronavirus protein is a coronavirus spike protein. In some embodiments, the spike protein shares at least 70% identity, at least 75%, at least 80%, at least 85%, at least 90%, at least 95%, at least 96%, at least 97%, at least 98%, at least 99%, at least 99.5% or 100% identity to SEQ ID NO: 13. In some embodiments, the spike protein is SEQ ID NO: 13. In some embodiments, the coronavirus protein is a coronavirus membrane (M) protein. In some embodiments, the M protein shares at least 80%, at least 85%, at least 90%, at least 95%, at least 96%, at least 97%, at least 98%, at least 99%, at least 99.5% or 100% identity to SEQ ID NO: 33. In some embodiments, the M protein is SEQ ID NO: 33. In some embodiments, the coronavirus protein is a coronavirus envelope (E) protein. In some embodiments, the E protein shares at least 80%, at least 85%, at least 90%, at least 95%, at least 96%, at least 97%, at least 98%, at least 99%, at least 99.5% or 100% identity to SEQ ID NO: 22. In some embodiments, the E protein is SEQ ID NO: 22. In some embodiments, the coronavirus protein is a coronavirus nucleocapsid (N) protein. In some embodiments, the N protein shares at least 80%, at least 85%, at least 90%, at least 95%, at least 96%, at least 97%, at least 98%, at least 99%, at least 99.5% or 100% identity to SEQ ID NO: 20. In some embodiments, the N protein is SEQ ID NO: 20. In some embodiments, the coronavirus protein forms a virus-like particle (VLP).

In some embodiments, the viral protein is derived from West Nile virus. In some embodiments, the viral protein is precursor membrane protein (preM), envelope glycoprotein (E), or a combination thereof.

The disclosure provides vectors for use as a vaccine, comprising an expression cassette comprising a polynucleotide, wherein the polynucleotide comprises a nucleic acid sequence having at least 70% identity, at least 75%, at least 80%, at least 85%, at least 90%, at least 95%, at least 96%, at least 97%, at least 98%, at least 99%, at least 99.5% or 100% identity to the nucleic acid sequence of SEQ ID NO: 30. In some embodiments, the polynucleotide comprises the nucleic acid sequence of SEQ ID NO: 30.

The disclosure provides vectors for use as a vaccine, comprising an expression cassette comprising a polynucleotide, wherein the polynucleotide comprises a nucleic acid sequence having at least 70% identity, at least 75%, at least 80%, at least 85%, at least 90%, at least 95%, at least 96%, at least 97%, at least 98%, at least 99%, at least 99.5% or 100% identity to the nucleic acid sequence of SEQ ID NO: 31. In some embodiments, the polynucleotide comprises the nucleic acid sequence of SEQ ID NO: 31.

The disclosure provides vectors for use as a vaccine, comprising a nucleic acid sequence having at least 70% identity, at least 75%, at least 80%, at least 85%, at least 90%, at least 95%, at least 96%, at least 97%, at least 98%, at least 99%, at least 99.5% or 100% identity to a nucleic acid sequence selected from the group consisting of SEQ ID NO: 35-49, 55 and 62.

In some embodiments, the vector is a naked polynucleotide. In some embodiments, the vector is a deoxyribonucleic acid (DNA) polynucleotide. In some embodiments, the vector is a ribonucleic acid (RNA) polynucleotide. In some embodiments, the vector comprises a plasmid. In some embodiments, the vector comprises linear DNA. Vectors include plasmids, viruses, bacteriophages, pro-viruses, phagemids, transposons, artificial chromosomes, and the like, that in some cases can replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating.

In some embodiments, the vector is an adeno-associated virus (AAV) vector, a lentivirus vector, a retrovirus vector, a replication competent adenovirus vector, a replication deficient adenovirus vector, a herpes virus vector, a baculovirus vector, a nonviral plasmid, a viral vector, a plasmid, a phage, a phagemid, a cosmid, a fosmid, a bacteriophage, an artificial chromosome, or an adenovirus vector. In some embodiments, the vector is a bacterial artificial chromosome (BAC), a plasmid, a bacteriophage P1-derived vector (PAC), a yeast artificial chromosome (YAC), or a mammalian artificial chromosome (MAC).

In some embodiments, the expression cassette comprises a promoter operatively linked to each of the polynucleotide sequences of the expression cassette. In some embodiments, the vector comprises a DNA polynucleotide, said DNA polynucleotide encoding a viral packaging signal. In some embodiments, the viral packaging signal is a RNA polynucleotide. In some embodiments, the viral packaging signal is derived from a coronavirus.

The disclosure provides vaccine compositions, comprising any one of the vectors disclosed herein, and a pharmaceutically acceptable carrier. In some embodiments, the vaccine composition comprises an adjuvant. In some embodiments, the adjuvant is alum. In some embodiments, the adjuvant is monophosphoryl lipid A (MPL).

The disclosure provides methods of expressing a viral antigen in a eukaryotic cell, comprising contacting the cell with any one of the vectors disclosed herein. In some embodiments, contacting the cell with the vector results in: (i) expression of the antigen at a higher expression level; and/or (ii) expression of the antigen for a longer period of time; and/or (iii) expression of the antigen with better protein quality, than a vector lacking the enhancer protein. In some embodiments, contacting the cell with the vector results in: (i) expression of a virus like particle (VLP) comprising the antigen at a higher expression level; and/or (ii) expression of a VLP comprising the antigen for a longer period of time; and/or (iii) expression of a VLP comprising the antigen with better protein quality, than a vector lacking the enhancer protein.

In some embodiments, the vector comprises a polynucleotide encoding a viral packaging signal, wherein contacting the cell with the vector results in expression of the viral packaging signal, and wherein the VLPs encapsidate the viral packaging signal. In some embodiments, the vector results in the formation of a greater number of VLPs, as compared to a control vector lacking the polynucleotide encoding the viral packaging signal.

The disclosure provides methods of eliciting an immune response in a subject, comprising administering an effective amount of any one of the vaccine compositions disclosed herein to the subject. In some embodiments, tissue at an administration site of the subject expresses the antigen and/or a VLP comprising the antigen. In some embodiments, tissue at an administration site of the subject: (i) expresses the antigen and/or a VLP comprising the antigen at a higher expression level; and/or (ii) expresses the antigen and/or a VLP comprising the antigen for a longer period of time; and/or (iii) expresses the antigen and/or a VLP comprising the antigen with better protein quality, than when a vector lacking the enhancer protein is administered. In some embodiments, the vector comprises a polynucleotide encoding a viral packaging signal, wherein tissue at an administration site of the subject expresses the viral packaging signal, and wherein the VLPs encapsidate the viral packaging signal. In some embodiments, the vector results in the expression of a greater number of VLPs, as compared to a control vector lacking the polynucleotide encoding the viral packaging signal. In some embodiments, the VLPs encapsidating the viral packaging signal are more immunogenic than control VLPs comprising the antigen but lacking the viral packaging signal.

In some embodiments, the method elicits an antibody response in the subject. In some embodiments, the antibody response is a neutralizing antibody response. In some embodiments, the method elicits a cellular immune response. In some embodiments, the method elicits a prophylactic, protective and/or therapeutic immune response in the subject. In some embodiments, the administration is intradermal administration, intramuscular administration, subcutaneous administration, or intranasal administration.

The disclosure provides polynucleotides comprising an expression cassette comprising a polynucleotide encoding a coronavirus protein and a polynucleotide encoding an enhancer protein, wherein the enhancer protein is a picornavirus leader (L) protein or a functional variant thereof. In some embodiments, the amino acid sequence of the enhancer protein has at least 95% identity to SEQ ID NO: 1, or at least 95% identity to SEQ ID NO: 2. In some embodiments, the amino acid sequence of the enhancer protein is SEQ ID NO: 1, or SEQ ID NO: 2.

In some embodiments, the polynucleotide encoding the enhancer protein is operatively linked to a polynucleotide encoding an internal ribosome entry site (IRES). In some embodiments, the polynucleotide encoding the IRES is SEQ ID NO: 24. In some embodiments, the coronavirus protein forms a virus-like particle (VLP).

The disclosure provides polynucleotides comprising a nucleic acid sequence having at least 70% identity, at least 75%, at least 80%, at least 85%, at least 90%, at least 95%, at least 96%, at least 97%, at least 98%, at least 99%, at least 99.5% or 100% identity to the nucleic acid sequence of SEQ ID NO: 30. In some embodiments, the polynucleotide comprises a nucleic acid sequence of SEQ ID NO: 30. The disclosure provides polynucleotides comprising a nucleic acid sequence having at least 70% identity, at least 75%, at least 80%, at least 85%, at least 90%, at least 95%, at least 96%, at least 97%, at least 98%, at least 99%, at least 99.5% or 100% identity to the nucleic acid sequence of SEQ ID NO: 31. In some embodiments, the polynucleotide comprises a nucleic acid sequence of SEQ ID NO: 31.

In some embodiments, the polynucleotide is a naked polynucleotide. In some embodiments, the polynucleotide is a deoxyribonucleic acid (DNA) polynucleotide. In some embodiments, the polynucleotide is a ribonucleic acid (RNA) polynucleotide. In some embodiments, the expression cassette comprises a promoter operatively linked to each of the polynucleotide sequences of the expression cassette.

The disclosure provides kits comprising a vector, wherein the vector comprises an expression cassette comprising a polynucleotide encoding a coronavirus protein and a polynucleotide encoding an enhancer protein, wherein the enhancer protein is a picornavirus leader (L) protein or a functional variant thereof.

The disclosure provides vectors, comprising an expression cassette, said expression cassette comprising a promoter linked to a target gene, wherein the vector comprises a nucleic acid sequence encoding a viral packaging element. In some embodiments, the viral packaging element is a RNA polynucleotide. In some embodiments, the viral packaging element is derived from a coronavirus. In some embodiments, the viral packaging element is derived from SARS-CoV2. In some embodiments, the nucleic acid sequence encoding the viral packaging element has at least about 70% identity to the nucleic acid sequence of SEQ ID NO: 34.

The disclosure provides methods of expressing a target protein in a eukaryotic cell, comprising contacting the cell with any one of the vectors disclosed herein. In some embodiments, contacting the cell with the vector results in the formation of virus-like particles (VLPs) comprising the target protein. In some embodiments, contacting the cell with the vector results in the formation of a greater number of virus-like particles (VLPs) comprising the target protein, as compared to a control vector comprising the expression cassette but lacking the nucleic acid sequence encoding the viral packaging element. In some embodiments, the nucleic acid sequence encoding the viral packaging element has at least about 70% identity to the nucleic acid sequence of SEQ ID NO: 34.

The disclosure provides vectors for use as vaccines, comprising an expression cassette, comprising the following elements in the 5′ to 3′ order: a promoter, a polynucleotide encoding an M protein, wherein the M protein comprises SEQ ID NO: 33 or an amino acid sequence at least 95% identical thereto, a polynucleotide encoding a first proteolytic cleavage site, a polynucleotide encoding an N protein, wherein the N protein comprises SEQ ID NO: 20 or an amino acid sequence at least 95% identical thereto, a polynucleotide encoding a second proteolytic cleavage site, a polynucleotide encoding an S protein wherein the S protein comprises SEQ ID NO: 13 or an amino acid sequence at least 95% identical thereto, a polynucleotide encoding a third proteolytic cleavage site, a polynucleotide encoding an E protein, wherein the E protein comprises SEQ ID NO: 22 or an amino acid sequence at least 95% identical thereto, a polynucleotide encoding an IRES sequence, wherein the IRES sequence comprises SEQ ID NO: 24 or a polynucleotide sequence at least 95% identical thereto, and a polynucleotide encoding an enhancer L protein, wherein the L protein comprises SEQ ID NO: 2 or an amino acid sequence at least 95% identical thereto.

The disclosure also provides vectors for use as a vaccine, comprising an expression cassette, comprising the following elements in the 5′ to 3′ order: a promoter, a polynucleotide encoding an M protein or an amino acid sequence at least 95% identical thereto, wherein the M protein comprises SEQ ID NO: 33 or an amino acid sequence at least 95% identical thereto, a polynucleotide encoding a first proteolytic cleavage site, a polynucleotide encoding an S protein wherein the S protein comprises SEQ ID NO: 13 or an amino acid sequence at least 95% identical thereto, a polynucleotide encoding a second proteolytic cleavage site, a polynucleotide encoding an E protein or a polynucleotide sequence at least 95% identical thereto, wherein the E protein comprises SEQ ID NO: 22 or an amino acid sequence at least 95% identical thereto, a polynucleotide encoding an IRES sequence, wherein the IRES sequence comprises SEQ ID NO: 24 or a polynucleotide sequence at least 95% identical thereto, a polynucleotide encoding an enhancer L protein wherein the L protein comprises SEQ ID NO: 2 or an amino acid sequence at least 95% identical thereto, a polynucleotide encoding an N protein, wherein the N protein comprises SEQ ID NO: 20 or an amino acid sequence at least 95% identical thereto, and a polynucleotide encoding a viral packaging signal, wherein the viral packaging signal comprises SEQ ID NO: 34 or a polynucleotide sequence at least 95% identical thereto.

The disclosure also provides vectors for use as vaccines, comprising an expression cassette, comprising the following elements in the 5′ to 3′ order: a promoter, a polynucleotide encoding an M protein, wherein the M protein comprises SEQ ID NO: 33 or an amino acid sequence at least 95% identical thereto, a polynucleotide encoding a proteolytic cleavage site, a polynucleotide encoding an N protein, wherein the N protein comprises SEQ ID NO: 20 or an amino acid sequence at least 95% identical thereto, a polynucleotide encoding a proteolytic cleavage site, a polynucleotide encoding a mutated S protein, wherein the mutated S protein comprise SEQ ID NO: 51 or SEQ ID NO: 52 or an amino acid sequence at least 95% identical thereto, a polynucleotide encoding a proteolytic cleavage site, a polynucleotide encoding a E protein, wherein the E protein comprises SEQ ID NO: 22 or an amino acid sequence at least 95% identical thereto, a polynucleotide encoding an IRES sequence, wherein the IRES sequence comprises SEQ ID NO: 24 or a polynucleotide sequence at least 95% identical thereto, and a polynucleotide encoding an enhancer L protein, wherein the L protein comprises SEQ ID NO: 2 or an amino acid sequence at least 95% identical thereto.

The disclosure provides vectors for use as vaccines, comprising an expression cassette, comprising the following elements in the 5′ to 3′ order: a promoter, a first polynucleotide encoding a viral packaging signal, wherein the viral packaging signal comprises SEQ ID NO: 34 or a polynucleotide sequence at least 95% identical thereto, a polynucleotide encoding an M protein, wherein the M protein comprises SEQ ID NO: 33 or an amino acid sequence at least 95% identical thereto, a polynucleotide encoding a proteolytic cleavage site, a polynucleotide encoding an N protein, wherein the N protein comprises SEQ ID NO: 20 or an amino acid sequence at least 95% identical thereto, a polynucleotide encoding a proteolytic cleavage site, a polynucleotide encoding a mutated S protein, wherein the S protein comprises SEQ ID NO: 51 or SEQ ID NO: 52 or an amino acid sequence at least 95% identical thereto, a polynucleotide encoding a proteolytic cleavage site, a polynucleotide encoding a E protein, wherein the E protein comprises SEQ ID NO: 22 or an amino acid sequence at least 95% identical thereto, a polynucleotide encoding an IRES sequence, wherein the IRES sequence comprises SEQ ID NO: 24 or a polynucleotide sequence at least 95% identical thereto, a polynucleotide encoding an enhancer L protein, wherein the L protein comprises SEQ ID NO: 2 or an amino acid sequence at least 95% identical thereto, and a second polynucleotide encoding a viral packaging signal, wherein the viral packaging signal comprises SEQ ID NO: 34 or a polynucleotide sequence at least 95% identical thereto.

### DETAILED DESCRIPTION

Virus-like particles (VLPs) are composed of viral structural proteins. Although VLPs are immunogenic, they are non-infectious. Therefore, VLPs have enormous potential for use in the development of vaccines. VLPs may be produced in vitro, and then administered to a subject in need of immunization. Alternatively, VLPs may be produced in vivo in the subject.

The production of VLPs is challenging primarily because it often requires the expression of more than one structural protein from more than one plasmid. In some cases, several plasmids carrying different structural proteins may need to be introduced into the host cell at defined ratios to support the formation of VLPs. This process can be unreliable and often fails to produce sufficient levels of VLPs of required quality. If the multiple structural proteins that are required for the formation of the VLPs can be expressed from a single plasmid or a single RNA transcript, that will greatly simplify the process of making VLPs and thus, provide a much-needed boost for the development of vaccines comprising VLPs.

The compositions and methods disclosed herein enable the reliable formation of high levels of VLPs in vivo and thus, enable a robust induction of immune response against the viral antigens on the VLPs. Furthermore, these compositions and methods may be used to induce immune response against different viruses, e.g., coronaviruses (e.g. SARS CoV-2), influenza viruses, and West Nile virus.

### Definitions

As used herein, and in the appended claims, the singular forms “a”, “an”, and “the” include plural referents unless the context clearly dictates otherwise. Thus, for example, reference to “an antigen” can refer to one or more antigens, and reference to “the method” includes reference to equivalent steps and/or methods known to those skilled in the art, and so forth.

As used herein, the term “about” or “approximately” when preceding a numerical value indicates the value plus or minus a range of 10%. For example, “about 100” encompasses 90 and 110.

As used herein, nucleotide sequences are listed in the 5′ to 3′ direction, and amino acid sequences are listed in the N-terminal to C-terminal direction, unless indicated otherwise.

The terms “polypeptide,” “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, e.g., conjugation with a labeling component. As used herein the term “amino acid” includes natural and/or unnatural or synthetic amino acids, including glycine and both the D or L optical isomers, and amino acid analogs and peptidomimetics.

As used herein, the term “subject” includes humans and other animals. Typically, the subject is a human. For example, the subject may be an adult, a teenager, a child (2 years to 14 years of age), an infant (1 month to 24 months), or a neonate (up to 1 month). In some embodiments, the adults are seniors about 65 years or older, or about 60 years or older. In some embodiments, the subject is a pregnant woman or a woman intending to become pregnant. In other embodiments, subject is not a human; for example a non-human primate; for example, a baboon, a chimpanzee, a gorilla, or a macaque. In certain embodiments, the subject may be a pet, e.g., a dog or cat.

As used herein, the terms “immunogen,” “antigen,” and “epitope” refer to substances e.g., proteins, including glycoproteins, and peptides that are capable of eliciting an immune response.

As used herein, an “immunogenic response” in a subject results in the development in the subject of a humoral and/or a cellular immune response to an antigen.

The term “effective amount” or “therapeutically effective amount” refers to the amount of an agent that is sufficient to achieve an outcome, for example, to affect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, the tissue into which it is administered, and the physical delivery system in which it is carried.

As used herein, the term “virus-like particle” (VLP) refers to a structure that in at least one attribute resembles a virus but which has not been demonstrated to be infectious. Virus-like particles in accordance with the disclosure do not carry genetic information encoding for the proteins of the virus-like particles. In general, virus-like particles lack a viral genome and, therefore, are noninfectious. In addition, virus-like particles can often be produced in large quantities by heterologous expression and can be easily purified.

As used herein, an amino acid substitution, interchangeably referred to as amino acid replacement, at a specific position on the protein sequence is denoted herein in the following manner: “one letter code of the WT amino acid residue-amino acid position-one letter code of the amino acid residue that replaces this WT residue”. For example, a Spike (S) protein which is a R682G mutant refers to an S protein in which the wild type residue at the 682^{nd} amino acid position (R or arginine) is replaced with G or glycine.

Vectors

The disclosure provides vectors comprising an expression cassette comprising a polynucleotide encoding an antigen and a polynucleotide encoding an enhancer protein. In some embodiments, the vector is used as a vaccine, or as part of a vaccine composition.

The term “vector” refers to the means by which a nucleic acid can be propagated and/or transferred between organisms, cells, or cellular components. A vector for use according to the present disclosure may comprise any vector known in the art. Vectors include plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating.

In some embodiments, the vector is an adeno-associated virus (AAV) vector, a lentivirus vector, a retrovirus vector, a replication competent adenovirus vector, a replication deficient adenovirus vector, a herpes virus vector, a baculovirus vector, a nonviral plasmid, a viral vector, a plasmid, a phage, a phagemid, a cosmid, a fosmid, a bacteriophage, an artificial chromosome, or an adenovirus vector. In some embodiments, the vector is a bacterial artificial chromosome (BAC), a plasmid, a bacteriophage P1-derived vector (PAC), a yeast artificial chromosome (YAC), or a mammalian artificial chromosome (MAC). In some embodiments, the vector is a naked polynucleotide. In some embodiments, the vector is a deoxyribonucleic acid (DNA) polynucleotide. In some embodiments, the vector is a ribonucleic acid (RNA) polynucleotide.

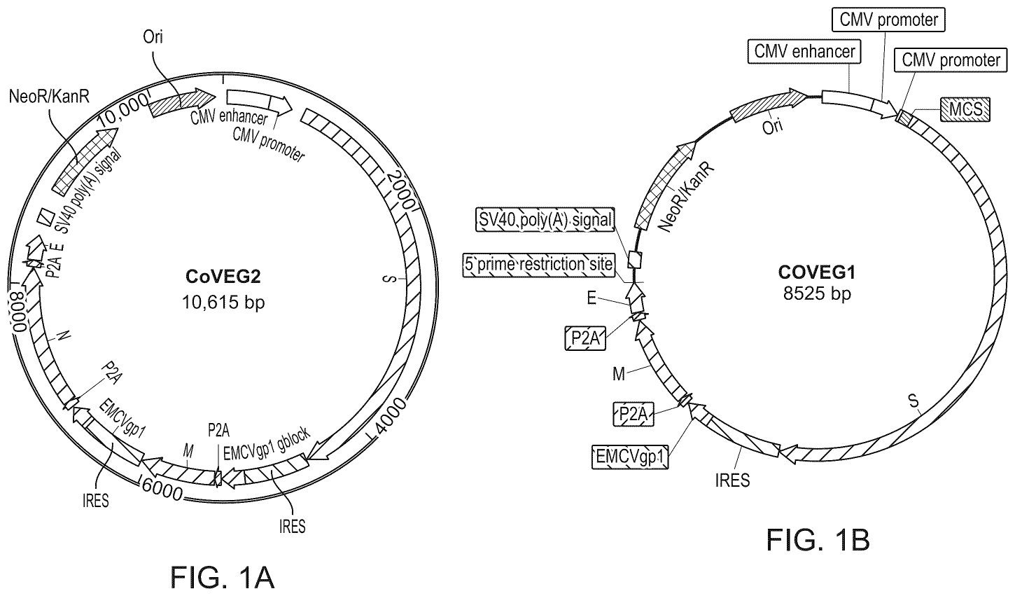

In some embodiments, the vector comprises a first polynucleotide encoding an antigen and a second polynucleotide encoding an enhancer protein. In some embodiments, the vector has a design as shown in FIG. 1A or FIG. 1B. In some embodiments, the vector is CoVEG1. In some embodiments, the vector is CoVEG2.

Table 1 shows the nucleic acid sequences of important regions of the CoVEG1 and CoVEG2, and amino acid sequences encoded by these regions.

TABLE 1

| | | | SEQ |
|---|---|---|---|
| | Type of | | ID |
| Name | sequence | Sequence | NO.: |
| SARS- | Amino | MFVFLVLLPLVSSQCVNLTT | 13 |
| CoV-2 | acid | RTQLPPAYTNSFTRGVYYPD | |
| Spike | | KVFRSSVLHSTQDLFLPFFS | |
| | | NVTWFHAIHVSGTNGTKRFD | |
| | | NPVLPFNDGVYFASTEKSNI | |
| | | IRGWIFGTTLDSKTQSLLIV | |
| | | NNATNVVIKVCEFQFCNDPF | |
| | | LGVYYHKNNKSWMESEFRVY | |
| | | SSANNCTFEYVSQPFLMDLE | |
| | | GKQGNFKNLREFVFKNIDGY | |
| | | FKIYSKHTPINLVRDLPQGF | |
| | | SALEPLVDLPIGINITRFQT | |
| | | LLALHRSYLTPGDSSSGWTA | |
| | | GAAAYYVGYLQPRTFLLKYN | |
| | | ENGTITDAVDCALDPLSETK | |
| | | CTLKSFTVEKGIYQTSNFRV | |
| | | QPTESIVRFPNITNLCPFGE | |
| | | VENATRFASVYAWNRKRISN | |
| | | CVADYSVLYNSASFSTFKCY | |
| | | GVSPTKLNDLCFTNVYADSF | |
| | | VIRGDEVRQIAPGQTGKIAD | |
| | | YNYKLPDDFTGCVIAWNSNN | |
| | | LDSKVGGNYNYLYRLFRKSN | |
| | | LKPFERDISTEIYQAGSTPC | |
| | | NGVEGENCYFPLQSYGFQPT | |
| | | NGVGYQPYRVVVLSFELLHA | |
| | | PATVCGPKKSTNLVKNKCVN | |
| | | FNFNGLTGTGVLTESNKKFL | |
| | | PFQQFGRDIADTTDAVRDPQ | |
| | | TLEILDITPCSFGGVSVITP | |
| | | GTNTSNQVAVLYQDVNCTEV | |
| | | PVAIHADQLTPTWRVYSTGS | |
| | | NVFQTRAGCLIGAEHVNNSY | |
| | | ECDIPIGAGICASYQTQTNS | |
| | | PRRARSVASQSIIAYTMSLG | |
| | | AENSVAYSNNSIAIPTNFTI | |
| | | SVTTEILPVSMTKTSVDCTM | |
| | | YICGDSTECSNLLLQYGSFC | |
| | | TQLNRALTGIAVEQDKNTQE | |
| | | VFAQVKQIYKTPPIKDFGGF | |
| | | NFSQILPDPSKPSKRSFIED | |
| | | LLFNKVTLADAGFIKQYGDC | |
| | | LGDIAARDLICAQKFNGLTV | |
| | | LPPLLTDEMIAQYTSALLAG | |
| | | TITSGWTFGAGAALQIPFAM | |
| | | QMAYRENGIGVTQNVLYENQ | |
| | | KLIANQFNSAIGKIQDSLSS | |
| | | TASALGKLQDVVNQNAQALN | |
| | | TLVKQLSSNFGAISSVLNDI | |
| | | LSRLDKVEAEVQIDRLITGR | |
| | | LQSLQTYVTQQLIRAAEIRA | |
| | | SANLAATKMSECVLGQSKRV | |
| | | DFCGKGYHLMSFPQSAPHGV | |
| | | VFLHVTYVPAQEKNFTTAPA | |
| | | ICHDGKAHFPREGVFVSNGT | |
| | | HWFVTQRNFYEPQIITTDNT | |
| | | FVSGNCDVVIGIVNNTVYDP | |
| | | LQPELDSFKEELDKYFKNHT | |
| | | SPDVDLGDISGINASVVNIQ | |
| | | KEIDRLNEVAKNLNESLIDL | |
| | | QELGKYEQYIKWPWYIWLGF | |
| | | IAGLIAIVMVTIMLCCMTSC | |
| | | CSCLKGCCSCGSCCKFDEDD | |
| | | SEPVLKGVKLHYT* | |
| | Nucleic | ATGTTTGTTTTTCTTGTTTT | 14 |
| | acid | ATTGCCACTAGTCTCTAGTC | |
| | | AGTGTGTTAATCTTACAACC | |
| | | AGAACTCAATTACCCCCTGC | |
| | | ATACACTAATTCTTTCACAC | |
| | | GTGGTGTTTATTACCCTGAC | |
| | | AAAGTTTTCAGATCCTCAGT | |
| | | TTTACATTCAACTCAGGACT | |
| | | TGTTCTTACCTTTCTTTTCC | |
| | | AATGTTACTTGGTTCCATGC | |
| | | TATACATGTCTCTGGGACCA | |
| | | ATGGTACTAAGAGGTTTGAT | |
| | | AACCCTGTCCTACCATTTAA | |
| | | TGATGGTGTTTATTTTGCTT | |
| | | CCACTGAGAAGTCTAACATA | |
| | | ATAAGAGGCTGGATTTTTGG | |
| | | TACTACTTTAGATTCGAAGA | |
| | | CCCAGTCCCTACTTATTGTT | |
| | | AATAACGCTACTAATGTTGT | |
| | | TATTAAAGTCTGTGAATTTC | |
| | | AATTTTGTAATGATCCATTT | |
| | | TTGGGTGTTTATTACCACAA | |
| | | AAACAACAAAAGTTGGATGG | |
| | | AAAGTGAGTTCAGAGTTTAT | |
| | | TCTAGTGCGAATAATTGCAC | |
| | | TTTTGAATATGTCTCTCAGC | |
| | | CTTTTCTTATGGACCTTGAA | |
| | | GGAAAACAGGGTAATTTCAA | |
| | | AAATCTTAGGGAATTTGTGT | |
| | | TTAAGAATATTGATGGTTAT | |
| | | TTTAAAATATATTCTAAGCA | |
| | | CACGCCTATTAATTTAGTGC | |
| | | GTGATCTCCCTCAGGGTTTT | |
| | | TCGGCTTTAGAACCATTGGT | |
| | | AGATTTGCCAATAGGTATTA | |
| | | ACATCACTAGGTTTCAAACT | |
| | | TTACTTGCTTTACATAGAAG | |
| | | TTATTTGACTCCTGGTGATT | |
| | | CTTCTTCAGGTTGGACAGCT | |
| | | GGTGCTGCAGCTTATTATGT | |
| | | GGGTTATCTTCAACCTAGGA | |
| | | CTTTTCTATTAAAATATAAT | |
| | | GAAAATGGAACCATTACAGA | |
| | | TGCTGTAGACTGTGCACTTG | |
| | | ACCCTCTCTCAGAAACAAAG | |
| | | TGTACGTTGAAATCCTTCAC | |
| | | TGTAGAAAAAGGAATCTATC | |
| | | AAACTTCTAACTTTAGAGTC | |
| | | CAACCAACAGAATCTATTGT | |
| | | TAGATTTCCTAATATTACAA | |
| | | ACTTGTGCCCTTTTGGTGAA | |
| | | GTTTTTAACGCCACCAGATT | |
| | | TGCATCTGTTTATGCTTGGA | |
| | | ACAGGAAGAGAATCAGCAAC | |
| | | TGTGTTGCTGATTATTCTGT | |
| | | CCTATATAATTCCGCATCAT | |
| | | TTTCCACTTTTAAGTGTTAT | |
| | | GGAGTGTCTCCTACTAAATT | |
| | | AAATGATCTCTGCTTTACTA | |
| | | ATGTCTATGCAGATTCATTT | |
| | | GTAATTAGAGGTGATGAAGT | |
| | | CAGACAAATCGCTCCAGGGC | |
| | | AAACTGGAAAGATTGCTGAT | |
| | | TATAATTATAAATTACCAGA | |
| | | TGATTTTACAGGCTGCGTTA | |
| | | TAGCTTGGAATTCTAACAAT | |
| | | CTTGATTCTAAGGTTGGTGG | |
| | | TAATTATAATTACCTGTATA | |
| | | GATTGTTTAGGAAGTCTAAT | |
| | | CTCAAACCTTTTGAGAGAGA | |
| | | TATTTCAACTGAAATCTATC | |
| | | AGGCCGGTAGCACACCTTGT | |
| | | AATGGTGTTGAAGGTTTTAA | |
| | | TTGTTACTTTCCTTTACAAT | |
| | | CATATGGTTTCCAACCCACT | |
| | | AATGGTGTTGGTTACCAACC | |
| | | ATACAGAGTAGTAGTACTTT | |
| | | CTTTTGAACTTCTACATGCA | |
| | | CCAGCAACTGTTTGTGGACC | |
| | | TAAAAAGTCTACTAATTTGG | |
| | | TTAAAAACAAATGTGTCAAT | |
| | | TTCAACTTCAATGGTTTAAC | |
| | | AGGCACAGGTGTTCTTACTG | |
| | | AGTCTAACAAAAAGTTTCTG | |
| | | CCTTTCCAACAATTTGGCAG | |
| | | AGACATTGCTGACACTACTG | |
| | | ATGCTGTCCGTGATCCACAG | |
| | | ACACTTGAGATTCTTGACAT | |
| | | TACACCATGTTCTTTTGGTG | |
| | | GTGTCAGTGTTATAACACCA | |
| | | GGAACAAATACTTCTAACCA | |
| | | GGTTGCTGTTCTTTATCAGG | |
| | | ATGTTAACTGCACAGAAGTC | |
| | | CCTGTTGCTATTCATGCAGA | |
| | | TCAACTTACTCCTACTTGGC | |
| | | GTGTTTATTCTACAGGTTCT | |
| | | AATGTTTTTCAAACACGTGC | |
| | | AGGCTGTTTAATAGGGGCTG | |
| | | AACATGTCAACAACTCATAT | |
| | | GAGTGTGACATACCCATTGG | |
| | | TGCAGGTATATGCGCTAGTT | |
| | | ATCAGACTCAGACTAATTCT | |
| | | CCTCGGCGGGCACGTAGTGT | |
| | | AGCTAGTCAATCCATCATTG | |
| | | CCTACACTATGTCACTTGGT | |
| | | GCAGAAAATTCAGTTGCTTA | |
| | | CTCTAATAACTCTATTGCCA | |
| | | TACCCACAAATTTTACTATT | |
| | | AGTGTTACCACAGAAATTCT | |
| | | ACCAGTGTCTATGACCAAGA | |
| | | CATCAGTAGATTGTACAATG | |
| | | TACATTTGTGGTGATTCAAC | |
| | | TGAATGCAGCAATCTTTTGT | |
| | | TGCAATATGGCAGTTTTTGT | |
| | | ACACAATTAAACCGTGCTTT | |
| | | AACTGGAATAGCTGTTGAAC | |
| | | AAGACAAAAACACCCAAGAA | |
| | | GTTTTTGCACAAGTCAAACA | |
| | | AATTTACAAAACACCACCAA | |
| | | TTAAAGATTTTGGTGGTTTT | |
| | | AATTTTTCACAAATATTACC | |
| | | AGATCCATCAAAACCAAGCA | |
| | | AGAGGTCATTTATTGAAGAT | |
| | | CTACTTTTCAACAAAGTGAC | |
| | | ACTTGCAGATGCTGGCTTCA | |
| | | TCAAACAATATGGTGATTGC | |
| | | CTTGGTGATATTGCTGCTAG | |
| | | AGACCTCATTTGTGCACAAA | |
| | | AGTTTAACGGCCTTACTGTT | |
| | | TTGCCACCTTTGCTCACAGA | |
| | | TGAAATGATTGCTCAATACA | |
| | | CTTCTGCACTGTTAGCGGGT | |
| | | ACAATCACTTCTGGTTGGAC | |
| | | CTTTGGTGCAGGTGCTGCAT | |
| | | TACAAATACCATTTGCTATG | |
| | | CAAATGGCTTATAGGTTTAA | |
| | | TGGTA | |
| | | TTGGAGTTACACAGAATGTT | |
| | | CTCTATGAGAACCAAAAATT | |
| | | GATTGCCAACCAATTTAATA | |
| | | GTGCTATTGGCAAAATTCAA | |
| | | GACTCACTTTCTTCCACAGC | |
| | | AAGTGCACTTGGAAAACTTC | |
| | | AAGATGTGGTCAACCAAAAT | |
| | | GCACAAGCTTTAAACACGCT | |
| | | TGTTAAACAACTTAGCTCCA | |
| | | ATTTTGGTGCAATTTCAAGT | |
| | | GTTTTAAATGATATCCTTTC | |
| | | ACGTCTTGACAAAGTTGAGG | |
| | | CTGAAGTGCAAATTGATAGG | |
| | | TTGATCACAGGCAGACTTCA | |
| | | AAGTTTGCAGACATATGTGA | |
| | | CTCAACAATTAATTAGAGCT | |
| | | GCAGAAATCAGAGCTTCTGC | |
| | | TAATCTTGCTGCTACTAAAA | |
| | | TGTCAGAGTGTGTACTTGGA | |
| | | CAATCAAAAAGAGTTGATTT | |
| | | TTGTGGAAAGGGCTATCATC | |
| | | TTATGTCCTTCCCTCAGTCA | |
| | | GCACCTCATGGTGTAGTCTT | |
| | | CTTGCATGTGACTTATGTCC | |
| | | CTGCACAAGAAAAGAACTTC | |
| | | ACAACTGCTCCTGCCATTTG | |
| | | TCATGATGGAAAAGCACACT | |
| | | TTCCTCGTGAAGGTGTCTTT | |
| | | GTTTCAAATGGCACACACTG | |
| | | GTTTGTAACACAAAGGAATT | |
| | | TTTATGAACCACAAATCATT | |
| | | ACTACAGACAACACATTTGT | |
| | | GTCTGGTAACTGTGATGTTG | |
| | | TAATAGGAATTGTCAACAAC | |
| | | ACAGTTTATGATCCTTTGCA | |
| | | ACCTGAATTAGACTCATTCA | |
| | | AGGAGGAGTTAGATAAATAT | |
| | | TTTAAGAATCATACATCACC | |
| | | AGATGTTGATTTAGGTGACA | |
| | | TCTCTGGCATTAATGCTTCA | |
| | | GTTGTAAACATTCAAAAAGA | |
| | | AATTGACCGCCTCAATGAGG | |
| | | TTGCCAAGAATTTAAATGAA | |
| | | TCTCTCATCGATCTCCAAGA | |
| | | ACTTGGAAAGTATGAGCAGT | |
| | | ATATAAAATGGCCATGGTAC | |
| | | ATTTGGCTAGGTTTTATAGC | |
| | | TGGCTTGATTGCCATAGTAA | |
| | | TGGTGACAATTATGCTTTGC | |
| | | TGTATGACCAGTTGCTGTAG | |
| | | TTGTCTCAAGGGCTGTTGTT | |
| | | CTTGTGGATCCTGCTGCAAA | |
| | | TTTGATGAAGACGACTCTGA | |
| | | GCCAGTGCTCAAAGGAGTCA | |
| | | AATTACATTACACATAA | |
| L protein | Amino | MATTMEQETCAHSLTFEECP | 2 |
| from | acid | KCSALQYRNGFYLLKYDEEW | |
| EMCV | | YPEELLTDGEDDVFDPELDM | |
| | | EVVFELQ | |
| | Nucleic | ATGGCCACAACCATGGAACA | 16 |
| | acid | AGAGACTTGCGCGCACTCTC | |
| | | TCACTTTTGAGGAATGCCCA | |
| | | AAATGCTCTGCTCTACAATA | |
| | | CCGTAATGGATTTTACCTGC | |
| | | TAAAGTATGATGAAGAATGG | |
| | | TACCCAGAGGAGTTATTGAC | |
| | | TGATGGAGAGGATGATGTCT | |
| | | TTGATCCCGAATTAGACATG | |
| | | GAAGTCGTTTTCGAGTTACA | |
| | | G | |
| 2A site | Amino | GSGATNFSLLKQAGDVEENP | 17 |
| | acid | GP | |
| | Nucleic | GGAAGCGGAGCTACTAACTT | 18 |
| | acid | CAGCCTGCTGAAGCAGGCTG | |
| | | GAGATGTGGAGGAGAACCCT | |
| | | GGACCT | |
| SARS-CoV-2 | Amino | MADSNGTITVEELKKLLEQW | 33 |
| M protein | acid | NLVIGFLFLTWICLLQFAYA | 19 |
| | | NRNRFLYIIKLIFLWLLWPV | |
| | | TLACFVLAAVYRINWITGGI | |
| | | AIAMACLVGLMWLSYFIASF | |
| | | RLFARTRSMWSFNPETNILL | |
| | | NVPLHGTILTRPLLESELVI | |
| | | GAVILRGHLRIAGHHLGRCD | |
| | | IKDLPKEITVATSRTLSYYK | |
| | | LGASQRVAGDSGFAAYSRYR | |
| | | IGNYKLNTDHSSSSDNIALL | |
| | | VQ* | |
| | Nucleic | ATGGCAGATTCCAACGGTAC | |
| | acid | TATTACCGTTGAAGAGCTTA | |
| | | AAAAGCTCCTTGAACAATGG | |
| | | AACCTAGTAATAGGTTTCCT | |
| | | ATTCCTTACATGGATTTGTC | |
| | | TTCTACAATTTGCCTATGCC | |
| | | AACAGGAATAGGTTTTTGTA | |
| | | TATAATTAAGTTAATTTTCC | |
| | | TCTGGCTGTTATGGCCAGTA | |
| | | ACTTTAGCTTGTTTTGTGCT | |
| | | TGCTGCTGTTTACAGAATAA | |
| | | ATTGGATCACCGGTGGAATT | |
| | | GCTATCGCAATGGCTTGTCT | |
| | | TGTAGGCTTGATGTGGCTCA | |
| | | GCTACTTCATTGCTTCTTTC | |
| | | AGACTGTTTGCGCGTACGCG | |
| | | TTCCATGTGGTCATTCAATC | |
| | | CAGAAACTAACATTCTTCTC | |
| | | AACGTGCCACTCCATGGCAC | |
| | | TATTCTGACCAGACCGCTTC | |
| | | TAGAAAGTGAACTCGTAATC | |
| | | GGAGCTGTGATCCTTCGTGG | |
| | | ACATCTTCGTATTGCTGGAC | |
| | | ACCATCTAGGACGCTGTGAC | |
| | | ATCAAGGACCTGCCTAAAGA | |
| | | AATCACTGTTGCTACATCAC | |
| | | GAACGCTTTCTTATTACAAA | |
| | | TTGGGAGCTTCGCAGCGTGT | |
| | | AGCAGGTGACTCAGGTTTTG | |
| | | CTGCATACAGTCGCTACAGG | |
| | | ATTGGCAACTATAAATTAAA | |
| | | CACAGACCATTCCAGTAGCA | |
| | | GTGACAATATTGCTTTGCTT | |
| | | GTACAGTAA | |
| SARS-CoV-2 | Amino | MSDNGPQNQRNAPRITFGGP | 20 |
| N protein | acid | SDSTGSNQNGERSGARSKQR | |
| | | RPQGLPNNTASWFTALTQHG | |
| | | KEDLKFPRGQGVPINTNSSP | |
| | | DDQIGYYRRATRRIRGGDGK | |
| | | MKDLSPRWYFYYLGTGPEAG | |
| | | LPYGANKDGIIWVATEGALN | |
| | | TPKDHIGTRNPANNAAIVLQ | |
| | | LPQGTTLPKGFYAEGSRGGS | |
| | | QASSRSSSRSRNSSRNSTPG | |
| | | SSRGTSPARMAGNGGDAALA | |
| | | LLLLDRLNQLESKMSGKGQQ | |
| | | QQGQTVTKKSAAEASKKPRQ | |
| | | KRTATKAYNVTQAFGRRGPE | |
| | | QTQGNFGDQELIRQGTDYKH | |
| | | WPQIAQFAPSASAFFGMSRI | |
| | | GMEVTPSGTWLTYTGAIKLD | |
| | | DKDPNFKDQVILLNKHIDAY | |
| | | KTFPPTEPKKDKKKKADETQ | |
| | | ALPQRQKKQQTVTLLPAADL | |
| | | DDESKQLQQSMSSADSTQA | |
| | Nucleic | ATGTCTGATAATGGACCCCA | 21 |
| | acid | AAATCAGCGAAATGCACCCC | |
| | | GCATTACGTTTGGTGGACCC | |
| | | TCAGATTCAACTGGCAGTAA | |
| | | CCAGAATGGAGAACGCAGTG | |
| | | GGGCGCGATCAAAACAACGT | |
| | | CGGCCCCAAGGTTTACCCAA | |
| | | TAATACTGCGTCTTGGTTCA | |
| | | CCGCTCTCACTCAACATGGC | |
| | | AAGGAAGACCTTAAATTCCC | |
| | | TCGAGGACAAGGCGTTCCAA | |
| | | TTAACACCAATAGCAGTCCA | |
| | | GATGACCAAATTGGCTACTA | |
| | | CCGAAGAGCTACCAGACGAA | |
| | | TTCGTGGTGGTGACGGTAAA | |
| | | ATGAAAGATCTCAGTCCAAG | |
| | | ATGGTATTTCTACTACCTAG | |
| | | GAACTGGGCCAGAAGCTGGA | |
| | | CTTCCCTATGGTGCTAACAA | |
| | | AGACGGCATCATATGGGTTG | |
| | | CAACTGAGGGAGCCTTGAAT | |
| | | ACACCAAAAGATCACATTGG | |
| | | CACCCGCAATCCTGCTAACA | |
| | | ATGCTGCAATCGTGCTACAA | |
| | | CTTCCTCAAGGAACAACATT | |
| | | GCCAAAAGGCTTCTACGCAG | |
| | | AAGGGAGCAGAGGCGGCAGT | |
| | | CAAGCCTCTTCTCGTTCCTC | |
| | | ATCACGTAGTCGCAACAGTT | |
| | | CAAGAAATTCAACTCCAGGC | |
| | | AGCAGTAGGGGAACTTCTCC | |
| | | TGCTAGAATGGCTGGCAATG | |
| | | GCGGTGATGCTGCTCTTGCT | |
| | | TTGCTGCTGCTTGACAGATT | |
| | | GAACCAGCTTGAGAGCAAAA | |
| | | TGTCTGGTAAAGGCCAACAA | |
| | | CAACAAGGCCAAACTGTCAC | |
| | | TAAGAAATCTGCTGCTGAGG | |
| | | CTTCTAAGAAGCCTCGGCAA | |
| | | AAACGTACTGCCACTAAAGC | |
| | | ATACAATGTAACACAAGCTT | |
| | | TCGGCAGACGTGGTCCAGAA | |
| | | CAAACCCAAGGAAATTTTGG | |
| | | GGACCAGGAACTAATCAGAC | |
| | | AAGGAACTGATTACAAACAT | |
| | | TGGCCGCAAATTGCACAATT | |
| | | TGCCCCCAGCGCTTCAGCGT | |
| | | TCTTCGGAATGTCGCGCATT | |
| | | GGCATGGAAGTCACACCTTC | |
| | | GGGAACGTGGTTGACCTACA | |
| | | CAGGTGCCATCAAATTGGAT | |
| | | GACAAAGATCCAAATTTCAA | |
| | | AGATCAAGTCATTTTGCTGA | |
| | | ATAAGCATATTGACGCATAC | |
| | | AAAACATTCCCACCAACAGA | |
| | | GCCTAAAAAGGACAAAAAGA | |
| | | AGAAGGCTGATGAAACTCAA | |
| | | GCCTTACCGCAGAGACAGAA | |
| | | GAAACAGCAAACTGTGACTC | |
| | | TTCTTCCTGCTGCAGATTTG | |
| | | GATGATTTCTCCAAACAATT | |
| | | GCAACAATCCATGAGCAGTG | |
| | | CTGACTCAACTCAGGCC | |
| SARS-CoV-2 | Amino | MYSFVSEETGTLIVNSVLLF | 22 |
| E protein | acid | LAFVVFLLVTLAILTALRLC | |
| | | AYCCNIVNVSLVKPSFYVYS | |
| | | RVKNLNSSRVPDLLV* | |
| | Nucleic | ATGTACTCATTCGTTTCGGA | 23 |
| | acid | AGAGACAGGTACGTTAATAG | |
| | | TTAATAGCGTACTTCTTTTT | |
| | | CTTGCTTTCGTGGTATTCTT | |
| | | GCTAGTTACACTAGCCATCC | |
| | | TTACTGCGCTTCGATTGTGT | |
| | | GCGTACTGCTGCAATATTGT | |
| | | TAACGTGAGTCTTGTAAAAC | |
| | | CTTCTTTTTACGTTTACTCT | |
| | | CGTGTTAAAAATCTGAATTC | |
| | | TTCTAGAGTTCCTGATCTTC | |
| | | TGGTCTAA | |
| IRES | Nucleic | TCCCCCCCCCCTAACGTTAC | 24 |
| | acid | TGGCCGAAGCCGCTTGGAAT | |
| | | AAGGCCGGTGTGCGTTTGTC | |
| | | TATATGTTATTTTCCACCAT | |
| | | ATTGCCGTCTTTTGGCAATG | |
| | | TGAGGGCCCGGAAACCTGGC | |
| | | CCTGTCTTCTTGACGAGCAT | |
| | | TCCTAGGGGTCTTTCCCCTC | |
| | | TCGCCAAAGGAATGCAAGGT | |
| | | CTGTTGAATGTCGTGAAGGA | |
| | | AGCAGTTCCTCTGGAAGCTT | |
| | | CTTGAAGACAAACAACGTCT | |
| | | GTAGCGACCCTTTGCAGGCA | |
| | | GCGGAACCCCCCACCTGGCG | |
| | | ACAGGTGCCTCTGCGGCCAA | |
| | | AAGCCACGTGTATAAGATAC | |
| | | ACCTGCAAAGGCGGCACAAC | |
| | | CCCAGTGCCACGTTGTGAGT | |
| | | TGGATAGTTGTGGAAAGAGT | |
| | | CAAATGGCTCTCCTCAAGCG | |
| | | TATTCAACAAGGGGCTGAAG | |
| | | GATGCCCAGAAGGTACCCCA | |
| | | TTGTATGGGATCTGATCTGG | |
| | | GGCCTCGGTGCACATGCTTT | |
| | | ACATGTGTTTAGTCGAGGTT | |
| | | AAAAAAACGTCTAGGCCCCC | |
| | | CGAACCACGGGGACGTGGTT | |
| | | TTCCTTTGAAAAACACGATG | |
| | | ATAAT | |
| CoVEG2 | Amino | MATTMEQETCAHSLTFEECP | 25 |
| polypeptide 1 | acid | KCSALQYRNGFYLLKYDEEW | |
| (fusion of | | YPEELLTDGEDDVFDPELDM | |
| EMCV L | | EVVFELQGSGATNFSLLKQA | |
| protein, 2A | | GDVEENPGPMADSNGTITVE | |
| site, M | | ELKKLLEQWNLVIGFLFLTW | |
| protein) | | ICLLQFAYANRNRFLYIIKL | |
| | | IFLWLLWPVTLACFVLAAVY | |
| | | RINWITGGIAIAMACLVGLM | |
| | | WLSYFIASFRLFARTRSMWS | |
| | | FNPETNILLNVPLHGTILTR | |
| | | PLLESELVIGAVILRGHLRI | |
| | | AGHHLGRCDIKDLPKEITVA | |
| | | TSRTLSYYKLGASQRVAGDS | |
| | | GFAAYSRYRIGNYKLNTDHS | |
| | | SSSDNIALLVQ* | |
| CoVEG2 | Amino | MATTMEQETCAHSLTFEECP | 26 |
| polypeptide 2 | acid | KCSALQYRNGFYLLKYDEEW | |
| (fusion of | | YPEELLTDGEDDVFDPELDM | |
| EMCV L | | EVVFELQGSGATNFSLLKQA | |
| protein, 2A | | GDVEENPGPMSDNGPQNQRN | |
| site, | | APRITFGGPSDSTGSNQNGE | |
| N protein, | | RSGARSKQRRPQGLPNNTAS | |
| 2A site and E | | WFTALTQHGKEDLKFPRGQG | |
| protein) | | VPINTNSSPDDQIGYYRRAT | |
| | | RRIRGGDGKMKDLSPRWYFY | |
| | | YLGTGPEAGLPYGANKDGII | |
| | | WVATEGALNTPKDHIGTRNP | |
| | | ANNAAIVLQLPQGTTLPKGF | |
| | | YAEGSRGGSQASSRSSSRSR | |
| | | NSSRNSTPGSSRGTSPARMA | |
| | | GNGGDAALALLLLDRLNQLE | |
| | | SKMSGKGQQQQGQTVTKKSA | |
| | | AEASKKPRQKRTATKAYNVT | |
| | | QAFGRRGPEQTQGNFGDQEL | |
| | | IRQGTDYKHWPQIAQFAPSA | |
| | | SAFFGMSRIGMEVTPSGTWL | |
| | | TYTGAIKLDDKDPNFKDQVI | |
| | | LLNKHIDAYKTFPPTEPKKD | |
| | | KKKKADETQALPQRQKKQQT | |
| | | VTLLPAADLDDFSKQLQQSM | |
| | | SSADSTQAGSGATNFSLLKQ | |
| | | AGDVEENPGPMYSFVSEETG | |
| | | TLIVNSVLLFLAFVVFLLVT | |
| | | LAILTALRLCAYCCNIVNVS | |
| | | LVKPSFYVYSRVKNLNSSRV | |
| | | PDLLV* | |
| CoVEG1 | Amino | MATTMEQETCAHSLTFEECP | 32 |
| polypeptide | acid | KCSALQYRNGFYLLKYDEEW | |
| (fusion of | | YPEELLTDGEDDVFDPELDM | |
| EMCV L | | EVVFELQGSGATNFSLLKQA | |
| protein, 2A | | GDVEENPGPMADSNGTITVE | |
| site, | | ELKKLLEQWNLVIGFLFLTW | |
| M protein, | | ICLLQFAYANRNRFLYIIKL | |
| 2A site and E | | IFLWLLWPVTLACFVLAAVY | |
| protein) | | RINWITGGIAIAMACLVGLM | |
| | | WLSYFIASFRLFARTRSMWS | |
| | | FNPETNILLNVPLHGTILTR | |
| | | PLLESELVIGAVILRGHLRI | |
| | | AGHHLGRCDIKDLPKEITVA | |
| | | TSRTLSYYKLGASQRVAGDS | |
| | | GFAAYSRYRIGNYKLNTDHS | |
| | | SSSDNIALLVQGSGATNFSL | |
| | | LKQAGDVEENPGPMYSFVSE | |
| | | ETGTLIVNSVLLFLAFVVFL | |
| | | LVTLAILTALRLCAYCCNIV | |
| | | NVSLVKPSFYVYSRVKNLNS | |
| | | SRVPDLLV* | |
| CMV | Nucleic | GACATTGATTATTGACTAGT | 27 |
| enhancer | acid | TATTAATAGTAATCAATTAC | |
| | | GGGGTCATTAGTTCATAGCC | |
| | | CATATATGGAGTTCCGCGTT | |
| | | ACATAACTTACGGTAAATGG | |
| | | CCCGCCTGGCTGACCGCCCA | |
| | | ACGACCCCCGCCCATTGACG | |
| | | TCAATAATGACGTATGTTCC | |
| | | CATAGTAACGCCAATAGGGA | |
| | | CTTTCCATTGACGTCAATGG | |
| | | GTGGAGTATTTACGGTAAAC | |
| | | TGCCCACTTGGCAGTACATC | |
| | | AAGTGTATCATATGCCAAGT | |
| | | ACGCCCCCTATTGACGTCAA | |
| | | TGACGGTAAATGGCCCGCCT | |
| | | GGCATTATGCCCAGTACATG | |
| | | ACCTTATGGGACTTTCCTAC | |
| | | TTGGCAGTACATCTACGTAT | |
| | | TAGTCATCGCTATTACCATG | |
| CMV | Nucleic | GTGATGCGGTTTTGGCAGTA | 28 |
| promoter | acid | CATCAATGGGCGTGGATAGC | |
| | | GGTTTGACTCACGGGGATTT | |
| | | CCAAGTCTCCACCCCATTGA | |
| | | CGTCAATGGGAGTTTGTTTT | |
| | | GGCACCAAAATCAACGGGAC | |
| | | TTTCCAAAATGTCGTAACAA | |
| | | CTCCGCCCCATTGACGCAAA | |
| | | TGGGCGGTAGGCGTGTACGG | |
| | | TGGGAGGTCTATATAAGCAG | |
| | | AGCT | |
| Cloning | Nucleic | GGTTTAGTGAACCGTCAGAT | 29 |
| site | acid | CCGCTAGCGCTACCGGACTC | |
| | | AGATCTCGAGCTCAAGCTTC | |
| | | GAATTCTGCAGTCGACGGTA | |
| | | CCGCGGGCCCGGGATCCACC | |
| | | GGTCGCCACG | |
| CoVEG2 | Nucleic | GACATTGATTATTGACTAGT | 30 |
| insert | acid | TATTAATAGTAATCAATTAC | |
| sequence | | GGGGTCATTAGTTCATAGCC | |
| | | CATATATGGAGTTCCGCGTT | |
| | | ACATAACTTACGGTAAATGG | |
| | | CCCGCCTGGCTGACCGCCCA | |
| | | ACGACCCCCGCCCATTGACG | |
| | | TCAATAATGACGTATGTTCC | |
| | | CATAGTAACGCCAATAGGGA | |
| | | CTTTCCATTGACGTCAATGG | |
| | | GTGGAGTATTTACGGTAAAC | |
| | | TGCCCACTTGGCAGTACATC | |
| | | AAGTGTATCATATGCCAAGT | |
| | | ACGCCCCCTATTGACGTCAA | |
| | | TGACGGTAAATGGCCCGCCT | |
| | | GGCATTATGCCCAGTACATG | |
| | | ACCTTATGGGACTTTCCTAC | |
| | | TTGGCAGTACATCTACGTAT | |
| | | TAGTCATCGCTATTACCATG | |
| | | GTGATGCGGTTTTGGCAGTA | |
| | | CATCAATGGGCGTGGATAGC | |
| | | GGTTTGACTCACGGGGATTT | |
| | | CCAAGTCTCCACCCCATTGA | |
| | | CGTCAATGGGAGTTTGTTTT | |
| | | GGCACCAAAATCAACGGGAC | |
| | | TTTCCAAAATGTCGTAACAA | |
| | | CTCCGCCCCATTGACGCAAA | |
| | | TGGGCGGTAGGCGTGTACGG | |
| | | TGGGAGGTCTATATAAGCAG | |
| | | AGCTGGTTTAGTGAACCGTC | |
| | | AGATCCGCTAGCGCTACCGG | |
| | | ACTCAGATCTCGAGCTCAAG | |
| | | CTTCGAATTCTGCAGTCGAC | |
| | | GGTACCGCGGGCCCGGGATC | |
| | | CACCGGTCGCCACGATGTTT | |
| | | GTTTTTCTTGTTTTATTGCC | |
| | | ACTAGTCTCTAGTCAGTGTG | |
| | | TTAATCTTACAACCAGAACT | |
| | | CAATTACCCCCTGCATACAC | |
| | | TAATTCTTTCACACGTGGTG | |
| | | TTTATTACCCTGACAAAGTT | |
| | | TTCAGATCCTCAGTTTTACA | |
| | | TTCAACTCAGGACTTGTTCT | |
| | | TACCTTTCTTTTCCAATGTT | |
| | | ACTTGGTTCCATGCTATACA | |
| | | TGTCTCTGGGACCAATGGTA | |
| | | CTAAGAGGTTTGATAACCCT | |
| | | GTCCTACCATTTAATGATGG | |
| | | TGTTTATTTTGCTTCCACTG | |
| | | AGAAGTCTAACATAATAAGA | |
| | | GGCTGGATTTTTGGTACTAC | |
| | | TTTAGATTCGAAGACCCAGT | |
| | | CCCTACTTATTGTTAATAAC | |
| | | GCTACTAATGTTGTTATTAA | |
| | | AGTCTGTGAATTTCAATTTT | |
| | | GTAATGATCCATTTTTGGGT | |
| | | GTTTATTACCACAAAAACAA | |
| | | CAAAAGTTGGATGGAAAGTG | |
| | | AGTTCAGAGTTTATTCTAGT | |
| | | GCGAATAATTGCACTTTTGA | |
| | | ATATGTCTCTCAGCCTTTTC | |
| | | TTATGGACCTTGAAGGAAAA | |
| | | CAGGGTAATTTCAAAAATCT | |
| | | TAGGGAATTTGTGTTTAAGA | |
| | | ATATTGATGGTTATTTTAAA | |
| | | ATATATTCTAAGCACACGCC | |
| | | TATTAATTTAGTGCGTGATC | |
| | | TCCCTCAGGGTTTTTCGGCT | |
| | | TTAGAACCATTGGTAGATTT | |
| | | GCCAATAGGTATTAACATCA | |
| | | CTAGGTTTCAAACTTTACTT | |
| | | GCTTTACATAGAAGTTATTT | |
| | | GACTCCTGGTGATTCTTCTT | |
| | | CAGGTTGGACAGCTGGTGCT | |
| | | GCAGCTTATTATGTGGGTTA | |
| | | TCTTCAACCTAGGACTTTTC | |
| | | TATTAAAATATAATGAAAAT | |
| | | GGAACCATTACAGATGCTGT | |
| | | AGACTGTGCACTTGACCCTC | |
| | | TCTCAGAAACAAAGTGTACG | |
| | | TTGAAATCCTTCACTGTAGA | |
| | | AAAAGGAATCTATCAAACTT | |
| | | CTAACTTTAGAGTCCAACCA | |
| | | ACAGAATCTATTGTTAGATT | |
| | | TCCTAATATTACAAACTTGT | |
| | | GCCCTTTTGGTGAAGTTTTT | |
| | | AACGCCACCAGATTTGCATC | |
| | | TGTTTATGCTTGGAACAGGA | |
| | | AGAGAATCAGCAACTGTGTT | |
| | | GCTGATTATTCTGTCCTATA | |
| | | TAATTCCGCATCATTTTCCA | |
| | | CTTTTAAGTGTTATGGAGTG | |
| | | TCTCCTACTAAATTAAATGA | |
| | | TCTCTGCTTTACTAATGTCT | |
| | | ATGCAGATTCATTTGTAATT | |
| | | AGAGGTGATGAAGTCAGACA | |
| | | AATCGCTCCAGGGCAAACTG | |
| | | GAAAGATTGCTGATTATAAT | |
| | | TATAAATTACCAGATGATTT | |
| | | TACAGGCTGCGTTATAGCTT | |
| | | GGAATTCTAACAATCTTGAT | |
| | | TCTAAGGTTGGTGGTAATTA | |
| | | TAATTACCTGTATAGATTGT | |
| | | TTAGGAAGTCTAATCTCAAA | |
| | | CCTTTTGAGAGAGATATTTC | |
| | | AACTGAAATCTATCAGGCCG | |
| | | GTAGCACACCTTGTAATGGT | |
| | | GTTGAAGGTTTTAATTGTTA | |
| | | CTTTCCTTTACAATCATATG | |
| | | GTTTCCAACCCACTAATGGT | |
| | | GTTGGTTACCAACCATACAG | |
| | | AGTAGTAGTACTTTCTTTTG | |
| | | AACTTCTACATGCACCAGCA | |
| | | ACTGTTTGTGGACCTAAAAA | |
| | | GTCTACTAATTTGGTTAAAA | |
| | | ACAAATGTGTCAATTTCAAC | |
| | | TTCAATGGTTTAACAGGCAC | |
| | | AGGTGTTCTTACTGAGTCTA | |
| | | ACAAAAAGTTTCTGCCTTTC | |
| | | CAACAATTTGGCAGAGACAT | |
| | | TGCTGACACTACTGATGCTG | |
| | | TCCGTGATCCACAGACACTT | |
| | | GAGATTCTTGACATTACACC | |
| | | ATGTTCTTTTGGTGGTGTCA | |
| | | GTGTTATAACACCAGGAACA | |
| | | AATACTTCTAACCAGGTTGC | |
| | | TGTTCTTTATCAGGATGTTA | |
| | | ACTGCACAGAAGTCCCTGTT | |
| | | GCTATTCATGCAGATCAACT | |
| | | TACTCCTACTTGGCGTGTTT | |
| | | ATTCTACAGGTTCTAATGTT | |
| | | TTTCAAACACGTGCAGGCTG | |
| | | TTTAATAGGGGCTGAACATG | |
| | | TCAACAACTCATATGAGTGT | |
| | | GACATACCCATTGGTGCAGG | |
| | | TATATGCGCTAGTTATCAGA | |
| | | CTCAGACTAATTCTCCTCGG | |
| | | CGGGCACGTAGTGTAGCTAG | |
| | | TCAATCCATCATTGCCTACA | |
| | | CTATGTCACTTGGTGCAGAA | |
| | | AATTCAGTTGCTTACTCTAA | |
| | | TAACTCTATTGCCATACCCA | |
| | | CAAATTTTACTATTAGTGTT | |
| | | ACCACAGAAATTCTACCAGT | |
| | | GTCTATGACCAAGACATCAG | |
| | | TAGATTGTACAATGTACATT | |
| | | TGTGGTGATTCAACTGAATG | |
| | | CAGCAATCTTTTGTTGCAAT | |
| | | ATGGCAGTTTTTGTACACAA | |
| | | TTAAACCGTGCTTTAACTGG | |
| | | AATAGCTGTTGAACAAGACA | |
| | | AAAACACCCAAGAAGTTTTT | |
| | | GCACAAGTCAAACAAATTTA | |
| | | CAAAACACCACCAATTAAAG | |
| | | ATTTTGGTGGTTTTAATTTT | |
| | | TCACAAATATTACCAGATCC | |
| | | ATCAAAACCAAGCAAGAGGT | |
| | | CATTTATTGAAGATCTACTT | |
| | | TTCAACAAAGTGACACTTGC | |
| | | AGATGCTGGCTTCATCAAAC | |
| | | AATATGGTGATTGCCTTGGT | |
| | | GATATTGCTGCTAGAGACCT | |
| | | CATTTGTGCACAAAAGTTTA | |
| | | ACGGCCTTACTGTTTTGCCA | |
| | | CCTTTGCTCACAGATGAAAT | |
| | | GATTGCTCAATACACTTCTG | |
| | | CACTGTTAGCGGGTACAATC | |
| | | ACTTCTGGTTGGACCTTTGG | |
| | | TGCAGGTGCTGCATTACAAA | |
| | | TACCATTTGCTATGCAAATG | |
| | | GCTTATAGGTTTAATGGTAT | |
| | | TGGAGTTACACAGAATGTTC | |
| | | TCTATGAGAACCAAAAATTG | |
| | | ATTGCCAACCAATTTAATAG | |
| | | TGCTATTGGCAAAATTCAAG | |
| | | ACTCACTTTCTTCCACAGCA | |
| | | AGTGCACTTGGAAAACTTCA | |
| | | AGATGTGGTCAACCAAAATG | |
| | | CACAAGCTTTAAACACGCTT | |
| | | GTTAAACAACTTAGCTCCAA | |
| | | TTTTGGTGCAATTTCAAGTG | |
| | | TTTTAAATGATATCCTTTCA | |
| | | CGTCTTGACAAAGTTGAGGC | |
| | | TGAAGTGCAAATTGATAGGT | |
| | | TGATCACAGGCAGACTTCAA | |
| | | AGTTTGCAGACATATGTGAC | |
| | | TCAACAATTAATTAGAGCTG | |
| | | CAGAAATCAGAGCTTCTGCT | |
| | | AATCTTGCTGCTACTAAAAT | |
| | | GTCAGAGTGTGTACTTGGAC | |
| | | AATCAAAAAGAGTTGATTTT | |
| | | TGTGGAAAGGGCTATCATCT | |
| | | TATGTCCTTCCCTCAGTCAG | |
| | | CACCTCATGGTGTAGTCTTC | |
| | | TTGCATGTGACTTATGTCCC | |
| | | TGCACAAGAAAAGAACTTCA | |
| | | CAACTGCTCCTGCCATTTGT | |
| | | CATGATGGAAAAGCACACTT | |
| | | TCCTCGTGAAGGTGTCTTTG | |
| | | TTTCAAATGGCACACACTGG | |
| | | TTTGTAACACAAAGGAATTT | |
| | | TTATGAACCACAAATCATTA | |
| | | CTACAGACAACACATTTGTG | |
| | | TCTGGTAACTGTGATGTTGT | |
| | | AATAGGAATTGTCAACAACA | |
| | | CAGTTTATGATCCTTTGCAA | |
| | | CCTGAATTAGACTCATTCAA | |
| | | GGAGGAGTTAGATAAATATT | |
| | | TTAAGAATCATACATCACCA | |
| | | GATGTTGATTTAGGTGACAT | |
| | | CTCTGGCATTAATGCTTCAG | |
| | | TTGTAAACATTCAAAAAGAA | |
| | | ATTGACCGCCTCAATGAGGT | |
| | | TGCCAAGAATTTAAATGAAT | |
| | | CTCTCATCGATCTCCAAGAA | |
| | | CTTGGAAAGTATGAGCAGTA | |
| | | TATAAAATGGCCATGGTACA | |
| | | TTTGGCTAGGTTTTATAGCT | |
| | | GGCTTGATTGCCATAGTAAT | |
| | | GGTGACAATTATGCTTTGCT | |
| | | GTATGACCAGTTGCTGTAGT | |
| | | TGTCTCAAGGGCTGTTGTTC | |
| | | TTGTGGATCCTGCTGCAAAT | |
| | | TTGATGAAGACGACTCTGAG | |
| | | CCAGTGCTCAAAGGAGTCAA | |
| | | ATTACATTACACATAATCCC | |
| | | CCCCCCCTAACGTTACTGGC | |
| | | CGAAGCCGCTTGGAATAAGG | |
| | | CCGGTGTGCGTTTGTCTATA | |
| | | TGTTATTTTCCACCATATTG | |
| | | CCGTCTTTTGGCAATGTGAG | |
| | | GGCCCGGAAACCTGGCCCTG | |
| | | TCTTCTTGACGAGCATTCCT | |
| | | AGGGGTCTTTCCCCTCTCGC | |
| | | CAAAGGAATGCAAGGTCTGT | |
| | | TGAATGTCGTGAAGGAAGCA | |
| | | GTTCCTCTGGAAGCTTCTTG | |
| | | AAGACAAACAACGTCTGTAG | |
| | | CGACCCTTTGCAGGCAGCGG | |
| | | AACCCCCCACCTGGCGACAG | |
| | | GTGCCTCTGCGGCCAAAAGC | |
| | | CACGTGTATAAGATACACCT | |
| | | GCAAAGGCGGCACAACCCCA | |
| | | GTGCCACGTTGTGAGTTGGA | |
| | | TAGTTGTGGAAAGAGTCAAA | |
| | | TGGCTCTCCTCAAGCGTATT | |
| | | CAACAAGGGGCTGAAGGATG | |
| | | CCCAGAAGGTACCCCATTGT | |
| | | ATGGGATCTGATCTGGGGCC | |
| | | TCGGTGCACATGCTTTACAT | |
| | | GTGTTTAGTCGAGGTTAAAA | |
| | | AAACGTCTAGGCCCCCCGAA | |
| | | CCACGGGGACGTGGTTTTCC | |
| | | TTTGAAAAACACGATGATAA | |
| | | TATGGCCACAACCATGGAAC | |
| | | AAGAGACTTGCGCGCACTCT | |
| | | CTCACTTTTGAGGAATGCCC | |
| | | AAAATGCTCTGCTCTACAAT | |
| | | ACCGTAATGGATTTTACCTG | |
| | | CTAAAGTATGATGAAGAATG | |
| | | GTACCCAGAGGAGTTATTGA | |
| | | CTGATGGAGAGGATGATGTC | |
| | | TTTGATCCCGAATTAGACAT | |
| | | GGAAGTCGTTTTCGAGTTAC | |
| | | AGGGAAGCGGAGCTACTAAC | |
| | | TTCAGCCTGCTGAAGCAGGC | |
| | | TGGAGATGTGGAGGAGAACC | |
| | | CTGGACCTATGGCAGATTCC | |
| | | AACGGTACTATTACCGTTGA | |
| | | AGAGCTTAAAAAGCTCCTTG | |
| | | AACAATGGAACCTAGTAATA | |
| | | GGTTTCCTATTCCTTACATG | |
| | | GATTTGTCTTCTACAATTTG | |
| | | CCTATGCCAACAGGAATAGG | |
| | | TTTTTGTATATAATTAAGTT | |
| | | AATTTTCCTCTGGCTGTTAT | |
| | | GGCCAGTAACTTTAGCTTGT | |
| | | TTTGTGCTTGCTGCTGTTTA | |
| | | CAGAATAAATTGGATCACCG | |
| | | GTGGAATTGCTATCGCAATG | |
| | | GCTTGTCTTGTAGGCTTGAT | |
| | | GTGGCTCAGCTACTTCATTG | |
| | | CTTCTTTCAGACTGTTTGCG | |
| | | CGTACGCGTTCCATGTGGTC | |
| | | ATTCAATCCAGAAACTAACA | |
| | | TTCTTCTCAACGTGCCACTC | |
| | | CATGGCACTATTCTGACCAG | |
| | | ACCGCTTCTAGAAAGTGAAC | |
| | | TCGTAATCGGAGCTGTGATC | |
| | | CTTCGTGGACATCTTCGTAT | |
| | | TGCTGGACACCATCTAGGAC | |
| | | GCTGTGACATCAAGGACCTG | |
| | | CCTAAAGAAATCACTGTTGC | |
| | | TACATCACGAACGCTTTCTT | |
| | | ATTACAAATTGGGAGCTTCG | |
| | | CAGCGTGTAGCAGGTGACTC | |
| | | AGGTTTTGCTGCATACAGTC | |
| | | GCTACAGGATTGGCAACTAT | |
| | | AAATTAAACACAGACCATTC | |
| | | CAGTAGCAGTGACAATATTG | |
| | | CTTTGCTTGTACAGTAACCC | |
| | | CCCCCCCTAACGTTACTGGC | |
| | | CGAAGCCGCTTGGAATAAGG | |
| | | CCGGTGTGCGTTTGTCTATA | |
| | | TGTTATTTTCCACCATATTG | |
| | | CCGTCTTTTGGCAATGTGAG | |
| | | GGCCCGGAAACCTGGCCCTG | |
| | | TCTTCTTGACGAGCATTCCT | |
| | | AGGGGTCTTTCCCCTCTCGC | |
| | | CAAAGGAATGCAAGGTCTGT | |
| | | TGAATGTCGTGAAGGAAGCA | |
| | | GTTCCTCTGGAAGCTTCTTG | |
| | | AAGACAAACAACGTCTGTAG | |
| | | CGACCCTTTGCAGGCAGCGG | |
| | | AACCCCCCACCTGGCGACAG | |
| | | GTGCCTCTGCGGCCAAAAGC | |
| | | CACGTGTATAAGATACACCT | |
| | | GCAAAGGCGGCACAACCCCA | |
| | | GTGCCACGTTGTGAGTTGGA | |
| | | TAGTTGTGGAAAGAGTCAAA | |
| | | TGGCTCTCCTCAAGCGTATT | |
| | | CAACAAGGGGCTGAAGGATG | |
| | | CCCAGAAGGTACCCCATTGT | |
| | | ATGGGATCTGATCTGGGGCC | |
| | | TCGGTGCACATGCTTTACAT | |
| | | GTGTTTAGTCGAGGTTAAAA | |
| | | AAACGTCTAGGCCCCCCGAA | |
| | | CCACGGGGACGTGGTTTTCC | |
| | | TTTGAAAAACACGATGATAA | |
| | | TATGGCCACAACCATGGAAC | |
| | | AAGAGACTTGCGCGCACTCT | |
| | | CTCACTTTTGAGGAATGCCC | |
| | | AAAATGCTCTGCTCTACAAT | |
| | | ACCGTAATGGATTTTACCTG | |
| | | CTAAAGTATGATGAAGAATG | |
| | | GTACCCAGAGGAGTTATTGA | |
| | | CTGATGGAGAGGATGATGTC | |
| | | TTTGATCCCGAATTAGACAT | |
| | | GGAAGTCGTTTTCGAGTTAC | |
| | | AGGGAAGCGGAGCTACTAAC | |
| | | TTCAGCCTGCTGAAGCAGGC | |
| | | TGGAGATGTGGAGGAGAACC | |
| | | CTGGACCTATGTCTGATAAT | |
| | | GGACCCCAAAATCAGCGAAA | |
| | | TGCACCCCGCATTACGTTTG | |
| | | GTGGACCCTCAGATTCAACT | |
| | | GGCAGTAACCAGAATGGAGA | |
| | | ACGCAGTGGGGCGCGATCAA | |
| | | AACAACGTCGGCCCCAAGGT | |
| | | TTACCCAATAATACTGCGTC | |
| | | TTGGTTCACCGCTCTCACTC | |
| | | AACATGGCAAGGAAGACCTT | |
| | | AAATTCCCTCGAGGACAAGG | |
| | | CGTTCCAATTAACACCAATA | |
| | | GCAGTCCAGATGACCAAATT | |
| | | GGCTACTACCGAAGAGCTAC | |
| | | CAGACGAATTCGTGGTGGTG | |
| | | ACGGTAAAATGAAAGATCTC | |
| | | AGTCCAAGATGGTATTTCTA | |
| | | CTACCTAGGAACTGGGCCAG | |
| | | AAGCTGGACTTCCCTATGGT | |
| | | GCTAACAAAGACGGCATCAT | |
| | | ATGGGTTGCAACTGAGGGAG | |
| | | CCTTGAATACACCAAAAGAT | |
| | | CACATTGGCACCCGCAATCC | |
| | | TGCTAACAATGCTGCAATCG | |
| | | TGCTACAACTTCCTCAAGGA | |
| | | ACAACATTGCCAAAAGGCTT | |
| | | CTACGCAGAAGGGAGCAGAG | |
| | | GCGGCAGTCAAGCCTCTTCT | |
| | | CGTTCCTCATCACGTAGTCG | |
| | | CAACAGTTCAAGAAATTCAA | |
| | | CTCCAGGCAGCAGTAGGGGA | |
| | | ACTTCTCCTGCTAGAATGGC | |
| | | TGGCAATGGCGGTGATGCTG | |
| | | CTCTTGCTTTGCTGCTGCTT | |
| | | GACAGATTGAACCAGCTTGA | |
| | | GAGCAAAATGTCTGGTAAAG | |
| | | GCCAACAACAACAAGGCCAA | |
| | | ACTGTCACTAAGAAATCTGC | |
| | | TGCTGAGGCTTCTAAGAAGC | |
| | | CTCGGCAAAAACGTACTGCC | |
| | | ACTAAAGCATACAATGTAAC | |
| | | ACAAGCTTTCGGCAGACGTG | |
| | | GTCCAGAACAAACCCAAGGA | |
| | | AATTTTGGGGACCAGGAACT | |
| | | AATCAGACAAGGAACTGATT | |
| | | ACAAACATTGGCCGCAAATT | |
| | | GCACAATTTGCCCCCAGCGC | |
| | | TTCAGCGTTCTTCGGAATGT | |
| | | CGCGCATTGGCATGGAAGTC | |
| | | ACACCTTCGGGAACGTGGTT | |
| | | GACCTACACAGGTGCCATCA | |
| | | AATTGGATGACAAAGATCCA | |
| | | AATTTCAAAGATCAAGTCAT | |
| | | TTTGCTGAATAAGCATATTG | |
| | | ACGCATACAAAACATTCCCA | |
| | | CCAACAGAGCCTAAAAAGGA | |
| | | CAAAAAGAAGAAGGCTGATG | |
| | | AAACTCAAGCCTTACCGCAG | |
| | | AGACAGAAGAAACAGCAAAC | |
| | | TGTGACTCTTCTTCCTGCTG | |
| | | CAGATTTGGATGATTTCTCC | |
| | | AAACAATTGCAACAATCCAT | |
| | | GAGCAGTGCTGACTCAACTC | |
| | | AGGCCGGAAGCGGAGCTACT | |
| | | AACTTCAGCCTGCTGAAGCA | |
| | | GGCTGGAGATGTGGAGGAGA | |
| | | ACCCTGGACCTATGTACTCA | |
| | | TTCGTTTCGGAAGAGACAGG | |
| | | TACGTTAATAGTTAATAGCG | |
| | | TACTTCTTTTTCTTGCTTTC | |
| | | GTGGTATTCTTGCTAGTTAC | |
| | | ACTAGCCATCCTTACTGCGC | |
| | | TTCGATTGTGTGCGTACTGC | |
| | | TGCAATATTGTTAACGTGAG | |
| | | TCTTGTAAAACCTTCTTTTT | |
| | | ACGTTTACTCTCGTGTTAAA | |
| | | AATCTGAATTCTTCTAGAGT | |
| | | TCCTGATCTTCTGGTCTAA | |
| CoVEG1 | Nucleic | GACATTGATTATTGACTAGT | 31 |
| insert | acid | TATTAATAGTAATCAATTAC | |
| sequence | | GGGGTCATTAGTTCATAGCC | |
| | | CATATATGGAGTTCCGCGTT | |
| | | ACATAACTTACGGTAAATGG | |
| | | CCCGCCTGGCTGACCGCCCA | |
| | | ACGACCCCCGCCCATTGACG | |
| | | TCAATAATGACGTATGTTCC | |
| | | CATAGTAACGCCAATAGGGA | |
| | | CTTTCCATTGACGTCAATGG | |
| | | GTGGAGTATTTACGGTAAAC | |
| | | TGCCCACTTGGCAGTACATC | |
| | | AAGTGTATCATATGCCAAGT | |
| | | ACGCCCCCTATTGACGTCAA | |
| | | TGACGGTAAATGGCCCGCCT | |
| | | GGCATTATGCCCAGTACATG | |
| | | ACCTTATGGGACTTTCCTAC | |
| | | TTGGCAGTACATCTACGTAT | |
| | | TAGTCATCGCTATTACCATG | |
| | | GTGATGCGGTTTTGGCAGTA | |
| | | CATCAATGGGCGTGGATAGC | |
| | | GGTTTGACTCACGGGGATTT | |
| | | CCAAGTCTCCACCCCATTGA | |
| | | CGTCAATGGGAGTTTGTTTT | |
| | | GGCACCAAAATCAACGGGAC | |
| | | TTTCCAAAATGTCGTAACAA | |
| | | CTCCGCCCCATTGACGCAAA | |
| | | TGGGCGGTAGGCGTGTACGG | |
| | | TGGGAGGTCTATATAAGCAG | |
| | | AGCTGGTTTAGTGAACCGTC | |
| | | AGATCCGCTAGCGCTACCGG | |
| | | ACTCAGATCTCGAGCTCAAG | |
| | | CTTCGAATTCTGCAGTCGAC | |
| | | GGTACCGCGGGCCCGGGATC | |
| | | CACCGGTCGCCACGATGTTT | |
| | | GTTTTTCTTGTTTTATTGCC | |
| | | ACTAGTCTCTAGTCAGTGTG | |
| | | TTAATCTTACAACCAGAACT | |
| | | CAATTACCCCCTGCATACAC | |
| | | TAATTCTTTCACACGTGGTG | |
| | | TTTATTACCCTGACAAAGTT | |
| | | TTCAGATCCTCAGTTTTACA | |
| | | TTCAACTCAGGACTTGTTCT | |
| | | TACCTTTCTTTTCCAATGTT | |
| | | ACTTGGTTCCATGCTATACA | |
| | | TGTCTCTGGGACCAATGGTA | |
| | | CTAAGAGGTTTGATAACCCT | |
| | | GTCCTACCATTTAATGATGG | |
| | | TGTTTATTTTGCTTCCACTG | |
| | | AGAAGTCTAACATAATAAGA | |
| | | GGCTGGATTTTTGGTACTAC | |
| | | TTTAGATTCGAAGACCCAGT | |
| | | CCCTACTTATTGTTAATAAC | |
| | | GCTACTAATGTTGTTATTAA | |
| | | AGTCTGTGAATTTCAATTTT | |
| | | GTAATGATCCATTTTTGGGT | |
| | | GTTTATTACCACAAAAACAA | |
| | | CAAAAGTTGGATGGAAAGTG | |
| | | AGTTCAGAGTTTATTCTAGT | |
| | | GCGAATAATTGCACTTTTGA | |
| | | ATATGTCTCTCAGCCTTTTC | |
| | | TTATGGACCTTGAAGGAAAA | |
| | | CAGGGTAATTTCAAAAATCT | |
| | | TAGGGAATTTGTGTTTAAGA | |
| | | ATATTGATGGTTATTTTAAA | |
| | | ATATATTCTAAGCACACGCC | |
| | | TATTAATTTAGTGCGTGATC | |
| | | TCCCTCAGGGTTTTTCGGCT | |
| | | TTAGAACCATTGGTAGATTT | |
| | | GCCAATAGGTATTAACATCA | |
| | | CTAGGTTTCAAACTTTACTT | |
| | | GCTTTACATAGAAGTTATTT | |
| | | GACTCCTGGTGATTCTTCTT | |
| | | CAGGTTGGACAGCTGGTGCT | |
| | | GCAGCTTATTATGTGGGTTA | |
| | | TCTTCAACCTAGGACTTTTC | |
| | | TATTAAAATATAATGAAAAT | |
| | | GGAACCATTACAGATGCTGT | |
| | | AGACTGTGCACTTGACCCTC | |
| | | TCTCAGAAACAAAGTGTACG | |
| | | TTGAAATCCTTCACTGTAGA | |
| | | AAAAGGAATCTATCAAACTT | |
| | | CTAACTTTAGAGTCCAACCA | |
| | | ACAGAATCTATTGTTAGATT | |
| | | TCCTAATATTACAAACTTGT | |
| | | GCCCTTTTGGTGAAGTTTTT | |
| | | AACGCCACCAGATTTGCATC | |
| | | TGTTTATGCTTGGAACAGGA | |
| | | AGAGAATCAGCAACTGTGTT | |
| | | GCTGATTATTCTGTCCTATA | |
| | | TAATTCCGCATCATTTTCCA | |
| | | CTTTTAAGTGTTATGGAGTG | |
| | | TCTCCTACTAAATTAAATGA | |
| | | TCTCTGCTTTACTAATGTCT | |
| | | ATGCAGATTCATTTGTAATT | |
| | | AGAGGTGATGAAGTCAGACA | |
| | | AATCGCTCCAGGGCAAACTG | |
| | | GAAAGATTGCTGATTATAAT | |
| | | TATAAATTACCAGATGATTT | |
| | | TACAGGCTGCGTTATAGCTT | |
| | | GGAATTCTAACAATCTTGAT | |
| | | TCTAAGGTTGGTGGTAATTA | |
| | | TAATTACCTGTATAGATTGT | |
| | | TTAGGAAGTCTAATCTCAAA | |
| | | CCTTTTGAGAGAGATATTTC | |
| | | AACTGAAATCTATCAGGCCG | |
| | | GTAGCACACCTTGTAATGGT | |
| | | GTTGAAGGTTTTAATTGTTA | |
| | | CTTTCCTTTACAATCATATG | |
| | | GTTTCCAACCCACTAATGGT | |
| | | GTTGGTTACCAACCATACAG | |
| | | AGTAGTAGTACTTTCTTTTG | |
| | | AACTTCTACATGCACCAGCA | |
| | | ACTGTTTGTGGACCTAAAAA | |
| | | GTCTACTAATTTGGTTAAAA | |
| | | ACAAATGTGTCAATTTCAAC | |
| | | TTCAATGGTTTAACAGGCAC | |
| | | AGGTGTTCTTACTGAGTCTA | |
| | | ACAAAAAGTTTCTGCCTTTC | |
| | | CAACAATTTGGCAGAGACAT | |
| | | TGCTGACACTACTGATGCTG | |
| | | TCCGTGATCCACAGACACTT | |
| | | GAGATTCTTGACATTACACC | |
| | | ATGTTCTTTTGGTGGTGTCA | |
| | | GTGTTATAACACCAGGAACA | |
| | | AATACTTCTAACCAGGTTGC | |
| | | TGTTCTTTATCAGGATGTTA | |
| | | ACTGCACAGAAGTCCCTGTT | |
| | | GCTATTCATGCAGATCAACT | |
| | | TACTCCTACTTGGCGTGTTT | |
| | | ATTCTACAGGTTCTAATGTT | |
| | | TTTCAAACACGTGCAGGCTG | |
| | | TTTAATAGGGGCTGAACATG | |
| | | TCAACAACTCATATGAGTGT | |
| | | GACATACCCATTGGTGCAGG | |
| | | TATATGCGCTAGTTATCAGA | |
| | | CTCAGACTAATTCTCCTCGG | |
| | | CGGGCACGTAGTGTAGCTAG | |
| | | TCAATCCATCATTGCCTACA | |
| | | CTATGTCACTTGGTGCAGAA | |
| | | AATTCAGTTGCTTACTCTAA | |
| | | TAACTCTATTGCCATACCCA | |
| | | CAAATTTTACTATTAGTGTT | |
| | | ACCACAGAAATTCTACCAGT | |
| | | GTCTATGACCAAGACATCAG | |
| | | TAGATTGTACAATGTACATT | |
| | | TGTGGTGATTCAACTGAATG | |
| | | CAGCAATCTTTTGTTGCAAT | |
| | | ATGGCAGTTTTTGTACACAA | |
| | | TTAAACCGTGCTTTAACTGG | |
| | | AATAGCTGTTGAACAAGACA | |
| | | AAAACACCCAAGAAGTTTTT | |
| | | GCACAAGTCAAACAAATTTA | |
| | | CAAAACACCACCAATTAAAG | |
| | | ATTTTGGTGGTTTTAATTTT | |
| | | TCACAAATATTACCAGATCC | |
| | | ATCAAAACCAAGCAAGAGGT | |
| | | CATTTATTGAAGATCTACTT | |
| | | TTCAACAAAGTGACACTTGC | |
| | | AGATGCTGGCTTCATCAAAC | |
| | | AATATGGTGATTGCCTTGGT | |
| | | GATATTGCTGCTAGAGACCT | |
| | | CATTTGTGCACAAAAGTTTA | |
| | | ACGGCCTTACTGTTTTGCCA | |
| | | CCTTTGCTCACAGATGAAAT | |
| | | GATTGCTCAATACACTTCTG | |
| | | CACTGTTAGCGGGTACAATC | |
| | | ACTTCTGGTTGGACCTTTGG | |
| | | TGCAGGTGCTGCATTACAAA | |
| | | TACCATTTGCTATGCAAATG | |
| | | GCTTATAGGTTTAATGGTAT | |
| | | TGGAGTTACACAGAATGTTC | |
| | | TCTATGAGAACCAAAAATTG | |
| | | ATTGCCAACCAATTTAATAG | |
| | | TGCTATTGGCAAAATTCAAG | |
| | | ACTCACTTTCTTCCACAGCA | |
| | | AGTGCACTTGGAAAACTTCA | |
| | | AGATGTGGTCAACCAAAATG | |
| | | CACAAGCTTTAAACACGCTT | |
| | | GTTAAACAACTTAGCTCCAA | |
| | | TTTTGGTGCAATTTCAAGTG | |
| | | TTTTAAATGATATCCTTTCA | |
| | | CGTCTTGACAAAGTTGAGGC | |
| | | TGAAGTGCAAATTGATAGGT | |
| | | TGATCACAGGCAGACTTCAA | |
| | | AGTTTGCAGACATATGTGAC | |
| | | TCAACAATTAATTAGAGCTG | |
| | | CAGAAATCAGAGCTTCTGCT | |
| | | AATCTTGCTGCTACTAAAAT | |
| | | GTCAGAGTGTGTACTTGGAC | |
| | | AATCAAAAAGAGTTGATTTT | |
| | | TGTGGAAAGGGCTATCATCT | |
| | | TATGTCCTTCCCTCAGTCAG | |
| | | CACCTCATGGTGTAGTCTTC | |
| | | TTGCATGTGACTTATGTCCC | |
| | | TGCACAAGAAAAGAACTTCA | |
| | | CAACTGCTCCTGCCATTTGT | |
| | | CATGATGGAAAAGCACACTT | |
| | | TCCTCGTGAAGGTGTCTTTG | |
| | | TTTCAAATGGCACACACTGG | |
| | | TTTGTAACACAAAGGAATTT | |
| | | TTATGAACCACAAATCATTA | |
| | | CTACAGACAACACATTTGTG | |
| | | TCTGGTAACTGTGATGTTGT | |
| | | AATAGGAATTGTCAACAACA | |
| | | CAGTTTATGATCCTTTGCAA | |
| | | CCTGAATTAGACTCATTCAA | |
| | | GGAGGAGTTAGATAAATATT | |
| | | TTAAGAATCATACATCACCA | |
| | | GATGTTGATTTAGGTGACAT | |
| | | CTCTGGCATTAATGCTTCAG | |
| | | TTGTAAACATTCAAAAAGAA | |
| | | ATTGACCGCCTCAATGAGGT | |
| | | TGCCAAGAATTTAAATGAAT | |
| | | CTCTCATCGATCTCCAAGAA | |
| | | CTTGGAAAGTATGAGCAGTA | |
| | | TATAAAATGGCCATGGTACA | |
| | | TTTGGCTAGGTTTTATAGCT | |
| | | GGCTTGATTGCCATAGTAAT | |
| | | GGTGACAATTATGCTTTGCT | |
| | | GTATGACCAGTTGCTGTAGT | |
| | | TGTCTCAAGGGCTGTTGTTC | |
| | | TTGTGGATCCTGCTGCAAAT | |
| | | TTGATGAAGACGACTCTGAG | |
| | | CCAGTGCTCAAAGGAGTCAA | |
| | | ATTACATTACACATAATCCC | |
| | | CCCCCCCTAACGTTACTGGC | |
| | | CGAAGCCGCTTGGAATAAGG | |
| | | CCGGTGTGCGTTTGTCTATA | |
| | | TGTTATTTTCCACCATATTG | |
| | | CCGTCTTTTGGCAATGTGAG | |
| | | GGCCCGGAAACCTGGCCCTG | |
| | | TCTTCTTGACGAGCATTCCT | |
| | | AGGGGTCTTTCCCCTCTCGC | |
| | | CAAAGGAATGCAAGGTCTGT | |
| | | TGAATGTCGTGAAGGAAGCA | |
| | | GTTCCTCTGGAAGCTTCTTG | |
| | | AAGACAAACAACGTCTGTAG | |
| | | CGACCCTTTGCAGGCAGCGG | |
| | | AACCCCCCACCTGGCGACAG | |
| | | GTGCCTCTGCGGCCAAAAGC | |
| | | CACGTGTATAAGATACACCT | |
| | | GCAAAGGCGGCACAACCCCA | |
| | | GTGCCACGTTGTGAGTTGGA | |
| | | TAGTTGTGGAAAGAGTCAAA | |
| | | TGGCTCTCCTCAAGCGTATT | |
| | | CAACAAGGGGCTGAAGGATG | |
| | | CCCAGAAGGTACCCCATTGT | |
| | | ATGGGATCTGATCTGGGGCC | |
| | | TCGGTGCACATGCTTTACAT | |
| | | GTGTTTAGTCGAGGTTAAAA | |
| | | AAACGTCTAGGCCCCCCGAA | |
| | | CCACGGGGACGTGGTTTTCC | |
| | | TTTGAAAAACACGATGATAA | |
| | | TATGGCCACAACCATGGAAC | |
| | | AAGAGACTTGCGCGCACTCT | |
| | | CTCACTTTTGAGGAATGCCC | |
| | | AAAATGCTCTGCTCTACAAT | |
| | | ACCGTAATGGATTTTACCTG | |
| | | CTAAAGTATGATGAAGAATG | |
| | | GTACCCAGAGGAGTTATTGA | |
| | | CTGATGGAGAGGATGATGTC | |
| | | TTTGATCCCGAATTAGACAT | |
| | | GGAAGTCGTTTTCGAGTTAC | |
| | | AGGGAAGCGGAGCTACTAAC | |
| | | TTCAGCCTGCTGAAGCAGGC | |
| | | TGGAGATGTGGAGGAGAACC | |
| | | CTGGACCTATGGCAGATTCC | |
| | | AACGGTACTATTACCGTTGA | |
| | | AGAGCTTAAAAAGCTCCTTG | |
| | | AACAATGGAACCTAGTAATA | |
| | | GGTTTCCTATTCCTTACATG | |
| | | GATTTGTCTTCTACAATTTG | |
| | | CCTATGCCAACAGGAATAGG | |
| | | TTTTTGTATATAATTAAGTT | |
| | | AATTTTCCTCTGGCTGTTAT | |
| | | GGCCAGTAACTTTAGCTTGT | |
| | | TTTGTGCTTGCTGCTGTTTA | |
| | | CAGAATAAATTGGATCACCG | |
| | | GTGGAATTGCTATCGCAATG | |
| | | GCTTGTCTTGTAGGCTTGAT | |
| | | GTGGCTCAGCTACTTCATTG | |
| | | CTTCTTTCAGACTGTTTGCG | |
| | | CGTACGCGTTCCATGTGGTC | |
| | | ATTCAATCCAGAAACTAACA | |
| | | TTCTTCTCAACGTGCCACTC | |
| | | CATGGCACTATTCTGACCAG | |
| | | ACCGCTTCTAGAAAGTGAAC | |
| | | TCGTAATCGGAGCTGTGATC | |
| | | CTTCGTGGACATCTTCGTAT | |
| | | TGCTGGACACCATCTAGGAC | |
| | | GCTGTGACATCAAGGACCTG | |
| | | CCTAAAGAAATCACTGTTGC | |
| | | TACATCACGAACGCTTTCTT | |
| | | ATTACAAATTGGGAGCTTCG | |
| | | CAGCGTGTAGCAGGTGACTC | |
| | | AGGTTTTGCTGCATACAGTC | |
| | | GCTACAGGATTGGCAACTAT | |
| | | AAATTAAACACAGACCATTC | |
| | | CAGTAGCAGTGACAATATTG | |
| | | CTTTGCTTGTACAGGGAAGC | |
| | | GGAGCTACTAACTTCAGCCT | |
| | | GCTGAAGCAGGCTGGAGATG | |
| | | TGGAGGAGAACCCTGGACCT | |
| | | ATGTACTCATTCGTTTCGGA | |
| | | AGAGACAGGTACGTTAATAG | |
| | | TTAATAGCGTACTTCTTTTT | |
| | | CTTGCTTTCGTGGTATTCTT | |
| | | GCTAGTTACACTAGCCATCC | |
| | | TTACTGCGCTTCGATTGTGT | |
| | | GCGTACTGCTGCAATATTGT | |
| | | TAACGTGAGTCTTGTAAAAC | |
| | | CTTCTTTTTACGTTTACTCT | |
| | | CGTGTTAAAAATCTGAATTC | |
| | | TTCTAGAGTTCCTGATCTTC | |
| | | TGGTCTAA | |

### i) Expression Cassettes

The vectors disclosed herein may comprise one or more expression cassettes. The phrase “expression cassette” as used herein refers to a defined segment of a nucleic acid molecule that comprises the minimum elements needed for production of another nucleic acid or protein encoded by that nucleic acid molecule. In some embodiments, the expression cassette comprises a promoter. In some embodiments, the promoter is operatively linked to each of the polynucleotide sequences of the expression cassette.

In some embodiments, a vector may comprise an expression cassette, the expression cassette comprising a first polynucleotide encoding an antigen, and a second polynucleotide encoding an enhancer protein. In some embodiments, the expression cassette comprises a first promoter, operatively linked to the first polynucleotide; and a second promoter, operatively linked to the second polynucleotide. In some embodiments, the expression cassette comprises a shared promoter operatively linked to both the first polynucleotide and the second polynucleotide.

In some embodiments, the expression cassette comprises a coding polynucleotide comprising the first polynucleotide and the second polynucleotide linked by a polynucleotide encoding a separating element (e.g., a ribosome skipping site or 2A element), the coding polynucleotide operatively linked to the shared promoter.

In some embodiments, the expression cassette comprises a coding polynucleotide, the coding polynucleotide encoding an enhancer protein and an antigen linked to by a separating element (e.g., a ribosome skipping site or 2A element), the coding polynucleotide operatively linked to the shared promoter.

In some embodiments, the expression cassette is configured for transcription of a single messenger RNA encoding both the antigen and the enhancer protein, linked by a separating element (e.g., a ribosome skipping site or 2A element); wherein translation of the messenger RNA results in expression of the antigen and the enhancer protein (e.g., the L protein) as distinct polypeptides. In some embodiments, the expression cassettes disclosed herein comprise one or more proteolytic cleavage sites, for example, 1, 2, 3, 4, or 5 proteolytic cleavage sites. In some embodiments, the proteolytic cleavage site is located between a polynucleotide encoding a first antigen, and another polynucleotide encoding a second antigen.

In some embodiments, the proteolytic cleavage site is located between a polynucleotide encoding an antigen, and a polynucleotide encoding an enhancer protein. In some embodiments, the proteolytic cleavage site comprises the nucleic acid sequence of SEQ ID NO: 50. In some embodiments, the proteolytic cleavage site is a furin cleavage site. In some embodiments, the expression cassettes disclosed herein comprise a nucleic acid sequence encoding a viral accessory protein, for example ORF3a protein. In some embodiments, the polynucleotide encoding the ORF3 protein has a nucleic acid sequence with at least 70% sequence identity—for instance, at least 75%, at least 80%, at least 85%, at least 90%, at least 95%, at least 96%, at least 97%, at least 98%, at least 99%, at least 99.5%, including any values and subranges that lie there between-to the nucleic acid sequence of SEQ ID NO: 54. In some embodiments, the polynucleotide encoding the ORF3 protein has a nucleic acid sequence of SEQ ID NO: 54. In some embodiments, ORF3 protein has a amino acid sequence with at least 70% sequence identity—for instance, at least 75%, at least 80%, at least 85%, at least 90%, at least 95%, at least 96%, at least 97%, at least 98%, at least 99%, at least 99.5%, including any values and subranges that lie there between—to the amino acid sequence of SEQ ID NO: 53. In some embodiments, the ORF3 protein has an amino acid sequence of SEQ ID NO: 53.

In some embodiments, the vector is selected from the group consisting of CoVEG3-17. In some embodiments, the vector comprises a nucleic acid sequence having at least about 70% identity, at least about 75%, at least about 80%, at least about 85%, at least about 90%, at least about 95%, at least about 96%, at least about 97%, at least about 98%, at least about 99%, at least about 99.5% or about 100% identity to a nucleic acid sequence selected from the group consisting of SEQ ID NO: 35-49. In some embodiments, the vector comprises a nucleic acid sequence having at least 70% (for example, about 75%, about 80%, about 85%, about 90%, about 95%, about 96%, about 97%, about 98%, about 99%, about 99.5%, or about 100%) identity to a nucleic acid sequence selected from the group consisting of SEQ ID NO: 35-49.

The nucleic acid sequence of the expression cassette of CoVEG3-17 and the genetic elements therein are listed in Table 2.

TABLE 2

| | SEQ | |
|---|---|---|
| Name of | ID | |
| sequence | NO: | Sequence |
| CoVEG3 | 35 | ATGGCCGACAGCAACGGCACAATCA |
| Expression | | CCGTGGAAGAGCTGAAGAAACTGCT |
| Cassette | | GGAACAGTGGAACCTGGTCATCGGC |
| | | TTCCTGTTTCTGACCTGGATCTGTC |
| | | TGCTGCAGTTCGCTTATGCCAATCG |
| | | GAACAGATTCCTGTACATCATCAAG |
| | | CTGATCTTCCTGTGGCTGCTGTGGC |
| | | CTGTGACCCTGGCTTGCTTCGTGCT |
| | | GGCCGCTGTGTACCGGATCAACTGG |
| | | ATCACAGGCGGAATCGCCATCGCCA |
| | | TGGCCTGCCTGGTGGGCCTGATGTG |
| | | GCTGAGCTACTTCATCGCTTCTTTC |
| | | AGACTGTTCGCCAGAACCCGGAGCA |
| | | TGTGGTCCTTCAACCCCGAGACAAA |
| | | CATCCTGCTGAACGTGCCTCTGCAC |
| | | GGCACCATCCTGACAAGACCTCTGC |
| | | TCGAGAGCGAGCTGGTGATTGGCGC |
| | | AGTGATTCTGAGAGGCCATCTGAGG |
| | | ATCGCCGGACACCACCTGGGCAGAT |
| | | GCGACATCAAGGACCTTCCAAAGGA |
| | | AATCACCGTTGCCACCAGCCGGACC |
| | | CTGTCCTACTACAAACTGGGCGCCA |
| | | GCCAAAGAGTGGCCGGCGATAGCGG |
| | | CTTTGCCGCCTACAGCAGATACCGC |
| | | ATCGGAAATTACAAGCTCAACACCG |
| | | ACCACAGCAGCTCTTCTGATAACAT |
| | | CGCCCTGCTGGTGCAGtaatccccc |
| | | ccccctaacgttactggccgaagcc |
| | | gcttggaataaggccggtgtgcgtt |
| | | tgtctatatgttattttccaccata |
| | | ttgccgtcttttggcaatgtgaggg |
| | | cccggaaacctggccctgtcttctt |
| | | gacgagcattcctaggggtctttcc |
| | | cctctcgccaaaggaatgcaaggtc |
| | | tgttgaatgtcgtgaaggaagcagt |
| | | tcctctggaagcttcttgaagacaa |
| | | acaacgtctgtagcgaccctttgca |
| | | ggcagcggaaccccccacctggcga |
| | | caggtgcctctgcggccaaaagcca |
| | | cgtgtataagatacacctgcaaagg |
| | | cggcacaaccccagtgccacgttgt |
| | | gagttggatagttgtggaaagagtc |
| | | aaatggctctcctcaagcgtattca |
| | | acaaggggctgaaggatgcccagaa |
| | | ggtaccccattgtatgggatctgat |
| | | ctggggcctcggtgcacatgcttta |
| | | catgtgtttagtcgaggttaaaaaa |
| | | acgtctaggccccccgaaccacggg |
| | | gacgtggttttcctttgaaaaacac |
| | | gatgataatatggccacaaccatgg |
| | | aacaagagacttgcgcgcactctct |
| | | cacttttgaggaatgcccaaaatgc |
| | | tctgctctacaataccgtaatggat |
| | | tttacctgctaaagtatgatgaaga |
| | | atggtacccagaggagttattgact |
| | | gatggagaggatgatgtctttgatc |
| | | ccgaattagacatggaagtcgtttt |
| | | cgagttacagggaagcggagctact |
| | | aacttcagcctgctgaagcaggctg |
| | | gagatgtggaggagaaccctggacc |
| | | tATGTTCGTGTTCCTGGTGCTGCTG |
| | | CCTCTGGTCAGCTCCCAGTGTGTGA |
| | | ACCTGACCACCAGAACCCAGCTGCC |
| | | ACCTGCTTATACAAACTCCTTCACT |
| | | CGGGGGGTATACTACCCCGACAAGG |
| | | TGTTCAGATCTAGCGTGCTGCATTC |
| | | TACACAAGACCTGTTCCTGCCCTTC |
| | | TTCAGCAACGTGACCTGGTTCCACG |
| | | CCATCCACGTGTCTGGAACCAACGG |
| | | AACCAAGAGATTCGACAACCCCGTG |
| | | CTGCCTTTCAACGACGGCGTGTACT |
| | | TCGCCAGCACCGAGAAGTCCAACAT |
| | | CATCAGAGGATGGATTTTCGGCACC |
| | | ACACTGGACAGCAAAACCCAGAGCC |
| | | TGCTGATCGTGAACAACGCCACCAA |
| | | CGTGGTGATCAAGGTGTGCGAGTTC |
| | | CAGTTCTGCAATGATCCCTTCCTGG |
| | | GCGTGTACTACCACAAGAACAACAA |
| | | GTCTTGGATGGAAAGCGAGTTCAGA |
| | | GTGTATTCCAGCGCCAACAATTGCA |
| | | CCTTCGAGTACGTGAGCCAACCCTT |
| | | TCTGATGGACCTTGAAGGCAAGCAG |
| | | GGCAACTTCAAAAATCTGCGAGAAT |
| | | TTGTGTTCAAGAACATCGACGGATA |
| | | CTTCAAGATCTACTCTAAGCACACG |
| | | CCAATCAACCTGGTGAGAGATCTGC |
| | | CCCAGGGCTTTAGCGCTTTGGAACC |
| | | TCTGGTGGACCTGCCTATCGGAATC |
| | | AACATCACCAGATTTCAAACTCTCC |
| | | TGGCCCTGCACAGATCTTATCTGAC |
| | | CCCTGGGGACAGTAGTAGCGGCTGG |
| | | ACAGCCGGCGCCGCCGCCTACTACG |
| | | TGGGATACCTGCAGCCTAGAACATT |
| | | CCTGCTGAAGTACAATGAGAACGGA |
| | | ACAATCACAGACGCCGTGGACTGCG |
| | | CCCTGGATCCTTTGAGCGAGACAAA |
| | | GTGCACCCTGAAGTCGTTCACCGTC |
| | | GAAAAAGGCATCTACCAGACCAGCA |
| | | ACTTCCGCGTGCAGCCTACGGAATC |
| | | TATCGTGCGGTTCCCCAACATCACC |
| | | AACCTGTGCCCTTTCGGCGAGGTGT |
| | | TTAACGCTACAAGGTTCGCCAGCGT |
| | | GTATGCCTGGAACAGAAAGAGAATC |
| | | AGCAATTGCGTGGCCGATTATAGCG |
| | | TTCTGTACAACAGCGCTTCCTTCAG |
| | | CACCTTCAAGTGCTACGGCGTGTCT |
| | | CCAACCAAGCTGAACGACCTCTGCT |
| | | TCACCAATGTCTACGCTGACTCTTT |
| | | CGTGATTAGAGGCGATGAGGTTAGA |
| | | CAGATCGCACCTGGCCAGACCGGCA |
| | | AAATCGCTGACTACAACTACAAGCT |
| | | GCCTGATGACTTCACAGGCTGTGTC |
| | | ATTGCCTGGAACTCAAATAACCTGG |
| | | ACTCTAAAGTGGGCGGCAACTACAA |
| | | CTACCTGTACCGGCTGTTCCGGAAG |
| | | AGCAATCTGAAACCTTTTGAGCGGG |
| | | ACATCTCTACAGAGATCTACCAGGC |
| | | CGGCAGCACACCCTGCAACGGCGTT |
| | | GAGGGCTTCAACTGCTACTTCCCTC |
| | | TGCAGAGCTACGGCTTTCAGCCAAC |
| | | AAATGGAGTGGGCTACCAGCCGTAC |
| | | AGAGTGGTGGTGCTGAGCTTCGAAC |
| | | TGCTGCATGCCCCAGCCACAGTGTG |
| | | TGGACCTAAGAAGTCTACCAACCTG |
| | | GTGAAGAACAAGTGCGTGAACTTTA |
| | | ACTTTAACGGCCTGACCGGCACAGG |
| | | CGTGCTGACCGAATCCAACAAAAAG |
| | | TTCCTGCCCTTCCAACAGTTCGGCA |
| | | GAGACATCGCCGATACAACCGATGC |
| | | CGTGCGGGACCCCCAGACCTTAGAA |
| | | ATCCTAGATATCACCCCGTGCAGCT |
| | | TCGGCGGAGTCTCTGTTATTACTCC |
| | | TGGCACCAACACCAGCAACCAAGTG |
| | | GCTGTTCTGTACCAAggcGTGAACT |
| | | GCACCGAAGTGCCTGTGGCTATCCA |
| | | CGCCGATCAGCTGACCCCAACCTGG |
| | | CGGGTGTATAGCACCGGCTCTAACG |
| | | TGTTCCAGACCCGGGCTGGCTGCCT |
| | | GATCGGCGCCGAACACGTCAACAAC |
| | | TCCTATGAATGTGACATCCCCATCG |
| | | GGGCTGGCATCTGCGCCAGTTACCA |
| | | GACACAGACAAATAGCCCTAGACGG |
| | | GCCAGAAGCGTGGCCTCCCAGAGTA |
| | | TCATTGCCTACACCATGAGCCTGGG |
| | | CGCCGAGAACAGCGTGGCCTATTCT |
| | | AACAATAGCATCGCAATCCCTACCA |
| | | ACTTTACCATCTCTGTGACAACCGA |
| | | GATCCTGCCTGTGAGCATGACCAAA |
| | | ACCAGCGTGGACTGCACGATGTACA |
| | | TCTGTGGCGACAGCACAGAATGCAG |
| | | TAATCTGTTGCTGCAGTACGGCAGC |
| | | TTTTGCACCCAGTTGAATAGAGCCC |
| | | TGACCGGAATCGCCGTAGAGCAGGA |
| | | CAAAAATACCCAGGAGGTGTTCGCC |
| | | CAGGTGAAACAGATCTACAAGACAC |
| | | CTCCCATTAAGGACTTCGGAGGTTT |
| | | TAACTTCAGCCAGATCCTGCCCGAC |
| | | CCTTCCAAGCCTAGCAAACGCTCCT |
| | | TCATCGAGGACCTGCTCTTCAACAA |
| | | GGTGACACTGGCTGATGCCGGCTTC |
| | | ATCAAGCAGTACGGAGATTGTCTGG |
| | | GAGACATCGCCGCTAGAGATCTGAT |
| | | CTGCGCCCAAAAGTTCAACGGCCTG |
| | | ACCGTGCTGCCTCCTCTGCTTACAG |
| | | ACGAGATGATCGCCCAGTACACCAG |
| | | CGCCCTGCTGGCTGGCACCATCACA |
| | | AGCGGCTGGACCTTCGGAGCCGGAG |
| | | CCGCTCTGCAAATCCCCTTTGCCAT |
| | | GCAGATGGCCTACCGGTTCAACGGC |
| | | ATCGGCGTGACACAGAATGTGCTGT |
| | | ACGAGAACCAGAAGCTGATCGCTAA |
| | | CCAGTTTAACAGCGCTATCGGCAAG |
| | | ATCCAGGACTCGCTGAGTAGCACCG |
| | | CCTCTGCCCTGGGCAAGCTGCAGGA |
| | | CGTCGTGAACCAGAACGCCCAAGCC |
| | | CTGAACACACTGGTGAAACAGCTGA |
| | | GCAGCAACTTCGGCGCCATCAGCTC |
| | | TGTGCTGAACGATATCCTGAGCAGA |
| | | CTGGACAAGGTGGAAGCCGAGGTCC |
| | | AGATCGACAGACTGATCACAGGAAG |
| | | ACTGCAGAGCCTGCAAACGTACGTG |
| | | ACACAGCAGCTGATCCGGGCAGCCG |
| | | AAATCCGGGCCAGCGCCAATCTGGC |
| | | CGCTACCAAGATGAGCGAGTGCGTG |
| | | TTAGGCCAGAGCAAGCGGGTGGATT |
| | | TCTGCGGTAAGGGATACCACCTGAT |
| | | GAGCTTTCCCCAGAGCGCTCCTCAC |
| | | GGCGTGGTGTTTCTGCACGTGACCT |
| | | ACGTTCCTGCCCAGGAAAAGAACTT |
| | | CACCACCGCCCCTGCTATCTGCCAC |
| | | GATGGCAAGGCCCACTTCCCTAGAG |
| | | AGGGCGTTTTCGTGTCTAACGGCAC |
| | | ACACTGGTTTGTGACCCAGAGAAAC |
| | | TTCTACGAGCCTCAGATCATCACCA |
| | | CAGACAACACCTTTGTGAGCGGCAA |
| | | TTGCGACGTGGTGATCGGAATTGTT |
| | | AATAATACCGTGTACGACCCTCTGC |
| | | AGCCTGAGCTCGACAGCTTCAAGGA |
| | | AGAGCTGGACAAGTACTTCAAGAAC |
| | | CACACCTCCCCAGATGTGGACCTGG |
| | | GCGATATTTCAGGCATCAACGCCTC |
| | | CGTCGTGAATATCCAGAAGGAGATC |
| | | GACCGGCTCAACGAGGTGGCCAAGA |
| | | ACCTTAACGAGAGCCTGATCGACCT |
| | | GCAGGAACTGGGCAAATATGAGCAG |
| | | TACATCAAGTGGCCTTGGTACATCT |
| | | GGCTGGGCTTTATCGCAGGCCTGAT |
| | | CGCTATCGTGATGGTGACCATTATG |
| | | CTGTGTTGTATGACCAGCTGTTGTA |
| | | GTTGTCTGAAGGGCTGCTGTTCTTG |
| | | CGGCAGCTGCTGCAAGTTCGACGAA |
| | | GACGACTCAGAGCCCGTGCTGAAAG |
| | | GCGTGAAGCTGCACTACACCtaacc |
| | | cccccccctaacgttactggccgaa |
| | | gccgcttggaataaggccggtgtgc |
| | | gtttgtctatatgttattttccacc |
| | | atattgccgtcttttggcaatgtga |
| | | gggcccggaaacctggccctgtctt |
| | | cttgacgagcattcctaggggtctt |
| | | tcccctctcgccaaaggaatgcaag |
| | | gtctgttgaatgtcgtgaaggaagc |
| | | agttcctctggaagcttcttgaaga |
| | | caaacaacgtctgtagcgacccttt |
| | | gcaggcagcggaaccccccacctgg |
| | | cgacaggtgcctctgcggccaaaag |
| | | ccacgtgtataagatacacctgcaa |
| | | aggcggcacaaccccagtgccacgt |
| | | tgtgagttggatagttgtggaaaga |
| | | gtcaaatggctctcctcaagcgtat |
| | | tcaacaaggggctgaaggatgccca |
| | | gaaggtaccccattgtatgggatct |
| | | gatctggggcctcggtgcacatgct |
| | | ttacatgtgtttagtcgaggttaaa |
| | | aaaacgtctaggccccccgaaccac |
| | | ggggacgtggttttcctttgaaaaa |
| | | cacgatgataatatggccacaacca |
| | | tggaacaagagacttgcgcgcactc |
| | | tctcacttttgaggaatgcccaaaa |
| | | tgctctgctctacaataccgtaatg |
| | | gattttacctgctaaagtatgatga |
| | | agaatggtacccagaggagttattg |
| | | actgatggagaggatgatgtctttg |
| | | atcccgaattagacatggaagtcgt |
| | | tttcgagttacagggaagcggagct |
| | | actaacttcagcctgctgaagcagg |
| | | ctggagatgtggaggagaaccctgg |
| | | acctATGAGCGACAACGGCCCTCAA |
| | | AACCAGAGAAATGCCCCTCGGATCA |
| | | CATTTGGCGGACCTAGCGACAGCAC |
| | | CGGCAGCAACCAGAATGGAGAAAGA |
| | | AGCGGCGCCAGATCCAAGCAGCGGA |
| | | GACCTCAGGGACTGCCCAACAACAC |
| | | CGCTAGCTGGTTCACCGCCCTGACC |
| | | CAACACGGCAAGGAAGATCTGAAGT |
| | | TCCCCAGAGGCCAGGGCGTGCCTAT |
| | | CAACACAAACTCTTCTCCCGACGAC |
| | | CAGATCGGATACTATAGACGGGCCA |
| | | CTCGGAGAATTCGGGGCGGCGACGG |
| | | AAAAATGAAGGACCTTTCTCCAAGA |
| | | TGGTACTTCTACTACCTCGGCACAG |
| | | GCCCTGAGGCCGGCCTGCCTTACGG |
| | | CGCCAACAAGGATGGCATCATCTGG |
| | | GTCGCCACCGAGGGCGCCCTGAACA |
| | | CCCCTAAGGACCACATCGGCACAAG |
| | | AAACCCCGCTAACAACGCCGCAATC |
| | | GTGCTGCAGCTGCCTCAGGGCACCA |
| | | CCCTGCCCAAGGGCTTCTACGCCGA |
| | | GGGCTCTAGAGGTGGCTCCCAGGCT |
| | | TCTAGCCGCTCCTCCAGCCGCAGCA |
| | | GAAACAGCAGCAGGAACAGCACCCC |
| | | CGGCAGCTCCCGGGGCACCAGCCCC |
| | | GCCAGAATGGCCGGAAATGGCGGCG |
| | | ATGCCGCCCTGGCCCTGCTCCTGCT |
| | | GGACAGACTGAATCAGCTGGAAAGC |
| | | AAGATGAGCGGCAAAGGACAGCAGC |
| | | AGCAAGGCCAGACCGTGACCAAGAA |
| | | AAGCGCTGCTGAAGCCTCCAAGAAA |
| | | CCTAGACAAAAGCGGACCGCCACAA |
| | | AGGCCTACAACGTGACCCAAGCCTT |
| | | TGGAAGAAGAGGCCCCGAGCAGACA |
| | | CAGGGCAATTTCGGCGACCAGGAGC |
| | | TGATCCGGCAGGGAACCGACTACAA |
| | | GCACTGGCCTCAGATCGCCCAGTTC |
| | | GCCCCTAGCGCCAGCGCCTTCTTCG |
| | | GCATGAGCAGAATCGGCATGGAAGT |
| | | GACCCCTTCTGGCACCTGGCTGACC |
| | | TACACCGGCGCTATCAAGCTGGACG |
| | | ATAAGGATCCTAACTTCAAGGACCA |
| | | AGTGATCCTGCTGAACAAGCATATC |
| | | GACGCCTATAAGACCTTTCCACCTA |
| | | CAGAGCCTAAGAAAGATAAGAAGAA |
| | | GAAAGCCGACGAGACACAGGCCCTG |
| | | CCTCAGAGACAGAAAAAGCAGCAGA |
| | | CAGTGACACTGCTGCCAGCCGCTGA |
| | | CCTGGATGACTTCAGCAAGCAGCTG |
| | | CAGCAGAGCATGTCTTCTGCTGATA |
| | | GCACCCAGGCCggaagcggagctac |
| | | taacttcagcctgctgaagcaggct |
| | | ggagatgtggaggagaaccctggac |
| | | ctATGTATTCTTTTGTGTCCGAGGA |
| | | AACCGGCACACTGATCGTTAATAGC |
| | | GTGCTGCTCTTCCTGGCCTTCGTGG |
| | | TGTTCCTGCTGGTGACCCTGGCTAT |
| | | CCTGACCGCCCTGAGACTGTGTGCC |
| | | TACTGCTGCAACATCGTGAACGTGT |
| | | CTCTGGTCAAGCCTAGCTTCTACGT |
| | | GTACAGCCGGGTGAAGAACCTGAAC |
| | | AGCAGCAGAGTGCCCGACCTGCTGG |
| | | TGTGA |
| SARS | 66 | ATGGCCGACAGCAACGGCACAATCA |
| CoV2 M | | CCGTGGAAGAGCTGAAGAAACTGCT |
| gene | | GGAACAGTGGAACCTGGTCATCGGC |
| | | TTCCTGTTTCTGACCTGGATCTGTC |
| | | TGCTGCAGTTCGCTTATGCCAATCG |
| | | GAACAGATTCCTGTACATCATCAAG |
| | | CTGATCTTCCTGTGGCTGCTGTGGC |
| | | CTGTGACCCTGGCTTGCTTCGTGCT |
| | | GGCCGCTGTGTACCGGATCAACTGG |
| | | ATCACAGGCGGAATCGCCATCGCCA |
| | | TGGCCTGCCTGGTGGGCCTGATGTG |
| | | GCTGAGCTACTTCATCGCTTCTTTC |
| | | AGACTGTTCGCCAGAACCCGGAGCA |
| | | TGTGGTCCTTCAACCCCGAGACAAA |
| | | CATCCTGCTGAACGTGCCTCTGCAC |
| | | GGCACCATCCTGACAAGACCTCTGC |
| | | TCGAGAGCGAGCTGGTGATTGGCGC |
| | | AGTGATTCTGAGAGGCCATCTGAGG |
| | | ATCGCCGGACACCACCTGGGCAGAT |
| | | GCGACATCAAGGACCTTCCAAAGGA |
| | | AATCACCGTTGCCACCAGCCGGACC |
| | | CTGTCCTACTACAAACTGGGCGCCA |
| | | GCCAAAGAGTGGCCGGCGATAGCGG |
| | | CTTTGCCGCCTACAGCAGATACCGC |
| | | ATCGGAAATTACAAGCTCAACACCG |
| | | ACCACAGCAGCTCTTCTGATAACAT |
| | | CGCCCTGCTGGTGCAGtaa |
| IRES | 67 | tcccccccccctaacgttactggcc |
| encoding | | gaagccgcttggaataaggccggtg |
| sequence | | tgcgtttgtctatatgttattttcc |
| | | accatattgccgtcttttggcaatg |
| | | tgagggcccggaaacctggccctgt |
| | | cttcttgacgagcattcctaggggt |
| | | ctttcccctctcgccaaaggaatgc |
| | | aaggtctgttgaatgtcgtgaagga |
| | | agcagttcctctggaagcttcttga |
| | | agacaaacaacgtctgtagcgaccc |
| | | tttgcaggcageggaaccccccacc |
| | | tggcgacaggtgcctctgcggccaa |
| | | aagccacgtgtataagatacacctg |
| | | caaaggcggcacaaccccagtgcca |
| | | cgttgtgagttggatagttgtggaa |
| | | agagtcaaatggctctcctcaagcg |
| | | tattcaacaaggggctgaaggatgc |
| | | ccagaaggtaccccattgtatggga |
| | | tctgatctggggcctcggtgcacat |
| | | gctttacatgtgtttagtcgaggtt |
| | | aaaaaaacgtctaggccccccgaac |
| | | cacggggacgtggttttcctttgaa |
| | | aaacacgatgataat |
| L peptide | 68 | atggccacaaccatggaacaagaga |
| from | | cttgcgcgcactctctcacttttga |
| ECMV | | ggaatgcccaaaatgctctgctcta |
| encoding | | caataccgtaatggattttacctgc |
| sequence | | taaagtatgatgaagaatggtaccc |
| | | agaggagttattgactgatggagag |
| | | gatgatgtctttgatcccgaattag |
| | | acatggaagtcgttttcgagttaca |
| | | g |
| P2A | 69 | ggaagcggagctactaacttcagcc |
| skipping | | tgctgaagcaggctggagatgtgga |
| site | | ggagaaccctggacct |
| encoding | | |
| sequence | | |
| SARS | 70 | ATGTTCGTGTTCCTGGTGCTGCTGC |
| CoV2 Spike | | CTCTGGTCAGCTCCCAGTGTGTGAA |
| gene | | CCTGACCACCAGAACCCAGCTGCCA |
| | | CCTGCTTATACAAACTCCTTCACTC |
| | | GGGGGGTATACTACCCCGACAAGGT |
| | | GTTCAGATCTAGCGTGCTGCATTCT |
| | | ACACAAGACCTGTTCCTGCCCTTCT |
| | | TCAGCAACGTGACCTGGTTCCACGC |
| | | CATCCACGTGTCTGGAACCAACGGA |
| | | ACCAAGAGATTCGACAACCCCGTGC |
| | | TGCCTTTCAACGACGGCGTGTACTT |
| | | CGCCAGCACCGAGAAGTCCAACATC |
| | | ATCAGAGGATGGATTTTCGGCACCA |
| | | CACTGGACAGCAAAACCCAGAGCCT |
| | | GCTGATCGTGAACAACGCCACCAAC |
| | | GTGGTGATCAAGGTGTGCGAGTTCC |
| | | AGTTCTGCAATGATCCCTTCCTGGG |
| | | CGTGTACTACCACAAGAACAACAAG |
| | | TCTTGGATGGAAAGCGAGTTCAGAG |
| | | TGTATTCCAGCGCCAACAATTGCAC |
| | | CTTCGAGTACGTGAGCCAACCCTTT |
| | | CTGATGGACCTTGAAGGCAAGCAGG |
| | | GCAACTTCAAAAATCTGCGAGAATT |
| | | TGTGTTCAAGAACATCGACGGATAC |
| | | TTCAAGATCTACTCTAAGCACACGC |
| | | CAATCAACCTGGTGAGAGATCTGCC |
| | | CCAGGGCTTTAGCGCTTTGGAACCT |
| | | CTGGTGGACCTGCCTATCGGAATCA |
| | | ACATCACCAGATTTCAAACTCTCCT |
| | | GGCCCTGCACAGATCTTATCTGACC |
| | | CCTGGGGACAGTAGTAGCGGCTGGA |
| | | CAGCCGGCGCCGCCGCCTACTACGT |
| | | GGGATACCTGCAGCCTAGAACATTC |
| | | CTGCTGAAGTACAATGAGAACGGAA |
| | | CAATCACAGACGCCGTGGACTGCGC |
| | | CCTGGATCCTTTGAGCGAGACAAAG |
| | | TGCACCCTGAAGTCGTTCACCGTCG |
| | | AAAAAGGCATCTACCAGACCAGCAA |
| | | CTTCCGCGTGCAGCCTACGGAATCT |
| | | ATCGTGCGGTTCCCCAACATCACCA |
| | | ACCTGTGCCCTTTCGGCGAGGTGTT |
| | | TAACGCTACAAGGTTCGCCAGCGTG |
| | | TATGCCTGGAACAGAAAGAGAATCA |
| | | GCAATTGCGTGGCCGATTATAGCGT |
| | | TCTGTACAACAGCGCTTCCTTCAGC |
| | | ACCTTCAAGTGCTACGGCGTGTCTC |
| | | CAACCAAGCTGAACGACCTCTGCTT |
| | | CACCAATGTCTACGCTGACTCTTTC |
| | | GTGATTAGAGGCGATGAGGTTAGAC |
| | | AGATCGCACCTGGCCAGACCGGCAA |
| | | AATCGCTGACTACAACTACAAGCTG |
| | | CCTGATGACTTCACAGGCTGTGTCA |
| | | TTGCCTGGAACTCAAATAACCTGGA |
| | | CTCTAAAGTGGGCGGCAACTACAAC |
| | | TACCTGTACCGGCTGTTCCGGAAGA |
| | | GCAATCTGAAACCTTTTGAGCGGGA |
| | | CATCTCTACAGAGATCTACCAGGCC |
| | | GGCAGCACACCCTGCAACGGCGTTG |
| | | AGGGCTTCAACTGCTACTTCCCTCT |
| | | GCAGAGCTACGGCTTTCAGCCAACA |
| | | AATGGAGTGGGCTACCAGCCGTACA |
| | | GAGTGGTGGTGCTGAGCTTCGAACT |
| | | GCTGCATGCCCCAGCCACAGTGTGT |
| | | GGACCTAAGAAGTCTACCAACCTGG |
| | | TGAAGAACAAGTGCGTGAACTTTAA |
| | | CTTTAACGGCCTGACCGGCACAGGC |
| | | GTGCTGACCGAATCCAACAAAAAGT |
| | | TCCTGCCCTTCCAACAGTTCGGCAG |
| | | AGACATCGCCGATACAACCGATGCC |
| | | GTGCGGGACCCCCAGACCTTAGAAA |
| | | TCCTAGATATCACCCCGTGCAGCTT |
| | | CGGCGGAGTCTCTGTTATTACTCCT |
| | | GGCACCAACACCAGCAACCAAGTGG |
| | | CTGTTCTGTACCAAggcGTGAACTG |
| | | CACCGAAGTGCCTGTGGCTATCCAC |
| | | GCCGATCAGCTGACCCCAACCTGGC |
| | | GGGTGTATAGCACCGGCTCTAACGT |
| | | GTTCCAGACCCGGGCTGGCTGCCTG |
| | | ATCGGCGCCGAACACGTCAACAACT |
| | | CCTATGAATGTGACATCCCCATCGG |
| | | GGCTGGCATCTGCGCCAGTTACCAG |
| | | ACACAGACAAATAGCCCTAGACGGG |
| | | CCAGAAGCGTGGCCTCCCAGAGTAT |
| | | CATTGCCTACACCATGAGCCTGGGC |
| | | GCCGAGAACAGCGTGGCCTATTCTA |
| | | ACAATAGCATCGCAATCCCTACCAA |
| | | CTTTACCATCTCTGTGACAACCGAG |
| | | ATCCTGCCTGTGAGCATGACCAAAA |
| | | CCAGCGTGGACTGCACGATGTACAT |
| | | CTGTGGCGACAGCACAGAATGCAGT |
| | | AATCTGTTGCTGCAGTACGGCAGCT |
| | | TTTGCACCCAGTTGAATAGAGCCCT |
| | | GACCGGAATCGCCGTAGAGCAGGAC |
| | | AAAAATACCCAGGAGGTGTTCGCCC |
| | | AGGTGAAACAGATCTACAAGACACC |
| | | TCCCATTAAGGACTTCGGAGGTTTT |
| | | AACTTCAGCCAGATCCTGCCCGACC |
| | | CTTCCAAGCCTAGCAAACGCTCCTT |
| | | CATCGAGGACCTGCTCTTCAACAAG |
| | | GTGACACTGGCTGATGCCGGCTTCA |
| | | TCAAGCAGTACGGAGATTGTCTGGG |
| | | AGACATCGCCGCTAGAGATCTGATC |
| | | TGCGCCCAAAAGTTCAACGGCCTGA |
| | | CCGTGCTGCCTCCTCTGCTTACAGA |
| | | CGAGATGATCGCCCAGTACACCAGC |
| | | GCCCTGCTGGCTGGCACCATCACAA |
| | | GCGGCTGGACCTTCGGAGCCGGAGC |
| | | CGCTCTGCAAATCCCCTTTGCCATG |
| | | CAGATGGCCTACCGGTTCAACGGCA |
| | | TCGGCGTGACACAGAATGTGCTGTA |
| | | CGAGAACCAGAAGCTGATCGCTAAC |
| | | CAGTTTAACAGCGCTATCGGCAAGA |
| | | TCCAGGACTCGCTGAGTAGCACCGC |
| | | CTCTGCCCTGGGCAAGCTGCAGGAC |
| | | GTCGTGAACCAGAACGCCCAAGCCC |
| | | TGAACACACTGGTGAAACAGCTGAG |
| | | CAGCAACTTCGGCGCCATCAGCTCT |
| | | GTGCTGAACGATATCCTGAGCAGAC |
| | | TGGACAAGGTGGAAGCCGAGGTCCA |
| | | GATCGACAGACTGATCACAGGAAGA |
| | | CTGCAGAGCCTGCAAACGTACGTGA |
| | | CACAGCAGCTGATCCGGGCAGCCGA |
| | | AATCCGGGCCAGCGCCAATCTGGCC |
| | | GCTACCAAGATGAGCGAGTGCGTGT |
| | | TAGGCCAGAGCAAGCGGGTGGATTT |
| | | CTGCGGTAAGGGATACCACCTGATG |
| | | AGCTTTCCCCAGAGCGCTCCTCACG |
| | | GCGTGGTGTTTCTGCACGTGACCTA |
| | | CGTTCCTGCCCAGGAAAAGAACTTC |
| | | ACCACCGCCCCTGCTATCTGCCACG |
| | | ATGGCAAGGCCCACTTCCCTAGAGA |
| | | GGGCGTTTTCGTGTCTAACGGCACA |
| | | CACTGGTTTGTGACCCAGAGAAACT |
| | | TCTACGAGCCTCAGATCATCACCAC |
| | | AGACAACACCTTTGTGAGCGGCAAT |
| | | TGCGACGTGGTGATCGGAATTGTTA |
| | | ATAATACCGTGTACGACCCTCTGCA |
| | | GCCTGAGCTCGACAGCTTCAAGGAA |
| | | GAGCTGGACAAGTACTTCAAGAACC |
| | | ACACCTCCCCAGATGTGGACCTGGG |
| | | CGATATTTCAGGCATCAACGCCTCC |
| | | GTCGTGAATATCCAGAAGGAGATCG |
| | | ACCGGCTCAACGAGGTGGCCAAGAA |
| | | CCTTAACGAGAGCCTGATCGACCTG |
| | | CAGGAACTGGGCAAATATGAGCAGT |
| | | ACATCAAGTGGCCTTGGTACATCTG |
| | | GCTGGGCTTTATCGCAGGCCTGATC |
| | | GCTATCGTGATGGTGACCATTATGC |
| | | TGTGTTGTATGACCAGCTGTTGTAG |
| | | TTGTCTGAAGGGCTGCTGTTCTTGC |
| | | GGCAGCTGCTGCAAGTTCGACGAAG |
| | | ACGACTCAGAGCCCGTGCTGAAAGG |
| | | CGTGAAGCTGCACTACACCtaa |
| SARS CoV2 | 71 | ATGAGCGACAACGGCCCTCAAAACC |
| N gene | | AGAGAAATGCCCCTCGGATCACATT |
| | | TGGCGGACCTAGCGACAGCACCGGC |
| | | AGCAACCAGAATGGAGAAAGAAGCG |
| | | GCGCCAGATCCAAGCAGCGGAGACC |
| | | TCAGGGACTGCCCAACAACACCGCT |
| | | AGCTGGTTCACCGCCCTGACCCAAC |
| | | ACGGCAAGGAAGATCTGAAGTTCCC |
| | | CAGAGGCCAGGGCGTGCCTATCAAC |
| | | ACAAACTCTTCTCCCGACGACCAGA |
| | | TCGGATACTATAGACGGGCCACTCG |
| | | GAGAATTCGGGGCGGCGACGGAAAA |
| | | ATGAAGGACCTTTCTCCAAGATGGT |
| | | ACTTCTACTACCTCGGCACAGGCCC |
| | | TGAGGCCGGCCTGCCTTACGGCGCC |
| | | AACAAGGATGGCATCATCTGGGTCG |
| | | CCACCGAGGGCGCCCTGAACACCCC |
| | | TAAGGACCACATCGGCACAAGAAAC |
| | | CCCGCTAACAACGCCGCAATCGTGC |
| | | TGCAGCTGCCTCAGGGCACCACCCT |
| | | GCCCAAGGGCTTCTACGCCGAGGGC |
| | | TCTAGAGGTGGCTCCCAGGCTTCTA |
| | | GCCGCTCCTCCAGCCGCAGCAGAAA |
| | | CAGCAGCAGGAACAGCACCCCCGGC |
| | | AGCTCCCGGGGCACCAGCCCCGCCA |
| | | GAATGGCCGGAAATGGCGGCGATGC |
| | | CGCCCTGGCCCTGCTCCTGCTGGAC |
| | | AGACTGAATCAGCTGGAAAGCAAGA |
| | | TGAGCGGCAAAGGACAGCAGCAGCA |
| | | AGGCCAGACCGTGACCAAGAAAAGC |
| | | GCTGCTGAAGCCTCCAAGAAACCTA |
| | | GACAAAAGCGGACCGCCACAAAGGC |
| | | CTACAACGTGACCCAAGCCTTTGGA |
| | | AGAAGAGGCCCCGAGCAGACACAGG |
| | | GCAATTTCGGCGACCAGGAGCTGAT |
| | | CCGGCAGGGAACCGACTACAAGCAC |
| | | TGGCCTCAGATCGCCCAGTTCGCCC |
| | | CTAGCGCCAGCGCCTTCTTCGGCAT |
| | | GAGCAGAATCGGCATGGAAGTGACC |
| | | CCTTCTGGCACCTGGCTGACCTACA |
| | | CCGGCGCTATCAAGCTGGACGATAA |
| | | GGATCCTAACTTCAAGGACCAAGTG |
| | | ATCCTGCTGAACAAGCATATCGACG |
| | | CCTATAAGACCTTTCCACCTACAGA |
| | | GCCTAAGAAAGATAAGAAGAAGAAA |
| | | GCCGACGAGACACAGGCCCTGCCTC |
| | | AGAGACAGAAAAAGCAGCAGACAGT |
| | | GACACTGCTGCCAGCCGCTGACCTG |
| | | GATGACTTCAGCAAGCAGCTGCAGC |
| | | AGAGCATGTCTTCTGCTGATAGCAC |
| | | CCAGGCC |
| SARS CoV2 | 72 | ATGTATTCTTTTGTGTCCGAGGAAA |
| Envelope | | CCGGCACACTGATCGTTAATAGCGT |
| gene | | GCTGCTCTTCCTGGCCTTCGTGGTG |
| | | TTCCTGCTGGTGACCCTGGCTATCC |
| | | TGACCGCCCTGAGACTGTGTGCCTA |
| | | CTGCTGCAACATCGTGAACGTGTCT |
| | | CTGGTCAAGCCTAGCTTCTACGTGT |
| | | ACAGCCGGGTGAAGAACCTGAACAG |
| | | CAGCAGAGTGCCCGACCTGCTGGTG |
| | | TGA |
| CoVEG4 | 36 | ATGGCCGACAGCAACGGCACAATCA |
| expression | | CCGTGGAAGAGCTGAAGAAACTGCT |
| cassette | | GGAACAGTGGAACCTGGTCATCGGC |
| | | TTCCTGTTTCTGACCTGGATCTGTC |
| | | TGCTGCAGTTCGCTTATGCCAATCG |
| | | GAACAGATTCCTGTACATCATCAAG |
| | | CTGATCTTCCTGTGGCTGCTGTGGC |
| | | CTGTGACCCTGGCTTGCTTCGTGCT |
| | | GGCCGCTGTGTACCGGATCAACTGG |
| | | ATCACAGGCGGAATCGCCATCGCCA |
| | | TGGCCTGCCTGGTGGGCCTGATGTG |
| | | GCTGAGCTACTTCATCGCTTCTTTC |
| | | AGACTGTTCGCCAGAACCCGGAGCA |
| | | TGTGGTCCTTCAACCCCGAGACAAA |
| | | CATCCTGCTGAACGTGCCTCTGCAC |
| | | GGCACCATCCTGACAAGACCTCTGC |
| | | TCGAGAGCGAGCTGGTGATTGGCGC |
| | | AGTGATTCTGAGAGGCCATCTGAGG |
| | | ATCGCCGGACACCACCTGGGCAGAT |
| | | GCGACATCAAGGACCTTCCAAAGGA |
| | | AATCACCGTTGCCACCAGCCGGACC |
| | | CTGTCCTACTACAAACTGGGCGCCA |
| | | GCCAAAGAGTGGCCGGCGATAGCGG |
| | | CTTTGCCGCCTACAGCAGATACCGC |
| | | ATCGGAAATTACAAGCTCAACACCG |
| | | ACCACAGCAGCTCTTCTGATAACAT |
| | | CGCCCTGCTGGTGCAGCGAAAACGG |
| | | CGCggaagcggaggaagcggagcta |
| | | ctaacttcagcctgctgaagcaggc |
| | | tggagatgtggaggagaaccctgga |
| | | cctATGTTCGTGTTCCTGGTGCTGC |
| | | TGCCTCTGGTCAGCTCCCAGTGTGT |
| | | GAACCTGACCACCAGAACCCAGCTG |
| | | CCACCTGCTTATACAAACTCCTTCA |
| | | CTCGGGGGGTATACTACCCCGACAA |
| | | GGTGTTCAGATCTAGCGTGCTGCAT |
| | | TCTACACAAGACCTGTTCCTGCCCT |
| | | TCTTCAGCAACGTGACCTGGTTCCA |
| | | CGCCATCCACGTGTCTGGAACCAAC |
| | | GGAACCAAGAGATTCGACAACCCCG |
| | | TGCTGCCTTTCAACGACGGCGTGTA |
| | | CTTCGCCAGCACCGAGAAGTCCAAC |
| | | ATCATCAGAGGATGGATTTTCGGCA |
| | | CCACACTGGACAGCAAAACCCAGAG |
| | | CCTGCTGATCGTGAACAACGCCACC |
| | | AACGTGGTGATCAAGGTGTGCGAGT |
| | | TCCAGTTCTGCAATGATCCCTTCCT |
| | | GGGCGTGTACTACCACAAGAACAAC |
| | | AAGTCTTGGATGGAAAGCGAGTTCA |
| | | GAGTGTATTCCAGCGCCAACAATTG |
| | | CACCTTCGAGTACGTGAGCCAACCC |
| | | TTTCTGATGGACCTTGAAGGCAAGC |
| | | AGGGCAACTTCAAAAATCTGCGAGA |
| | | ATTTGTGTTCAAGAACATCGACGGA |
| | | TACTTCAAGATCTACTCTAAGCACA |
| | | CGCCAATCAACCTGGTGAGAGATCT |
| | | GCCCCAGGGCTTTAGCGCTTTGGAA |
| | | CCTCTGGTGGACCTGCCTATCGGAA |
| | | TCAACATCACCAGATTTCAAACTCT |
| | | CCTGGCCCTGCACAGATCTTATCTG |
| | | ACCCCTGGGGACAGTAGTAGCGGCT |
| | | GGACAGCCGGCGCCGCCGCCTACTA |
| | | CGTGGGATACCTGCAGCCTAGAACA |
| | | TTCCTGCTGAAGTACAATGAGAACG |
| | | GAACAATCACAGACGCCGTGGACTG |
| | | CGCCCTGGATCCTTTGAGCGAGACA |
| | | AAGTGCACCCTGAAGTCGTTCACCG |
| | | TCGAAAAAGGCATCTACCAGACCAG |
| | | CAACTTCCGCGTGCAGCCTACGGAA |
| | | TCTATCGTGCGGTTCCCCAACATCA |
| | | CCAACCTGTGCCCTTTCGGCGAGGT |
| | | GTTTAACGCTACAAGGTTCGCCAGC |
| | | GTGTATGCCTGGAACAGAAAGAGAA |
| | | TCAGCAATTGCGTGGCCGATTATAG |
| | | CGTTCTGTACAACAGCGCTTCCTTC |
| | | AGCACCTTCAAGTGCTACGGCGTGT |
| | | CTCCAACCAAGCTGAACGACCTCTG |
| | | CTTCACCAATGTCTACGCTGACTCT |
| | | TTCGTGATTAGAGGCGATGAGGTTA |
| | | GACAGATCGCACCTGGCCAGACCGG |
| | | CAAAATCGCTGACTACAACTACAAG |
| | | CTGCCTGATGACTTCACAGGCTGTG |
| | | TCATTGCCTGGAACTCAAATAACCT |
| | | GGACTCTAAAGTGGGCGGCAACTAC |
| | | AACTACCTGTACCGGCTGTTCCGGA |
| | | AGAGCAATCTGAAACCTTTTGAGCG |
| | | GGACATCTCTACAGAGATCTACCAG |
| | | GCCGGCAGCACACCCTGCAACGGCG |
| | | TTGAGGGCTTCAACTGCTACTTCCC |
| | | TCTGCAGAGCTACGGCTTTCAGCCA |
| | | ACAAATGGAGTGGGCTACCAGCCGT |
| | | ACAGAGTGGTGGTGCTGAGCTTCGA |
| | | ACTGCTGCATGCCCCAGCCACAGTG |
| | | TGTGGACCTAAGAAGTCTACCAACC |
| | | TGGTGAAGAACAAGTGCGTGAACTT |
| | | TAACTTTAACGGCCTGACCGGCACA |
| | | GGCGTGCTGACCGAATCCAACAAAA |
| | | AGTTCCTGCCCTTCCAACAGTTCGG |
| | | CAGAGACATCGCCGATACAACCGAT |
| | | GCCGTGCGGGACCCCCAGACCTTAG |
| | | AAATCCTAGATATCACCCCGTGCAG |
| | | CTTCGGCGGAGTCTCTGTTATTACT |
| | | CCTGGCACCAACACCAGCAACCAAG |
| | | TGGCTGTTCTGTACCAAggcGTGAA |
| | | CTGCACCGAAGTGCCTGTGGCTATC |
| | | CACGCCGATCAGCTGACCCCAACCT |
| | | GGCGGGTGTATAGCACCGGCTCTAA |
| | | CGTGTTCCAGACCCGGGCTGGCTGC |
| | | CTGATCGGCGCCGAACACGTCAACA |
| | | ACTCCTATGAATGTGACATCCCCAT |
| | | CGGGGCTGGCATCTGCGCCAGTTAC |
| | | CAGACACAGACAAATAGCCCTAGAC |
| | | GGGCCAGAAGCGTGGCCTCCCAGAG |
| | | TATCATTGCCTACACCATGAGCCTG |
| | | GGCGCCGAGAACAGCGTGGCCTATT |
| | | CTAACAATAGCATCGCAATCCCTAC |
| | | CAACTTTACCATCTCTGTGACAACC |
| | | GAGATCCTGCCTGTGAGCATGACCA |
| | | AAACCAGCGTGGACTGCACGATGTA |
| | | CATCTGTGGCGACAGCACAGAATGC |
| | | AGTAATCTGTTGCTGCAGTACGGCA |
| | | GCTTTTGCACCCAGTTGAATAGAGC |
| | | CCTGACCGGAATCGCCGTAGAGCAG |
| | | GACAAAAATACCCAGGAGGTGTTCG |
| | | CCCAGGTGAAACAGATCTACAAGAC |
| | | ACCTCCCATTAAGGACTTCGGAGGT |
| | | TTTAACTTCAGCCAGATCCTGCCCG |
| | | ACCCTTCCAAGCCTAGCAAACGCTC |
| | | CTTCATCGAGGACCTGCTCTTCAAC |
| | | AAGGTGACACTGGCTGATGCCGGCT |
| | | TCATCAAGCAGTACGGAGATTGTCT |
| | | GGGAGACATCGCCGCTAGAGATCTG |
| | | ATCTGCGCCCAAAAGTTCAACGGCC |
| | | TGACCGTGCTGCCTCCTCTGCTTAC |
| | | AGACGAGATGATCGCCCAGTACACC |
| | | AGCGCCCTGCTGGCTGGCACCATCA |
| | | CAAGCGGCTGGACCTTCGGAGCCGG |
| | | AGCCGCTCTGCAAATCCCCTTTGCC |
| | | ATGCAGATGGCCTACCGGTTCAACG |
| | | GCATCGGCGTGACACAGAATGTGCT |
| | | GTACGAGAACCAGAAGCTGATCGCT |
| | | AACCAGTTTAACAGCGCTATCGGCA |
| | | AGATCCAGGACTCGCTGAGTAGCAC |
| | | CGCCTCTGCCCTGGGCAAGCTGCAG |
| | | GACGTCGTGAACCAGAACGCCCAAG |
| | | CCCTGAACACACTGGTGAAACAGCT |
| | | GAGCAGCAACTTCGGCGCCATCAGC |
| | | TCTGTGCTGAACGATATCCTGAGCA |
| | | GACTGGACAAGGTGGAAGCCGAGGT |
| | | CCAGATCGACAGACTGATCACAGGA |
| | | AGACTGCAGAGCCTGCAAACGTACG |
| | | TGACACAGCAGCTGATCCGGGCAGC |
| | | CGAAATCCGGGCCAGCGCCAATCTG |
| | | GCCGCTACCAAGATGAGCGAGTGCG |
| | | TGTTAGGCCAGAGCAAGCGGGTGGA |
| | | TTTCTGCGGTAAGGGATACCACCTG |
| | | ATGAGCTTTCCCCAGAGCGCTCCTC |
| | | ACGGCGTGGTGTTTCTGCACGTGAC |
| | | CTACGTTCCTGCCCAGGAAAAGAAC |
| | | TTCACCACCGCCCCTGCTATCTGCC |
| | | ACGATGGCAAGGCCCACTTCCCTAG |
| | | AGAGGGCGTTTTCGTGTCTAACGGC |
| | | ACACACTGGTTTGTGACCCAGAGAA |
| | | ACTTCTACGAGCCTCAGATCATCAC |
| | | CACAGACAACACCTTTGTGAGCGGC |
| | | AATTGCGACGTGGTGATCGGAATTG |
| | | TTAATAATACCGTGTACGACCCTCT |
| | | GCAGCCTGAGCTCGACAGCTTCAAG |
| | | GAAGAGCTGGACAAGTACTTCAAGA |
| | | ACCACACCTCCCCAGATGTGGACCT |
| | | GGGCGATATTTCAGGCATCAACGCC |
| | | TCCGTCGTGAATATCCAGAAGGAGA |
| | | TCGACCGGCTCAACGAGGTGGCCAA |
| | | GAACCTTAACGAGAGCCTGATCGAC |
| | | CTGCAGGAACTGGGCAAATATGAGC |
| | | AGTACATCAAGTGGCCTTGGTACAT |
| | | CTGGCTGGGCTTTATCGCAGGCCTG |
| | | ATCGCTATCGTGATGGTGACCATTA |
| | | TGCTGTGTTGTATGACCAGCTGTTG |
| | | TAGTTGTCTGAAGGGCTGCTGTTCT |
| | | TGCGGCAGCTGCTGCAAGTTCGACG |
| | | AAGACGACTCAGAGCCCGTGCTGAA |
| | | AGGCGTGAAGCTGCACTACACCCGA |
| | | AAACGGCGCggaagcggaggaagcg |
| | | gagctactaacttcagcctgctgaa |
| | | gcaggctggagatgtggaggagaac |
| | | cctggacctATGTATTCTTTTGTGT |
| | | CCGAGGAAACCGGCACACTGATCGT |
| | | TAATAGCGTGCTGCTCTTCCTGGCC |
| | | TTCGTGGTGTTCCTGCTGGTGACCC |
| | | TGGCTATCCTGACCGCCCTGAGACT |
| | | GTGTGCCTACTGCTGCAACATCGTG |
| | | AACGTGTCTCTGGTCAAGCCTAGCT |
| | | TCTACGTGTACAGCCGGGTGAAGAA |
| | | CCTGAACAGCAGCAGAGTGCCCGAC |
| | | CTGCTGGTGtaatccccccccccta |
| | | acgttactggccgaagccgcttgga |
| | | ataaggccggtgtgcgtttgtctat |
| | | atgttattttccaccatattgccgt |
| | | cttttggcaatgtgagggcccggaa |
| | | acctggccctgtcttcttgacgagc |
| | | attcctaggggtctttcccctctcg |
| | | ccaaaggaatgcaaggtctgttgaa |
| | | tgtcgtgaaggaagcagttcctctg |
| | | gaagcttcttgaagacaaacaacgt |
| | | ctgtagcgaccctttgcaggcagcg |
| | | gaaccccccacctggcgacaggtgc |
| | | ctctgcggccaaaagccacgtgtat |
| | | aagatacacctgcaaaggcggcaca |
| | | accccagtgccacgttgtgagttgg |
| | | atagttgtggaaagagtcaaatggc |
| | | tctcctcaagcgtattcaacaaggg |
| | | gctgaaggatgcccagaaggtaccc |
| | | cattgtatgggatctgatctggggc |
| | | ctcggtgcacatgctttacatgtgt |
| | | ttagtcgaggttaaaaaaacgtcta |
| | | ggccccccgaaccacggggacgtgg |
| | | ttttcctttgaaaaacacgatgata |
| | | atatggccacaaccatggaacaaga |
| | | gacttgcgcgcactctctcactttt |
| | | gaggaatgcccaaaatgctctgctc |
| | | tacaataccgtaatggattttacct |
| | | gctaaagtatgatgaagaatggtac |
| | | ccagaggagttattgactgatggag |
| | | aggatgatgtctttgatcccgaatt |
| | | agacatggaagtcgttttcgagtta |
| | | cagtaa |
| CoVEG5 | 37 | ATGGCCGACAGCAACGGCACAATCA |
| Expression | | CCGTGGAAGAGCTGAAGAAACTGCT |
| cassette | | GGAACAGTGGAACCTGGTCATCGGC |
| | | TTCCTGTTTCTGACCTGGATCTGTC |
| | | TGCTGCAGTTCGCTTATGCCAATCG |
| | | GAACAGATTCCTGTACATCATCAAG |
| | | CTGATCTTCCTGTGGCTGCTGTGGC |
| | | CTGTGACCCTGGCTTGCTTCGTGCT |
| | | GGCCGCTGTGTACCGGATCAACTGG |
| | | ATCACAGGCGGAATCGCCATCGCCA |
| | | TGGCCTGCCTGGTGGGCCTGATGTG |
| | | GCTGAGCTACTTCATCGCTTCTTTC |
| | | AGACTGTTCGCCAGAACCCGGAGCA |
| | | TGTGGTCCTTCAACCCCGAGACAAA |
| | | CATCCTGCTGAACGTGCCTCTGCAC |
| | | GGCACCATCCTGACAAGACCTCTGC |
| | | TCGAGAGCGAGCTGGTGATTGGCGC |
| | | AGTGATTCTGAGAGGCCATCTGAGG |
| | | ATCGCCGGACACCACCTGGGCAGAT |
| | | GCGACATCAAGGACCTTCCAAAGGA |
| | | AATCACCGTTGCCACCAGCCGGACC |
| | | CTGTCCTACTACAAACTGGGCGCCA |
| | | GCCAAAGAGTGGCCGGCGATAGCGG |
| | | CTTTGCCGCCTACAGCAGATACCGC |
| | | ATCGGAAATTACAAGCTCAACACCG |
| | | ACCACAGCAGCTCTTCTGATAACAT |
| | | CGCCCTGCTGGTGCAGCGAAAACGG |
| | | CGCggaagcggaggaagcggagcta |
| | | ctaacttcagcctgctgaagcaggc |
| | | tggagatgtggaggagaaccctgga |
| | | cctATGAGCGACAACGGCCCTCAAA |
| | | ACCAGAGAAATGCCCCTCGGATCAC |
| | | ATTTGGCGGACCTAGCGACAGCACC |
| | | GGCAGCAACCAGAATGGAGAAAGAA |
| | | GCGGCGCCAGATCCAAGCAGCGGAG |
| | | ACCTCAGGGACTGCCCAACAACACC |
| | | GCTAGCTGGTTCACCGCCCTGACCC |
| | | AACACGGCAAGGAAGATCTGAAGTT |
| | | CCCCAGAGGCCAGGGCGTGCCTATC |
| | | AACACAAACTCTTCTCCCGACGACC |
| | | AGATCGGATACTATAGACGGGCCAC |
| | | TCGGAGAATTCGGGGCGGCGACGGA |
| | | AAAATGAAGGACCTTTCTCCAAGAT |
| | | GGTACTTCTACTACCTCGGCACAGG |
| | | CCCTGAGGCCGGCCTGCCTTACGGC |
| | | GCCAACAAGGATGGCATCATCTGGG |
| | | TCGCCACCGAGGGCGCCCTGAACAC |
| | | CCCTAAGGACCACATCGGCACAAGA |
| | | AACCCCGCTAACAACGCCGCAATCG |
| | | TGCTGCAGCTGCCTCAGGGCACCAC |
| | | CCTGCCCAAGGGCTTCTACGCCGAG |
| | | GGCTCTAGAGGTGGCTCCCAGGCTT |
| | | CTAGCCGCTCCTCCAGCCGCAGCAG |
| | | AAACAGCAGCAGGAACAGCACCCCC |
| | | GGCAGCTCCCGGGGCACCAGCCCCG |
| | | CCAGAATGGCCGGAAATGGCGGCGA |
| | | TGCCGCCCTGGCCCTGCTCCTGCTG |
| | | GACAGACTGAATCAGCTGGAAAGCA |
| | | AGATGAGCGGCAAAGGACAGCAGCA |
| | | GCAAGGCCAGACCGTGACCAAGAAA |
| | | AGCGCTGCTGAAGCCTCCAAGAAAC |
| | | CTAGACAAAAGCGGACCGCCACAAA |
| | | GGCCTACAACGTGACCCAAGCCTTT |
| | | GGAAGAAGAGGCCCCGAGCAGACAC |
| | | AGGGCAATTTCGGCGACCAGGAGCT |
| | | GATCCGGCAGGGAACCGACTACAAG |
| | | CACTGGCCTCAGATCGCCCAGTTCG |
| | | CCCCTAGCGCCAGCGCCTTCTTCGG |
| | | CATGAGCAGAATCGGCATGGAAGTG |
| | | ACCCCTTCTGGCACCTGGCTGACCT |
| | | ACACCGGCGCTATCAAGCTGGACGA |
| | | TAAGGATCCTAACTTCAAGGACCAA |
| | | GTGATCCTGCTGAACAAGCATATCG |
| | | ACGCCTATAAGACCTTTCCACCTAC |
| | | AGAGCCTAAGAAAGATAAGAAGAAG |
| | | AAAGCCGACGAGACACAGGCCCTGC |
| | | CTCAGAGACAGAAAAAGCAGCAGAC |
| | | AGTGACACTGCTGCCAGCCGCTGAC |
| | | CTGGATGACTTCAGCAAGCAGCTGC |
| | | AGCAGAGCATGTCTTCTGCTGATAG |
| | | CACCCAGGCCCGAAAACGGCGCgga |
| | | agcggaggaagcggagctactaact |
| | | tcagcctgctgaagcaggctggaga |
| | | tgtggaggagaaccctggacctATG |
| | | TTCGTGTTCCTGGTGCTGCTGCCTC |
| | | TGGTCAGCTCCCAGTGTGTGAACCT |
| | | GACCACCAGAACCCAGCTGCCACCT |
| | | GCTTATACAAACTCCTTCACTCGGG |
| | | GGGTATACTACCCCGACAAGGTGTT |
| | | CAGATCTAGCGTGCTGCATTCTACA |
| | | CAAGACCTGTTCCTGCCCTTCTTCA |
| | | GCAACGTGACCTGGTTCCACGCCAT |
| | | CCACGTGTCTGGAACCAACGGAACC |
| | | AAGAGATTCGACAACCCCGTGCTGC |
| | | CTTTCAACGACGGCGTGTACTTCGC |
| | | CAGCACCGAGAAGTCCAACATCATC |
| | | AGAGGATGGATTTTCGGCACCACAC |
| | | TGGACAGCAAAACCCAGAGCCTGCT |
| | | GATCGTGAACAACGCCACCAACGTG |
| | | GTGATCAAGGTGTGCGAGTTCCAGT |
| | | TCTGCAATGATCCCTTCCTGGGCGT |
| | | GTACTACCACAAGAACAACAAGTCT |
| | | TGGATGGAAAGCGAGTTCAGAGTGT |
| | | ATTCCAGCGCCAACAATTGCACCTT |
| | | CGAGTACGTGAGCCAACCCTTTCTG |
| | | ATGGACCTTGAAGGCAAGCAGGGCA |
| | | ACTTCAAAAATCTGCGAGAATTTGT |
| | | GTTCAAGAACATCGACGGATACTTC |
| | | AAGATCTACTCTAAGCACACGCCAA |
| | | TCAACCTGGTGAGAGATCTGCCCCA |
| | | GGGCTTTAGCGCTTTGGAACCTCTG |
| | | GTGGACCTGCCTATCGGAATCAACA |
| | | TCACCAGATTTCAAACTCTCCTGGC |
| | | CCTGCACAGATCTTATCTGACCCCT |
| | | GGGGACAGTAGTAGCGGCTGGACAG |
| | | CCGGCGCCGCCGCCTACTACGTGGG |
| | | ATACCTGCAGCCTAGAACATTCCTG |
| | | CTGAAGTACAATGAGAACGGAACAA |
| | | TCACAGACGCCGTGGACTGCGCCCT |
| | | GGATCCTTTGAGCGAGACAAAGTGC |
| | | ACCCTGAAGTCGTTCACCGTCGAAA |
| | | AAGGCATCTACCAGACCAGCAACTT |
| | | CCGCGTGCAGCCTACGGAATCTATC |
| | | GTGCGGTTCCCCAACATCACCAACC |
| | | TGTGCCCTTTCGGCGAGGTGTTTAA |
| | | CGCTACAAGGTTCGCCAGCGTGTAT |
| | | GCCTGGAACAGAAAGAGAATCAGCA |
| | | ATTGCGTGGCCGATTATAGCGTTCT |
| | | GTACAACAGCGCTTCCTTCAGCACC |
| | | TTCAAGTGCTACGGCGTGTCTCCAA |
| | | CCAAGCTGAACGACCTCTGCTTCAC |
| | | CAATGTCTACGCTGACTCTTTCGTG |
| | | ATTAGAGGCGATGAGGTTAGACAGA |
| | | TCGCACCTGGCCAGACCGGCAAAAT |
| | | CGCTGACTACAACTACAAGCTGCCT |
| | | GATGACTTCACAGGCTGTGTCATTG |
| | | CCTGGAACTCAAATAACCTGGACTC |
| | | TAAAGTGGGCGGCAACTACAACTAC |
| | | CTGTACCGGCTGTTCCGGAAGAGCA |
| | | ATCTGAAACCTTTTGAGCGGGACAT |
| | | CTCTACAGAGATCTACCAGGCCGGC |
| | | AGCACACCCTGCAACGGCGTTGAGG |
| | | GCTTCAACTGCTACTTCCCTCTGCA |
| | | GAGCTACGGCTTTCAGCCAACAAAT |
| | | GGAGTGGGCTACCAGCCGTACAGAG |
| | | TGGTGGTGCTGAGCTTCGAACTGCT |
| | | GCATGCCCCAGCCACAGTGTGTGGA |
| | | CCTAAGAAGTCTACCAACCTGGTGA |
| | | AGAACAAGTGCGTGAACTTTAACTT |
| | | TAACGGCCTGACCGGCACAGGCGTG |
| | | CTGACCGAATCCAACAAAAAGTTCC |
| | | TGCCCTTCCAACAGTTCGGCAGAGA |
| | | CATCGCCGATACAACCGATGCCGTG |
| | | CGGGACCCCCAGACCTTAGAAATCC |
| | | TAGATATCACCCCGTGCAGCTTCGG |
| | | CGGAGTCTCTGTTATTACTCCTGGC |
| | | ACCAACACCAGCAACCAAGTGGCTG |
| | | TTCTGTACCAAggcGTGAACTGCAC |
| | | CGAAGTGCCTGTGGCTATCCACGCC |
| | | GATCAGCTGACCCCAACCTGGCGGG |
| | | TGTATAGCACCGGCTCTAACGTGTT |
| | | CCAGACCCGGGCTGGCTGCCTGATC |
| | | GGCGCCGAACACGTCAACAACTCCT |
| | | ATGAATGTGACATCCCCATCGGGGC |
| | | TGGCATCTGCGCCAGTTACCAGACA |
| | | CAGACAAATAGCCCTGGCAGCGCCA |
| | | GCAGCGTGGCCTCCCAGAGTATCAT |
| | | TGCCTACACCATGAGCCTGGGCGCC |
| | | GAGAACAGCGTGGCCTATTCTAACA |
| | | ATAGCATCGCAATCCCTACCAACTT |
| | | TACCATCTCTGTGACAACCGAGATC |
| | | CTGCCTGTGAGCATGACCAAAACCA |
| | | GCGTGGACTGCACGATGTACATCTG |
| | | TGGCGACAGCACAGAATGCAGTAAT |
| | | CTGTTGCTGCAGTACGGCAGCTTTT |
| | | GCACCCAGTTGAATAGAGCCCTGAC |
| | | CGGAATCGCCGTAGAGCAGGACAAA |
| | | AATACCCAGGAGGTGTTCGCCCAGG |
| | | TGAAACAGATCTACAAGACACCTCC |
| | | CATTAAGGACTTCGGAGGTTTTAAC |
| | | TTCAGCCAGATCCTGCCCGACCCTT |
| | | CCAAGCCTAGCAAACGCTCCTTCAT |
| | | CGAGGACCTGCTCTTCAACAAGGTG |
| | | ACACTGGCTGATGCCGGCTTCATCA |
| | | AGCAGTACGGAGATTGTCTGGGAGA |
| | | CATCGCCGCTAGAGATCTGATCTGC |
| | | GCCCAAAAGTTCAACGGCCTGACCG |
| | | TGCTGCCTCCTCTGCTTACAGACGA |
| | | GATGATCGCCCAGTACACCAGCGCC |
| | | CTGCTGGCTGGCACCATCACAAGCG |
| | | GCTGGACCTTCGGAGCCGGAGCCGC |
| | | TCTGCAAATCCCCTTTGCCATGCAG |
| | | ATGGCCTACCGGTTCAACGGCATCG |
| | | GCGTGACACAGAATGTGCTGTACGA |
| | | GAACCAGAAGCTGATCGCTAACCAG |
| | | TTTAACAGCGCTATCGGCAAGATCC |
| | | AGGACTCGCTGAGTAGCACCGCCTC |
| | | TGCCCTGGGCAAGCTGCAGGACGTC |
| | | GTGAACCAGAACGCCCAAGCCCTGA |
| | | ACACACTGGTGAAACAGCTGAGCAG |
| | | CAACTTCGGCGCCATCAGCTCTGTG |
| | | CTGAACGATATCCTGAGCAGACTGG |
| | | ACCCTcccGAAGCCGAGGTCCAGAT |
| | | CGACAGACTGATCACAGGAAGACTG |
| | | CAGAGCCTGCAAACGTACGTGACAC |
| | | AGCAGCTGATCCGGGCAGCCGAAAT |
| | | CCGGGCCAGCGCCAATCTGGCCGCT |
| | | ACCAAGATGAGCGAGTGCGTGTTAG |
| | | GCCAGAGCAAGCGGGTGGATTTCTG |
| | | CGGTAAGGGATACCACCTGATGAGC |
| | | TTTCCCCAGAGCGCTCCTCACGGCG |
| | | TGGTGTTTCTGCACGTGACCTACGT |
| | | TCCTGCCCAGGAAAAGAACTTCACC |
| | | ACCGCCCCTGCTATCTGCCACGATG |
| | | GCAAGGCCCACTTCCCTAGAGAGGG |
| | | CGTTTTCGTGTCTAACGGCACACAC |
| | | TGGTTTGTGACCCAGAGAAACTTCT |
| | | ACGAGCCTCAGATCATCACCACAGA |
| | | CAACACCTTTGTGAGCGGCAATTGC |
| | | GACGTGGTGATCGGAATTGTTAATA |
| | | ATACCGTGTACGACCCTCTGCAGCC |
| | | TGAGCTCGACAGCTTCAAGGAAGAG |
| | | CTGGACAAGTACTTCAAGAACCACA |
| | | CCTCCCCAGATGTGGACCTGGGCGA |
| | | TATTTCAGGCATCAACGCCTCCGTC |
| | | GTGAATATCCAGAAGGAGATCGACC |
| | | GGCTCAACGAGGTGGCCAAGAACCT |
| | | TAACGAGAGCCTGATCGACCTGCAG |
| | | GAACTGGGCAAATATGAGCAGTACA |
| | | TCAAGTGGCCTTGGTACATCTGGCT |
| | | GGGCTTTATCGCAGGCCTGATCGCT |
| | | ATCGTGATGGTGACCATTATGCTGT |
| | | GTTGTATGACCAGCTGTTGTAGTTG |
| | | TCTGAAGGGCTGCTGTTCTTGCGGC |
| | | AGCTGCTGCAAGTTCGACGAAGACG |
| | | ACTCAGAGCCCGTGCTGAAAGGCGT |
| | | GAAGCTGCACTACACCCGAAAACGG |
| | | CGCggaagcggaggaagcggagcta |
| | | ctaacttcagcctgctgaagcaggc |
| | | tggagatgtggaggagaaccctgga |
| | | cctATGTATTCTTTTGTGTCCGAGG |
| | | AAACCGGCACACTGATCGTTAATAG |
| | | CGTGCTGCTCTTCCTGGCCTTCGTG |
| | | GTGTTCCTGCTGGTGACCCTGGCTA |
| | | TCCTGACCGCCCTGAGACTGTGTGC |
| | | CTACTGCTGCAACATCGTGAACGTG |
| | | TCTCTGGTCAAGCCTAGCTTCTACG |
| | | TGTACAGCCGGGTGAAGAACCTGAA |
| | | CAGCAGCAGAGTGCCCGACCTGCTG |
| | | GTGtaatcccccccccctaacgtta |
| | | ctggccgaagccgcttggaataagg |
| | | ccggtgtgcgtttgtctatatgtta |
| | | ttttccaccatattgccgtcttttg |
| | | gcaatgtgagggcccggaaacctgg |
| | | ccctgtcttcttgacgagcattcct |
| | | aggggtctttcccctctcgccaaag |
| | | gaatgcaaggtctgttgaatgtcgt |
| | | gaaggaagcagttcctctggaagct |
| | | tcttgaagacaaacaacgtctgtag |
| | | cgaccctttgcaggcagcggaaccc |
| | | cccacctggcgacaggtgcctctgc |
| | | ggccaaaagccacgtgtataagata |
| | | cacctgcaaaggcggcacaacccca |
| | | gtgccacgttgtgagttggatagtt |
| | | gtggaaagagtcaaatggctctcct |
| | | caagcgtattcaacaaggggctgaa |
| | | ggatgcccagaaggtaccccattgt |
| | | atgggatctgatctggggcctcggt |
| | | gcacatgctttacatgtgtttagtc |
| | | gaggttaaaaaaacgtctaggcccc |
| | | ccgaaccacggggacgtggttttcc |
| | | tttgaaaaacacgatgataatatgg |
| | | ccacaaccatggaacaagagacttg |
| | | cgcgcactctctcacttttgaggaa |
| | | tgcccaaaatgctctgctctacaat |
| | | accgtaatggattttacctgctaaa |
| | | gtatgatgaagaatggtacccagag |
| | | gagttattgactgatggagaggatg |
| | | atgtctttgatcccgaattagacat |
| | | ggaagtcgttttcgagttacagtaa |
| CoVEG6 | 38 | ATGTTCGTGTTCCTGGTGCTGCTGC |
| expression | | CTCTGGTCAGCTCCCAGTGTGTGAA |
| cassette | | CCTGACCACCAGAACCCAGCTGCCA |
| | | CCTGCTTATACAAACTCCTTCACTC |
| | | GGGGGGTATACTACCCCGACAAGGT |
| | | GTTCAGATCTAGCGTGCTGCATTCT |
| | | ACACAAGACCTGTTCCTGCCCTTCT |
| | | TCAGCAACGTGACCTGGTTCCACGC |
| | | CATCCACGTGTCTGGAACCAACGGA |
| | | ACCAAGAGATTCGACAACCCCGTGC |
| | | TGCCTTTCAACGACGGCGTGTACTT |
| | | CGCCAGCACCGAGAAGTCCAACATC |
| | | ATCAGAGGATGGATTTTCGGCACCA |
| | | CACTGGACAGCAAAACCCAGAGCCT |
| | | GCTGATCGTGAACAACGCCACCAAC |
| | | GTGGTGATCAAGGTGTGCGAGTTCC |
| | | AGTTCTGCAATGATCCCTTCCTGGG |
| | | CGTGTACTACCACAAGAACAACAAG |
| | | TCTTGGATGGAAAGCGAGTTCAGAG |
| | | TGTATTCCAGCGCCAACAATTGCAC |
| | | CTTCGAGTACGTGAGCCAACCCTTT |
| | | CTGATGGACCTTGAAGGCAAGCAGG |
| | | GCAACTTCAAAAATCTGCGAGAATT |
| | | TGTGTTCAAGAACATCGACGGATAC |
| | | TTCAAGATCTACTCTAAGCACACGC |
| | | CAATCAACCTGGTGAGAGATCTGCC |
| | | CCAGGGCTTTAGCGCTTTGGAACCT |
| | | CTGGTGGACCTGCCTATCGGAATCA |
| | | ACATCACCAGATTTCAAACTCTCCT |
| | | GGCCCTGCACAGATCTTATCTGACC |
| | | CCTGGGGACAGTAGTAGCGGCTGGA |
| | | CAGCCGGCGCCGCCGCCTACTACGT |
| | | GGGATACCTGCAGCCTAGAACATTC |
| | | CTGCTGAAGTACAATGAGAACGGAA |
| | | CAATCACAGACGCCGTGGACTGCGC |
| | | CCTGGATCCTTTGAGCGAGACAAAG |
| | | TGCACCCTGAAGTCGTTCACCGTCG |
| | | AAAAAGGCATCTACCAGACCAGCAA |
| | | CTTCCGCGTGCAGCCTACGGAATCT |
| | | ATCGTGCGGTTCCCCAACATCACCA |
| | | ACCTGTGCCCTTTCGGCGAGGTGTT |
| | | TAACGCTACAAGGTTCGCCAGCGTG |
| | | TATGCCTGGAACAGAAAGAGAATCA |
| | | GCAATTGCGTGGCCGATTATAGCGT |
| | | TCTGTACAACAGCGCTTCCTTCAGC |
| | | ACCTTCAAGTGCTACGGCGTGTCTC |
| | | CAACCAAGCTGAACGACCTCTGCTT |
| | | CACCAATGTCTACGCTGACTCTTTC |
| | | GTGATTAGAGGCGATGAGGTTAGAC |
| | | AGATCGCACCTGGCCAGACCGGCAA |
| | | AATCGCTGACTACAACTACAAGCTG |
| | | CCTGATGACTTCACAGGCTGTGTCA |
| | | TTGCCTGGAACTCAAATAACCTGGA |
| | | CTCTAAAGTGGGCGGCAACTACAAC |
| | | TACCTGTACCGGCTGTTCCGGAAGA |
| | | GCAATCTGAAACCTTTTGAGCGGGA |
| | | CATCTCTACAGAGATCTACCAGGCC |
| | | GGCAGCACACCCTGCAACGGCGTTG |
| | | AGGGCTTCAACTGCTACTTCCCTCT |
| | | GCAGAGCTACGGCTTTCAGCCAACA |
| | | AATGGAGTGGGCTACCAGCCGTACA |
| | | GAGTGGTGGTGCTGAGCTTCGAACT |
| | | GCTGCATGCCCCAGCCACAGTGTGT |
| | | GGACCTAAGAAGTCTACCAACCTGG |
| | | TGAAGAACAAGTGCGTGAACTTTAA |
| | | CTTTAACGGCCTGACCGGCACAGGC |
| | | GTGCTGACCGAATCCAACAAAAAGT |
| | | TCCTGCCCTTCCAACAGTTCGGCAG |
| | | AGACATCGCCGATACAACCGATGCC |
| | | GTGCGGGACCCCCAGACCTTAGAAA |
| | | TCCTAGATATCACCCCGTGCAGCTT |
| | | CGGCGGAGTCTCTGTTATTACTCCT |
| | | GGCACCAACACCAGCAACCAAGTGG |
| | | CTGTTCTGTACCAAggcGTGAACTG |
| | | CACCGAAGTGCCTGTGGCTATCCAC |
| | | GCCGATCAGCTGACCCCAACCTGGC |
| | | GGGTGTATAGCACCGGCTCTAACGT |
| | | GTTCCAGACCCGGGCTGGCTGCCTG |
| | | ATCGGCGCCGAACACGTCAACAACT |
| | | CCTATGAATGTGACATCCCCATCGG |
| | | GGCTGGCATCTGCGCCAGTTACCAG |
| | | ACACAGACAAATAGCCCTAGACGGG |
| | | CCAGAAGCGTGGCCTCCCAGAGTAT |
| | | CATTGCCTACACCATGAGCCTGGGC |
| | | GCCGAGAACAGCGTGGCCTATTCTA |
| | | ACAATAGCATCGCAATCCCTACCAA |
| | | CTTTACCATCTCTGTGACAACCGAG |
| | | ATCCTGCCTGTGAGCATGACCAAAA |
| | | CCAGCGTGGACTGCACGATGTACAT |
| | | CTGTGGCGACAGCACAGAATGCAGT |
| | | AATCTGTTGCTGCAGTACGGCAGCT |
| | | TTTGCACCCAGTTGAATAGAGCCCT |
| | | GACCGGAATCGCCGTAGAGCAGGAC |
| | | AAAAATACCCAGGAGGTGTTCGCCC |
| | | AGGTGAAACAGATCTACAAGACACC |
| | | TCCCATTAAGGACTTCGGAGGTTTT |
| | | AACTTCAGCCAGATCCTGCCCGACC |
| | | CTTCCAAGCCTAGCAAACGCTCCTT |
| | | CATCGAGGACCTGCTCTTCAACAAG |
| | | GTGACACTGGCTGATGCCGGCTTCA |
| | | TCAAGCAGTACGGAGATTGTCTGGG |
| | | AGACATCGCCGCTAGAGATCTGATC |
| | | TGCGCCCAAAAGTTCAACGGCCTGA |
| | | CCGTGCTGCCTCCTCTGCTTACAGA |
| | | CGAGATGATCGCCCAGTACACCAGC |
| | | GCCCTGCTGGCTGGCACCATCACAA |
| | | GCGGCTGGACCTTCGGAGCCGGAGC |
| | | CGCTCTGCAAATCCCCTTTGCCATG |
| | | CAGATGGCCTACCGGTTCAACGGCA |
| | | TCGGCGTGACACAGAATGTGCTGTA |
| | | CGAGAACCAGAAGCTGATCGCTAAC |
| | | CAGTTTAACAGCGCTATCGGCAAGA |
| | | TCCAGGACTCGCTGAGTAGCACCGC |
| | | CTCTGCCCTGGGCAAGCTGCAGGAC |
| | | GTCGTGAACCAGAACGCCCAAGCCC |
| | | TGAACACACTGGTGAAACAGCTGAG |
| | | CAGCAACTTCGGCGCCATCAGCTCT |
| | | GTGCTGAACGATATCCTGAGCAGAC |
| | | TGGACAAGGTGGAAGCCGAGGTCCA |
| | | GATCGACAGACTGATCACAGGAAGA |
| | | CTGCAGAGCCTGCAAACGTACGTGA |
| | | CACAGCAGCTGATCCGGGCAGCCGA |
| | | AATCCGGGCCAGCGCCAATCTGGCC |
| | | GCTACCAAGATGAGCGAGTGCGTGT |
| | | TAGGCCAGAGCAAGCGGGTGGATTT |
| | | CTGCGGTAAGGGATACCACCTGATG |
| | | AGCTTTCCCCAGAGCGCTCCTCACG |
| | | GCGTGGTGTTTCTGCACGTGACCTA |
| | | CGTTCCTGCCCAGGAAAAGAACTTC |
| | | ACCACCGCCCCTGCTATCTGCCACG |
| | | ATGGCAAGGCCCACTTCCCTAGAGA |
| | | GGGCGTTTTCGTGTCTAACGGCACA |
| | | CACTGGTTTGTGACCCAGAGAAACT |
| | | TCTACGAGCCTCAGATCATCACCAC |
| | | AGACAACACCTTTGTGAGCGGCAAT |
| | | TGCGACGTGGTGATCGGAATTGTTA |
| | | ATAATACCGTGTACGACCCTCTGCA |
| | | GCCTGAGCTCGACAGCTTCAAGGAA |
| | | GAGCTGGACAAGTACTTCAAGAACC |
| | | ACACCTCCCCAGATGTGGACCTGGG |
| | | CGATATTTCAGGCATCAACGCCTCC |
| | | GTCGTGAATATCCAGAAGGAGATCG |
| | | ACCGGCTCAACGAGGTGGCCAAGAA |
| | | CCTTAACGAGAGCCTGATCGACCTG |
| | | CAGGAACTGGGCAAATATGAGCAGT |
| | | ACATCAAGTGGCCTTGGTACATCTG |
| | | GCTGGGCTTTATCGCAGGCCTGATC |
| | | GCTATCGTGATGGTGACCATTATGC |
| | | TGTGTTGTATGACCAGCTGTTGTAG |
| | | TTGTCTGAAGGGCTGCTGTTCTTGC |
| | | GGCAGCTGCTGCAAGTTCGACGAAG |
| | | ACGACTCAGAGCCCGTGCTGAAAGG |
| | | CGTGAAGCTGCACTACACCtaatcc |
| | | cccccccctaacgttactggccgaa |
| | | gccgcttggaataaggccggtgtgc |
| | | gtttgtctatatgttattttccacc |
| | | atattgccgtcttttggcaatgtga |
| | | gggcccggaaacctggccctgtctt |
| | | cttgacgagcattcctaggggtctt |
| | | tcccctctcgccaaaggaatgcaag |
| | | gtctgttgaatgtcgtgaaggaagc |
| | | agttcctctggaagcttcttgaaga |
| | | caaacaacgtctgtagcgacccttt |
| | | gcaggcagcggaaccccccacctgg |
| | | cgacaggtgcctctgcggccaaaag |
| | | ccacgtgtataagatacacctgcaa |
| | | aggcggcacaaccccagtgccacgt |
| | | tgtgagttggatagttgtggaaaga |
| | | gtcaaatggctctcctcaagcgtat |
| | | tcaacaaggggctgaaggatgccca |
| | | gaaggtaccccattgtatgggatct |
| | | gatctggggcctcggtgcacatgct |
| | | ttacatgtgtttagtcgaggttaaa |
| | | aaaacgtctaggccccccgaaccac |
| | | ggggacgtggttttcctttgaaaaa |
| | | cacgatgataatatggccacaacca |
| | | tggaacaagagacttgcgcgcactc |
| | | tctcacttttgaggaatgcccaaaa |
| | | tgctctgctctacaataccgtaatg |
| | | gattttacctgctaaagtatgatga |
| | | agaatggtacccagaggagttattg |
| | | actgatggagaggatgatgtctttg |
| | | atcccgaattagacatggaagtcgt |
| | | tttcgagttacagggaagcggagct |
| | | actaacttcagcctgctgaagcagg |
| | | ctggagatgtggaggagaaccctgg |
| | | acctATGGCCGACAGCAACGGCACA |
| | | ATCACCGTGGAAGAGCTGAAGAAAC |
| | | TGCTGGAACAGTGGAACCTGGTCAT |
| | | CGGCTTCCTGTTTCTGACCTGGATC |
| | | TGTCTGCTGCAGTTCGCTTATGCCA |
| | | ATCGGAACAGATTCCTGTACATCAT |
| | | CAAGCTGATCTTCCTGTGGCTGCTG |
| | | TGGCCTGTGACCCTGGCTTGCTTCG |
| | | TGCTGGCCGCTGTGTACCGGATCAA |
| | | CTGGATCACAGGCGGAATCGCCATC |
| | | GCCATGGCCTGCCTGGTGGGCCTGA |
| | | TGTGGCTGAGCTACTTCATCGCTTC |
| | | TTTCAGACTGTTCGCCAGAACCCGG |
| | | AGCATGTGGTCCTTCAACCCCGAGA |
| | | CAAACATCCTGCTGAACGTGCCTCT |
| | | GCACGGCACCATCCTGACAAGACCT |
| | | CTGCTCGAGAGCGAGCTGGTGATTG |
| | | GCGCAGTGATTCTGAGAGGCCATCT |
| | | GAGGATCGCCGGACACCACCTGGGC |
| | | AGATGCGACATCAAGGACCTTCCAA |
| | | AGGAAATCACCGTTGCCACCAGCCG |
| | | GACCCTGTCCTACTACAAACTGGGC |
| | | GCCAGCCAAAGAGTGGCCGGCGATA |
| | | GCGGCTTTGCCGCCTACAGCAGATA |
| | | CCGCATCGGAAATTACAAGCTCAAC |
| | | ACCGACCACAGCAGCTCTTCTGATA |
| | | ACATCGCCCTGCTGGTGCAGtaacc |
| | | cccccccctaacgttactggccgaa |
| | | gccgcttggaataaggccggtgtgc |
| | | gtttgtctatatgttattttccacc |
| | | atattgccgtcttttggcaatgtga |
| | | gggcccggaaacctggccctgtctt |
| | | cttgacgagcattcctaggggtctt |
| | | tcccctctcgccaaaggaatgcaag |
| | | gtctgttgaatgtcgtgaaggaagc |
| | | agttcctctggaagcttcttgaaga |
| | | caaacaacgtctgtagcgacccttt |
| | | gcaggcagcggaaccccccacctgg |
| | | cgacaggtgcctctgcggccaaaag |
| | | ccacgtgtataagatacacctgcaa |
| | | aggcggcacaaccccagtgccacgt |
| | | tgtgagttggatagttgtggaaaga |
| | | gtcaaatggctctcctcaagcgtat |
| | | tcaacaaggggctgaaggatgccca |
| | | gaaggtaccccattgtatgggatct |
| | | gatctggggcctcggtgcacatgct |
| | | ttacatgtgtttagtcgaggttaaa |
| | | aaaacgtctaggccccccgaaccac |
| | | ggggacgtggttttcctttgaaaaa |
| | | cacgatgataatatggccacaacca |
| | | tggaacaagagacttgcgcgcactc |
| | | tctcacttttgaggaatgcccaaaa |
| | | tgctctgctctacaataccgtaatg |
| | | gattttacctgctaaagtatgatga |
| | | agaatggtacccagaggagttattg |
| | | actgatggagaggatgatgtctttg |
| | | atcccgaattagacatggaagtcgt |
| | | tttcgagttacagggaagcggagct |
| | | actaacttcagcctgctgaagcagg |
| | | ctggagatgtggaggagaaccctgg |
| | | acctATGAGCGACAACGGCCCTCAA |
| | | AACCAGAGAAATGCCCCTCGGATCA |
| | | CATTTGGCGGACCTAGCGACAGCAC |
| | | CGGCAGCAACCAGAATGGAGAAAGA |
| | | AGCGGCGCCAGATCCAAGCAGCGGA |
| | | GACCTCAGGGACTGCCCAACAACAC |
| | | CGCTAGCTGGTTCACCGCCCTGACC |
| | | CAACACGGCAAGGAAGATCTGAAGT |
| | | TCCCCAGAGGCCAGGGCGTGCCTAT |
| | | CAACACAAACTCTTCTCCCGACGAC |
| | | CAGATCGGATACTATAGACGGGCCA |
| | | CTCGGAGAATTCGGGGCGGCGACGG |
| | | AAAAATGAAGGACCTTTCTCCAAGA |
| | | TGGTACTTCTACTACCTCGGCACAG |
| | | GCCCTGAGGCCGGCCTGCCTTACGG |
| | | CGCCAACAAGGATGGCATCATCTGG |
| | | GTCGCCACCGAGGGCGCCCTGAACA |
| | | CCCCTAAGGACCACATCGGCACAAG |
| | | AAACCCCGCTAACAACGCCGCAATC |
| | | GTGCTGCAGCTGCCTCAGGGCACCA |
| | | CCCTGCCCAAGGGCTTCTACGCCGA |
| | | GGGCTCTAGAGGTGGCTCCCAGGCT |
| | | TCTAGCCGCTCCTCCAGCCGCAGCA |
| | | GAAACAGCAGCAGGAACAGCACCCC |
| | | CGGCAGCTCCCGGGGCACCAGCCCC |
| | | GCCAGAATGGCCGGAAATGGCGGCG |
| | | ATGCCGCCCTGGCCCTGCTCCTGCT |
| | | GGACAGACTGAATCAGCTGGAAAGC |
| | | AAGATGAGCGGCAAAGGACAGCAGC |
| | | AGCAAGGCCAGACCGTGACCAAGAA |
| | | AAGCGCTGCTGAAGCCTCCAAGAAA |
| | | CCTAGACAAAAGCGGACCGCCACAA |
| | | AGGCCTACAACGTGACCCAAGCCTT |
| | | TGGAAGAAGAGGCCCCGAGCAGACA |
| | | CAGGGCAATTTCGGCGACCAGGAGC |
| | | TGATCCGGCAGGGAACCGACTACAA |
| | | GCACTGGCCTCAGATCGCCCAGTTC |
| | | GCCCCTAGCGCCAGCGCCTTCTTCG |
| | | GCATGAGCAGAATCGGCATGGAAGT |
| | | GACCCCTTCTGGCACCTGGCTGACC |
| | | TACACCGGCGCTATCAAGCTGGACG |
| | | ATAAGGATCCTAACTTCAAGGACCA |
| | | AGTGATCCTGCTGAACAAGCATATC |
| | | GACGCCTATAAGACCTTTCCACCTA |
| | | CAGAGCCTAAGAAAGATAAGAAGAA |
| | | GAAAGCCGACGAGACACAGGCCCTG |
| | | CCTCAGAGACAGAAAAAGCAGCAGA |
| | | CAGTGACACTGCTGCCAGCCGCTGA |
| | | CCTGGATGACTTCAGCAAGCAGCTG |
| | | CAGCAGAGCATGTCTTCTGCTGATA |
| | | GCACCCAGGCCggaagcggagctac |
| | | taacttcagcctgctgaagcaggct |
| | | ggagatgtggaggagaaccctggac |
| | | ctATGTATTCTTTTGTGTCCGAGGA |
| | | AACCGGCACACTGATCGTTAATAGC |
| | | GTGCTGCTCTTCCTGGCCTTCGTGG |
| | | TGTTCCTGCTGGTGACCCTGGCTAT |
| | | CCTGACCGCCCTGAGACTGTGTGCC |
| | | TACTGCTGCAACATCGTGAACGTGT |
| | | CTCTGGTCAAGCCTAGCTTCTACGT |
| | | GTACAGCCGGGTGAAGAACCTGAAC |
| | | AGCAGCAGAGTGCCCGACCTGCTGG |
| | | TGTGA |
| CoVEG7 | 39 | ATGGCCGACAGCAACGGCACAATCA |
| Expression | | CCGTGGAAGAGCTGAAGAAACTGCT |
| cassette | | GGAACAGTGGAACCTGGTCATCGGC |
| | | TTCCTGTTTCTGACCTGGATCTGTC |
| | | TGCTGCAGTTCGCTTATGCCAATCG |
| | | GAACAGATTCCTGTACATCATCAAG |
| | | CTGATCTTCCTGTGGCTGCTGTGGC |
| | | CTGTGACCCTGGCTTGCTTCGTGCT |
| | | GGCCGCTGTGTACCGGATCAACTGG |
| | | ATCACAGGCGGAATCGCCATCGCCA |
| | | TGGCCTGCCTGGTGGGCCTGATGTG |
| | | GCTGAGCTACTTCATCGCTTCTTTC |
| | | AGACTGTTCGCCAGAACCCGGAGCA |
| | | TGTGGTCCTTCAACCCCGAGACAAA |
| | | CATCCTGCTGAACGTGCCTCTGCAC |
| | | GGCACCATCCTGACAAGACCTCTGC |
| | | TCGAGAGCGAGCTGGTGATTGGCGC |
| | | AGTGATTCTGAGAGGCCATCTGAGG |
| | | ATCGCCGGACACCACCTGGGCAGAT |
| | | GCGACATCAAGGACCTTCCAAAGGA |
| | | AATCACCGTTGCCACCAGCCGGACC |
| | | CTGTCCTACTACAAACTGGGCGCCA |
| | | GCCAAAGAGTGGCCGGCGATAGCGG |
| | | CTTTGCCGCCTACAGCAGATACCGC |
| | | ATCGGAAATTACAAGCTCAACACCG |
| | | ACCACAGCAGCTCTTCTGATAACAT |
| | | CGCCCTGCTGGTGCAGCGAAAACGG |
| | | CGCggaagcggaggaagcggagcta |
| | | ctaacttcagcctgctgaagcaggc |
| | | tggagatgtggaggagaaccctgga |
| | | cctATGTTCGTGTTCCTGGTGCTGC |
| | | TGCCTCTGGTCAGCTCCCAGTGTGT |
| | | GAACCTGACCACCAGAACCCAGCTG |
| | | CCACCTGCTTATACAAACTCCTTCA |
| | | CTCGGGGGGTATACTACCCCGACAA |
| | | GGTGTTCAGATCTAGCGTGCTGCAT |
| | | TCTACACAAGACCTGTTCCTGCCCT |
| | | TCTTCAGCAACGTGACCTGGTTCCA |
| | | CGCCATCCACGTGTCTGGAACCAAC |
| | | GGAACCAAGAGATTCGACAACCCCG |
| | | TGCTGCCTTTCAACGACGGCGTGTA |
| | | CTTCGCCAGCACCGAGAAGTCCAAC |
| | | ATCATCAGAGGATGGATTTTCGGCA |
| | | CCACACTGGACAGCAAAACCCAGAG |
| | | CCTGCTGATCGTGAACAACGCCACC |
| | | AACGTGGTGATCAAGGTGTGCGAGT |
| | | TCCAGTTCTGCAATGATCCCTTCCT |
| | | GGGCGTGTACTACCACAAGAACAAC |
| | | AAGTCTTGGATGGAAAGCGAGTTCA |
| | | GAGTGTATTCCAGCGCCAACAATTG |
| | | CACCTTCGAGTACGTGAGCCAACCC |
| | | TTTCTGATGGACCTTGAAGGCAAGC |
| | | AGGGCAACTTCAAAAATCTGCGAGA |
| | | ATTTGTGTTCAAGAACATCGACGGA |
| | | TACTTCAAGATCTACTCTAAGCACA |
| | | CGCCAATCAACCTGGTGAGAGATCT |
| | | GCCCCAGGGCTTTAGCGCTTTGGAA |
| | | CCTCTGGTGGACCTGCCTATCGGAA |
| | | TCAACATCACCAGATTTCAAACTCT |
| | | CCTGGCCCTGCACAGATCTTATCTG |
| | | ACCCCTGGGGACAGTAGTAGCGGCT |
| | | GGACAGCCGGCGCCGCCGCCTACTA |
| | | CGTGGGATACCTGCAGCCTAGAACA |
| | | TTCCTGCTGAAGTACAATGAGAACG |
| | | GAACAATCACAGACGCCGTGGACTG |
| | | CGCCCTGGATCCTTTGAGCGAGACA |
| | | AAGTGCACCCTGAAGTCGTTCACCG |
| | | TCGAAAAAGGCATCTACCAGACCAG |
| | | CAACTTCCGCGTGCAGCCTACGGAA |
| | | TCTATCGTGCGGTTCCCCAACATCA |
| | | CCAACCTGTGCCCTTTCGGCGAGGT |
| | | GTTTAACGCTACAAGGTTCGCCAGC |
| | | GTGTATGCCTGGAACAGAAAGAGAA |
| | | TCAGCAATTGCGTGGCCGATTATAG |
| | | CGTTCTGTACAACAGCGCTTCCTTC |
| | | AGCACCTTCAAGTGCTACGGCGTGT |
| | | CTCCAACCAAGCTGAACGACCTCTG |
| | | CTTCACCAATGTCTACGCTGACTCT |
| | | TTCGTGATTAGAGGCGATGAGGTTA |
| | | GACAGATCGCACCTGGCCAGACCGG |
| | | CAAAATCGCTGACTACAACTACAAG |
| | | CTGCCTGATGACTTCACAGGCTGTG |
| | | TCATTGCCTGGAACTCAAATAACCT |
| | | GGACTCTAAAGTGGGCGGCAACTAC |
| | | AACTACCTGTACCGGCTGTTCCGGA |
| | | AGAGCAATCTGAAACCTTTTGAGCG |
| | | GGACATCTCTACAGAGATCTACCAG |
| | | GCCGGCAGCACACCCTGCAACGGCG |
| | | TTGAGGGCTTCAACTGCTACTTCCC |
| | | TCTGCAGAGCTACGGCTTTCAGCCA |
| | | ACAAATGGAGTGGGCTACCAGCCGT |
| | | ACAGAGTGGTGGTGCTGAGCTTCGA |
| | | ACTGCTGCATGCCCCAGCCACAGTG |
| | | TGTGGACCTAAGAAGTCTACCAACC |
| | | TGGTGAAGAACAAGTGCGTGAACTT |
| | | TAACTTTAACGGCCTGACCGGCACA |
| | | GGCGTGCTGACCGAATCCAACAAAA |
| | | AGTTCCTGCCCTTCCAACAGTTCGG |
| | | CAGAGACATCGCCGATACAACCGAT |
| | | GCCGTGCGGGACCCCCAGACCTTAG |
| | | AAATCCTAGATATCACCCCGTGCAG |
| | | CTTCGGCGGAGTCTCTGTTATTACT |
| | | CCTGGCACCAACACCAGCAACCAAG |
| | | TGGCTGTTCTGTACCAAggcGTGAA |
| | | CTGCACCGAAGTGCCTGTGGCTATC |
| | | CACGCCGATCAGCTGACCCCAACCT |
| | | GGCGGGTGTATAGCACCGGCTCTAA |
| | | CGTGTTCCAGACCCGGGCTGGCTGC |
| | | CTGATCGGCGCCGAACACGTCAACA |
| | | ACTCCTATGAATGTGACATCCCCAT |
| | | CGGGGCTGGCATCTGCGCCAGTTAC |
| | | CAGACACAGACAAATAGCCCTAGAC |
| | | GGGCCAGAAGCGTGGCCTCCCAGAG |
| | | TATCATTGCCTACACCATGAGCCTG |
| | | GGCGCCGAGAACAGCGTGGCCTATT |
| | | CTAACAATAGCATCGCAATCCCTAC |
| | | CAACTTTACCATCTCTGTGACAACC |
| | | GAGATCCTGCCTGTGAGCATGACCA |
| | | AAACCAGCGTGGACTGCACGATGTA |
| | | CATCTGTGGCGACAGCACAGAATGC |
| | | AGTAATCTGTTGCTGCAGTACGGCA |
| | | GCTTTTGCACCCAGTTGAATAGAGC |
| | | CCTGACCGGAATCGCCGTAGAGCAG |
| | | GACAAAAATACCCAGGAGGTGTTCG |
| | | CCCAGGTGAAACAGATCTACAAGAC |
| | | ACCTCCCATTAAGGACTTCGGAGGT |
| | | TTTAACTTCAGCCAGATCCTGCCCG |
| | | ACCCTTCCAAGCCTAGCAAACGCTC |
| | | CTTCATCGAGGACCTGCTCTTCAAC |
| | | AAGGTGACACTGGCTGATGCCGGCT |
| | | TCATCAAGCAGTACGGAGATTGTCT |
| | | GGGAGACATCGCCGCTAGAGATCTG |
| | | ATCTGCGCCCAAAAGTTCAACGGCC |
| | | TGACCGTGCTGCCTCCTCTGCTTAC |
| | | AGACGAGATGATCGCCCAGTACACC |
| | | AGCGCCCTGCTGGCTGGCACCATCA |
| | | CAAGCGGCTGGACCTTCGGAGCCGG |
| | | AGCCGCTCTGCAAATCCCCTTTGCC |
| | | ATGCAGATGGCCTACCGGTTCAACG |
| | | GCATCGGCGTGACACAGAATGTGCT |
| | | GTACGAGAACCAGAAGCTGATCGCT |
| | | AACCAGTTTAACAGCGCTATCGGCA |
| | | AGATCCAGGACTCGCTGAGTAGCAC |
| | | CGCCTCTGCCCTGGGCAAGCTGCAG |
| | | GACGTCGTGAACCAGAACGCCCAAG |
| | | CCCTGAACACACTGGTGAAACAGCT |
| | | GAGCAGCAACTTCGGCGCCATCAGC |
| | | TCTGTGCTGAACGATATCCTGAGCA |
| | | GACTGGACAAGGTGGAAGCCGAGGT |
| | | CCAGATCGACAGACTGATCACAGGA |
| | | AGACTGCAGAGCCTGCAAACGTACG |
| | | TGACACAGCAGCTGATCCGGGCAGC |
| | | CGAAATCCGGGCCAGCGCCAATCTG |
| | | GCCGCTACCAAGATGAGCGAGTGCG |
| | | TGTTAGGCCAGAGCAAGCGGGTGGA |
| | | TTTCTGCGGTAAGGGATACCACCTG |
| | | ATGAGCTTTCCCCAGAGCGCTCCTC |
| | | ACGGCGTGGTGTTTCTGCACGTGAC |
| | | CTACGTTCCTGCCCAGGAAAAGAAC |
| | | TTCACCACCGCCCCTGCTATCTGCC |
| | | ACGATGGCAAGGCCCACTTCCCTAG |
| | | AGAGGGCGTTTTCGTGTCTAACGGC |
| | | ACACACTGGTTTGTGACCCAGAGAA |
| | | ACTTCTACGAGCCTCAGATCATCAC |
| | | CACAGACAACACCTTTGTGAGCGGC |
| | | AATTGCGACGTGGTGATCGGAATTG |
| | | TTAATAATACCGTGTACGACCCTCT |
| | | GCAGCCTGAGCTCGACAGCTTCAAG |
| | | GAAGAGCTGGACAAGTACTTCAAGA |
| | | ACCACACCTCCCCAGATGTGGACCT |
| | | GGGCGATATTTCAGGCATCAACGCC |
| | | TCCGTCGTGAATATCCAGAAGGAGA |
| | | TCGACCGGCTCAACGAGGTGGCCAA |
| | | GAACCTTAACGAGAGCCTGATCGAC |
| | | CTGCAGGAACTGGGCAAATATGAGC |
| | | AGTACATCAAGTGGCCTTGGTACAT |
| | | CTGGCTGGGCTTTATCGCAGGCCTG |
| | | ATCGCTATCGTGATGGTGACCATTA |
| | | TGCTGTGTTGTATGACCAGCTGTTG |
| | | TAGTTGTCTGAAGGGCTGCTGTTCT |
| | | TGCGGCAGCTGCTGCAAGTTCGACG |
| | | AAGACGACTCAGAGCCCGTGCTGAA |
| | | AGGCGTGAAGCTGCACTACACCCGA |
| | | AAACGGCGCggaagcggaggaagcg |
| | | gagctactaacttcagcctgctgaa |
| | | gcaggctggagatgtggaggagaac |
| | | cctggacctATGTATTCTTTTGTGT |
| | | CCGAGGAAACCGGCACACTGATCGT |
| | | TAATAGCGTGCTGCTCTTCCTGGCC |
| | | TTCGTGGTGTTCCTGCTGGTGACCC |
| | | TGGCTATCCTGACCGCCCTGAGACT |
| | | GTGTGCCTACTGCTGCAACATCGTG |
| | | AACGTGTCTCTGGTCAAGCCTAGCT |
| | | TCTACGTGTACAGCCGGGTGAAGAA |
| | | CCTGAACAGCAGCAGAGTGCCCGAC |
| | | CTGCTGGTGtaatccccccccccta |
| | | acgttactggccgaagccgcttgga |
| | | ataaggccggtgtgcgtttgtctat |
| | | atgttattttccaccatattgccgt |
| | | cttttggcaatgtgagggcccggaa |
| | | acctggccctgtcttcttgacgagc |
| | | attcctaggggtctttcccctctcg |
| | | ccaaaggaatgcaaggtctgttgaa |
| | | tgtcgtgaaggaagcagttcctctg |
| | | gaagcttcttgaagacaaacaacgt |
| | | ctgtagcgaccctttgcaggcagcg |
| | | gaaccccccacctggcgacaggtgc |
| | | ctctgcggccaaaagccacgtgtat |
| | | aagatacacctgcaaaggcggcaca |
| | | accccagtgccacgttgtgagttgg |
| | | atagttgtggaaagagtcaaatggc |
| | | tctcctcaagcgtattcaacaaggg |
| | | gctgaaggatgcccagaaggtaccc |
| | | cattgtatgggatctgatctggggc |
| | | ctcggtgcacatgctttacatgtgt |
| | | ttagtcgaggttaaaaaaacgtcta |
| | | ggccccccgaaccacggggacgtgg |
| | | ttttcctttgaaaaacacgatgata |
| | | atatggccacaaccatggaacaaga |
| | | gacttgcgcgcactctctcactttt |
| | | gaggaatgcccaaaatgctctgctc |
| | | tacaataccgtaatggattttacct |
| | | gctaaagtatgatgaagaatggtac |
| | | ccagaggagttattgactgatggag |
| | | aggatgatgtctttgatcccgaatt |
| | | agacatggaagtcgttttcgagtta |
| | | cagggaagcggagctactaacttca |
| | | gcctgctgaagcaggctggagatgt |
| | | ggaggagaaccctggacctATGAGC |
| | | GACAACGGCCCTCAAAACCAGAGAA |
| | | ATGCCCCTCGGATCACATTTGGCGG |
| | | ACCTAGCGACAGCACCGGCAGCAAC |
| | | CAGAATGGAGAAAGAAGCGGCGCCA |
| | | GATCCAAGCAGCGGAGACCTCAGGG |
| | | ACTGCCCAACAACACCGCTAGCTGG |
| | | TTCACCGCCCTGACCCAACACGGCA |
| | | AGGAAGATCTGAAGTTCCCCAGAGG |
| | | CCAGGGCGTGCCTATCAACACAAAC |
| | | TCTTCTCCCGACGACCAGATCGGAT |
| | | ACTATAGACGGGCCACTCGGAGAAT |
| | | TCGGGGCGGCGACGGAAAAATGAAG |
| | | GACCTTTCTCCAAGATGGTACTTCT |
| | | ACTACCTCGGCACAGGCCCTGAGGC |
| | | CGGCCTGCCTTACGGCGCCAACAAG |
| | | GATGGCATCATCTGGGTCGCCACCG |
| | | AGGGCGCCCTGAACACCCCTAAGGA |
| | | CCACATCGGCACAAGAAACCCCGCT |
| | | AACAACGCCGCAATCGTGCTGCAGC |
| | | TGCCTCAGGGCACCACCCTGCCCAA |
| | | GGGCTTCTACGCCGAGGGCTCTAGA |
| | | GGTGGCTCCCAGGCTTCTAGCCGCT |
| | | CCTCCAGCCGCAGCAGAAACAGCAG |
| | | CAGGAACAGCACCCCCGGCAGCTCC |
| | | CGGGGCACCAGCCCCGCCAGAATGG |
| | | CCGGAAATGGCGGCGATGCCGCCCT |
| | | GGCCCTGCTCCTGCTGGACAGACTG |
| | | AATCAGCTGGAAAGCAAGATGAGCG |
| | | GCAAAGGACAGCAGCAGCAAGGCCA |
| | | GACCGTGACCAAGAAAAGCGCTGCT |
| | | GAAGCCTCCAAGAAACCTAGACAAA |
| | | AGCGGACCGCCACAAAGGCCTACAA |
| | | CGTGACCCAAGCCTTTGGAAGAAGA |
| | | GGCCCCGAGCAGACACAGGGCAATT |
| | | TCGGCGACCAGGAGCTGATCCGGCA |
| | | GGGAACCGACTACAAGCACTGGCCT |
| | | CAGATCGCCCAGTTCGCCCCTAGCG |
| | | CCAGCGCCTTCTTCGGCATGAGCAG |
| | | AATCGGCATGGAAGTGACCCCTTCT |
| | | GGCACCTGGCTGACCTACACCGGCG |
| | | CTATCAAGCTGGACGATAAGGATCC |
| | | TAACTTCAAGGACCAAGTGATCCTG |
| | | CTGAACAAGCATATCGACGCCTATA |
| | | AGACCTTTCCACCTACAGAGCCTAA |
| | | GAAAGATAAGAAGAAGAAAGCCGAC |
| | | GAGACACAGGCCCTGCCTCAGAGAC |
| | | AGAAAAAGCAGCAGACAGTGACACT |
| | | GCTGCCAGCCGCTGACCTGGATGAC |
| | | TTCAGCAAGCAGCTGCAGCAGAGCA |
| | | TGTCTTCTGCTGATAGCACCCAGGC |
| | | C |
| CoVEG8 | 40 | ATGGCCGACAGCAACGGCACAATCA |
| expression | | CCGTGGAAGAGCTGAAGAAACTGCT |
| cassette | | GGAACAGTGGAACCTGGTCATCGGC |
| | | TTCCTGTTTCTGACCTGGATCTGTC |
| | | TGCTGCAGTTCGCTTATGCCAATCG |
| | | GAACAGATTCCTGTACATCATCAAG |
| | | CTGATCTTCCTGTGGCTGCTGTGGC |
| | | CTGTGACCCTGGCTTGCTTCGTGCT |
| | | GGCCGCTGTGTACCGGATCAACTGG |
| | | ATCACAGGCGGAATCGCCATCGCCA |
| | | TGGCCTGCCTGGTGGGCCTGATGTG |
| | | GCTGAGCTACTTCATCGCTTCTTTC |
| | | AGACTGTTCGCCAGAACCCGGAGCA |
| | | TGTGGTCCTTCAACCCCGAGACAAA |
| | | CATCCTGCTGAACGTGCCTCTGCAC |
| | | GGCACCATCCTGACAAGACCTCTGC |
| | | TCGAGAGCGAGCTGGTGATTGGCGC |
| | | AGTGATTCTGAGAGGCCATCTGAGG |
| | | ATCGCCGGACACCACCTGGGCAGAT |
| | | GCGACATCAAGGACCTTCCAAAGGA |
| | | AATCACCGTTGCCACCAGCCGGACC |
| | | CTGTCCTACTACAAACTGGGCGCCA |
| | | GCCAAAGAGTGGCCGGCGATAGCGG |
| | | CTTTGCCGCCTACAGCAGATACCGC |
| | | ATCGGAAATTACAAGCTCAACACCG |
| | | ACCACAGCAGCTCTTCTGATAACAT |
| | | CGCCCTGCTGGTGCAGCGAAAACGG |
| | | CGCggaagcggaggaagcggagcta |
| | | ctaacttcagcctgctgaagcaggc |
| | | tggagatgtggaggagaaccctgga |
| | | cctATGTTCGTGTTCCTGGTGCTGC |
| | | TGCCTCTGGTCAGCTCCCAGTGTGT |
| | | GAACCTGACCACCAGAACCCAGCTG |
| | | CCACCTGCTTATACAAACTCCTTCA |
| | | CTCGGGGGGTATACTACCCCGACAA |
| | | GGTGTTCAGATCTAGCGTGCTGCAT |
| | | TCTACACAAGACCTGTTCCTGCCCT |
| | | TCTTCAGCAACGTGACCTGGTTCCA |
| | | CGCCATCCACGTGTCTGGAACCAAC |
| | | GGAACCAAGAGATTCGACAACCCCG |
| | | TGCTGCCTTTCAACGACGGCGTGTA |
| | | CTTCGCCAGCACCGAGAAGTCCAAC |
| | | ATCATCAGAGGATGGATTTTCGGCA |
| | | CCACACTGGACAGCAAAACCCAGAG |
| | | CCTGCTGATCGTGAACAACGCCACC |
| | | AACGTGGTGATCAAGGTGTGCGAGT |
| | | TCCAGTTCTGCAATGATCCCTTCCT |
| | | GGGCGTGTACTACCACAAGAACAAC |
| | | AAGTCTTGGATGGAAAGCGAGTTCA |
| | | GAGTGTATTCCAGCGCCAACAATTG |
| | | CACCTTCGAGTACGTGAGCCAACCC |
| | | TTTCTGATGGACCTTGAAGGCAAGC |
| | | AGGGCAACTTCAAAAATCTGCGAGA |
| | | ATTTGTGTTCAAGAACATCGACGGA |
| | | TACTTCAAGATCTACTCTAAGCACA |
| | | CGCCAATCAACCTGGTGAGAGATCT |
| | | GCCCCAGGGCTTTAGCGCTTTGGAA |
| | | CCTCTGGTGGACCTGCCTATCGGAA |
| | | TCAACATCACCAGATTTCAAACTCT |
| | | CCTGGCCCTGCACAGATCTTATCTG |
| | | ACCCCTGGGGACAGTAGTAGCGGCT |
| | | GGACAGCCGGCGCCGCCGCCTACTA |
| | | CGTGGGATACCTGCAGCCTAGAACA |
| | | TTCCTGCTGAAGTACAATGAGAACG |
| | | GAACAATCACAGACGCCGTGGACTG |
| | | CGCCCTGGATCCTTTGAGCGAGACA |
| | | AAGTGCACCCTGAAGTCGTTCACCG |
| | | TCGAAAAAGGCATCTACCAGACCAG |
| | | CAACTTCCGCGTGCAGCCTACGGAA |
| | | TCTATCGTGCGGTTCCCCAACATCA |
| | | CCAACCTGTGCCCTTTCGGCGAGGT |
| | | GTTTAACGCTACAAGGTTCGCCAGC |
| | | GTGTATGCCTGGAACAGAAAGAGAA |
| | | TCAGCAATTGCGTGGCCGATTATAG |
| | | CGTTCTGTACAACAGCGCTTCCTTC |
| | | AGCACCTTCAAGTGCTACGGCGTGT |
| | | CTCCAACCAAGCTGAACGACCTCTG |
| | | CTTCACCAATGTCTACGCTGACTCT |
| | | TTCGTGATTAGAGGCGATGAGGTTA |
| | | GACAGATCGCACCTGGCCAGACCGG |
| | | CAAAATCGCTGACTACAACTACAAG |
| | | CTGCCTGATGACTTCACAGGCTGTG |
| | | TCATTGCCTGGAACTCAAATAACCT |
| | | GGACTCTAAAGTGGGCGGCAACTAC |
| | | AACTACCTGTACCGGCTGTTCCGGA |
| | | AGAGCAATCTGAAACCTTTTGAGCG |
| | | GGACATCTCTACAGAGATCTACCAG |
| | | GCCGGCAGCACACCCTGCAACGGCG |
| | | TTGAGGGCTTCAACTGCTACTTCCC |
| | | TCTGCAGAGCTACGGCTTTCAGCCA |
| | | ACAAATGGAGTGGGCTACCAGCCGT |
| | | ACAGAGTGGTGGTGCTGAGCTTCGA |
| | | ACTGCTGCATGCCCCAGCCACAGTG |
| | | TGTGGACCTAAGAAGTCTACCAACC |
| | | TGGTGAAGAACAAGTGCGTGAACTT |
| | | TAACTTTAACGGCCTGACCGGCACA |
| | | GGCGTGCTGACCGAATCCAACAAAA |
| | | AGTTCCTGCCCTTCCAACAGTTCGG |
| | | CAGAGACATCGCCGATACAACCGAT |
| | | GCCGTGCGGGACCCCCAGACCTTAG |
| | | AAATCCTAGATATCACCCCGTGCAG |
| | | CTTCGGCGGAGTCTCTGTTATTACT |
| | | CCTGGCACCAACACCAGCAACCAAG |
| | | TGGCTGTTCTGTACCAAggcGTGAA |
| | | CTGCACCGAAGTGCCTGTGGCTATC |
| | | CACGCCGATCAGCTGACCCCAACCT |
| | | GGCGGGTGTATAGCACCGGCTCTAA |
| | | CGTGTTCCAGACCCGGGCTGGCTGC |
| | | CTGATCGGCGCCGAACACGTCAACA |
| | | ACTCCTATGAATGTGACATCCCCAT |
| | | CGGGGCTGGCATCTGCGCCAGTTAC |
| | | CAGACACAGACAAATAGCCCTAGAC |
| | | GGGCCAGAAGCGTGGCCTCCCAGAG |
| | | TATCATTGCCTACACCATGAGCCTG |
| | | GGCGCCGAGAACAGCGTGGCCTATT |
| | | CTAACAATAGCATCGCAATCCCTAC |
| | | CAACTTTACCATCTCTGTGACAACC |
| | | GAGATCCTGCCTGTGAGCATGACCA |
| | | AAACCAGCGTGGACTGCACGATGTA |
| | | CATCTGTGGCGACAGCACAGAATGC |
| | | AGTAATCTGTTGCTGCAGTACGGCA |
| | | GCTTTTGCACCCAGTTGAATAGAGC |
| | | CCTGACCGGAATCGCCGTAGAGCAG |
| | | GACAAAAATACCCAGGAGGTGTTCG |
| | | CCCAGGTGAAACAGATCTACAAGAC |
| | | ACCTCCCATTAAGGACTTCGGAGGT |
| | | TTTAACTTCAGCCAGATCCTGCCCG |
| | | ACCCTTCCAAGCCTAGCAAACGCTC |
| | | CTTCATCGAGGACCTGCTCTTCAAC |
| | | AAGGTGACACTGGCTGATGCCGGCT |
| | | TCATCAAGCAGTACGGAGATTGTCT |
| | | GGGAGACATCGCCGCTAGAGATCTG |
| | | ATCTGCGCCCAAAAGTTCAACGGCC |
| | | TGACCGTGCTGCCTCCTCTGCTTAC |
| | | AGACGAGATGATCGCCCAGTACACC |
| | | AGCGCCCTGCTGGCTGGCACCATCA |
| | | CAAGCGGCTGGACCTTCGGAGCCGG |
| | | AGCCGCTCTGCAAATCCCCTTTGCC |
| | | ATGCAGATGGCCTACCGGTTCAACG |
| | | GCATCGGCGTGACACAGAATGTGCT |
| | | GTACGAGAACCAGAAGCTGATCGCT |
| | | AACCAGTTTAACAGCGCTATCGGCA |
| | | AGATCCAGGACTCGCTGAGTAGCAC |
| | | CGCCTCTGCCCTGGGCAAGCTGCAG |
| | | GACGTCGTGAACCAGAACGCCCAAG |
| | | CCCTGAACACACTGGTGAAACAGCT |
| | | GAGCAGCAACTTCGGCGCCATCAGC |
| | | TCTGTGCTGAACGATATCCTGAGCA |
| | | GACTGGACAAGGTGGAAGCCGAGGT |
| | | CCAGATCGACAGACTGATCACAGGA |
| | | AGACTGCAGAGCCTGCAAACGTACG |
| | | TGACACAGCAGCTGATCCGGGCAGC |
| | | CGAAATCCGGGCCAGCGCCAATCTG |
| | | GCCGCTACCAAGATGAGCGAGTGCG |
| | | TGTTAGGCCAGAGCAAGCGGGTGGA |
| | | TTTCTGCGGTAAGGGATACCACCTG |
| | | ATGAGCTTTCCCCAGAGCGCTCCTC |
| | | ACGGCGTGGTGTTTCTGCACGTGAC |
| | | CTACGTTCCTGCCCAGGAAAAGAAC |
| | | TTCACCACCGCCCCTGCTATCTGCC |
| | | ACGATGGCAAGGCCCACTTCCCTAG |
| | | AGAGGGCGTTTTCGTGTCTAACGGC |
| | | ACACACTGGTTTGTGACCCAGAGAA |
| | | ACTTCTACGAGCCTCAGATCATCAC |
| | | CACAGACAACACCTTTGTGAGCGGC |
| | | AATTGCGACGTGGTGATCGGAATTG |
| | | TTAATAATACCGTGTACGACCCTCT |
| | | GCAGCCTGAGCTCGACAGCTTCAAG |
| | | GAAGAGCTGGACAAGTACTTCAAGA |
| | | ACCACACCTCCCCAGATGTGGACCT |
| | | GGGCGATATTTCAGGCATCAACGCC |
| | | TCCGTCGTGAATATCCAGAAGGAGA |
| | | TCGACCGGCTCAACGAGGTGGCCAA |
| | | GAACCTTAACGAGAGCCTGATCGAC |
| | | CTGCAGGAACTGGGCAAATATGAGC |
| | | AGTACATCAAGTGGCCTTGGTACAT |
| | | CTGGCTGGGCTTTATCGCAGGCCTG |
| | | ATCGCTATCGTGATGGTGACCATTA |
| | | TGCTGTGTTGTATGACCAGCTGTTG |
| | | TAGTTGTCTGAAGGGCTGCTGTTCT |
| | | TGCGGCAGCTGCTGCAAGTTCGACG |
| | | AAGACGACTCAGAGCCCGTGCTGAA |
| | | AGGCGTGAAGCTGCACTACACCCGA |
| | | AAACGGCGCggaagcggaggaagcg |
| | | gagctactaacttcagcctgctgaa |
| | | gcaggctggagatgtggaggagaac |
| | | cctggacctATGTATTCTTTTGTGT |
| | | CCGAGGAAACCGGCACACTGATCGT |
| | | TAATAGCGTGCTGCTCTTCCTGGCC |
| | | TTCGTGGTGTTCCTGCTGGTGACCC |
| | | TGGCTATCCTGACCGCCCTGAGACT |
| | | GTGTGCCTACTGCTGCAACATCGTG |
| | | AACGTGTCTCTGGTCAAGCCTAGCT |
| | | TCTACGTGTACAGCCGGGTGAAGAA |
| | | CCTGAACAGCAGCAGAGTGCCCGAC |
| | | CTGCTGGTGtaatccccccccccta |
| | | acgttactggccgaagccgcttgga |
| | | ataaggccggtgtgcgtttgtctat |
| | | atgttattttccaccatattgccgt |
| | | cttttggcaatgtgagggcccggaa |
| | | acctggccctgtcttcttgacgagc |
| | | attcctaggggtctttcccctctcg |
| | | ccaaaggaatgcaaggtctgttgaa |
| | | tgtcgtgaaggaagcagttcctctg |
| | | gaagcttcttgaagacaaacaacgt |
| | | ctgtagcgaccctttgcaggcagcg |
| | | gaaccccccacctggcgacaggtgc |
| | | ctctgcggccaaaagccacgtgtat |
| | | aagatacacctgcaaaggcggcaca |
| | | accccagtgccacgttgtgagttgg |
| | | atagttgtggaaagagtcaaatggc |
| | | tctcctcaagcgtattcaacaaggg |
| | | gctgaaggatgcccagaaggtaccc |
| | | cattgtatgggatctgatctggggc |
| | | ctcggtgcacatgctttacatgtgt |
| | | ttagtcgaggttaaaaaaacgtcta |
| | | ggccccccgaaccacggggacgtgg |
| | | ttttcctttgaaaaacacgatgata |
| | | atatggccacaaccatggaacaaga |
| | | gacttgcgcgcactctctcactttt |
| | | gaggaatgcccaaaatgctctgctc |
| | | tacaataccgtaatggattttacct |
| | | gctaaagtatgatgaagaatggtac |
| | | ccagaggagttattgactgatggag |
| | | aggatgatgtctttgatcccgaatt |
| | | agacatggaagtcgttttcgagtta |
| | | cagggaagcggagctactaacttca |
| | | gcctgctgaagcaggctggagatgt |
| | | ggaggagaaccctggacctATGAGC |
| | | GACAACGGCCCTCAAAACCAGAGAA |
| | | ATGCCCCTCGGATCACATTTGGCGG |
| | | ACCTAGCGACAGCACCGGCAGCAAC |
| | | CAGAATGGAGAAAGAAGCGGCGCCA |
| | | GATCCAAGCAGCGGAGACCTCAGGG |
| | | ACTGCCCAACAACACCGCTAGCTGG |
| | | TTCACCGCCCTGACCCAACACGGCA |
| | | AGGAAGATCTGAAGTTCCCCAGAGG |
| | | CCAGGGCGTGCCTATCAACACAAAC |
| | | TCTTCTCCCGACGACCAGATCGGAT |
| | | ACTATAGACGGGCCACTCGGAGAAT |
| | | TCGGGGCGGCGACGGAAAAATGAAG |
| | | GACCTTTCTCCAAGATGGTACTTCT |
| | | ACTACCTCGGCACAGGCCCTGAGGC |
| | | CGGCCTGCCTTACGGCGCCAACAAG |
| | | GATGGCATCATCTGGGTCGCCACCG |
| | | AGGGCGCCCTGAACACCCCTAAGGA |
| | | CCACATCGGCACAAGAAACCCCGCT |
| | | AACAACGCCGCAATCGTGCTGCAGC |
| | | TGCCTCAGGGCACCACCCTGCCCAA |
| | | GGGCTTCTACGCCGAGGGCTCTAGA |
| | | GGTGGCTCCCAGGCTTCTAGCCGCT |
| | | CCTCCAGCCGCAGCAGAAACAGCAG |
| | | CAGGAACAGCACCCCCGGCAGCTCC |
| | | CGGGGCACCAGCCCCGCCAGAATGG |
| | | CCGGAAATGGCGGCGATGCCGCCCT |
| | | GGCCCTGCTCCTGCTGGACAGACTG |
| | | AATCAGCTGGAAAGCAAGATGAGCG |
| | | GCAAAGGACAGCAGCAGCAAGGCCA |
| | | GACCGTGACCAAGAAAAGCGCTGCT |
| | | GAAGCCTCCAAGAAACCTAGACAAA |
| | | AGCGGACCGCCACAAAGGCCTACAA |
| | | CGTGACCCAAGCCTTTGGAAGAAGA |
| | | GGCCCCGAGCAGACACAGGGCAATT |
| | | TCGGCGACCAGGAGCTGATCCGGCA |
| | | GGGAACCGACTACAAGCACTGGCCT |
| | | CAGATCGCCCAGTTCGCCCCTAGCG |
| | | CCAGCGCCTTCTTCGGCATGAGCAG |
| | | AATCGGCATGGAAGTGACCCCTTCT |
| | | GGCACCTGGCTGACCTACACCGGCG |
| | | CTATCAAGCTGGACGATAAGGATCC |
| | | TAACTTCAAGGACCAAGTGATCCTG |
| | | CTGAACAAGCATATCGACGCCTATA |
| | | AGACCTTTCCACCTACAGAGCCTAA |
| | | GAAAGATAAGAAGAAGAAAGCCGAC |
| | | GAGACACAGGCCCTGCCTCAGAGAC |
| | | AGAAAAAGCAGCAGACAGTGACACT |
| | | GCTGCCAGCCGCTGACCTGGATGAC |
| | | TTCAGCAAGCAGCTGCAGCAGAGCA |
| | | TGTCTTCTGCTGATAGCACCCAGGC |
| | | CtaaGTGTCaAAGcGCGAGGAACTG |
| | | TTCACCGGAGTttTGCCCATCCTGG |
| | | TCGAGCTGGACGGCGATGTGAACGG |
| | | CCACAAGTTCAGCGTTTCTGGCGAG |
| | | GGtgagctttgggctaagcgcaaca |
| | | ttaaaccagtaccagaggtgaaaat |
| | | actcaataatttgggtgtggacatt |
| | | gctgctaatactgtgatctgggact |
| | | acaaaagagatgctccagcacatat |
| | | atctactattggtgtttgttctatg |
| | | actgacatagccaagaaaccaactg |
| | | aaacgatttgtgcaccactcactgt |
| | | cttttttgatggtagagttgatggt |
| | | caagtagacttatttagaaatgccc |
| | | gtaatggtgttcttattacagaagg |
| | | tagtgttaaaggtttacaaccatct |
| | | gtaggtcccaaacaagctagtctta |
| | | atggagtcacattaattggagaagc |
| | | cgtaaaaacacagttcaattattat |
| | | aagaaagttgatggtgttgtccaac |
| | | aattacctgaaacttactttactca |
| | | gagtagaaatttacaagaatttaaa |
| | | cccaggagtcaaatggaaattgatt |
| | | tcttagaattagctatggatgaatt |
| | | cattgaacggtataaattagaaggc |
| | | tatgccttcgaacatatcgtttatg |
| | | gagattttagtcata |
| CoVEG9 | 41 | ATGGCCGACAGCAACGGCACAATCA |
| expression | | CCGTGGAAGAGCTGAAGAAACTGCT |
| cassette | | GGAACAGTGGAACCTGGTCATCGGC |
| | | TTCCTGTTTCTGACCTGGATCTGTC |
| | | TGCTGCAGTTCGCTTATGCCAATCG |
| | | GAACAGATTCCTGTACATCATCAAG |
| | | CTGATCTTCCTGTGGCTGCTGTGGC |
| | | CTGTGACCCTGGCTTGCTTCGTGCT |
| | | GGCCGCTGTGTACCGGATCAACTGG |
| | | ATCACAGGCGGAATCGCCATCGCCA |
| | | TGGCCTGCCTGGTGGGCCTGATGTG |
| | | GCTGAGCTACTTCATCGCTTCTTTC |
| | | AGACTGTTCGCCAGAACCCGGAGCA |
| | | TGTGGTCCTTCAACCCCGAGACAAA |
| | | CATCCTGCTGAACGTGCCTCTGCAC |
| | | GGCACCATCCTGACAAGACCTCTGC |
| | | TCGAGAGCGAGCTGGTGATTGGCGC |
| | | AGTGATTCTGAGAGGCCATCTGAGG |
| | | ATCGCCGGACACCACCTGGGCAGAT |
| | | GCGACATCAAGGACCTTCCAAAGGA |
| | | AATCACCGTTGCCACCAGCCGGACC |
| | | CTGTCCTACTACAAACTGGGCGCCA |
| | | GCCAAAGAGTGGCCGGCGATAGCGG |
| | | CTTTGCCGCCTACAGCAGATACCGC |
| | | ATCGGAAATTACAAGCTCAACACCG |
| | | ACCACAGCAGCTCTTCTGATAACAT |
| | | CGCCCTGCTGGTGCAGCGAAAACGG |
| | | CGCggaagcggaggaagcggagcta |
| | | ctaacttcagcctgctgaagcaggc |
| | | tggagatgtggaggagaaccctgga |
| | | cctATGAGCGACAACGGCCCTCAAA |
| | | ACCAGAGAAATGCCCCTCGGATCAC |
| | | ATTTGGCGGACCTAGCGACAGCACC |
| | | GGCAGCAACCAGAATGGAGAAAGAA |
| | | GCGGCGCCAGATCCAAGCAGCGGAG |
| | | ACCTCAGGGACTGCCCAACAACACC |
| | | GCTAGCTGGTTCACCGCCCTGACCC |
| | | AACACGGCAAGGAAGATCTGAAGTT |
| | | CCCCAGAGGCCAGGGCGTGCCTATC |
| | | AACACAAACTCTTCTCCCGACGACC |
| | | AGATCGGATACTATAGACGGGCCAC |
| | | TCGGAGAATTCGGGGCGGCGACGGA |
| | | AAAATGAAGGACCTTTCTCCAAGAT |
| | | GGTACTICTACTACCTCGGCACAGG |
| | | CCCTGAGGCCGGCCTGCCTTACGGC |
| | | GCCAACAAGGATGGCATCATCTGGG |
| | | TCGCCACCGAGGGCGCCCTGAACAC |
| | | CCCTAAGGACCACATCGGCACAAGA |
| | | AACCCCGCTAACAACGCCGCAATCG |
| | | TGCTGCAGCTGCCTCAGGGCACCAC |
| | | CCTGCCCAAGGGCTTCTACGCCGAG |
| | | GGCTCTAGAGGTGGCTCCCAGGCTT |
| | | CTAGCCGCTCCTCCAGCCGCAGCAG |
| | | AAACAGCAGCAGGAACAGCACCCCC |
| | | GGCAGCTCCCGGGGCACCAGCCCCG |
| | | CCAGAATGGCCGGAAATGGCGGCGA |
| | | TGCCGCCCTGGCCCTGCTCCTGCTG |
| | | GACAGACTGAATCAGCTGGAAAGCA |
| | | AGATGAGCGGCAAAGGACAGCAGCA |
| | | GCAAGGCCAGACCGTGACCAAGAAA |
| | | AGCGCTGCTGAAGCCTCCAAGAAAC |
| | | CTAGACAAAAGCGGACCGCCACAAA |
| | | GGCCTACAACGTGACCCAAGCCTTT |
| | | GGAAGAAGAGGCCCCGAGCAGACAC |
| | | AGGGCAATTTCGGCGACCAGGAGCT |
| | | GATCCGGCAGGGAACCGACTACAAG |
| | | CACTGGCCTCAGATCGCCCAGTTCG |
| | | CCCCTAGCGCCAGCGCCTTCTTCGG |
| | | CATGAGCAGAATCGGCATGGAAGTG |
| | | ACCCCTTCTGGCACCTGGCTGACCT |
| | | ACACCGGCGCTATCAAGCTGGACGA |
| | | TAAGGATCCTAACTTCAAGGACCAA |
| | | GTGATCCTGCTGAACAAGCATATCG |
| | | ACGCCTATAAGACCTTTCCACCTAC |
| | | AGAGCCTAAGAAAGATAAGAAGAAG |
| | | AAAGCCGACGAGACACAGGCCCTGC |
| | | CTCAGAGACAGAAAAAGCAGCAGAC |
| | | AGTGACACTGCTGCCAGCCGCTGAC |
| | | CTGGATGACTTCAGCAAGCAGCTGC |
| | | AGCAGAGCATGTCTTCTGCTGATAG |
| | | CACCCAGGCCCGAAAACGGCGCgga |
| | | agcggaggaagcggagctactaact |
| | | tcagcctgctgaagcaggctggaga |
| | | tgtggaggagaaccctggacctATG |
| | | TTCGTGTTCCTGGTGCTGCTGCCTC |
| | | TGGTCAGCTCCCAGTGTGTGAACCT |
| | | GACCACCAGAACCCAGCTGCCACCT |
| | | GCTTATACAAACTCCTTCACTCGGG |
| | | GGGTATACTACCCCGACAAGGTGTT |
| | | CAGATCTAGCGTGCTGCATTCTACA |
| | | CAAGACCTGTTCCTGCCCTTCTTCA |
| | | GCAACGTGACCTGGTTCCACGCCAT |
| | | CCACGTGTCTGGAACCAACGGAACC |
| | | AAGAGATTCGACAACCCCGTGCTGC |
| | | CTTTCAACGACGGCGTGTACTTCGC |
| | | CAGCACCGAGAAGTCCAACATCATC |
| | | AGAGGATGGATTTTCGGCACCACAC |
| | | TGGACAGCAAAACCCAGAGCCTGCT |
| | | GATCGTGAACAACGCCACCAACGTG |
| | | GTGATCAAGGTGTGCGAGTTCCAGT |
| | | TCTGCAATGATCCCTTCCTGGGCGT |
| | | GTACTACCACAAGAACAACAAGTCT |
| | | TGGATGGAAAGCGAGTTCAGAGTGT |
| | | ATTCCAGCGCCAACAATTGCACCTT |
| | | CGAGTACGTGAGCCAACCCTTTCTG |
| | | ATGGACCTTGAAGGCAAGCAGGGCA |
| | | ACTTCAAAAATCTGCGAGAATTTGT |
| | | GTTCAAGAACATCGACGGATACTTC |
| | | AAGATCTACTCTAAGCACACGCCAA |
| | | TCAACCTGGTGAGAGATCTGCCCCA |
| | | GGGCTTTAGCGCTTTGGAACCTCTG |
| | | GTGGACCTGCCTATCGGAATCAACA |
| | | TCACCAGATTTCAAACTCTCCTGGC |
| | | CCTGCACAGATCTTATCTGACCCCT |
| | | GGGGACAGTAGTAGCGGCTGGACAG |
| | | CCGGCGCCGCCGCCTACTACGTGGG |
| | | ATACCTGCAGCCTAGAACATTCCTG |
| | | CTGAAGTACAATGAGAACGGAACAA |
| | | TCACAGACGCCGTGGACTGCGCCCT |
| | | GGATCCTTTGAGCGAGACAAAGTGC |
| | | ACCCTGAAGTCGTTCACCGTCGAAA |
| | | AAGGCATCTACCAGACCAGCAACTT |
| | | CCGCGTGCAGCCTACGGAATCTATC |
| | | GTGCGGTTCCCCAACATCACCAACC |
| | | TGTGCCCTTTCGGCGAGGTGTTTAA |
| | | CGCTACAAGGTTCGCCAGCGTGTAT |
| | | GCCTGGAACAGAAAGAGAATCAGCA |
| | | ATTGCGTGGCCGATTATAGCGTTCT |
| | | GTACAACAGCGCTTCCTTCAGCACC |
| | | TTCAAGTGCTACGGCGTGTCTCCAA |
| | | CCAAGCTGAACGACCTCTGCTTCAC |
| | | CAATGTCTACGCTGACTCTTTCGTG |
| | | ATTAGAGGCGATGAGGTTAGACAGA |
| | | TCGCACCTGGCCAGACCGGCAAAAT |
| | | CGCTGACTACAACTACAAGCTGCCT |
| | | GATGACTTCACAGGCTGTGTCATTG |
| | | CCTGGAACTCAAATAACCTGGACTC |
| | | TAAAGTGGGCGGCAACTACAACTAC |
| | | CTGTACCGGCTGTTCCGGAAGAGCA |
| | | ATCTGAAACCTTTTGAGCGGGACAT |
| | | CTCTACAGAGATCTACCAGGCCGGC |
| | | AGCACACCCTGCAACGGCGTTGAGG |
| | | GCTTCAACTGCTACTTCCCTCTGCA |
| | | GAGCTACGGCTTTCAGCCAACAAAT |
| | | GGAGTGGGCTACCAGCCGTACAGAG |
| | | TGGTGGTGCTGAGCTTCGAACTGCT |
| | | GCATGCCCCAGCCACAGTGTGTGGA |
| | | CCTAAGAAGTCTACCAACCTGGTGA |
| | | AGAACAAGTGCGTGAACTTTAACTT |
| | | TAACGGCCTGACCGGCACAGGCGTG |
| | | CTGACCGAATCCAACAAAAAGTTCC |
| | | TGCCCTTCCAACAGTTCGGCAGAGA |
| | | CATCGCCGATACAACCGATGCCGTG |
| | | CGGGACCCCCAGACCTTAGAAATCC |
| | | TAGATATCACCCCGTGCAGCTTCGG |
| | | CGGAGTCTCTGTTATTACTCCTGGC |
| | | ACCAACACCAGCAACCAAGTGGCTG |
| | | TTCTGTACCAAggcGTGAACTGCAC |
| | | CGAAGTGCCTGTGGCTATCCACGCC |
| | | GATCAGCTGACCCCAACCTGGCGGG |
| | | TGTATAGCACCGGCTCTAACGTGTT |
| | | CCAGACCCGGGCTGGCTGCCTGATC |
| | | GGCGCCGAACACGTCAACAACTCCT |
| | | ATGAATGTGACATCCCCATCGGGGC |
| | | TGGCATCTGCGCCAGTTACCAGACA |
| | | CAGACAAATAGCCCTGGCAGCGCCA |
| | | GCAGCGTGGCCTCCCAGAGTATCAT |
| | | TGCCTACACCATGAGCCTGGGCGCC |
| | | GAGAACAGCGTGGCCTATTCTAACA |
| | | ATAGCATCGCAATCCCTACCAACTT |
| | | TACCATCTCTGTGACAACCGAGATC |
| | | CTGCCTGTGAGCATGACCAAAACCA |
| | | GCGTGGACTGCACGATGTACATCTG |
| | | TGGCGACAGCACAGAATGCAGTAAT |
| | | CTGTTGCTGCAGTACGGCAGCTTTT |
| | | GCACCCAGTTGAATAGAGCCCTGAC |
| | | CGGAATCGCCGTAGAGCAGGACAAA |
| | | AATACCCAGGAGGTGTTCGCCCAGG |
| | | TGAAACAGATCTACAAGACACCTCC |
| | | CATTAAGGACTTCGGAGGTTTTAAC |
| | | TTCAGCCAGATCCTGCCCGACCCTT |
| | | CCAAGCCTAGCAAACGCTCCTTCAT |
| | | CGAGGACCTGCTCTTCAACAAGGTG |
| | | ACACTGGCTGATGCCGGCTTCATCA |
| | | AGCAGTACGGAGATTGTCTGGGAGA |
| | | CATCGCCGCTAGAGATCTGATCTGC |
| | | GCCCAAAAGTTCAACGGCCTGACCG |
| | | TGCTGCCTCCTCTGCTTACAGACGA |
| | | GATGATCGCCCAGTACACCAGCGCC |
| | | CTGCTGGCTGGCACCATCACAAGCG |
| | | GCTGGACCTTCGGAGCCGGAGCCGC |
| | | TCTGCAAATCCCCTTTGCCATGCAG |
| | | ATGGCCTACCGGTTCAACGGCATCG |
| | | GCGTGACACAGAATGTGCTGTACGA |
| | | GAACCAGAAGCTGATCGCTAACCAG |
| | | TTTAACAGCGCTATCGGCAAGATCC |
| | | AGGACTCGCTGAGTAGCACCGCCTC |
| | | TGCCCTGGGCAAGCTGCAGGACGTC |
| | | GTGAACCAGAACGCCCAAGCCCTGA |
| | | ACACACTGGTGAAACAGCTGAGCAG |
| | | CAACTTCGGCGCCATCAGCTCTGTG |
| | | CTGAACGATATCCTGAGCAGACTGG |
| | | ACCCTcccGAAGCCGAGGTCCAGAT |
| | | CGACAGACTGATCACAGGAAGACTG |
| | | CAGAGCCTGCAAACGTACGTGACAC |
| | | AGCAGCTGATCCGGGCAGCCGAAAT |
| | | CCGGGCCAGCGCCAATCTGGCCGCT |
| | | ACCAAGATGAGCGAGTGCGTGTTAG |
| | | GCCAGAGCAAGCGGGTGGATTTCTG |
| | | CGGTAAGGGATACCACCTGATGAGC |
| | | TTTCCCCAGAGCGCTCCTCACGGCG |
| | | TGGTGTTTCTGCACGTGACCTACGT |
| | | TCCTGCCCAGGAAAAGAACTTCACC |
| | | ACCGCCCCTGCTATCTGCCACGATG |
| | | GCAAGGCCCACTTCCCTAGAGAGGG |
| | | CGTTTTCGTGTCTAACGGCACACAC |
| | | TGGTTTGTGACCCAGAGAAACTTCT |
| | | ACGAGCCTCAGATCATCACCACAGA |
| | | CAACACCTTTGTGAGCGGCAATTGC |
| | | GACGTGGTGATCGGAATTGTTAATA |
| | | ATACCGTGTACGACCCTCTGCAGCC |
| | | TGAGCTCGACAGCTTCAAGGAAGAG |
| | | CTGGACAAGTACTTCAAGAACCACA |
| | | CCTCCCCAGATGTGGACCTGGGCGA |
| | | TATTTCAGGCATCAACGCCTCCGTC |
| | | GTGAATATCCAGAAGGAGATCGACC |
| | | GGCTCAACGAGGTGGCCAAGAACCT |
| | | TAACGAGAGCCTGATCGACCTGCAG |
| | | GAACTGGGCAAATATGAGCAGTACA |
| | | TCAAGTGGCCTTGGTACATCTGGCT |
| | | GGGCTTTATCGCAGGCCTGATCGCT |
| | | ATCGTGATGGTGACCATTATGCTGT |
| | | GTTGTATGACCAGCTGTTGTAGTTG |
| | | TCTGAAGGGCTGCTGTTCTTGCGGC |
| | | AGCTGCTGCAAGTTCGACGAAGACG |
| | | ACTCAGAGCCCGTGCTGAAAGGCGT |
| | | GAAGCTGCACTACACCCGAAAACGG |
| | | CGCggaagcggaggaagcggagcta |
| | | ctaacttcagcctgctgaagcaggc |
| | | tggagatgtggaggagaaccctgga |
| | | cctATGTATTCTTTTGTGTCCGAGG |
| | | AAACCGGCACACTGATCGTTAATAG |
| | | CGTGCTGCTCTTCCTGGCCTTCGTG |
| | | GTGTTCCTGCTGGTGACCCTGGCTA |
| | | TCCTGACCGCCCTGAGACTGTGTGC |
| | | CTACTGCTGCAACATCGTGAACGTG |
| | | TCTCTGGTCAAGCCTAGCTTCTACG |
| | | TGTACAGCCGGGTGAAGAACCTGAA |
| | | CAGCAGCAGAGTGCCCGACCTGCTG |
| | | GTGtaatcccccccccctaacgtta |
| | | ctggccgaagccgcttggaataagg |
| | | ccggtgtgcgtttgtctatatgtta |
| | | ttttccaccatattgccgtcttttg |
| | | gcaatgtgagggcccggaaacctgg |
| | | ccctgtcttcttgacgagcattcct |
| | | aggggtctttcccctctcgccaaag |
| | | gaatgcaaggtctgttgaatgtcgt |
| | | gaaggaagcagttcctctggaagct |
| | | tcttgaagacaaacaacgtctgtag |
| | | cgaccctttgcaggcagcggaaccc |
| | | cccacctggcgacaggtgcctctgc |
| | | ggccaaaagccacgtgtataagata |
| | | cacctgcaaaggcggcacaacccca |
| | | gtgccacgttgtgagttggatagtt |
| | | gtggaaagagtcaaatggctctcct |
| | | caagcgtattcaacaaggggctgaa |
| | | ggatgcccagaaggtaccccattgt |
| | | atgggatctgatctggggcctcggt |
| | | gcacatgctttacatgtgtttagtc |
| | | gaggttaaaaaaacgtctaggcccc |
| | | ccgaaccacggggacgtggttttcc |
| | | tttgaaaaacacgatgataatatgg |
| | | ccacaaccatggaacaagagacttg |
| | | cgcgcactctctcacttttgaggaa |
| | | tgcccaaaatgctctgctctacaat |
| | | accgtaatggattttacctgctaaa |
| | | gtatgatgaagaatggtacccagag |
| | | gagttattgactgatggagaggatg |
| | | atgtctttgatcccgaattagacat |
| | | ggaagtcgttttcgagttacagtaa |
| CoVEG10 | 42 | ATGGCCGACAGCAACGGCACAATCA |
| expression | | CCGTGGAAGAGCTGAAGAAACTGCT |
| cassette | | GGAACAGTGGAACCTGGTCATCGGC |
| | | TTCCTGTTTCTGACCTGGATCTGTC |
| | | TGCTGCAGTTCGCTTATGCCAATCG |
| | | GAACAGATTCCTGTACATCATCAAG |
| | | CTGATCTTCCTGTGGCTGCTGTGGC |
| | | CTGTGACCCTGGCTTGCTTCGTGCT |
| | | GGCCGCTGTGTACCGGATCAACTGG |
| | | ATCACAGGCGGAATCGCCATCGCCA |
| | | TGGCCTGCCTGGTGGGCCTGATGTG |
| | | GCTGAGCTACTTCATCGCTTCTTTC |
| | | AGACTGTTCGCCAGAACCCGGAGCA |
| | | TGTGGTCCTTCAACCCCGAGACAAA |
| | | CATCCTGCTGAACGTGCCTCTGCAC |
| | | GGCACCATCCTGACAAGACCTCTGC |
| | | TCGAGAGCGAGCTGGTGATTGGCGC |
| | | AGTGATTCTGAGAGGCCATCTGAGG |
| | | ATCGCCGGACACCACCTGGGCAGAT |
| | | GCGACATCAAGGACCTTCCAAAGGA |
| | | AATCACCGTTGCCACCAGCCGGACC |
| | | CTGTCCTACTACAAACTGGGCGCCA |
| | | GCCAAAGAGTGGCCGGCGATAGCGG |
| | | CTTTGCCGCCTACAGCAGATACCGC |
| | | ATCGGAAATTACAAGCTCAACACCG |
| | | ACCACAGCAGCTCTTCTGATAACAT |
| | | CGCCCTGCTGGTGCAGCGAAAACGG |
| | | CGCggaagcggaggaagcggagcta |
| | | ctaacttcagcctgctgaagcaggc |
| | | tggagatgtggaggagaaccctgga |
| | | cctATGAGCGACAACGGCCCTCAAA |
| | | ACCAGAGAAATGCCCCTCGGATCAC |
| | | ATTTGGCGGACCTAGCGACAGCACC |
| | | GGCAGCAACCAGAATGGAGAAAGAA |
| | | GCGGCGCCAGATCCAAGCAGCGGAG |
| | | ACCTCAGGGACTGCCCAACAACACC |
| | | GCTAGCTGGTTCACCGCCCTGACCC |
| | | AACACGGCAAGGAAGATCTGAAGTT |
| | | CCCCAGAGGCCAGGGCGTGCCTATC |
| | | AACACAAACTCTTCTCCCGACGACC |
| | | AGATCGGATACTATAGACGGGCCAC |
| | | TCGGAGAATTCGGGGCGGCGACGGA |
| | | AAAATGAAGGACCTTTCTCCAAGAT |
| | | GGTACTTCTACTACCTCGGCACAGG |
| | | CCCTGAGGCCGGCCTGCCTTACGGC |
| | | GCCAACAAGGATGGCATCATCTGGG |
| | | TCGCCACCGAGGGCGCCCTGAACAC |
| | | CCCTAAGGACCACATCGGCACAAGA |
| | | AACCCCGCTAACAACGCCGCAATCG |
| | | TGCTGCAGCTGCCTCAGGGCACCAC |
| | | CCTGCCCAAGGGCTTCTACGCCGAG |
| | | GGCTCTAGAGGTGGCTCCCAGGCTT |
| | | CTAGCCGCTCCTCCAGCCGCAGCAG |
| | | AAACAGCAGCAGGAACAGCACCCCC |
| | | GGCAGCTCCCGGGGCACCAGCCCCG |
| | | CCAGAATGGCCGGAAATGGCGGCGA |
| | | TGCCGCCCTGGCCCTGCTCCTGCTG |
| | | GACAGACTGAATCAGCTGGAAAGCA |
| | | AGATGAGCGGCAAAGGACAGCAGCA |
| | | GCAAGGCCAGACCGTGACCAAGAAA |
| | | AGCGCTGCTGAAGCCTCCAAGAAAC |
| | | CTAGACAAAAGCGGACCGCCACAAA |
| | | GGCCTACAACGTGACCCAAGCCTTT |
| | | GGAAGAAGAGGCCCCGAGCAGACAC |
| | | AGGGCAATTTCGGCGACCAGGAGCT |
| | | GATCCGGCAGGGAACCGACTACAAG |
| | | CACTGGCCTCAGATCGCCCAGTTCG |
| | | CCCCTAGCGCCAGCGCCTTCTTCGG |
| | | CATGAGCAGAATCGGCATGGAAGTG |
| | | ACCCCTTCTGGCACCTGGCTGACCT |
| | | ACACCGGCGCTATCAAGCTGGACGA |
| | | TAAGGATCCTAACTTCAAGGACCAA |
| | | GTGATCCTGCTGAACAAGCATATCG |
| | | ACGCCTATAAGACCTTTCCACCTAC |
| | | AGAGCCTAAGAAAGATAAGAAGAAG |
| | | AAAGCCGACGAGACACAGGCCCTGC |
| | | CTCAGAGACAGAAAAAGCAGCAGAC |
| | | AGTGACACTGCTGCCAGCCGCTGAC |
| | | CTGGATGACTTCAGCAAGCAGCTGC |
| | | AGCAGAGCATGTCTTCTGCTGATAG |
| | | CACCCAGGCCCGAAAACGGCGCgga |
| | | agcggaggaagcggagctactaact |
| | | tcagcctgctgaagcaggctggaga |
| | | tgtggaggagaaccctggacctATG |
| | | TTCGTGTTCCTGGTGCTGCTGCCTC |
| | | TGGTCAGCTCCCAGTGTGTGAACCT |
| | | GACCACCAGAACCCAGCTGCCACCT |
| | | GCTTATACAAACTCCTTCACTCGGG |
| | | GGGTATACTACCCCGACAAGGTGTT |
| | | CAGATCTAGCGTGCTGCATTCTACA |
| | | CAAGACCTGTTCCTGCCCTTCTTCA |
| | | GCAACGTGACCTGGTTCCACGCCAT |
| | | CCACGTGTCTGGAACCAACGGAACC |
| | | AAGAGATTCGACAACCCCGTGCTGC |
| | | CTTTCAACGACGGCGTGTACTTCGC |
| | | CAGCACCGAGAAGTCCAACATCATC |
| | | AGAGGATGGATTTTCGGCACCACAC |
| | | TGGACAGCAAAACCCAGAGCCTGCT |
| | | GATCGTGAACAACGCCACCAACGTG |
| | | GTGATCAAGGTGTGCGAGTTCCAGT |
| | | TCTGCAATGATCCCTTCCTGGGCGT |
| | | GTACTACCACAAGAACAACAAGTCT |
| | | TGGATGGAAAGCGAGTTCAGAGTGT |
| | | ATTCCAGCGCCAACAATTGCACCTT |
| | | CGAGTACGTGAGCCAACCCTTTCTG |
| | | ATGGACCTTGAAGGCAAGCAGGGCA |
| | | ACTTCAAAAATCTGCGAGAATTTGT |
| | | GTTCAAGAACATCGACGGATACTTC |
| | | AAGATCTACTCTAAGCACACGCCAA |
| | | TCAACCTGGTGAGAGATCTGCCCCA |
| | | GGGCTTTAGCGCTTTGGAACCTCTG |
| | | GTGGACCTGCCTATCGGAATCAACA |
| | | TCACCAGATTTCAAACTCTCCTGGC |
| | | CCTGCACAGATCTTATCTGACCCCT |
| | | GGGGACAGTAGTAGCGGCTGGACAG |
| | | CCGGCGCCGCCGCCTACTACGTGGG |
| | | ATACCTGCAGCCTAGAACATTCCTG |
| | | CTGAAGTACAATGAGAACGGAACAA |
| | | TCACAGACGCCGTGGACTGCGCCCT |
| | | GGATCCTTTGAGCGAGACAAAGTGC |
| | | ACCCTGAAGTCGTTCACCGTCGAAA |
| | | AAGGCATCTACCAGACCAGCAACTT |
| | | CCGCGTGCAGCCTACGGAATCTATC |
| | | GTGCGGTTCCCCAACATCACCAACC |
| | | TGTGCCCTTTCGGCGAGGTGTTTAA |
| | | CGCTACAAGGTTCGCCAGCGTGTAT |
| | | GCCTGGAACAGAAAGAGAATCAGCA |
| | | ATTGCGTGGCCGATTATAGCGTTCT |
| | | GTACAACAGCGCTTCCTTCAGCACC |
| | | TTCAAGTGCTACGGCGTGTCTCCAA |
| | | CCAAGCTGAACGACCTCTGCTTCAC |
| | | CAATGTCTACGCTGACTCTTTCGTG |
| | | ATTAGAGGCGATGAGGTTAGACAGA |
| | | TCGCACCTGGCCAGACCGGCAAAAT |
| | | CGCTGACTACAACTACAAGCTGCCT |
| | | GATGACTTCACAGGCTGTGTCATTG |
| | | CCTGGAACTCAAATAACCTGGACTC |
| | | TAAAGTGGGCGGCAACTACAACTAC |
| | | CTGTACCGGCTGTTCCGGAAGAGCA |
| | | ATCTGAAACCTTTTGAGCGGGACAT |
| | | CTCTACAGAGATCTACCAGGCCGGC |
| | | AGCACACCCTGCAACGGCGTTGAGG |
| | | GCTTCAACTGCTACTTCCCTCTGCA |
| | | GAGCTACGGCTTTCAGCCAACAAAT |
| | | GGAGTGGGCTACCAGCCGTACAGAG |
| | | TGGTGGTGCTGAGCTTCGAACTGCT |
| | | GCATGCCCCAGCCACAGTGTGTGGA |
| | | CCTAAGAAGTCTACCAACCTGGTGA |
| | | AGAACAAGTGCGTGAACTTTAACTT |
| | | TAACGGCCTGACCGGCACAGGCGTG |
| | | CTGACCGAATCCAACAAAAAGTTCC |
| | | TGCCCTTCCAACAGTTCGGCAGAGA |
| | | CATCGCCGATACAACCGATGCCGTG |
| | | CGGGACCCCCAGACCTTAGAAATCC |
| | | TAGATATCACCCCGTGCAGCTTCGG |
| | | CGGAGTCTCTGTTATTACTCCTGGC |
| | | ACCAACACCAGCAACCAAGTGGCTG |
| | | TTCTGTACCAAggcGTGAACTGCAC |
| | | CGAAGTGCCTGTGGCTATCCACGCC |
| | | GATCAGCTGACCCCAACCTGGCGGG |
| | | TGTATAGCACCGGCTCTAACGTGTT |
| | | CCAGACCCGGGCTGGCTGCCTGATC |
| | | GGCGCCGAACACGTCAACAACTCCT |
| | | ATGAATGTGACATCCCCATCGGGGC |
| | | TGGCATCTGCGCCAGTTACCAGACA |
| | | CAGACAAATAGCCCTGGCAGCGCCA |
| | | GCAGCGTGGCCTCCCAGAGTATCAT |
| | | TGCCTACACCATGAGCCTGGGCGCC |
| | | GAGAACAGCGTGGCCTATTCTAACA |
| | | ATAGCATCGCAATCCCTACCAACTT |
| | | TACCATCTCTGTGACAACCGAGATC |
| | | CTGCCTGTGAGCATGACCAAAACCA |
| | | GCGTGGACTGCACGATGTACATCTG |
| | | TGGCGACAGCACAGAATGCAGTAAT |
| | | CTGTTGCTGCAGTACGGCAGCTTTT |
| | | GCACCCAGTTGAATAGAGCCCTGAC |
| | | CGGAATCGCCGTAGAGCAGGACAAA |
| | | AATACCCAGGAGGTGTTCGCCCAGG |
| | | TGAAACAGATCTACAAGACACCTCC |
| | | CATTAAGGACTTCGGAGGTTTTAAC |
| | | TTCAGCCAGATCCTGCCCGACCCTT |
| | | CCAAGCCTAGCAAACGCTCCTTCAT |
| | | CGAGGACCTGCTCTTCAACAAGGTG |
| | | ACACTGGCTGATGCCGGCTTCATCA |
| | | AGCAGTACGGAGATTGTCTGGGAGA |
| | | CATCGCCGCTAGAGATCTGATCTGC |
| | | GCCCAAAAGTTCAACGGCCTGACCG |
| | | TGCTGCCTCCTCTGCTTACAGACGA |
| | | GATGATCGCCCAGTACACCAGCGCC |
| | | CTGCTGGCTGGCACCATCACAAGCG |
| | | GCTGGACCTTCGGAGCCGGAGCCGC |
| | | TCTGCAAATCCCCTTTGCCATGCAG |
| | | ATGGCCTACCGGTTCAACGGCATCG |
| | | GCGTGACACAGAATGTGCTGTACGA |
| | | GAACCAGAAGCTGATCGCTAACCAG |
| | | TTTAACAGCGCTATCGGCAAGATCC |
| | | AGGACTCGCTGAGTAGCACCGCCTC |
| | | TGCCCTGGGCAAGCTGCAGGACGTC |
| | | GTGAACCAGAACGCCCAAGCCCTGA |
| | | ACACACTGGTGAAACAGCTGAGCAG |
| | | CAACTTCGGCGCCATCAGCTCTGTG |
| | | CTGAACGATATCCTGAGCAGACTGG |
| | | ACCCTcccGAAGCCGAGGTCCAGAT |
| | | CGACAGACTGATCACAGGAAGACTG |
| | | CAGAGCCTGCAAACGTACGTGACAC |
| | | AGCAGCTGATCCGGGCAGCCGAAAT |
| | | CCGGGCCAGCGCCAATCTGGCCGCT |
| | | ACCAAGATGAGCGAGTGCGTGTTAG |
| | | GCCAGAGCAAGCGGGTGGATTTCTG |
| | | CGGTAAGGGATACCACCTGATGAGC |
| | | TTTCCCCAGAGCGCTCCTCACGGCG |
| | | TGGTGTTTCTGCACGTGACCTACGT |
| | | TCCTGCCCAGGAAAAGAACTTCACC |
| | | ACCGCCCCTGCTATCTGCCACGATG |
| | | GCAAGGCCCACTTCCCTAGAGAGGG |
| | | CGTTTTCGTGTCTAACGGCACACAC |
| | | TGGTTTGTGACCCAGAGAAACTTCT |
| | | ACGAGCCTCAGATCATCACCACAGA |
| | | CAACACCTTTGTGAGCGGCAATTGC |
| | | GACGTGGTGATCGGAATTGTTAATA |
| | | ATACCGTGTACGACCCTCTGCAGCC |
| | | TGAGCTCGACAGCTTCAAGGAAGAG |
| | | CTGGACAAGTACTTCAAGAACCACA |
| | | CCTCCCCAGATGTGGACCTGGGCGA |
| | | TATTTCAGGCATCAACGCCTCCGTC |
| | | GTGAATATCCAGAAGGAGATCGACC |
| | | GGCTCAACGAGGTGGCCAAGAACCT |
| | | TAACGAGAGCCTGATCGACCTGCAG |
| | | GAACTGGGCAAATATGAGCAGTACA |
| | | TCAAGTGGCCTTGGTACATCTGGCT |
| | | GGGCTTTATCGCAGGCCTGATCGCT |
| | | ATCGTGATGGTGACCATTATGCTGT |
| | | GTTGTATGACCAGCTGTTGTAGTTG |
| | | TCTGAAGGGCTGCTGTTCTTGCGGC |
| | | AGCTGCTGCAAGTTCGACGAAGACG |
| | | ACTCAGAGCCCGTGCTGAAAGGCGT |
| | | GAAGCTGCACTACACCCGAAAACGG |
| | | CGCggaagcggaggaagcggagcta |
| | | ctaacttcagcctgctgaagcaggc |
| | | tggagatgtggaggagaaccctgga |
| | | cctATGTATTCTTTTGTGTCCGAGG |
| | | AAACCGGCACACTGATCGTTAATAG |
| | | CGTGCTGCTCTTCCTGGCCTTCGTG |
| | | GTGTTCCTGCTGGTGACCCTGGCTA |
| | | TCCTGACCGCCCTGAGACTGTGTGC |
| | | CTACTGCTGCAACATCGTGAACGTG |
| | | TCTCTGGTCAAGCCTAGCTTCTACG |
| | | TGTACAGCCGGGTGAAGAACCTGAA |
| | | CAGCAGCAGAGTGCCCGACCTGCTG |
| | | GTGtaatcccccccccctaacgtta |
| | | ctggccgaagccgcttggaataagg |
| | | ccggtgtgcgtttgtctatatgtta |
| | | ttttccaccatattgccgtcttttg |
| | | gcaatgtgagggcccggaaacctgg |
| | | ccctgtcttcttgacgagcattcct |
| | | aggggtctttcccctctcgccaaag |
| | | gaatgcaaggtctgttgaatgtcgt |
| | | gaaggaagcagttcctctggaagct |
| | | tcttgaagacaaacaacgtctgtag |
| | | cgaccctttgcaggcagcggaaccc |
| | | cccacctggcgacaggtgcctctgc |
| | | ggccaaaagccacgtgtataagata |
| | | cacctgcaaaggcggcacaacccca |
| | | gtgccacgttgtgagttggatagtt |
| | | gtggaaagagtcaaatggctctcct |
| | | caagcgtattcaacaaggggctgaa |
| | | ggatgcccagaaggtaccccattgt |
| | | atgggatctgatctggggcctcggt |
| | | gcacatgctttacatgtgtttagtc |
| | | gaggttaaaaaaacgtctaggcccc |
| | | ccgaaccacggggacgtggttttcc |
| | | tttgaaaaacacgatgataatatgg |
| | | ccacaaccatggaacaagagacttg |
| | | cgcgcactctctcacttttgaggaa |
| | | tgcccaaaatgctctgctctacaat |
| | | accgtaatggattttacctgctaaa |
| | | gtatgatgaagaatggtacccagag |
| | | gagttattgactgatggagaggatg |
| | | atgtctttgatcccgaattagacat |
| | | ggaagtcgttttcgagttacagtaa |
| | | GTGTCaAAGcGCGAGGAACTGTTCA |
| | | CCGGAGTttTGCCCATCCTGGTCGA |
| | | GCTGGACGGCGATGTGAACGGCCAC |
| | | AAGTTCAGCGTTTCTGGCGAGGGtg |
| | | agctttgggctaagcgcaacattaa |
| | | accagtaccagaggtgaaaatactc |
| | | aataatttgggtgtggacattgctg |
| | | ctaatactgtgatctgggactacaa |
| | | aagagatgctccagcacatatatct |
| | | actattggtgtttgttctatgactg |
| | | acatagccaagaaaccaactgaaac |
| | | gatttgtgcaccactcactgtcttt |
| | | tttgatggtagagttgatggtcaag |
| | | tagacttatttagaaatgcccgtaa |
| | | tggtgttcttattacagaaggtagt |
| | | gttaaaggtttacaaccatctgtag |
| | | gtcccaaacaagctagtcttaatgg |
| | | agtcacattaattggagaagccgta |
| | | aaaacacagttcaattattataaga |
| | | aagttgatggtgttgtccaacaatt |
| | | acctgaaacttactttactcagagt |
| | | agaaatttacaagaatttaaaccca |
| | | ggagtcaaatggaaattgatttctt |
| | | agaattagctatggatgaattcatt |
| | | gaacggtataaattagaaggctatg |
| | | ccttcgaacatatcgtttatggaga |
| | | ttttagtcata |
| CoVEG11 | 43 | aaaaaaaaaaATTAAAGGTTTATAC |
| expression | | CTTCCCAGGTAACAAACCAACCAAC |
| cassette | | TTTCGATCTCTTGTAGATCTGTTCT |
| | | CTAAACGAACTTTAAAATCTGTGTG |
| | | GCTGTCACTCGGCTGCATGCTTAGT |
| | | GCACTCACGCAGTATAATTAATAAC |
| | | TAATTACTGTCGTTGACAGGACACG |
| | | AGTAACTCGTCTATCTTCTGCAGGC |
| | | TGCTTACGGTTTCGTCCGTGTTGCA |
| | | GCCGATCATCAGCACATCTAGGTTT |
| | | CGTCCGGGTGTGACCGAAAGGTAAg |
| | | ATGGCCGACAGCAACGGCACAATCA |
| | | CCGTGGAAGAGCTGAAGAAACTGCT |
| | | GGAACAGTGGAACCTGGTCATCGGC |
| | | TTCCTGTTTCTGACCTGGATCTGTC |
| | | TGCTGCAGTTCGCTTATGCCAATCG |
| | | GAACAGATTCCTGTACATCATCAAG |
| | | CTGATCTTCCTGTGGCTGCTGTGGC |
| | | CTGTGACCCTGGCTTGCTTCGTGCT |
| | | GGCCGCTGTGTACCGGATCAACTGG |
| | | ATCACAGGCGGAATCGCCATCGCCA |
| | | TGGCCTGCCTGGTGGGCCTGATGTG |
| | | GCTGAGCTACTTCATCGCTTCTTTC |
| | | AGACTGTTCGCCAGAACCCGGAGCA |
| | | TGTGGTCCTTCAACCCCGAGACAAA |
| | | CATCCTGCTGAACGTGCCTCTGCAC |
| | | GGCACCATCCTGACAAGACCTCTGC |
| | | TCGAGAGCGAGCTGGTGATTGGCGC |
| | | AGTGATTCTGAGAGGCCATCTGAGG |
| | | ATCGCCGGACACCACCTGGGCAGAT |
| | | GCGACATCAAGGACCTTCCAAAGGA |
| | | AATCACCGTTGCCACCAGCCGGACC |
| | | CTGTCCTACTACAAACTGGGCGCCA |
| | | GCCAAAGAGTGGCCGGCGATAGCGG |
| | | CTTTGCCGCCTACAGCAGATACCGC |
| | | ATCGGAAATTACAAGCTCAACACCG |
| | | ACCACAGCAGCTCTTCTGATAACAT |
| | | CGCCCTGCTGGTGCAGCGAAAACGG |
| | | CGCggaagcggaggaagcggagcta |
| | | ctaacttcagcctgctgaagcaggc |
| | | tggagatgtggaggagaaccctgga |
| | | cctATGAGCGACAACGGCCCTCAAA |
| | | ACCAGAGAAATGCCCCTCGGATCAC |
| | | ATTTGGCGGACCTAGCGACAGCACC |
| | | GGCAGCAACCAGAATGGAGAAAGAA |
| | | GCGGCGCCAGATCCAAGCAGCGGAG |
| | | ACCTCAGGGACTGCCCAACAACACC |
| | | GCTAGCTGGTTCACCGCCCTGACCC |
| | | AACACGGCAAGGAAGATCTGAAGTT |
| | | CCCCAGAGGCCAGGGCGTGCCTATC |
| | | AACACAAACTCTTCTCCCGACGACC |
| | | AGATCGGATACTATAGACGGGCCAC |
| | | TCGGAGAATTCGGGGCGGCGACGGA |
| | | AAAATGAAGGACCTTTCTCCAAGAT |
| | | GGTACTTCTACTACCTCGGCACAGG |
| | | CCCTGAGGCCGGCCTGCCTTACGGC |
| | | GCCAACAAGGATGGCATCATCTGGG |
| | | TCGCCACCGAGGGCGCCCTGAACAC |
| | | CCCTAAGGACCACATCGGCACAAGA |
| | | AACCCCGCTAACAACGCCGCAATCG |
| | | TGCTGCAGCTGCCTCAGGGCACCAC |
| | | CCTGCCCAAGGGCTTCTACGCCGAG |
| | | GGCTCTAGAGGTGGCTCCCAGGCTT |
| | | CTAGCCGCTCCTCCAGCCGCAGCAG |
| | | AAACAGCAGCAGGAACAGCACCCCC |
| | | GGCAGCTCCCGGGGCACCAGCCCCG |
| | | CCAGAATGGCCGGAAATGGCGGCGA |
| | | TGCCGCCCTGGCCCTGCTCCTGCTG |
| | | GACAGACTGAATCAGCTGGAAAGCA |
| | | AGATGAGCGGCAAAGGACAGCAGCA |
| | | GCAAGGCCAGACCGTGACCAAGAAA |
| | | AGCGCTGCTGAAGCCTCCAAGAAAC |
| | | CTAGACAAAAGCGGACCGCCACAAA |
| | | GGCCTACAACGTGACCCAAGCCTTT |
| | | GGAAGAAGAGGCCCCGAGCAGACAC |
| | | AGGGCAATTTCGGCGACCAGGAGCT |
| | | GATCCGGCAGGGAACCGACTACAAG |
| | | CACTGGCCTCAGATCGCCCAGTTCG |
| | | CCCCTAGCGCCAGCGCCTTCTTCGG |
| | | CATGAGCAGAATCGGCATGGAAGTG |
| | | ACCCCTTCTGGCACCTGGCTGACCT |
| | | ACACCGGCGCTATCAAGCTGGACGA |
| | | TAAGGATCCTAACTTCAAGGACCAA |
| | | GTGATCCTGCTGAACAAGCATATCG |
| | | ACGCCTATAAGACCTTTCCACCTAC |
| | | AGAGCCTAAGAAAGATAAGAAGAAG |
| | | AAAGCCGACGAGACACAGGCCCTGC |
| | | CTCAGAGACAGAAAAAGCAGCAGAC |
| | | AGTGACACTGCTGCCAGCCGCTGAC |
| | | CTGGATGACTTCAGCAAGCAGCTGC |
| | | AGCAGAGCATGTCTTCTGCTGATAG |
| | | CACCCAGGCCCGAAAACGGCGCgga |
| | | agcggaggaagcggagctactaact |
| | | tcagcctgctgaagcaggctggaga |
| | | tgtggaggagaaccctggacctATG |
| | | TTCGTGTTCCTGGTGCTGCTGCCTC |
| | | TGGTCAGCTCCCAGTGTGTGAACCT |
| | | GACCACCAGAACCCAGCTGCCACCT |
| | | GCTTATACAAACTCCTTCACTCGGG |
| | | GGGTATACTACCCCGACAAGGTGTT |
| | | CAGATCTAGCGTGCTGCATTCTACA |
| | | CAAGACCTGTTCCTGCCCTTCTTCA |
| | | GCAACGTGACCTGGTTCCACGCCAT |
| | | CCACGTGTCTGGAACCAACGGAACC |
| | | AAGAGATTCGACAACCCCGTGCTGC |
| | | CTTTCAACGACGGCGTGTACTTCGC |
| | | CAGCACCGAGAAGTCCAACATCATC |
| | | AGAGGATGGATTTTCGGCACCACAC |
| | | TGGACAGCAAAACCCAGAGCCTGCT |
| | | GATCGTGAACAACGCCACCAACGTG |
| | | GTGATCAAGGTGTGCGAGTTCCAGT |
| | | TCTGCAATGATCCCTTCCTGGGCGT |
| | | GTACTACCACAAGAACAACAAGTCT |
| | | TGGATGGAAAGCGAGTTCAGAGTGT |
| | | ATTCCAGCGCCAACAATTGCACCTT |
| | | CGAGTACGTGAGCCAACCCTTTCTG |
| | | ATGGACCTTGAAGGCAAGCAGGGCA |
| | | ACTTCAAAAATCTGCGAGAATTTGT |
| | | GTTCAAGAACATCGACGGATACTTC |
| | | AAGATCTACTCTAAGCACACGCCAA |
| | | TCAACCTGGTGAGAGATCTGCCCCA |
| | | GGGCTTTAGCGCTTTGGAACCTCTG |
| | | GTGGACCTGCCTATCGGAATCAACA |
| | | TCACCAGATTTCAAACTCTCCTGGC |
| | | CCTGCACAGATCTTATCTGACCCCT |
| | | GGGGACAGTAGTAGCGGCTGGACAG |
| | | CCGGCGCCGCCGCCTACTACGTGGG |
| | | ATACCTGCAGCCTAGAACATTCCTG |
| | | CTGAAGTACAATGAGAACGGAACAA |
| | | TCACAGACGCCGTGGACTGCGCCCT |
| | | GGATCCTTTGAGCGAGACAAAGTGC |
| | | ACCCTGAAGTCGTTCACCGTCGAAA |
| | | AAGGCATCTACCAGACCAGCAACTT |
| | | CCGCGTGCAGCCTACGGAATCTATC |
| | | GTGCGGTTCCCCAACATCACCAACC |
| | | TGTGCCCTTTCGGCGAGGTGTTTAA |
| | | CGCTACAAGGTTCGCCAGCGTGTAT |
| | | GCCTGGAACAGAAAGAGAATCAGCA |
| | | ATTGCGTGGCCGATTATAGCGTTCT |
| | | GTACAACAGCGCTTCCTTCAGCACC |
| | | TTCAAGTGCTACGGCGTGTCTCCAA |
| | | CCAAGCTGAACGACCTCTGCTTCAC |
| | | CAATGTCTACGCTGACTCTTTCGTG |
| | | ATTAGAGGCGATGAGGTTAGACAGA |
| | | TCGCACCTGGCCAGACCGGCAAAAT |
| | | CGCTGACTACAACTACAAGCTGCCT |
| | | GATGACTTCACAGGCTGTGTCATTG |
| | | CCTGGAACTCAAATAACCTGGACTC |
| | | TAAAGTGGGCGGCAACTACAACTAC |
| | | CTGTACCGGCTGTTCCGGAAGAGCA |
| | | ATCTGAAACCTTTTGAGCGGGACAT |
| | | CTCTACAGAGATCTACCAGGCCGGC |
| | | AGCACACCCTGCAACGGCGTTGAGG |
| | | GCTTCAACTGCTACTTCCCTCTGCA |
| | | GAGCTACGGCTTTCAGCCAACAAAT |
| | | GGAGTGGGCTACCAGCCGTACAGAG |
| | | TGGTGGTGCTGAGCTTCGAACTGCT |
| | | GCATGCCCCAGCCACAGTGTGTGGA |
| | | CCTAAGAAGTCTACCAACCTGGTGA |
| | | AGAACAAGTGCGTGAACTTTAACTT |
| | | TAACGGCCTGACCGGCACAGGCGTG |
| | | CTGACCGAATCCAACAAAAAGTTCC |
| | | TGCCCTTCCAACAGTTCGGCAGAGA |
| | | CATCGCCGATACAACCGATGCCGTG |
| | | CGGGACCCCCAGACCTTAGAAATCC |
| | | TAGATATCACCCCGTGCAGCTTCGG |
| | | CGGAGTCTCTGTTATTACTCCTGGC |
| | | ACCAACACCAGCAACCAAGTGGCTG |
| | | TTCTGTACCAAggcGTGAACTGCAC |
| | | CGAAGTGCCTGTGGCTATCCACGCC |
| | | GATCAGCTGACCCCAACCTGGCGGG |
| | | TGTATAGCACCGGCTCTAACGTGTT |
| | | CCAGACCCGGGCTGGCTGCCTGATC |
| | | GGCGCCGAACACGTCAACAACTCCT |
| | | ATGAATGTGACATCCCCATCGGGGC |
| | | TGGCATCTGCGCCAGTTACCAGACA |
| | | CAGACAAATAGCCCTGGCAGCGCCA |
| | | GCAGCGTGGCCTCCCAGAGTATCAT |
| | | TGCCTACACCATGAGCCTGGGCGCC |
| | | GAGAACAGCGTGGCCTATTCTAACA |
| | | ATAGCATCGCAATCCCTACCAACTT |
| | | TACCATCTCTGTGACAACCGAGATC |
| | | CTGCCTGTGAGCATGACCAAAACCA |
| | | GCGTGGACTGCACGATGTACATCTG |
| | | TGGCGACAGCACAGAATGCAGTAAT |
| | | CTGTTGCTGCAGTACGGCAGCTTTT |
| | | GCACCCAGTTGAATAGAGCCCTGAC |
| | | CGGAATCGCCGTAGAGCAGGACAAA |
| | | AATACCCAGGAGGTGTTCGCCCAGG |
| | | TGAAACAGATCTACAAGACACCTCC |
| | | CATTAAGGACTTCGGAGGTTTTAAC |
| | | TTCAGCCAGATCCTGCCCGACCCTT |
| | | CCAAGCCTAGCAAACGCTCCTTCAT |
| | | CGAGGACCTGCTCTTCAACAAGGTG |
| | | ACACTGGCTGATGCCGGCTTCATCA |
| | | AGCAGTACGGAGATTGTCTGGGAGA |
| | | CATCGCCGCTAGAGATCTGATCTGC |
| | | GCCCAAAAGTTCAACGGCCTGACCG |
| | | TGCTGCCTCCTCTGCTTACAGACGA |
| | | GATGATCGCCCAGTACACCAGCGCC |
| | | CTGCTGGCTGGCACCATCACAAGCG |
| | | GCTGGACCTTCGGAGCCGGAGCCGC |
| | | TCTGCAAATCCCCTTTGCCATGCAG |
| | | ATGGCCTACCGGTTCAACGGCATCG |
| | | GCGTGACACAGAATGTGCTGTACGA |
| | | GAACCAGAAGCTGATCGCTAACCAG |
| | | TTTAACAGCGCTATCGGCAAGATCC |
| | | AGGACTCGCTGAGTAGCACCGCCTC |
| | | TGCCCTGGGCAAGCTGCAGGACGTC |
| | | GTGAACCAGAACGCCCAAGCCCTGA |
| | | ACACACTGGTGAAACAGCTGAGCAG |
| | | CAACTTCGGCGCCATCAGCTCTGTG |
| | | CTGAACGATATCCTGAGCAGACTGG |
| | | ACCCTcccGAAGCCGAGGTCCAGAT |
| | | CGACAGACTGATCACAGGAAGACTG |
| | | CAGAGCCTGCAAACGTACGTGACAC |
| | | AGCAGCTGATCCGGGCAGCCGAAAT |
| | | CCGGGCCAGCGCCAATCTGGCCGCT |
| | | ACCAAGATGAGCGAGTGCGTGTTAG |
| | | GCCAGAGCAAGCGGGTGGATTTCTG |
| | | CGGTAAGGGATACCACCTGATGAGC |
| | | TTTCCCCAGAGCGCTCCTCACGGCG |
| | | TGGTGTTTCTGCACGTGACCTACGT |
| | | TCCTGCCCAGGAAAAGAACTTCACC |
| | | ACCGCCCCTGCTATCTGCCACGATG |
| | | GCAAGGCCCACTTCCCTAGAGAGGG |
| | | CGTTTTCGTGTCTAACGGCACACAC |
| | | TGGTTTGTGACCCAGAGAAACTTCT |
| | | ACGAGCCTCAGATCATCACCACAGA |
| | | CAACACCTTTGTGAGCGGCAATTGC |
| | | GACGTGGTGATCGGAATTGTTAATA |
| | | ATACCGTGTACGACCCTCTGCAGCC |
| | | TGAGCTCGACAGCTTCAAGGAAGAG |
| | | CTGGACAAGTACTTCAAGAACCACA |
| | | CCTCCCCAGATGTGGACCTGGGCGA |
| | | TATTTCAGGCATCAACGCCTCCGTC |
| | | GTGAATATCCAGAAGGAGATCGACC |
| | | GGCTCAACGAGGTGGCCAAGAACCT |
| | | TAACGAGAGCCTGATCGACCTGCAG |
| | | GAACTGGGCAAATATGAGCAGTACA |
| | | TCAAGTGGCCTTGGTACATCTGGCT |
| | | GGGCTTTATCGCAGGCCTGATCGCT |
| | | ATCGTGATGGTGACCATTATGCTGT |
| | | GTTGTATGACCAGCTGTTGTAGTTG |
| | | TCTGAAGGGCTGCTGTTCTTGCGGC |
| | | AGCTGCTGCAAGTTCGACGAAGACG |
| | | ACTCAGAGCCCGTGCTGAAAGGCGT |
| | | GAAGCTGCACTACACCCGAAAACGG |
| | | CGCggaagcggaggaagcggagcta |
| | | ctaacttcagcctgctgaagcaggc |
| | | tggagatgtggaggagaaccctgga |
| | | cctATGTATTCTTTTGTGTCCGAGG |
| | | AAACCGGCACACTGATCGTTAATAG |
| | | CGTGCTGCTCTTCCTGGCCTTCGTG |
| | | GTGTTCCTGCTGGTGACCCTGGCTA |
| | | TCCTGACCGCCCTGAGACTGTGTGC |
| | | CTACTGCTGCAACATCGTGAACGTG |
| | | TCTCTGGTCAAGCCTAGCTTCTACG |
| | | TGTACAGCCGGGTGAAGAACCTGAA |
| | | CAGCAGCAGAGTGCCCGACCTGCTG |
| | | GTGtaatcccccccccctaacgtta |
| | | ctggccgaagccgcttggaataagg |
| | | ccggtgtgcgtttgtctatatgtta |
| | | ttttccaccatattgccgtcttttg |
| | | gcaatgtgagggcccggaaacctgg |
| | | ccctgtcttcttgacgagcattcct |
| | | aggggtctttcccctctcgccaaag |
| | | gaatgcaaggtctgttgaatgtcgt |
| | | gaaggaagcagttcctctggaagct |
| | | tcttgaagacaaacaacgtctgtag |
| | | cgaccctttgcaggcageggaaccc |
| | | cccacctggcgacaggtgcctctgc |
| | | ggccaaaagccacgtgtataagata |
| | | cacctgcaaaggcggcacaacccca |
| | | gtgccacgttgtgagttggatagtt |
| | | gtggaaagagtcaaatggctctcct |
| | | caagcgtattcaacaaggggctgaa |
| | | ggatgcccagaaggtaccccattgt |
| | | atgggatctgatctggggcctcggt |
| | | gcacatgctttacatgtgtttagtc |
| | | gaggttaaaaaaacgtctaggcccc |
| | | ccgaaccacggggacgtggttttcc |
| | | tttgaaaaacacgatgataatatgg |
| | | ccacaaccatggaacaagagacttg |
| | | cgcgcactctctcacttttgaggaa |
| | | tgcccaaaatgctctgctctacaat |
| | | accgtaatggattttacctgctaaa |
| | | gtatgatgaagaatggtacccagag |
| | | gagttattgactgatggagaggatg |
| | | atgtctttgatcccgaattagacat |
| | | ggaagtcgttttcgagttacagtaa |
| | | GTGTttacctgttaatgtagcattt |
| | | gagctttgggctaagcgcaacatta |
| | | aaccagtaccagaggtgaaaatact |
| | | caataatttgggtgtggacattgct |
| | | gctaatactgtgatctgggactaca |
| | | aaagagatgctccagcacatatatc |
| | | tactattggtgtttgttctatgact |
| | | gacatagccaagaaaccaactgaaa |
| | | cgatttgtgcaccactcactgtctt |
| | | ttttgatggtagagttgatggtcaa |
| | | gtagacttatttagaaatgcccgta |
| | | atggtgttcttattacagaaggtag |
| | | tgttaaaggtttacaaccatctgta |
| | | ggtcccaaacaagctagtcttaatg |
| | | gagtcacattaattggagaagccgt |
| | | aaaaacacagttcaattattataag |
| | | aaagttgatggtgttgtccaacaat |
| | | tacctgaaacttactttactcagag |
| | | tagaaatttacaagaatttaaaccc |
| | | aggagtcaaatggaaattgatttct |
| | | tagaattagctatggatgaattcat |
| | | tgaacggtataaattagaaggctat |
| | | gccttcgaacatatcgtttatggag |
| | | attttagtcatagtcagttaggtgg |
| | | tGCGAaattgttgttgtt |
| CoVEG12 | 44 | ATGGCCGACAGCAACGGCACAATCA |
| expression | | CCGTGGAAGAGCTGAAGAAACTGCT |
| cassette | | GGAACAGTGGAACCTGGTCATCGGC |
| | | TTCCTGTTTCTGACCTGGATCTGTC |
| | | TGCTGCAGTTCGCTTATGCCAATCG |
| | | GAACAGATTCCTGTACATCATCAAG |
| | | CTGATCTTCCTGTGGCTGCTGTGGC |
| | | CTGTGACCCTGGCTTGCTTCGTGCT |
| | | GGCCGCTGTGTACCGGATCAACTGG |
| | | ATCACAGGCGGAATCGCCATCGCCA |
| | | TGGCCTGCCTGGTGGGCCTGATGTG |
| | | GCTGAGCTACTTCATCGCTTCTTTC |
| | | AGACTGTTCGCCAGAACCCGGAGCA |
| | | TGTGGTCCTTCAACCCCGAGACAAA |
| | | CATCCTGCTGAACGTGCCTCTGCAC |
| | | GGCACCATCCTGACAAGACCTCTGC |
| | | TCGAGAGCGAGCTGGTGATTGGCGC |
| | | AGTGATTCTGAGAGGCCATCTGAGG |
| | | ATCGCCGGACACCACCTGGGCAGAT |
| | | GCGACATCAAGGACCTTCCAAAGGA |
| | | AATCACCGTTGCCACCAGCCGGACC |
| | | CTGTCCTACTACAAACTGGGCGCCA |
| | | GCCAAAGAGTGGCCGGCGATAGCGG |
| | | CTTTGCCGCCTACAGCAGATACCGC |
| | | ATCGGAAATTACAAGCTCAACACCG |
| | | ACCACAGCAGCTCTTCTGATAACAT |
| | | CGCCCTGCTGGTGCAGCGAAAACGG |
| | | CGCggaagcggaggaagcggagcta |
| | | ctaacttcagcctgctgaagcaggc |
| | | tggagatgtggaggagaaccctgga |
| | | cctATGAGCGACAACGGCCCTCAAA |
| | | ACCAGAGAAATGCCCCTCGGATCAC |
| | | ATTTGGCGGACCTAGCGACAGCACC |
| | | GGCAGCAACCAGAATGGAGAAAGAA |
| | | GCGGCGCCAGATCCAAGCAGCGGAG |
| | | ACCTCAGGGACTGCCCAACAACACC |
| | | GCTAGCTGGTTCACCGCCCTGACCC |
| | | AACACGGCAAGGAAGATCTGAAGTT |
| | | CCCCAGAGGCCAGGGCGTGCCTATC |
| | | AACACAAACTCTTCTCCCGACGACC |
| | | AGATCGGATACTATAGACGGGCCAC |
| | | TCGGAGAATTCGGGGCGGCGACGGA |
| | | AAAATGAAGGACCTTTCTCCAAGAT |
| | | GGTACTTCTACTACCTCGGCACAGG |
| | | CCCTGAGGCCGGCCTGCCTTACGGC |
| | | GCCAACAAGGATGGCATCATCTGGG |
| | | TCGCCACCGAGGGCGCCCTGAACAC |
| | | CCCTAAGGACCACATCGGCACAAGA |
| | | AACCCCGCTAACAACGCCGCAATCG |
| | | TGCTGCAGCTGCCTCAGGGCACCAC |
| | | CCTGCCCAAGGGCTTCTACGCCGAG |
| | | GGCTCTAGAGGTGGCTCCCAGGCTT |
| | | CTAGCCGCTCCTCCAGCCGCAGCAG |
| | | AAACAGCAGCAGGAACAGCACCCCC |
| | | GGCAGCTCCCGGGGCACCAGCCCCG |
| | | CCAGAATGGCCGGAAATGGCGGCGA |
| | | TGCCGCCCTGGCCCTGCTCCTGCTG |
| | | GACAGACTGAATCAGCTGGAAAGCA |
| | | AGATGAGCGGCAAAGGACAGCAGCA |
| | | GCAAGGCCAGACCGTGACCAAGAAA |
| | | AGCGCTGCTGAAGCCTCCAAGAAAC |
| | | CTAGACAAAAGCGGACCGCCACAAA |
| | | GGCCTACAACGTGACCCAAGCCTTT |
| | | GGAAGAAGAGGCCCCGAGCAGACAC |
| | | AGGGCAATTTCGGCGACCAGGAGCT |
| | | GATCCGGCAGGGAACCGACTACAAG |
| | | CACTGGCCTCAGATCGCCCAGTTCG |
| | | CCCCTAGCGCCAGCGCCTTCTTCGG |
| | | CATGAGCAGAATCGGCATGGAAGTG |
| | | ACCCCTTCTGGCACCTGGCTGACCT |
| | | ACACCGGCGCTATCAAGCTGGACGA |
| | | TAAGGATCCTAACTTCAAGGACCAA |
| | | GTGATCCTGCTGAACAAGCATATCG |
| | | ACGCCTATAAGACCTTTCCACCTAC |
| | | AGAGCCTAAGAAAGATAAGAAGAAG |
| | | AAAGCCGACGAGACACAGGCCCTGC |
| | | CTCAGAGACAGAAAAAGCAGCAGAC |
| | | AGTGACACTGCTGCCAGCCGCTGAC |
| | | CTGGATGACTTCAGCAAGCAGCTGC |
| | | AGCAGAGCATGTCTTCTGCTGATAG |
| | | CACCCAGGCCCGAAAACGGCGCgga |
| | | agcggaggaagcggagctactaact |
| | | tcagcctgctgaagcaggctggaga |
| | | tgtggaggagaaccctggacctATG |
| | | TTCGTGTTCCTGGTGCTGCTGCCTC |
| | | TGGTCAGCTCCCAGTGTGTGAACCT |
| | | GACCACCAGAACCCAGCTGCCACCT |
| | | GCTTATACAAACTCCTTCACTCGGG |
| | | GGGTATACTACCCCGACAAGGTGTT |
| | | CAGATCTAGCGTGCTGCATTCTACA |
| | | CAAGACCTGTTCCTGCCCTTCTTCA |
| | | GCAACGTGACCTGGTTCCACGCCAT |
| | | CCACGTGTCTGGAACCAACGGAACC |
| | | AAGAGATTCGACAACCCCGTGCTGC |
| | | CTTTCAACGACGGCGTGTACTTCGC |
| | | CAGCACCGAGAAGTCCAACATCATC |
| | | AGAGGATGGATTTTCGGCACCACAC |
| | | TGGACAGCAAAACCCAGAGCCTGCT |
| | | GATCGTGAACAACGCCACCAACGTG |
| | | GTGATCAAGGTGTGCGAGTTCCAGT |
| | | TCTGCAATGATCCCTTCCTGGGCGT |
| | | GTACTACCACAAGAACAACAAGTCT |
| | | TGGATGGAAAGCGAGTTCAGAGTGT |
| | | ATTCCAGCGCCAACAATTGCACCTT |
| | | CGAGTACGTGAGCCAACCCTTTCTG |
| | | ATGGACCTTGAAGGCAAGCAGGGCA |
| | | ACTTCAAAAATCTGCGAGAATTTGT |
| | | GTTCAAGAACATCGACGGATACTTC |
| | | AAGATCTACTCTAAGCACACGCCAA |
| | | TCAACCTGGTGAGAGATCTGCCCCA |
| | | GGGCTTTAGCGCTTTGGAACCTCTG |
| | | GTGGACCTGCCTATCGGAATCAACA |
| | | TCACCAGATTTCAAACTCTCCTGGC |
| | | CCTGCACAGATCTTATCTGACCCCT |
| | | GGGGACAGTAGTAGCGGCTGGACAG |
| | | CCGGCGCCGCCGCCTACTACGTGGG |
| | | ATACCTGCAGCCTAGAACATTCCTG |
| | | CTGAAGTACAATGAGAACGGAACAA |
| | | TCACAGACGCCGTGGACTGCGCCCT |
| | | GGATCCTTTGAGCGAGACAAAGTGC |
| | | ACCCTGAAGTCGTTCACCGTCGAAA |
| | | AAGGCATCTACCAGACCAGCAACTT |
| | | CCGCGTGCAGCCTACGGAATCTATC |
| | | GTGCGGTTCCCCAACATCACCAACC |
| | | TGTGCCCTTTCGGCGAGGTGTTTAA |
| | | CGCTACAAGGTTCGCCAGCGTGTAT |
| | | GCCTGGAACAGAAAGAGAATCAGCA |
| | | ATTGCGTGGCCGATTATAGCGTTCT |
| | | GTACAACAGCGCTTCCTTCAGCACC |
| | | TTCAAGTGCTACGGCGTGTCTCCAA |
| | | CCAAGCTGAACGACCTCTGCTTCAC |
| | | CAATGTCTACGCTGACTCTTTCGTG |
| | | ATTAGAGGCGATGAGGTTAGACAGA |
| | | TCGCACCTGGCCAGACCGGCAAAAT |
| | | CGCTGACTACAACTACAAGCTGCCT |
| | | GATGACTTCACAGGCTGTGTCATTG |
| | | CCTGGAACTCAAATAACCTGGACTC |
| | | TAAAGTGGGCGGCAACTACAACTAC |
| | | CTGTACCGGCTGTTCCGGAAGAGCA |
| | | ATCTGAAACCTTTTGAGCGGGACAT |
| | | CTCTACAGAGATCTACCAGGCCGGC |
| | | AGCACACCCTGCAACGGCGTTGAGG |
| | | GCTTCAACTGCTACTTCCCTCTGCA |
| | | GAGCTACGGCTTTCAGCCAACAAAT |
| | | GGAGTGGGCTACCAGCCGTACAGAG |
| | | TGGTGGTGCTGAGCTTCGAACTGCT |
| | | GCATGCCCCAGCCACAGTGTGTGGA |
| | | CCTAAGAAGTCTACCAACCTGGTGA |
| | | AGAACAAGTGCGTGAACTTTAACTT |
| | | TAACGGCCTGACCGGCACAGGCGTG |
| | | CTGACCGAATCCAACAAAAAGTTCC |
| | | TGCCCTTCCAACAGTTCGGCAGAGA |
| | | CATCGCCGATACAACCGATGCCGTG |
| | | CGGGACCCCCAGACCTTAGAAATCC |
| | | TAGATATCACCCCGTGCAGCTTCGG |
| | | CGGAGTCTCTGTTATTACTCCTGGC |
| | | ACCAACACCAGCAACCAAGTGGCTG |
| | | TTCTGTACCAAggcGTGAACTGCAC |
| | | CGAAGTGCCTGTGGCTATCCACGCC |
| | | GATCAGCTGACCCCAACCTGGCGGG |
| | | TGTATAGCACCGGCTCTAACGTGTT |
| | | CCAGACCCGGGCTGGCTGCCTGATC |
| | | GGCGCCGAACACGTCAACAACTCCT |
| | | ATGAATGTGACATCCCCATCGGGGC |
| | | TGGCATCTGCGCCAGTTACCAGACA |
| | | CAGACAAATAGCCCTAGACGGGCCA |
| | | GAAGCGTGGCCTCCCAGAGTATCAT |
| | | TGCCTACACCATGAGCCTGGGCGCC |
| | | GAGAACAGCGTGGCCTATTCTAACA |
| | | ATAGCATCGCAATCCCTACCAACTT |
| | | TACCATCTCTGTGACAACCGAGATC |
| | | CTGCCTGTGAGCATGACCAAAACCA |
| | | GCGTGGACTGCACGATGTACATCTG |
| | | TGGCGACAGCACAGAATGCAGTAAT |
| | | CTGTTGCTGCAGTACGGCAGCTTTT |
| | | GCACCCAGTTGAATAGAGCCCTGAC |
| | | CGGAATCGCCGTAGAGCAGGACAAA |
| | | AATACCCAGGAGGTGTTCGCCCAGG |
| | | TGAAACAGATCTACAAGACACCTCC |
| | | CATTAAGGACTTCGGAGGTTTTAAC |
| | | TTCAGCCAGATCCTGCCCGACCCTT |
| | | CCAAGCCTAGCAAACGCTCCTTCAT |
| | | CGAGGACCTGCTCTTCAACAAGGTG |
| | | ACACTGGCTGATGCCGGCTTCATCA |
| | | AGCAGTACGGAGATTGTCTGGGAGA |
| | | CATCGCCGCTAGAGATCTGATCTGC |
| | | GCCCAAAAGTTCAACGGCCTGACCG |
| | | TGCTGCCTCCTCTGCTTACAGACGA |
| | | GATGATCGCCCAGTACACCAGCGCC |
| | | CTGCTGGCTGGCACCATCACAAGCG |
| | | GCTGGACCTTCGGAGCCGGAGCCGC |
| | | TCTGCAAATCCCCTTTGCCATGCAG |
| | | ATGGCCTACCGGTTCAACGGCATCG |
| | | GCGTGACACAGAATGTGCTGTACGA |
| | | GAACCAGAAGCTGATCGCTAACCAG |
| | | TTTAACAGCGCTATCGGCAAGATCC |
| | | AGGACTCGCTGAGTAGCACCGCCTC |
| | | TGCCCTGGGCAAGCTGCAGGACGTC |
| | | GTGAACCAGAACGCCCAAGCCCTGA |
| | | ACACACTGGTGAAACAGCTGAGCAG |
| | | CAACTTCGGCGCCATCAGCTCTGTG |
| | | CTGAACGATATCCTGAGCAGACTGG |
| | | ACAAGGTGGAAGCCGAGGTCCAGAT |
| | | CGACAGACTGATCACAGGAAGACTG |
| | | CAGAGCCTGCAAACGTACGTGACAC |
| | | AGCAGCTGATCCGGGCAGCCGAAAT |
| | | CCGGGCCAGCGCCAATCTGGCCGCT |
| | | ACCAAGATGAGCGAGTGCGTGTTAG |
| | | GCCAGAGCAAGCGGGTGGATTTCTG |
| | | CGGTAAGGGATACCACCTGATGAGC |
| | | TTTCCCCAGAGCGCTCCTCACGGCG |
| | | TGGTGTTTCTGCACGTGACCTACGT |
| | | TCCTGCCCAGGAAAAGAACTTCACC |
| | | ACCGCCCCTGCTATCTGCCACGATG |
| | | GCAAGGCCCACTTCCCTAGAGAGGG |
| | | CGTTTTCGTGTCTAACGGCACACAC |
| | | TGGTTTGTGACCCAGAGAAACTTCT |
| | | ACGAGCCTCAGATCATCACCACAGA |
| | | CAACACCTTTGTGAGCGGCAATTGC |
| | | GACGTGGTGATCGGAATTGTTAATA |
| | | ATACCGTGTACGACCCTCTGCAGCC |
| | | TGAGCTCGACAGCTTCAAGGAAGAG |
| | | CTGGACAAGTACTTCAAGAACCACA |
| | | CCTCCCCAGATGTGGACCTGGGCGA |
| | | TATTTCAGGCATCAACGCCTCCGTC |
| | | GTGAATATCCAGAAGGAGATCGACC |
| | | GGCTCAACGAGGTGGCCAAGAACCT |
| | | TAACGAGAGCCTGATCGACCTGCAG |
| | | GAACTGGGCAAATATGAGCAGTACA |
| | | TCAAGTGGCCTTGGTACATCTGGCT |
| | | GGGCTTTATCGCAGGCCTGATCGCT |
| | | ATCGTGATGGTGACCATTATGCTGT |
| | | GTTGTATGACCAGCTGTTGTAGTTG |
| | | TCTGAAGGGCTGCTGTTCTTGCGGC |
| | | AGCTGCTGCAAGTTCGACGAAGACG |
| | | ACTCAGAGCCCGTGCTGAAAGGCGT |
| | | GAAGCTGCACTACACCCGAAAACGG |
| | | CGCggaagcggaggaagcggagcta |
| | | ctaacttcagcctgctgaagcaggc |
| | | tggagatgtggaggagaaccctgga |
| | | cctATGTATTCTTTTGTGTCCGAGG |
| | | AAACCGGCACACTGATCGTTAATAG |
| | | CGTGCTGCTCTTCCTGGCCTTCGTG |
| | | GTGTTCCTGCTGGTGACCCTGGCTA |
| | | TCCTGACCGCCCTGAGACTGTGTGC |
| | | CTACTGCTGCAACATCGTGAACGTG |
| | | TCTCTGGTCAAGCCTAGCTTCTACG |
| | | TGTACAGCCGGGTGAAGAACCTGAA |
| | | CAGCAGCAGAGTGCCCGACCTGCTG |
| | | GTGtaatcccccccccctaacgtta |
| | | ctggccgaagccgcttggaataagg |
| | | ccggtgtgcgtttgtctatatgtta |
| | | ttttccaccatattgccgtcttttg |
| | | gcaatgtgagggcccggaaacctgg |
| | | ccctgtcttcttgacgagcattcct |
| | | aggggtctttcccctctcgccaaag |
| | | gaatgcaaggtctgttgaatgtcgt |
| | | gaaggaagcagttcctctggaagct |
| | | tcttgaagacaaacaacgtctgtag |
| | | cgaccctttgcaggcagcggaaccc |
| | | cccacctggcgacaggtgcctctgc |
| | | ggccaaaagccacgtgtataagata |
| | | cacctgcaaaggcggcacaacccca |
| | | gtgccacgttgtgagttggatagtt |
| | | gtggaaagagtcaaatggctctcct |
| | | caagcgtattcaacaaggggctgaa |
| | | ggatgcccagaaggtaccccattgt |
| | | atgggatctgatctggggcctcggt |
| | | gcacatgctttacatgtgtttagtc |
| | | gaggttaaaaaaacgtctaggcccc |
| | | ccgaaccacggggacgtggttttcc |
| | | tttgaaaaacacgatgataatatgg |
| | | ccacaaccatggaacaagagacttg |
| | | cgcgcactctctcacttttgaggaa |
| | | tgcccaaaatgctctgctctacaat |
| | | accgtaatggattttacctgctaaa |
| | | gtatgatgaagaatggtacccagag |
| | | gagttattgactgatggagaggatg |
| | | atgtctttgatcccgaattagacat |
| | | ggaagtcgttttcgagttacagtaa |
| | | GTGTttacctgttaatgtagcattt |
| | | gagctttgggctaagcgcaacatta |
| | | aaccagtaccagaggtgaaaatact |
| | | caataatttgggtgtggacattgct |
| | | gctaatactgtgatctgggactaca |
| | | aaagagatgctccagcacatatatc |
| | | tactattggtgtttgttctatgact |
| | | gacatagccaagaaaccaactgaaa |
| | | cgatttgtgcaccactcactgtctt |
| | | ttttgatggtagagttgatggtcaa |
| | | gtagacttatttagaaatgcccgta |
| | | atggtgttcttattacagaaggtag |
| | | tgttaaaggtttacaaccatctgta |
| | | ggtcccaaacaagctagtcttaatg |
| | | gagtcacattaattggagaagccgt |
| | | aaaaacacagttcaattattataag |
| | | aaagttgatggtgttgtccaacaat |
| | | tacctgaaacttactttactcagag |
| | | tagaaatttacaagaatttaaaccc |
| | | aggagtcaaatggaaattgatttct |
| | | tagaattagctatggatgaattcat |
| | | tgaacggtataaattagaaggctat |
| | | gccttcgaacatatcgtttatggag |
| | | attttagtcatagtcagttaggtgg |
| | | tGCGAaattgttgttgtt |
| CoVEG13 | 45 | ATTAAAGGTTTATACCTTCCCAGGT |
| expression | | AACAAACCAACCAACTTTCGATCTC |
| cassette | | TTGTAGATCTGTTCTCTAAACGAAC |
| | | TTTAAAATCTGTGTGGCTGTCACTC |
| | | GGCTGCATGCTTAGTGCACTCACGC |
| | | AGTATAATTAATAACTAATTACTGT |
| | | CGTTGACAGGACACGAGTAACTCGT |
| | | CTATCTTCTGCAGGCTGCTTACGGT |
| | | TTCGTCCGTGTTGCAGCCGATCATC |
| | | AGCACATCTAGGTTTCGTCCGGGTG |
| | | TGACCGAAAGGTAAATGGCCGACAG |
| | | CAACGGCACAATCACCGTGGAAGAG |
| | | CTGAAGAAACTGCTGGAACAGTGGA |
| | | ACCTGGTCATCGGCTTCCTGTTTCT |
| | | GACCTGGATCTGTCTGCTGCAGTTC |
| | | GCTTATGCCAATCGGAACAGATTCC |
| | | TGTACATCATCAAGCTGATCTTCCT |
| | | GTGGCTGCTGTGGCCTGTGACCCTG |
| | | GCTTGCTTCGTGCTGGCCGCTGTGT |
| | | ACCGGATCAACTGGATCACAGGCGG |
| | | AATCGCCATCGCCATGGCCTGCCTG |
| | | GTGGGCCTGATGTGGCTGAGCTACT |
| | | TCATCGCTTCTTTCAGACTGTTCGC |
| | | CAGAACCCGGAGCATGTGGTCCTTC |
| | | AACCCCGAGACAAACATCCTGCTGA |
| | | ACGTGCCTCTGCACGGCACCATCCT |
| | | GACAAGACCTCTGCTCGAGAGCGAG |
| | | CTGGTGATTGGCGCAGTGATTCTGA |
| | | GAGGCCATCTGAGGATCGCCGGACA |
| | | CCACCTGGGCAGATGCGACATCAAG |
| | | GACCTTCCAAAGGAAATCACCGTTG |
| | | CCACCAGCCGGACCCTGTCCTACTA |
| | | CAAACTGGGCGCCAGCCAAAGAGTG |
| | | GCCGGCGATAGCGGCTTTGCCGCCT |
| | | ACAGCAGATACCGCATCGGAAATTA |
| | | CAAGCTCAACACCGACCACAGCAGC |
| | | TCTTCTGATAACATCGCCCTGCTGG |
| | | TGCAGCGAAAACGGCGCggaagcgg |
| | | aggaagcggagctactaacttcagc |
| | | ctgctgaagcaggctggagatgtgg |
| | | aggagaaccctggacctATGAGCGA |
| | | CAACGGCCCTCAAAACCAGAGAAAT |
| | | GCCCCTCGGATCACATTTGGCGGAC |
| | | CTAGCGACAGCACCGGCAGCAACCA |
| | | GAATGGAGAAAGAAGCGGCGCCAGA |
| | | TCCAAGCAGCGGAGACCTCAGGGAC |
| | | TGCCCAACAACACCGCTAGCTGGTT |
| | | CACCGCCCTGACCCAACACGGCAAG |
| | | GAAGATCTGAAGTTCCCCAGAGGCC |
| | | AGGGCGTGCCTATCAACACAAACTC |
| | | TTCTCCCGACGACCAGATCGGATAC |
| | | TATAGACGGGCCACTCGGAGAATTC |
| | | GGGGCGGCGACGGAAAAATGAAGGA |
| | | CCTTTCTCCAAGATGGTACTTCTAC |
| | | TACCTCGGCACAGGCCCTGAGGCCG |
| | | GCCTGCCTTACGGCGCCAACAAGGA |
| | | TGGCATCATCTGGGTCGCCACCGAG |
| | | GGCGCCCTGAACACCCCTAAGGACC |
| | | ACATCGGCACAAGAAACCCCGCTAA |
| | | CAACGCCGCAATCGTGCTGCAGCTG |
| | | CCTCAGGGCACCACCCTGCCCAAGG |
| | | GCTTCTACGCCGAGGGCTCTAGAGG |
| | | TGGCTCCCAGGCTTCTAGCCGCTCC |
| | | TCCAGCCGCAGCAGAAACAGCAGCA |
| | | GGAACAGCACCCCCGGCAGCTCCCG |
| | | GGGCACCAGCCCCGCCAGAATGGCC |
| | | GGAAATGGCGGCGATGCCGCCCTGG |
| | | CCCTGCTCCTGCTGGACAGACTGAA |
| | | TCAGCTGGAAAGCAAGATGAGCGGC |
| | | AAAGGACAGCAGCAGCAAGGCCAGA |
| | | CCGTGACCAAGAAAAGCGCTGCTGA |
| | | AGCCTCCAAGAAACCTAGACAAAAG |
| | | CGGACCGCCACAAAGGCCTACAACG |
| | | TGACCCAAGCCTTTGGAAGAAGAGG |
| | | CCCCGAGCAGACACAGGGCAATTTC |
| | | GGCGACCAGGAGCTGATCCGGCAGG |
| | | GAACCGACTACAAGCACTGGCCTCA |
| | | GATCGCCCAGTTCGCCCCTAGCGCC |
| | | AGCGCCTTCTTCGGCATGAGCAGAA |
| | | TCGGCATGGAAGTGACCCCTTCTGG |
| | | CACCTGGCTGACCTACACCGGCGCT |
| | | ATCAAGCTGGACGATAAGGATCCTA |
| | | ACTTCAAGGACCAAGTGATCCTGCT |
| | | GAACAAGCATATCGACGCCTATAAG |
| | | ACCTTTCCACCTACAGAGCCTAAGA |
| | | AAGATAAGAAGAAGAAAGCCGACGA |
| | | GACACAGGCCCTGCCTCAGAGACAG |
| | | AAAAAGCAGCAGACAGTGACACTGC |
| | | TGCCAGCCGCTGACCTGGATGACTT |
| | | CAGCAAGCAGCTGCAGCAGAGCATG |
| | | TCTTCTGCTGATAGCACCCAGGCCC |
| | | GAAAACGGCGCggaagcggaggaag |
| | | cggagctactaacttcagcctgctg |
| | | aagcaggctggagatgtggaggaga |
| | | accctggacctATGTTCGTGTTCCT |
| | | GGTGCTGCTGCCTCTGGTCAGCTCC |
| | | CAGTGTGTGAACCTGACCACCAGAA |
| | | CCCAGCTGCCACCTGCTTATACAAA |
| | | CTCCTTCACTCGGGGGGTATACTAC |
| | | CCCGACAAGGTGTTCAGATCTAGCG |
| | | TGCTGCATTCTACACAAGACCTGTT |
| | | CCTGCCCTTCTTCAGCAACGTGACC |
| | | TGGTTCCACGCCATCCACGTGTCTG |
| | | GAACCAACGGAACCAAGAGATTCGA |
| | | CAACCCCGTGCTGCCTTTCAACGAC |
| | | GGCGTGTACTTCGCCAGCACCGAGA |
| | | AGTCCAACATCATCAGAGGATGGAT |
| | | TTTCGGCACCACACTGGACAGCAAA |
| | | ACCCAGAGCCTGCTGATCGTGAACA |
| | | ACGCCACCAACGTGGTGATCAAGGT |
| | | GTGCGAGTTCCAGTTCTGCAATGAT |
| | | CCCTTCCTGGGCGTGTACTACCACA |
| | | AGAACAACAAGTCTTGGATGGAAAG |
| | | CGAGTTCAGAGTGTATTCCAGCGCC |
| | | AACAATTGCACCTTCGAGTACGTGA |
| | | GCCAACCCTTTCTGATGGACCTTGA |
| | | AGGCAAGCAGGGCAACTTCAAAAAT |
| | | CTGCGAGAATTTGTGTTCAAGAACA |
| | | TCGACGGATACTTCAAGATCTACTC |
| | | TAAGCACACGCCAATCAACCTGGTG |
| | | AGAGATCTGCCCCAGGGCTTTAGCG |
| | | CTTTGGAACCTCTGGTGGACCTGCC |
| | | TATCGGAATCAACATCACCAGATTT |
| | | CAAACTCTCCTGGCCCTGCACAGAT |
| | | CTTATCTGACCCCTGGGGACAGTAG |
| | | TAGCGGCTGGACAGCCGGCGCCGCC |
| | | GCCTACTACGTGGGATACCTGCAGC |
| | | CTAGAACATTCCTGCTGAAGTACAA |
| | | TGAGAACGGAACAATCACAGACGCC |
| | | GTGGACTGCGCCCTGGATCCTTTGA |
| | | GCGAGACAAAGTGCACCCTGAAGTC |
| | | GTTCACCGTCGAAAAAGGCATCTAC |
| | | CAGACCAGCAACTTCCGCGTGCAGC |
| | | CTACGGAATCTATCGTGCGGTTCCC |
| | | CAACATCACCAACCTGTGCCCTTTC |
| | | GGCGAGGTGTTTAACGCTACAAGGT |
| | | TCGCCAGCGTGTATGCCTGGAACAG |
| | | AAAGAGAATCAGCAATTGCGTGGCC |
| | | GATTATAGCGTTCTGTACAACAGCG |
| | | CTTCCTTCAGCACCTTCAAGTGCTA |
| | | CGGCGTGTCTCCAACCAAGCTGAAC |
| | | GACCTCTGCTTCACCAATGTCTACG |
| | | CTGACTCTTTCGTGATTAGAGGCGA |
| | | TGAGGTTAGACAGATCGCACCTGGC |
| | | CAGACCGGCAAAATCGCTGACTACA |
| | | ACTACAAGCTGCCTGATGACTTCAC |
| | | AGGCTGTGTCATTGCCTGGAACTCA |
| | | AATAACCTGGACTCTAAAGTGGGCG |
| | | GCAACTACAACTACCTGTACCGGCT |
| | | GTTCCGGAAGAGCAATCTGAAACCT |
| | | TTTGAGCGGGACATCTCTACAGAGA |
| | | TCTACCAGGCCGGCAGCACACCCTG |
| | | CAACGGCGTTGAGGGCTTCAACTGC |
| | | TACTTCCCTCTGCAGAGCTACGGCT |
| | | TTCAGCCAACAAATGGAGTGGGCTA |
| | | CCAGCCGTACAGAGTGGTGGTGCTG |
| | | AGCTTCGAACTGCTGCATGCCCCAG |
| | | CCACAGTGTGTGGACCTAAGAAGTC |
| | | TACCAACCTGGTGAAGAACAAGTGC |
| | | GTGAACTTTAACTTTAACGGCCTGA |
| | | CCGGCACAGGCGTGCTGACCGAATC |
| | | CAACAAAAAGTTCCTGCCCTTCCAA |
| | | CAGTTCGGCAGAGACATCGCCGATA |
| | | CAACCGATGCCGTGCGGGACCCCCA |
| | | GACCTTAGAAATCCTAGATATCACC |
| | | CCGTGCAGCTTCGGCGGAGTCTCTG |
| | | TTATTACTCCTGGCACCAACACCAG |
| | | CAACCAAGTGGCTGTTCTGTACCAA |
| | | ggcGTGAACTGCACCGAAGTGCCTG |
| | | TGGCTATCCACGCCGATCAGCTGAC |
| | | CCCAACCTGGCGGGTGTATAGCACC |
| | | GGCTCTAACGTGTTCCAGACCCGGG |
| | | CTGGCTGCCTGATCGGCGCCGAACA |
| | | CGTCAACAACTCCTATGAATGTGAC |
| | | ATCCCCATCGGGGCTGGCATCTGCG |
| | | CCAGTTACCAGACACAGACAAATAG |
| | | CCCTAGACGGGCCAGAAGCGTGGCC |
| | | TCCCAGAGTATCATTGCCTACACCA |
| | | TGAGCCTGGGCGCCGAGAACAGCGT |
| | | GGCCTATTCTAACAATAGCATCGCA |
| | | ATCCCTACCAACTTTACCATCTCTG |
| | | TGACAACCGAGATCCTGCCTGTGAG |
| | | CATGACCAAAACCAGCGTGGACTGC |
| | | ACGATGTACATCTGTGGCGACAGCA |
| | | CAGAATGCAGTAATCTGTTGCTGCA |
| | | GTACGGCAGCTTTTGCACCCAGTTG |
| | | AATAGAGCCCTGACCGGAATCGCCG |
| | | TAGAGCAGGACAAAAATACCCAGGA |
| | | GGTGTTCGCCCAGGTGAAACAGATC |
| | | TACAAGACACCTCCCATTAAGGACT |
| | | TCGGAGGTTTTAACTTCAGCCAGAT |
| | | CCTGCCCGACCCTTCCAAGCCTAGC |
| | | AAACGCTCCTTCATCGAGGACCTGC |
| | | TCTTCAACAAGGTGACACTGGCTGA |
| | | TGCCGGCTTCATCAAGCAGTACGGA |
| | | GATTGTCTGGGAGACATCGCCGCTA |
| | | GAGATCTGATCTGCGCCCAAAAGTT |
| | | CAACGGCCTGACCGTGCTGCCTCCT |
| | | CTGCTTACAGACGAGATGATCGCCC |
| | | AGTACACCAGCGCCCTGCTGGCTGG |
| | | CACCATCACAAGCGGCTGGACCTTC |
| | | GGAGCCGGAGCCGCTCTGCAAATCC |
| | | CCTTTGCCATGCAGATGGCCTACCG |
| | | GTTCAACGGCATCGGCGTGACACAG |
| | | AATGTGCTGTACGAGAACCAGAAGC |
| | | TGATCGCTAACCAGTTTAACAGCGC |
| | | TATCGGCAAGATCCAGGACTCGCTG |
| | | AGTAGCACCGCCTCTGCCCTGGGCA |
| | | AGCTGCAGGACGTCGTGAACCAGAA |
| | | CGCCCAAGCCCTGAACACACTGGTG |
| | | AAACAGCTGAGCAGCAACTTCGGCG |
| | | CCATCAGCTCTGTGCTGAACGATAT |
| | | CCTGAGCAGACTGGACAAGGTGGAA |
| | | GCCGAGGTCCAGATCGACAGACTGA |
| | | TCACAGGAAGACTGCAGAGCCTGCA |
| | | AACGTACGTGACACAGCAGCTGATC |
| | | CGGGCAGCCGAAATCCGGGCCAGCG |
| | | CCAATCTGGCCGCTACCAAGATGAG |
| | | CGAGTGCGTGTTAGGCCAGAGCAAG |
| | | CGGGTGGATTTCTGCGGTAAGGGAT |
| | | ACCACCTGATGAGCTTTCCCCAGAG |
| | | CGCTCCTCACGGCGTGGTGTTTCTG |
| | | CACGTGACCTACGTTCCTGCCCAGG |
| | | AAAAGAACTTCACCACCGCCCCTGC |
| | | TATCTGCCACGATGGCAAGGCCCAC |
| | | TTCCCTAGAGAGGGCGTTTTCGTGT |
| | | CTAACGGCACACACTGGTTTGTGAC |
| | | CCAGAGAAACTTCTACGAGCCTCAG |
| | | ATCATCACCACAGACAACACCTTTG |
| | | TGAGCGGCAATTGCGACGTGGTGAT |
| | | CGGAATTGTTAATAATACCGTGTAC |
| | | GACCCTCTGCAGCCTGAGCTCGACA |
| | | GCTTCAAGGAAGAGCTGGACAAGTA |
| | | CTTCAAGAACCACACCTCCCCAGAT |
| | | GTGGACCTGGGCGATATTTCAGGCA |
| | | TCAACGCCTCCGTCGTGAATATCCA |
| | | GAAGGAGATCGACCGGCTCAACGAG |
| | | GTGGCCAAGAACCTTAACGAGAGCC |
| | | TGATCGACCTGCAGGAACTGGGCAA |
| | | ATATGAGCAGTACATCAAGTGGCCT |
| | | TGGTACATCTGGCTGGGCTTTATCG |
| | | CAGGCCTGATCGCTATCGTGATGGT |
| | | GACCATTATGCTGTGTTGTATGACC |
| | | AGCTGTTGTAGTTGTCTGAAGGGCT |
| | | GCTGTTCTTGCGGCAGCTGCTGCAA |
| | | GTTCGACGAAGACGACTCAGAGCCC |
| | | GTGCTGAAAGGCGTGAAGCTGCACT |
| | | ACACCCGAAAACGGCGCggaagcgg |
| | | aggaagcggagctactaacttcagc |
| | | ctgctgaagcaggctggagatgtgg |
| | | aggagaaccctggacctATGTATTC |
| | | TTTTGTGTCCGAGGAAACCGGCACA |
| | | CTGATCGTTAATAGCGTGCTGCTCT |
| | | TCCTGGCCTTCGTGGTGTTCCTGCT |
| | | GGTGACCCTGGCTATCCTGACCGCC |
| | | CTGAGACTGTGTGCCTACTGCTGCA |
| | | ACATCGTGAACGTGTCTCTGGTCAA |
| | | GCCTAGCTTCTACGTGTACAGCCGG |
| | | GTGAAGAACCTGAACAGCAGCAGAG |
| | | TGCCCGACCTGCTGGTGtaatcccc |
| | | cccccctaacgttactggccgaagc |
| | | cgcttggaataaggccggtgtgcgt |
| | | ttgtctatatgttattttccaccat |
| | | attgccgtcttttggcaatgtgagg |
| | | gcccggaaacctggccctgtcttct |
| | | tgacgagcattcctaggggtctttc |
| | | ccctctcgccaaaggaatgcaaggt |
| | | ctgttgaatgtcgtgaaggaagcag |
| | | ttcctctggaagcttcttgaagaca |
| | | aacaacgtctgtagcgaccctttgc |
| | | aggcagcggaaccccccacctggcg |
| | | acaggtgcctctgcggccaaaagcc |
| | | acgtgtataagatacacctgcaaag |
| | | gcggcacaaccccagtgccacgttg |
| | | tgagttggatagttgtggaaagagt |
| | | caaatggctctcctcaagcgtattc |
| | | aacaaggggctgaaggatgcccaga |
| | | aggtaccccattgtatgggatctga |
| | | tctggggcctcggtgcacatgcttt |
| | | acatgtgtttagtcgaggttaaaaa |
| | | aacgtctaggccccccgaaccacgg |
| | | ggacgtggttttcctttgaaaaaca |
| | | cgatgataatatggccacaaccatg |
| | | gaacaagagacttgcgcgcactctc |
| | | tcacttttgaggaatgcccaaaatg |
| | | ctctgctctacaataccgtaatgga |
| | | ttttacctgctaaagtatgatgaag |
| | | aatggtacccagaggagttattgac |
| | | tgatggagaggatgatgtctttgat |
| | | cccgaattagacatggaagtcgttt |
| | | tcgagttacagtaaGTGTttacctg |
| | | ttaatgtagcatttgagctttgggc |
| | | taagcgcaacattaaaccagtacca |
| | | gaggtgaaaatactcaataatttgg |
| | | gtgtggacattgctgctaatactgt |
| | | gatctgggactacaaaagagatgct |
| | | ccagcacatatatctactattggtg |
| | | tttgttctatgactgacatagccaa |
| | | gaaaccaactgaaacgatttgtgca |
| | | ccactcactgtcttttttgatggta |
| | | gagttgatggtcaagtagacttatt |
| | | tagaaatgcccgtaatggtgttctt |
| | | attacagaaggtagtgttaaaggtt |
| | | tacaaccatctgtaggtcccaaaca |
| | | agctagtcttaatggagtcacatta |
| | | attggagaagccgtaaaaacacagt |
| | | tcaattattataagaaagttgatgg |
| | | tgttgtccaacaattacctgaaact |
| | | tactttactcagagtagaaatttac |
| | | aagaatttaaacccaggagtcaaat |
| | | ggaaattgatttcttagaattagct |
| | | atggatgaattcattgaacggtata |
| | | aattagaaggctatgccttcgaaca |
| | | tatcgtttatggagattttagtcat |
| | | agtcagttaggtggtGCGAaattgt |
| | | tgttgtt |
| CoVEG14 | 46 | ATTAAAGGTTTATACCTTCCCAGGT |
| expression | | AACAAACCAACCAACTTTCGATCTC |
| cassette | | TTGTAGATCTGTTCTCTAAACGAAC |
| | | TTTAAAATCTGTGTGGCTGTCACTC |
| | | GGCTGCATGCTTAGTGCACTCACGC |
| | | AGTATAATTAATAACTAATTACTGT |
| | | CGTTGACAGGACACGAGTAACTCGT |
| | | CTATCTTCTGCAGGCTGCTTACGGT |
| | | TTCGTCCGTGTTGCAGCCGATCATC |
| | | AGCACATCTAGGTTTCGTCCGGGTG |
| | | TGACCGAAAGGTAAATGGCCGACAG |
| | | CAACGGCACAATCACCGTGGAAGAG |
| | | CTGAAGAAACTGCTGGAACAGTGGA |
| | | ACCTGGTCATCGGCTTCCTGTTTCT |
| | | GACCTGGATCTGTCTGCTGCAGTTC |
| | | GCTTATGCCAATCGGAACAGATTCC |
| | | TGTACATCATCAAGCTGATCTTCCT |
| | | GTGGCTGCTGTGGCCTGTGACCCTG |
| | | GCTTGCTTCGTGCTGGCCGCTGTGT |
| | | ACCGGATCAACTGGATCACAGGCGG |
| | | AATCGCCATCGCCATGGCCTGCCTG |
| | | GTGGGCCTGATGTGGCTGAGCTACT |
| | | TCATCGCTTCTTTCAGACTGTTCGC |
| | | CAGAACCCGGAGCATGTGGTCCTTC |
| | | AACCCCGAGACAAACATCCTGCTGA |
| | | ACGTGCCTCTGCACGGCACCATCCT |
| | | GACAAGACCTCTGCTCGAGAGCGAG |
| | | CTGGTGATTGGCGCAGTGATTCTGA |
| | | GAGGCCATCTGAGGATCGCCGGACA |
| | | CCACCTGGGCAGATGCGACATCAAG |
| | | GACCTTCCAAAGGAAATCACCGTTG |
| | | CCACCAGCCGGACCCTGTCCTACTA |
| | | CAAACTGGGCGCCAGCCAAAGAGTG |
| | | GCCGGCGATAGCGGCTTTGCCGCCT |
| | | ACAGCAGATACCGCATCGGAAATTA |
| | | CAAGCTCAACACCGACCACAGCAGC |
| | | TCTTCTGATAACATCGCCCTGCTGG |
| | | TGCAGCGAAAACGGCGCggaagcgg |
| | | aggaagcggagctactaacttcagc |
| | | ctgctgaagcaggctggagatgtgg |
| | | aggagaaccctggacctATGAGCGA |
| | | CAACGGCCCTCAAAACCAGAGAAAT |
| | | GCCCCTCGGATCACATTTGGCGGAC |
| | | CTAGCGACAGCACCGGCAGCAACCA |
| | | GAATGGAGAAAGAAGCGGCGCCAGA |
| | | TCCAAGCAGCGGAGACCTCAGGGAC |
| | | TGCCCAACAACACCGCTAGCTGGTT |
| | | CACCGCCCTGACCCAACACGGCAAG |
| | | GAAGATCTGAAGTTCCCCAGAGGCC |
| | | AGGGCGTGCCTATCAACACAAACTC |
| | | TTCTCCCGACGACCAGATCGGATAC |
| | | TATAGACGGGCCACTCGGAGAATTC |
| | | GGGGCGGCGACGGAAAAATGAAGGA |
| | | CCTTTCTCCAAGATGGTACTTCTAC |
| | | TACCTCGGCACAGGCCCTGAGGCCG |
| | | GCCTGCCTTACGGCGCCAACAAGGA |
| | | TGGCATCATCTGGGTCGCCACCGAG |
| | | GGCGCCCTGAACACCCCTAAGGACC |
| | | ACATCGGCACAAGAAACCCCGCTAA |
| | | CAACGCCGCAATCGTGCTGCAGCTG |
| | | CCTCAGGGCACCACCCTGCCCAAGG |
| | | GCTTCTACGCCGAGGGCTCTAGAGG |
| | | TGGCTCCCAGGCTTCTAGCCGCTCC |
| | | TCCAGCCGCAGCAGAAACAGCAGCA |
| | | GGAACAGCACCCCCGGCAGCTCCCG |
| | | GGGCACCAGCCCCGCCAGAATGGCC |
| | | GGAAATGGCGGCGATGCCGCCCTGG |
| | | CCCTGCTCCTGCTGGACAGACTGAA |
| | | TCAGCTGGAAAGCAAGATGAGCGGC |
| | | AAAGGACAGCAGCAGCAAGGCCAGA |
| | | CCGTGACCAAGAAAAGCGCTGCTGA |
| | | AGCCTCCAAGAAACCTAGACAAAAG |
| | | CGGACCGCCACAAAGGCCTACAACG |
| | | TGACCCAAGCCTTTGGAAGAAGAGG |
| | | CCCCGAGCAGACACAGGGCAATTTC |
| | | GGCGACCAGGAGCTGATCCGGCAGG |
| | | GAACCGACTACAAGCACTGGCCTCA |
| | | GATCGCCCAGTTCGCCCCTAGCGCC |
| | | AGCGCCTTCTTCGGCATGAGCAGAA |
| | | TCGGCATGGAAGTGACCCCTTCTGG |
| | | CACCTGGCTGACCTACACCGGCGCT |
| | | ATCAAGCTGGACGATAAGGATCCTA |
| | | ACTTCAAGGACCAAGTGATCCTGCT |
| | | GAACAAGCATATCGACGCCTATAAG |
| | | ACCTTTCCACCTACAGAGCCTAAGA |
| | | AAGATAAGAAGAAGAAAGCCGACGA |
| | | GACACAGGCCCTGCCTCAGAGACAG |
| | | AAAAAGCAGCAGACAGTGACACTGC |
| | | TGCCAGCCGCTGACCTGGATGACTT |
| | | CAGCAAGCAGCTGCAGCAGAGCATG |
| | | TCTTCTGCTGATAGCACCCAGGCCC |
| | | GAAAACGGCGCggaagcggaggaag |
| | | cggagctactaacttcagcctgctg |
| | | aagcaggctggagatgtggaggaga |
| | | accctggacctATGTTCGTGTTCCT |
| | | GGTGCTGCTGCCTCTGGTCAGCTCC |
| | | CAGTGTGTGAACCTGACCACCAGAA |
| | | CCCAGCTGCCACCTGCTTATACAAA |
| | | CTCCTTCACTCGGGGGGTATACTAC |
| | | CCCGACAAGGTGTTCAGATCTAGCG |
| | | TGCTGCATTCTACACAAGACCTGTT |
| | | CCTGCCCTTCTTCAGCAACGTGACC |
| | | TGGTTCCACGCCATCCACGTGTCTG |
| | | GAACCAACGGAACCAAGAGATTCGA |
| | | CAACCCCGTGCTGCCTTTCAACGAC |
| | | GGCGTGTACTTCGCCAGCACCGAGA |
| | | AGTCCAACATCATCAGAGGATGGAT |
| | | TTTCGGCACCACACTGGACAGCAAA |
| | | ACCCAGAGCCTGCTGATCGTGAACA |
| | | ACGCCACCAACGTGGTGATCAAGGT |
| | | GTGCGAGTTCCAGTTCTGCAATGAT |
| | | CCCTTCCTGGGCGTGTACTACCACA |
| | | AGAACAACAAGTCTTGGATGGAAAG |
| | | CGAGTTCAGAGTGTATTCCAGCGCC |
| | | AACAATTGCACCTTCGAGTACGTGA |
| | | GCCAACCCTTTCTGATGGACCTTGA |
| | | AGGCAAGCAGGGCAACTTCAAAAAT |
| | | CTGCGAGAATTTGTGTTCAAGAACA |
| | | TCGACGGATACTTCAAGATCTACTC |
| | | TAAGCACACGCCAATCAACCTGGTG |
| | | AGAGATCTGCCCCAGGGCTTTAGCG |
| | | CTTTGGAACCTCTGGTGGACCTGCC |
| | | TATCGGAATCAACATCACCAGATTT |
| | | CAAACTCTCCTGGCCCTGCACAGAT |
| | | CTTATCTGACCCCTGGGGACAGTAG |
| | | TAGCGGCTGGACAGCCGGCGCCGCC |
| | | GCCTACTACGTGGGATACCTGCAGC |
| | | CTAGAACATTCCTGCTGAAGTACAA |
| | | TGAGAACGGAACAATCACAGACGCC |
| | | GTGGACTGCGCCCTGGATCCTTTGA |
| | | GCGAGACAAAGTGCACCCTGAAGTC |
| | | GTTCACCGTCGAAAAAGGCATCTAC |
| | | CAGACCAGCAACTTCCGCGTGCAGC |
| | | CTACGGAATCTATCGTGCGGTTCCC |
| | | CAACATCACCAACCTGTGCCCTTTC |
| | | GGCGAGGTGTTTAACGCTACAAGGT |
| | | TCGCCAGCGTGTATGCCTGGAACAG |
| | | AAAGAGAATCAGCAATTGCGTGGCC |
| | | GATTATAGCGTTCTGTACAACAGCG |
| | | CTTCCTTCAGCACCTTCAAGTGCTA |
| | | CGGCGTGTCTCCAACCAAGCTGAAC |
| | | GACCTCTGCTTCACCAATGTCTACG |
| | | CTGACTCTTTCGTGATTAGAGGCGA |
| | | TGAGGTTAGACAGATCGCACCTGGC |
| | | CAGACCGGCAAAATCGCTGACTACA |
| | | ACTACAAGCTGCCTGATGACTTCAC |
| | | AGGCTGTGTCATTGCCTGGAACTCA |
| | | AATAACCTGGACTCTAAAGTGGGCG |
| | | GCAACTACAACTACCTGTACCGGCT |
| | | GTTCCGGAAGAGCAATCTGAAACCT |
| | | TTTGAGCGGGACATCTCTACAGAGA |
| | | TCTACCAGGCCGGCAGCACACCCTG |
| | | CAACGGCGTTGAGGGCTTCAACTGC |
| | | TACTTCCCTCTGCAGAGCTACGGCT |
| | | TTCAGCCAACAAATGGAGTGGGCTA |
| | | CCAGCCGTACAGAGTGGTGGTGCTG |
| | | AGCTTCGAACTGCTGCATGCCCCAG |
| | | CCACAGTGTGTGGACCTAAGAAGTC |
| | | TACCAACCTGGTGAAGAACAAGTGC |
| | | GTGAACTTTAACTTTAACGGCCTGA |
| | | CCGGCACAGGCGTGCTGACCGAATC |
| | | CAACAAAAAGTTCCTGCCCTTCCAA |
| | | CAGTTCGGCAGAGACATCGCCGATA |
| | | CAACCGATGCCGTGCGGGACCCCCA |
| | | GACCTTAGAAATCCTAGATATCACC |
| | | CCGTGCAGCTTCGGCGGAGTCTCTG |
| | | TTATTACTCCTGGCACCAACACCAG |
| | | CAACCAAGTGGCTGTTCTGTACCAA |
| | | ggcGTGAACTGCACCGAAGTGCCTG |
| | | TGGCTATCCACGCCGATCAGCTGAC |
| | | CCCAACCTGGCGGGTGTATAGCACC |
| | | GGCTCTAACGTGTTCCAGACCCGGG |
| | | CTGGCTGCCTGATCGGCGCCGAACA |
| | | CGTCAACAACTCCTATGAATGTGAC |
| | | ATCCCCATCGGGGCTGGCATCTGCG |
| | | CCAGTTACCAGACACAGACAAATAG |
| | | CCCTAGACGGGCCAGAAGCGTGGCC |
| | | TCCCAGAGTATCATTGCCTACACCA |
| | | TGAGCCTGGGCGCCGAGAACAGCGT |
| | | GGCCTATTCTAACAATAGCATCGCA |
| | | ATCCCTACCAACTTTACCATCTCTG |
| | | TGACAACCGAGATCCTGCCTGTGAG |
| | | CATGACCAAAACCAGCGTGGACTGC |
| | | ACGATGTACATCTGTGGCGACAGCA |
| | | CAGAATGCAGTAATCTGTTGCTGCA |
| | | GTACGGCAGCTTTTGCACCCAGTTG |
| | | AATAGAGCCCTGACCGGAATCGCCG |
| | | TAGAGCAGGACAAAAATACCCAGGA |
| | | GGTGTTCGCCCAGGTGAAACAGATC |
| | | TACAAGACACCTCCCATTAAGGACT |
| | | TCGGAGGTTTTAACTTCAGCCAGAT |
| | | CCTGCCCGACCCTTCCAAGCCTAGC |
| | | AAACGCTCCTTCATCGAGGACCTGC |
| | | TCTTCAACAAGGTGACACTGGCTGA |
| | | TGCCGGCTTCATCAAGCAGTACGGA |
| | | GATTGTCTGGGAGACATCGCCGCTA |
| | | GAGATCTGATCTGCGCCCAAAAGTT |
| | | CAACGGCCTGACCGTGCTGCCTCCT |
| | | CTGCTTACAGACGAGATGATCGCCC |
| | | AGTACACCAGCGCCCTGCTGGCTGG |
| | | CACCATCACAAGCGGCTGGACCTTC |
| | | GGAGCCGGAGCCGCTCTGCAAATCC |
| | | CCTTTGCCATGCAGATGGCCTACCG |
| | | GTTCAACGGCATCGGCGTGACACAG |
| | | AATGTGCTGTACGAGAACCAGAAGC |
| | | TGATCGCTAACCAGTTTAACAGCGC |
| | | TATCGGCAAGATCCAGGACTCGCTG |
| | | AGTAGCACCGCCTCTGCCCTGGGCA |
| | | AGCTGCAGGACGTCGTGAACCAGAA |
| | | CGCCCAAGCCCTGAACACACTGGTG |
| | | AAACAGCTGAGCAGCAACTTCGGCG |
| | | CCATCAGCTCTGTGCTGAACGATAT |
| | | CCTGAGCAGACTGGACAAGGTGGAA |
| | | GCCGAGGTCCAGATCGACAGACTGA |
| | | TCACAGGAAGACTGCAGAGCCTGCA |
| | | AACGTACGTGACACAGCAGCTGATC |
| | | CGGGCAGCCGAAATCCGGGCCAGCG |
| | | CCAATCTGGCCGCTACCAAGATGAG |
| | | CGAGTGCGTGTTAGGCCAGAGCAAG |
| | | CGGGTGGATTTCTGCGGTAAGGGAT |
| | | ACCACCTGATGAGCTTTCCCCAGAG |
| | | CGCTCCTCACGGCGTGGTGTTTCTG |
| | | CACGTGACCTACGTTCCTGCCCAGG |
| | | AAAAGAACTTCACCACCGCCCCTGC |
| | | TATCTGCCACGATGGCAAGGCCCAC |
| | | TTCCCTAGAGAGGGCGTTTTCGTGT |
| | | CTAACGGCACACACTGGTTTGTGAC |
| | | CCAGAGAAACTTCTACGAGCCTCAG |
| | | ATCATCACCACAGACAACACCTTTG |
| | | TGAGCGGCAATTGCGACGTGGTGAT |
| | | CGGAATTGTTAATAATACCGTGTAC |
| | | GACCCTCTGCAGCCTGAGCTCGACA |
| | | GCTTCAAGGAAGAGCTGGACAAGTA |
| | | CTTCAAGAACCACACCTCCCCAGAT |
| | | GTGGACCTGGGCGATATTTCAGGCA |
| | | TCAACGCCTCCGTCGTGAATATCCA |
| | | GAAGGAGATCGACCGGCTCAACGAG |
| | | GTGGCCAAGAACCTTAACGAGAGCC |
| | | TGATCGACCTGCAGGAACTGGGCAA |
| | | ATATGAGCAGTACATCAAGTGGCCT |
| | | TGGTACATCTGGCTGGGCTTTATCG |
| | | CAGGCCTGATCGCTATCGTGATGGT |
| | | GACCATTATGCTGTGTTGTATGACC |
| | | AGCTGTTGTAGTTGTCTGAAGGGCT |
| | | GCTGTTCTTGCGGCAGCTGCTGCAA |
| | | GTTCGACGAAGACGACTCAGAGCCC |
| | | GTGCTGAAAGGCGTGAAGCTGCACT |
| | | ACACCCGAAAACGGCGCggaagcgg |
| | | aggaagcggagctactaacttcagc |
| | | ctgctgaagcaggctggagatgtgg |
| | | aggagaaccctggacctATGTATTC |
| | | TTTTGTGTCCGAGGAAACCGGCACA |
| | | CTGATCGTTAATAGCGTGCTGCTCT |
| | | TCCTGGCCTTCGTGGTGTTCCTGCT |
| | | GGTGACCCTGGCTATCCTGACCGCC |
| | | CTGAGACTGTGTGCCTACTGCTGCA |
| | | ACATCGTGAACGTGTCTCTGGTCAA |
| | | GCCTAGCTTCTACGTGTACAGCCGG |
| | | GTGAAGAACCTGAACAGCAGCAGAG |
| | | TGCCCGACCTGCTGGTGtaatcccc |
| | | cccccctaacgttactggccgaagc |
| | | cgcttggaataaggccggtgtgcgt |
| | | ttgtctatatgttattttccaccat |
| | | attgccgtcttttggcaatgtgagg |
| | | gcccggaaacctggccctgtcttct |
| | | tgacgagcattcctaggggtctttc |
| | | ccctctcgccaaaggaatgcaaggt |
| | | ctgttgaatgtcgtgaaggaagcag |
| | | ttcctctggaagcttcttgaagaca |
| | | aacaacgtctgtagcgaccctttgc |
| | | aggcagcggaaccccccacctggcg |
| | | acaggtgcctctgcggccaaaagcc |
| | | acgtgtataagatacacctgcaaag |
| | | gcggcacaaccccagtgccacgttg |
| | | tgagttggatagttgtggaaagagt |
| | | caaatggctctcctcaagcgtattc |
| | | aacaaggggctgaaggatgcccaga |
| | | aggtaccccattgtatgggatctga |
| | | tctggggcctcggtgcacatgcttt |
| | | acatgtgtttagtcgaggttaaaaa |
| | | aacgtctaggccccccgaaccacgg |
| | | ggacgtggttttcctttgaaaaaca |
| | | cgatgataatatggccacaaccatg |
| | | gaacaagagacttgcgcgcactctc |
| | | tcacttttgaggaatgcccaaaatg |
| | | ctctgctctacaataccgtaatgga |
| | | ttttacctgctaaagtatgatgaag |
| | | aatggtacccagaggagttattgac |
| | | tgatggagaggatgatgtctttgat |
| | | cccgaattagacatggaagtcgttt |
| | | tcgagttacagggaagcggagctac |
| | | taacttcagcctgctgaagcaggct |
| | | ggagatgtggaggagaaccctggac |
| | | ctATGGACCTGTTCATGAGAATCTT |
| | | CACCATCGGCACCGTGACACTGAAG |
| | | CAGGGCGAGATCAAGGATGCCACCC |
| | | CTAGCGACTTCGTGAGAGCCACCGC |
| | | CACAATTCCTATCCAGGCTAGCCTG |
| | | CCTTTTGGATGGCTGATCGTGGGCG |
| | | TCGCCCTGCTCGCCGTGTTCCAGAG |
| | | CGCCTCTAAGATCATTACACTGAAG |
| | | AAAAGATGGCAGCTGGCCCTCTCCA |
| | | AAGGCGTGCACTTCGTGTGTAATCT |
| | | GCTGCTGCTTTTTGTGACAGTGTAC |
| | | AGCCACCTGCTGCTGGTTGCTGCTG |
| | | GCCTGGAAGCCCCTTTCCTGTACCT |
| | | GTACGCCCTGGTCTACTTCCTGCAG |
| | | TCTATCAACTTCGTGCGGATCATCA |
| | | TGCGGCTGTGGCTGTGCTGGAAGTG |
| | | CAGAAGCAAGAACCCACTGCTGTAC |
| | | GACGCCAATTACTTCCTGTGTTGGC |
| | | ACACCAACTGCTACGACTACTGCAT |
| | | CCCCTACAACAGCGTGACCAGCAGC |
| | | ATCGTGATCACCTCTGGCGACGGAA |
| | | CAACCAGCCCTATCAGCGAGCATGA |
| | | TTACCAGATCGGCGGATATACAGAG |
| | | AAGTGGGAGAGCGGCGTGAAGGACT |
| | | GCGTGGTGCTGCACAGCTACTTTAC |
| | | CTCCGATTACTACCAACTGTATTCT |
| | | ACCCAGCTGAGCACCGACACCGGCG |
| | | TGGAACACGTGACCTTCTTCATCTA |
| | | CAACAAGATCGTGGACGAGCCTGAG |
| | | GAACACGTGCAGATCCACACTATCG |
| | | ACGGCAGCTCTGGCGTTGTGAACCC |
| | | TGTGATGGAACCCATCTACGATGAG |
| | | CCCACCACAACAACCTCCGTGCCCC |
| | | TGTaaGTGTttacctgttaatgtag |
| | | catttgagctttgggctaagcgcaa |
| | | cattaaaccagtaccagaggtgaaa |
| | | atactcaataatttgggtgtggaca |
| | | ttgctgctaatactgtgatctggga |
| | | ctacaaaagagatgctccagcacat |
| | | atatctactattggtgtttgttcta |
| | | tgactgacatagccaagaaaccaac |
| | | tgaaacgatttgtgcaccactcact |
| | | gtcttttttgatggtagagttgatg |
| | | gtcaagtagacttatttagaaatgc |
| | | ccgtaatggtgttcttattacagaa |
| | | ggtagtgttaaaggtttacaaccat |
| | | ctgtaggtcccaaacaagctagtct |
| | | taatggagtcacattaattggagaa |
| | | gccgtaaaaacacagttcaattatt |
| | | ataagaaagttgatggtgttgtcca |
| | | acaattacctgaaacttactttact |
| | | cagagtagaaatttacaagaattta |
| | | aacccaggagtcaaatggaaattga |
| | | tttcttagaattagctatggatgaa |
| | | ttcattgaacggtataaattagaag |
| | | gctatgccttcgaacatatcgttta |
| | | tggagattttagtcatagtcagtta |
| | | ggtggtGCGAaattgttgttgtt |
| CoVEG15 | 47 | ATTAAAGGTTTATACCTTCCCAGGT |
| expression | | AACAAACCAACCAACTTTCGATCTC |
| cassette | | TTGTAGATCTGTTCTCTAAACGAAC |
| | | TTTAAAATCTGTGTGGCTGTCACTC |
| | | GGCTGCATGCTTAGTGCACTCACGC |
| | | AGTATAATTAATAACTAATTACTGT |
| | | CGTTGACAGGACACGAGTAACTCGT |
| | | CTATCTTCTGCAGGCTGCTTACGGT |
| | | TTCGTCCGTGTTGCAGCCGATCATC |
| | | AGCACATCTAGGTTTCGTCCGGGTG |
| | | TGACCGAAAGGTAAATGGCCGACAG |
| | | CAACGGCACAATCACCGTGGAAGAG |
| | | CTGAAGAAACTGCTGGAACAGTGGA |
| | | ACCTGGTCATCGGCTTCCTGTTTCT |
| | | GACCTGGATCTGTCTGCTGCAGTTC |
| | | GCTTATGCCAATCGGAACAGATTCC |
| | | TGTACATCATCAAGCTGATCTTCCT |
| | | GTGGCTGCTGTGGCCTGTGACCCTG |
| | | GCTTGCTTCGTGCTGGCCGCTGTGT |
| | | ACCGGATCAACTGGATCACAGGCGG |
| | | AATCGCCATCGCCATGGCCTGCCTG |
| | | GTGGGCCTGATGTGGCTGAGCTACT |
| | | TCATCGCTTCTTTCAGACTGTTCGC |
| | | CAGAACCCGGAGCATGTGGTCCTTC |
| | | AACCCCGAGACAAACATCCTGCTGA |
| | | ACGTGCCTCTGCACGGCACCATCCT |
| | | GACAAGACCTCTGCTCGAGAGCGAG |
| | | CTGGTGATTGGCGCAGTGATTCTGA |
| | | GAGGCCATCTGAGGATCGCCGGACA |
| | | CCACCTGGGCAGATGCGACATCAAG |
| | | GACCTTCCAAAGGAAATCACCGTTG |
| | | CCACCAGCCGGACCCTGTCCTACTA |
| | | CAAACTGGGCGCCAGCCAAAGAGTG |
| | | GCCGGCGATAGCGGCTTTGCCGCCT |
| | | ACAGCAGATACCGCATCGGAAATTA |
| | | CAAGCTCAACACCGACCACAGCAGC |
| | | TCTTCTGATAACATCGCCCTGCTGG |
| | | TGCAGCGAAAACGGCGCggaagcgg |
| | | aggaagcggagctactaacttcagc |
| | | ctgctgaagcaggctggagatgtgg |
| | | aggagaaccctggacctATGAGCGA |
| | | CAACGGCCCTCAAAACCAGAGAAAT |
| | | GCCCCTCGGATCACATTTGGCGGAC |
| | | CTAGCGACAGCACCGGCAGCAACCA |
| | | GAATGGAGAAAGAAGCGGCGCCAGA |
| | | TCCAAGCAGCGGAGACCTCAGGGAC |
| | | TGCCCAACAACACCGCTAGCTGGTT |
| | | CACCGCCCTGACCCAACACGGCAAG |
| | | GAAGATCTGAAGTTCCCCAGAGGCC |
| | | AGGGCGTGCCTATCAACACAAACTC |
| | | TTCTCCCGACGACCAGATCGGATAC |
| | | TATAGACGGGCCACTCGGAGAATTC |
| | | GGGGCGGCGACGGAAAAATGAAGGA |
| | | CCTTTCTCCAAGATGGTACTTCTAC |
| | | TACCTCGGCACAGGCCCTGAGGCCG |
| | | GCCTGCCTTACGGCGCCAACAAGGA |
| | | TGGCATCATCTGGGTCGCCACCGAG |
| | | GGCGCCCTGAACACCCCTAAGGACC |
| | | ACATCGGCACAAGAAACCCCGCTAA |
| | | CAACGCCGCAATCGTGCTGCAGCTG |
| | | CCTCAGGGCACCACCCTGCCCAAGG |
| | | GCTTCTACGCCGAGGGCTCTAGAGG |
| | | TGGCTCCCAGGCTTCTAGCCGCTCC |
| | | TCCAGCCGCAGCAGAAACAGCAGCA |
| | | GGAACAGCACCCCCGGCAGCTCCCG |
| | | GGGCACCAGCCCCGCCAGAATGGCC |
| | | GGAAATGGCGGCGATGCCGCCCTGG |
| | | CCCTGCTCCTGCTGGACAGACTGAA |
| | | TCAGCTGGAAAGCAAGATGAGCGGC |
| | | AAAGGACAGCAGCAGCAAGGCCAGA |
| | | CCGTGACCAAGAAAAGCGCTGCTGA |
| | | AGCCTCCAAGAAACCTAGACAAAAG |
| | | CGGACCGCCACAAAGGCCTACAACG |
| | | TGACCCAAGCCTTTGGAAGAAGAGG |
| | | CCCCGAGCAGACACAGGGCAATTTC |
| | | GGCGACCAGGAGCTGATCCGGCAGG |
| | | GAACCGACTACAAGCACTGGCCTCA |
| | | GATCGCCCAGTTCGCCCCTAGCGCC |
| | | AGCGCCTTCTTCGGCATGAGCAGAA |
| | | TCGGCATGGAAGTGACCCCTTCTGG |
| | | CACCTGGCTGACCTACACCGGCGCT |
| | | ATCAAGCTGGACGATAAGGATCCTA |
| | | ACTTCAAGGACCAAGTGATCCTGCT |
| | | GAACAAGCATATCGACGCCTATAAG |
| | | ACCTTTCCACCTACAGAGCCTAAGA |
| | | AAGATAAGAAGAAGAAAGCCGACGA |
| | | GACACAGGCCCTGCCTCAGAGACAG |
| | | AAAAAGCAGCAGACAGTGACACTGC |
| | | TGCCAGCCGCTGACCTGGATGACTT |
| | | CAGCAAGCAGCTGCAGCAGAGCATG |
| | | TCTTCTGCTGATAGCACCCAGGCCC |
| | | GAAAACGGCGCggaagcggaggaag |
| | | cggagctactaacttcagcctgctg |
| | | aagcaggctggagatgtggaggaga |
| | | accctggacctATGTTCGTGTTCCT |
| | | GGTGCTGCTGCCTCTGGTCAGCTCC |
| | | CAGTGTGTGAACCTGACCACCAGAA |
| | | CCCAGCTGCCACCTGCTTATACAAA |
| | | CTCCTTCACTCGGGGGGTATACTAC |
| | | CCCGACAAGGTGTTCAGATCTAGCG |
| | | TGCTGCATTCTACACAAGACCTGTT |
| | | CCTGCCCTTCTTCAGCAACGTGACC |
| | | TGGTTCCACGCCATCCACGTGTCTG |
| | | GAACCAACGGAACCAAGAGATTCGA |
| | | CAACCCCGTGCTGCCTTTCAACGAC |
| | | GGCGTGTACTTCGCCAGCACCGAGA |
| | | AGTCCAACATCATCAGAGGATGGAT |
| | | TTTCGGCACCACACTGGACAGCAAA |
| | | ACCCAGAGCCTGCTGATCGTGAACA |
| | | ACGCCACCAACGTGGTGATCAAGGT |
| | | GTGCGAGTTCCAGTTCTGCAATGAT |
| | | CCCTTCCTGGGCGTGTACTACCACA |
| | | AGAACAACAAGTCTTGGATGGAAAG |
| | | CGAGTTCAGAGTGTATTCCAGCGCC |
| | | AACAATTGCACCTTCGAGTACGTGA |
| | | GCCAACCCTTTCTGATGGACCTTGA |
| | | AGGCAAGCAGGGCAACTTCAAAAAT |
| | | CTGCGAGAATTTGTGTTCAAGAACA |
| | | TCGACGGATACTTCAAGATCTACTC |
| | | TAAGCACACGCCAATCAACCTGGTG |
| | | AGAGATCTGCCCCAGGGCTTTAGCG |
| | | CTTTGGAACCTCTGGTGGACCTGCC |
| | | TATCGGAATCAACATCACCAGATTT |
| | | CAAACTCTCCTGGCCCTGCACAGAT |
| | | CTTATCTGACCCCTGGGGACAGTAG |
| | | TAGCGGCTGGACAGCCGGCGCCGCC |
| | | GCCTACTACGTGGGATACCTGCAGC |
| | | CTAGAACATTCCTGCTGAAGTACAA |
| | | TGAGAACGGAACAATCACAGACGCC |
| | | GTGGACTGCGCCCTGGATCCTTTGA |
| | | GCGAGACAAAGTGCACCCTGAAGTC |
| | | GTTCACCGTCGAAAAAGGCATCTAC |
| | | CAGACCAGCAACTTCCGCGTGCAGC |
| | | CTACGGAATCTATCGTGCGGTTCCC |
| | | CAACATCACCAACCTGTGCCCTTTC |
| | | GGCGAGGTGTTTAACGCTACAAGGT |
| | | TCGCCAGCGTGTATGCCTGGAACAG |
| | | AAAGAGAATCAGCAATTGCGTGGCC |
| | | GATTATAGCGTTCTGTACAACAGCG |
| | | CTTCCTTCAGCACCTTCAAGTGCTA |
| | | CGGCGTGTCTCCAACCAAGCTGAAC |
| | | GACCTCTGCTTCACCAATGTCTACG |
| | | CTGACTCTTTCGTGATTAGAGGCGA |
| | | TGAGGTTAGACAGATCGCACCTGGC |
| | | CAGACCGGCAAAATCGCTGACTACA |
| | | ACTACAAGCTGCCTGATGACTTCAC |
| | | AGGCTGTGTCATTGCCTGGAACTCA |
| | | AATAACCTGGACTCTAAAGTGGGCG |
| | | GCAACTACAACTACCTGTACCGGCT |
| | | GTTCCGGAAGAGCAATCTGAAACCT |
| | | TTTGAGCGGGACATCTCTACAGAGA |
| | | TCTACCAGGCCGGCAGCACACCCTG |
| | | CAACGGCGTTGAGGGCTTCAACTGC |
| | | TACTTCCCTCTGCAGAGCTACGGCT |
| | | TTCAGCCAACAAATGGAGTGGGCTA |
| | | CCAGCCGTACAGAGTGGTGGTGCTG |
| | | AGCTTCGAACTGCTGCATGCCCCAG |
| | | CCACAGTGTGTGGACCTAAGAAGTC |
| | | TACCAACCTGGTGAAGAACAAGTGC |
| | | GTGAACTTTAACTTTAACGGCCTGA |
| | | CCGGCACAGGCGTGCTGACCGAATC |
| | | CAACAAAAAGTTCCTGCCCTTCCAA |
| | | CAGTTCGGCAGAGACATCGCCGATA |
| | | CAACCGATGCCGTGCGGGACCCCCA |
| | | GACCTTAGAAATCCTAGATATCACC |
| | | CCGTGCAGCTTCGGCGGAGTCTCTG |
| | | TTATTACTCCTGGCACCAACACCAG |
| | | CAACCAAGTGGCTGTTCTGTACCAA |
| | | ggcGTGAACTGCACCGAAGTGCCTG |
| | | TGGCTATCCACGCCGATCAGCTGAC |
| | | CCCAACCTGGCGGGTGTATAGCACC |
| | | GGCTCTAACGTGTTCCAGACCCGGG |
| | | CTGGCTGCCTGATCGGCGCCGAACA |
| | | CGTCAACAACTCCTATGAATGTGAC |
| | | ATCCCCATCGGGGCTGGCATCTGCG |
| | | CCAGTTACCAGACACAGACAAATAG |
| | | CCCTAGACGGGCCAGAAGCGTGGCC |
| | | TCCCAGAGTATCATTGCCTACACCA |
| | | TGAGCCTGGGCGCCGAGAACAGCGT |
| | | GGCCTATTCTAACAATAGCATCGCA |
| | | ATCCCTACCAACTTTACCATCTCTG |
| | | TGACAACCGAGATCCTGCCTGTGAG |
| | | CATGACCAAAACCAGCGTGGACTGC |
| | | ACGATGTACATCTGTGGCGACAGCA |
| | | CAGAATGCAGTAATCTGTTGCTGCA |
| | | GTACGGCAGCTTTTGCACCCAGTTG |
| | | AATAGAGCCCTGACCGGAATCGCCG |
| | | TAGAGCAGGACAAAAATACCCAGGA |
| | | GGTGTTCGCCCAGGTGAAACAGATC |
| | | TACAAGACACCTCCCATTAAGGACT |
| | | TCGGAGGTTTTAACTTCAGCCAGAT |
| | | CCTGCCCGACCCTTCCAAGCCTAGC |
| | | AAACGCTCCTTCATCGAGGACCTGC |
| | | TCTTCAACAAGGTGACACTGGCTGA |
| | | TGCCGGCTTCATCAAGCAGTACGGA |
| | | GATTGTCTGGGAGACATCGCCGCTA |
| | | GAGATCTGATCTGCGCCCAAAAGTT |
| | | CAACGGCCTGACCGTGCTGCCTCCT |
| | | CTGCTTACAGACGAGATGATCGCCC |
| | | AGTACACCAGCGCCCTGCTGGCTGG |
| | | CACCATCACAAGCGGCTGGACCTTC |
| | | GGAGCCGGAGCCGCTCTGCAAATCC |
| | | CCTTTGCCATGCAGATGGCCTACCG |
| | | GTTCAACGGCATCGGCGTGACACAG |
| | | AATGTGCTGTACGAGAACCAGAAGC |
| | | TGATCGCTAACCAGTTTAACAGCGC |
| | | TATCGGCAAGATCCAGGACTCGCTG |
| | | AGTAGCACCGCCTCTGCCCTGGGCA |
| | | AGCTGCAGGACGTCGTGAACCAGAA |
| | | CGCCCAAGCCCTGAACACACTGGTG |
| | | AAACAGCTGAGCAGCAACTTCGGCG |
| | | CCATCAGCTCTGTGCTGAACGATAT |
| | | CCTGAGCAGACTGGACAAGGTGGAA |
| | | GCCGAGGTCCAGATCGACAGACTGA |
| | | TCACAGGAAGACTGCAGAGCCTGCA |
| | | AACGTACGTGACACAGCAGCTGATC |
| | | CGGGCAGCCGAAATCCGGGCCAGCG |
| | | CCAATCTGGCCGCTACCAAGATGAG |
| | | CGAGTGCGTGTTAGGCCAGAGCAAG |
| | | CGGGTGGATTTCTGCGGTAAGGGAT |
| | | ACCACCTGATGAGCTTTCCCCAGAG |
| | | CGCTCCTCACGGCGTGGTGTTTCTG |
| | | CACGTGACCTACGTTCCTGCCCAGG |
| | | AAAAGAACTTCACCACCGCCCCTGC |
| | | TATCTGCCACGATGGCAAGGCCCAC |
| | | TTCCCTAGAGAGGGCGTTTTCGTGT |
| | | CTAACGGCACACACTGGTTTGTGAC |
| | | CCAGAGAAACTTCTACGAGCCTCAG |
| | | ATCATCACCACAGACAACACCTTTG |
| | | TGAGCGGCAATTGCGACGTGGTGAT |
| | | CGGAATTGTTAATAATACCGTGTAC |
| | | GACCCTCTGCAGCCTGAGCTCGACA |
| | | GCTTCAAGGAAGAGCTGGACAAGTA |
| | | CTTCAAGAACCACACCTCCCCAGAT |
| | | GTGGACCTGGGCGATATTTCAGGCA |
| | | TCAACGCCTCCGTCGTGAATATCCA |
| | | GAAGGAGATCGACCGGCTCAACGAG |
| | | GTGGCCAAGAACCTTAACGAGAGCC |
| | | TGATCGACCTGCAGGAACTGGGCAA |
| | | ATATGAGCAGTACATCAAGTGGCCT |
| | | TGGTACATCTGGCTGGGCTTTATCG |
| | | CAGGCCTGATCGCTATCGTGATGGT |
| | | GACCATTATGCTGTGTTGTATGACC |
| | | AGCTGTTGTAGTTGTCTGAAGGGCT |
| | | GCTGTTCTTGCGGCAGCTGCTGCAA |
| | | GTTCGACGAAGACGACTCAGAGCCC |
| | | GTGCTGAAAGGCGTGAAGCTGCACT |
| | | ACACCCGAAAACGGCGCggaagcgg |
| | | aggaagcggagctactaacttcagc |
| | | ctgctgaagcaggctggagatgtgg |
| | | aggagaaccctggacctATGTATTC |
| | | TTTTGTGTCCGAGGAAACCGGCACA |
| | | CTGATCGTTAATAGCGTGCTGCTCT |
| | | TCCTGGCCTTCGTGGTGTTCCTGCT |
| | | GGTGACCCTGGCTATCCTGACCGCC |
| | | CTGAGACTGTGTGCCTACTGCTGCA |
| | | ACATCGTGAACGTGTCTCTGGTCAA |
| | | GCCTAGCTTCTACGTGTACAGCCGG |
| | | GTGAAGAACCTGAACAGCAGCAGAG |
| | | TGCCCGACCTGCTGGTGtaatcccc |
| | | cccccctaacgttactggccgaagc |
| | | cgcttggaataaggccggtgtgcgt |
| | | ttgtctatatgttattttccaccat |
| | | attgccgtcttttggcaatgtgagg |
| | | gcccggaaacctggccctgtcttct |
| | | tgacgagcattcctaggggtctttc |
| | | ccctctcgccaaaggaatgcaaggt |
| | | ctgttgaatgtcgtgaaggaagcag |
| | | ttcctctggaagcttcttgaagaca |
| | | aacaacgtctgtagcgaccctttgc |
| | | aggcagcggaaccccccacctggcg |
| | | acaggtgcctctgcggccaaaagcc |
| | | acgtgtataagatacacctgcaaag |
| | | gcggcacaaccccagtgccacgttg |
| | | tgagttggatagttgtggaaagagt |
| | | caaatggctctcctcaagcgtattc |
| | | aacaaggggctgaaggatgcccaga |
| | | aggtaccccattgtatgggatctga |
| | | tctggggcctcggtgcacatgcttt |
| | | acatgtgtttagtcgaggttaaaaa |
| | | aacgtctaggccccccgaaccacgg |
| | | ggacgtggttttcctttgaaaaaca |
| | | cgatgataaGTGTttacctgttaat |
| | | gtagcatttgagctttgggctaagc |
| | | gcaacattaaaccagtaccagaggt |
| | | gaaaatactcaataatttgggtgtg |
| | | gacattgctgctaatactgtgatct |
| | | gggactacaaaagagatgctccagc |
| | | acatatatctactattggtgtttgt |
| | | tctatgactgacatagccaagaaac |
| | | caactgaaacgatttgtgcaccact |
| | | cactgtcttttttgatggtagagtt |
| | | gatggtcaagtagacttatttagaa |
| | | atgcccgtaatggtgttcttattac |
| | | agaaggtagtgttaaaggtttacaa |
| | | ccatctgtaggtcccaaacaagcta |
| | | gtcttaatggagtcacattaattgg |
| | | agaagccgtaaaaacacagttcaat |
| | | tattataagaaagttgatggtgttg |
| | | tccaacaattacctgaaacttactt |
| | | tactcagagtagaaatttacaagaa |
| | | tttaaacccaggagtcaaatggaaa |
| | | ttgatttcttagaattagctatgga |
| | | tgaattcattgaacggtataaatta |
| | | gaaggctatgccttcgaacatatcg |
| | | tttatggagattttagtcatagtca |
| | | gttaggtggtGCGAaattgttgttg |
| | | tt |
| CoVEG16 | 48 | ATGGCCGACAGCAACGGCACAATCA |
| expression | | CCGTGGAAGAGCTGAAGAAACTGCT |
| cassette | | GGAACAGTGGAACCTGGTCATCGGC |
| | | TTCCTGTTTCTGACCTGGATCTGTC |
| | | TGCTGCAGTTCGCTTATGCCAATCG |
| | | GAACAGATTCCTGTACATCATCAAG |
| | | CTGATCTTCCTGTGGCTGCTGTGGC |
| | | CTGTGACCCTGGCTTGCTTCGTGCT |
| | | GGCCGCTGTGTACCGGATCAACTGG |
| | | ATCACAGGCGGAATCGCCATCGCCA |
| | | TGGCCTGCCTGGTGGGCCTGATGTG |
| | | GCTGAGCTACTTCATCGCTTCTTTC |
| | | AGACTGTTCGCCAGAACCCGGAGCA |
| | | TGTGGTCCTTCAACCCCGAGACAAA |
| | | CATCCTGCTGAACGTGCCTCTGCAC |
| | | GGCACCATCCTGACAAGACCTCTGC |
| | | TCGAGAGCGAGCTGGTGATTGGCGC |
| | | AGTGATTCTGAGAGGCCATCTGAGG |
| | | ATCGCCGGACACCACCTGGGCAGAT |
| | | GCGACATCAAGGACCTTCCAAAGGA |
| | | AATCACCGTTGCCACCAGCCGGACC |
| | | CTGTCCTACTACAAACTGGGCGCCA |
| | | GCCAAAGAGTGGCCGGCGATAGCGG |
| | | CTTTGCCGCCTACAGCAGATACCGC |
| | | ATCGGAAATTACAAGCTCAACACCG |
| | | ACCACAGCAGCTCTTCTGATAACAT |
| | | CGCCCTGCTGGTGCAGCGAAAACGG |
| | | CGCggaagcggaggaagcggagcta |
| | | ctaacttcagcctgctgaagcaggc |
| | | tggagatgtggaggagaaccctgga |
| | | cctATGAGCGACAACGGCCCTCAAA |
| | | ACCAGAGAAATGCCCCTCGGATCAC |
| | | ATTTGGCGGACCTAGCGACAGCACC |
| | | GGCAGCAACCAGAATGGAGAAAGAA |
| | | GCGGCGCCAGATCCAAGCAGCGGAG |
| | | ACCTCAGGGACTGCCCAACAACACC |
| | | GCTAGCTGGTTCACCGCCCTGACCC |
| | | AACACGGCAAGGAAGATCTGAAGTT |
| | | CCCCAGAGGCCAGGGCGTGCCTATC |
| | | AACACAAACTCTTCTCCCGACGACC |
| | | AGATCGGATACTATAGACGGGCCAC |
| | | TCGGAGAATTCGGGGCGGCGACGGA |
| | | AAAATGAAGGACCTTTCTCCAAGAT |
| | | GGTACTTCTACTACCTCGGCACAGG |
| | | CCCTGAGGCCGGCCTGCCTTACGGC |
| | | GCCAACAAGGATGGCATCATCTGGG |
| | | TCGCCACCGAGGGCGCCCTGAACAC |
| | | CCCTAAGGACCACATCGGCACAAGA |
| | | AACCCCGCTAACAACGCCGCAATCG |
| | | TGCTGCAGCTGCCTCAGGGCACCAC |
| | | CCTGCCCAAGGGCTTCTACGCCGAG |
| | | GGCTCTAGAGGTGGCTCCCAGGCTT |
| | | CTAGCCGCTCCTCCAGCCGCAGCAG |
| | | AAACAGCAGCAGGAACAGCACCCCC |
| | | GGCAGCTCCCGGGGCACCAGCCCCG |
| | | CCAGAATGGCCGGAAATGGCGGCGA |
| | | TGCCGCCCTGGCCCTGCTCCTGCTG |
| | | GACAGACTGAATCAGCTGGAAAGCA |
| | | AGATGAGCGGCAAAGGACAGCAGCA |
| | | GCAAGGCCAGACCGTGACCAAGAAA |
| | | AGCGCTGCTGAAGCCTCCAAGAAAC |
| | | CTAGACAAAAGCGGACCGCCACAAA |
| | | GGCCTACAACGTGACCCAAGCCTTT |
| | | GGAAGAAGAGGCCCCGAGCAGACAC |
| | | AGGGCAATTTCGGCGACCAGGAGCT |
| | | GATCCGGCAGGGAACCGACTACAAG |
| | | CACTGGCCTCAGATCGCCCAGTTCG |
| | | CCCCTAGCGCCAGCGCCTTCTTCGG |
| | | CATGAGCAGAATCGGCATGGAAGTG |
| | | ACCCCTTCTGGCACCTGGCTGACCT |
| | | ACACCGGCGCTATCAAGCTGGACGA |
| | | TAAGGATCCTAACTTCAAGGACCAA |
| | | GTGATCCTGCTGAACAAGCATATCG |
| | | ACGCCTATAAGACCTTTCCACCTAC |
| | | AGAGCCTAAGAAAGATAAGAAGAAG |
| | | AAAGCCGACGAGACACAGGCCCTGC |
| | | CTCAGAGACAGAAAAAGCAGCAGAC |
| | | AGTGACACTGCTGCCAGCCGCTGAC |
| | | CTGGATGACTTCAGCAAGCAGCTGC |
| | | AGCAGAGCATGTCTTCTGCTGATAG |
| | | CACCCAGGCCCGAAAACGGCGCgga |
| | | agcggaggaagcggagctactaact |
| | | tcagcctgctgaagcaggctggaga |
| | | tgtggaggagaaccctggacctATG |
| | | TTCGTGTTCCTGGTGCTGCTGCCTC |
| | | TGGTCAGCTCCCAGTGTGTGAACCT |
| | | GACCACCAGAACCCAGCTGCCACCT |
| | | GCTTATACAAACTCCTTCACTCGGG |
| | | GGGTATACTACCCCGACAAGGTGTT |
| | | CAGATCTAGCGTGCTGCATTCTACA |
| | | CAAGACCTGTTCCTGCCCTTCTTCA |
| | | GCAACGTGACCTGGTTCCACGCCAT |
| | | CCACGTGTCTGGAACCAACGGAACC |
| | | AAGAGATTCGACAACCCCGTGCTGC |
| | | CTTTCAACGACGGCGTGTACTTCGC |
| | | CAGCACCGAGAAGTCCAACATCATC |
| | | AGAGGATGGATTTTCGGCACCACAC |
| | | TGGACAGCAAAACCCAGAGCCTGCT |
| | | GATCGTGAACAACGCCACCAACGTG |
| | | GTGATCAAGGTGTGCGAGTTCCAGT |
| | | TCTGCAATGATCCCTTCCTGGGCGT |
| | | GTACTACCACAAGAACAACAAGTCT |
| | | TGGATGGAAAGCGAGTTCAGAGTGT |
| | | ATTCCAGCGCCAACAATTGCACCTT |
| | | CGAGTACGTGAGCCAACCCTTTCTG |
| | | ATGGACCTTGAAGGCAAGCAGGGCA |
| | | ACTTCAAAAATCTGCGAGAATTTGT |
| | | GTTCAAGAACATCGACGGATACTTC |
| | | AAGATCTACTCTAAGCACACGCCAA |
| | | TCAACCTGGTGAGAGATCTGCCCCA |
| | | GGGCTTTAGCGCTTTGGAACCTCTG |
| | | GTGGACCTGCCTATCGGAATCAACA |
| | | TCACCAGATTTCAAACTCTCCTGGC |
| | | CCTGCACAGATCTTATCTGACCCCT |
| | | GGGGACAGTAGTAGCGGCTGGACAG |
| | | CCGGCGCCGCCGCCTACTACGTGGG |
| | | ATACCTGCAGCCTAGAACATTCCTG |
| | | CTGAAGTACAATGAGAACGGAACAA |
| | | TCACAGACGCCGTGGACTGCGCCCT |
| | | GGATCCTTTGAGCGAGACAAAGTGC |
| | | ACCCTGAAGTCGTTCACCGTCGAAA |
| | | AAGGCATCTACCAGACCAGCAACTT |
| | | CCGCGTGCAGCCTACGGAATCTATC |
| | | GTGCGGTTCCCCAACATCACCAACC |
| | | TGTGCCCTTTCGGCGAGGTGTTTAA |
| | | CGCTACAAGGTTCGCCAGCGTGTAT |
| | | GCCTGGAACAGAAAGAGAATCAGCA |
| | | ATTGCGTGGCCGATTATAGCGTTCT |
| | | GTACAACAGCGCTTCCTTCAGCACC |
| | | TTCAAGTGCTACGGCGTGTCTCCAA |
| | | CCAAGCTGAACGACCTCTGCTTCAC |
| | | CAATGTCTACGCTGACTCTTTCGTG |
| | | ATTAGAGGCGATGAGGTTAGACAGA |
| | | TCGCACCTGGCCAGACCGGCAAAAT |
| | | CGCTGACTACAACTACAAGCTGCCT |
| | | GATGACTTCACAGGCTGTGTCATTG |
| | | CCTGGAACTCAAATAACCTGGACTC |
| | | TAAAGTGGGCGGCAACTACAACTAC |
| | | CTGTACCGGCTGTTCCGGAAGAGCA |
| | | ATCTGAAACCTTTTGAGCGGGACAT |
| | | CTCTACAGAGATCTACCAGGCCGGC |
| | | AGCACACCCTGCAACGGCGTTGAGG |
| | | GCTTCAACTGCTACTTCCCTCTGCA |
| | | GAGCTACGGCTTTCAGCCAACAAAT |
| | | GGAGTGGGCTACCAGCCGTACAGAG |
| | | TGGTGGTGCTGAGCTTCGAACTGCT |
| | | GCATGCCCCAGCCACAGTGTGTGGA |
| | | CCTAAGAAGTCTACCAACCTGGTGA |
| | | AGAACAAGTGCGTGAACTTTAACTT |
| | | TAACGGCCTGACCGGCACAGGCGTG |
| | | CTGACCGAATCCAACAAAAAGTTCC |
| | | TGCCCTTCCAACAGTTCGGCAGAGA |
| | | CATCGCCGATACAACCGATGCCGTG |
| | | CGGGACCCCCAGACCTTAGAAATCC |
| | | TAGATATCACCCCGTGCAGCTTCGG |
| | | CGGAGTCTCTGTTATTACTCCTGGC |
| | | ACCAACACCAGCAACCAAGTGGCTG |
| | | TTCTGTACCAAggcGTGAACTGCAC |
| | | CGAAGTGCCTGTGGCTATCCACGCC |
| | | GATCAGCTGACCCCAACCTGGCGGG |
| | | TGTATAGCACCGGCTCTAACGTGTT |
| | | CCAGACCCGGGCTGGCTGCCTGATC |
| | | GGCGCCGAACACGTCAACAACTCCT |
| | | ATGAATGTGACATCCCCATCGGGGC |
| | | TGGCATCTGCGCCAGTTACCAGACA |
| | | CAGACAAATAGCCCTGGCAGCGCCA |
| | | GCAGCGTGGCCTCCCAGAGTATCAT |
| | | TGCCTACACCATGAGCCTGGGCGCC |
| | | GAGAACAGCGTGGCCTATTCTAACA |
| | | ATAGCATCGCAATCCCTACCAACTT |
| | | TACCATCTCTGTGACAACCGAGATC |
| | | CTGCCTGTGAGCATGACCAAAACCA |
| | | GCGTGGACTGCACGATGTACATCTG |
| | | TGGCGACAGCACAGAATGCAGTAAT |
| | | CTGTTGCTGCAGTACGGCAGCTTTT |
| | | GCACCCAGTTGAATAGAGCCCTGAC |
| | | CGGAATCGCCGTAGAGCAGGACAAA |
| | | AATACCCAGGAGGTGTTCGCCCAGG |
| | | TGAAACAGATCTACAAGACACCTCC |
| | | CATTAAGGACTTCGGAGGTTTTAAC |
| | | TTCAGCCAGATCCTGCCCGACCCTT |
| | | CCAAGCCTAGCAAACGCTCCTTCAT |
| | | CGAGGACCTGCTCTTCAACAAGGTG |
| | | ACACTGGCTGATGCCGGCTTCATCA |
| | | AGCAGTACGGAGATTGTCTGGGAGA |
| | | CATCGCCGCTAGAGATCTGATCTGC |
| | | GCCCAAAAGTTCAACGGCCTGACCG |
| | | TGCTGCCTCCTCTGCTTACAGACGA |
| | | GATGATCGCCCAGTACACCAGCGCC |
| | | CTGCTGGCTGGCACCATCACAAGCG |
| | | GCTGGACCTTCGGAGCCGGAGCCGC |
| | | TCTGCAAATCCCCTTTGCCATGCAG |
| | | ATGGCCTACCGGTTCAACGGCATCG |
| | | GCGTGACACAGAATGTGCTGTACGA |
| | | GAACCAGAAGCTGATCGCTAACCAG |
| | | TTTAACAGCGCTATCGGCAAGATCC |
| | | AGGACTCGCTGAGTAGCACCGCCTC |
| | | TGCCCTGGGCAAGCTGCAGGACGTC |
| | | GTGAACCAGAACGCCCAAGCCCTGA |
| | | ACACACTGGTGAAACAGCTGAGCAG |
| | | CAACTTCGGCGCCATCAGCTCTGTG |
| | | CTGAACGATATCCTGAGCAGACTGG |
| | | ACCCTcccGAAGCCGAGGTCCAGAT |
| | | CGACAGACTGATCACAGGAAGACTG |
| | | CAGAGCCTGCAAACGTACGTGACAC |
| | | AGCAGCTGATCCGGGCAGCCGAAAT |
| | | CCGGGCCAGCGCCAATCTGGCCGCT |
| | | ACCAAGATGAGCGAGTGCGTGTTAG |
| | | GCCAGAGCAAGCGGGTGGATTTCTG |
| | | CGGTAAGGGATACCACCTGATGAGC |
| | | TTTCCCCAGAGCGCTCCTCACGGCG |
| | | TGGTGTTTCTGCACGTGACCTACGT |
| | | TCCTGCCCAGGAAAAGAACTTCACC |
| | | ACCGCCCCTGCTATCTGCCACGATG |
| | | GCAAGGCCCACTTCCCTAGAGAGGG |
| | | CGTTTTCGTGTCTAACGGCACACAC |
| | | TGGTTTGTGACCCAGAGAAACTTCT |
| | | ACGAGCCTCAGATCATCACCACAGA |
| | | CAACACCTTTGTGAGCGGCAATTGC |
| | | GACGTGGTGATCGGAATTGTTAATA |
| | | ATACCGTGTACGACCCTCTGCAGCC |
| | | TGAGCTCGACAGCTTCAAGGAAGAG |
| | | CTGGACAAGTACTTCAAGAACCACA |
| | | CCTCCCCAGATGTGGACCTGGGCGA |
| | | TATTTCAGGCATCAACGCCTCCGTC |
| | | GTGAATATCCAGAAGGAGATCGACC |
| | | GGCTCAACGAGGTGGCCAAGAACCT |
| | | TAACGAGAGCCTGATCGACCTGCAG |
| | | GAACTGGGCAAATATGAGCAGTACA |
| | | TCAAGTGGCCTTGGTACATCTGGCT |
| | | GGGCTTTATCGCAGGCCTGATCGCT |
| | | ATCGTGATGGTGACCATTATGCTGT |
| | | GTTGTATGACCAGCTGTTGTAGTTG |
| | | TCTGAAGGGCTGCTGTTCTTGCGGC |
| | | AGCTGCTGCAAGTTCGACGAAGACG |
| | | ACTCAGAGCCCGTGCTGAAAGGCGT |
| | | GAAGCTGCACTACACCCGAAAACGG |
| | | CGCggaagcggaggaagcggagcta |
| | | ctaacttcagcctgctgaagcaggc |
| | | tggagatgtggaggagaaccctgga |
| | | cctATGTATTCTTTTGTGTCCGAGG |
| | | AAACCGGCACACTGATCGTTAATAG |
| | | CGTGCTGCTCTTCCTGGCCTTCGTG |
| | | GTGTTCCTGCTGGTGACCCTGGCTA |
| | | TCCTGACCGCCCTGAGACTGTGTGC |
| | | CTACTGCTGCAACATCGTGAACGTG |
| | | TCTCTGGTCAAGCCTAGCTTCTACG |
| | | TGTACAGCCGGGTGAAGAACCTGAA |
| | | CAGCAGCAGAGTGCCCGACCTGCTG |
| | | GTGtaatcccccccccctaacgtta |
| | | ctggccgaagccgcttggaataagg |
| | | ccggtgtgcgtttgtctatatgtta |
| | | ttttccaccatattgccgtcttttg |
| | | gcaatgtgagggcccggaaacctgg |
| | | ccctgtcttcttgacgagcattcct |
| | | aggggtctttcccctctcgccaaag |
| | | gaatgcaaggtctgttgaatgtcgt |
| | | gaaggaagcagttcctctggaagct |
| | | tcttgaagacaaacaacgtctgtag |
| | | cgaccctttgcaggcagcggaaccc |
| | | cccacctggcgacaggtgcctctgc |
| | | ggccaaaagccacgtgtataagata |
| | | cacctgcaaaggcggcacaacccca |
| | | gtgccacgttgtgagttggatagtt |
| | | gtggaaagagtcaaatggctctcct |
| | | caagcgtattcaacaaggggctgaa |
| | | ggatgcccagaaggtaccccattgt |
| | | atgggatctgatctggggcctcggt |
| | | gcacatgctttacatgtgtttagtc |
| | | gaggttaaaaaaacgtctaggcccc |
| | | ccgaaccacggggacgtggttttcc |
| | | tttgaaaaacacgatgataatatgg |
| | | ccacaaccatggaacaagagacttg |
| | | cgcgcactctctcacttttgaggaa |
| | | tgcccaaaatgctctgctctacaat |
| | | accgtaatggattttacctgctaaa |
| | | gtatgatgaagaatggtacccagag |
| | | gagttattgactgatggagaggatg |
| | | atgtctttgatcccgaattagacat |
| | | ggaagtcgttttcgagttacaggga |
| | | agcggagctactaacttcagcctgc |
| | | tgaagcaggctggagatgtggagga |
| | | gaaccctggacctATGGACCTGTTC |
| | | ATGAGAATCTTCACCATCGGCACCG |
| | | TGACACTGAAGCAGGGCGAGATCAA |
| | | GGATGCCACCCCTAGCGACTTCGTG |
| | | AGAGCCACCGCCACAATTCCTATCC |
| | | AGGCTAGCCTGCCTTTTGGATGGCT |
| | | GATCGTGGGCGTCGCCCTGCTCGCC |
| | | GTGTTCCAGAGCGCCTCTAAGATCA |
| | | TTACACTGAAGAAAAGATGGCAGCT |
| | | GGCCCTCTCCAAAGGCGTGCACTTC |
| | | GTGTGTAATCTGCTGCTGCTTTTTG |
| | | TGACAGTGTACAGCCACCTGCTGCT |
| | | GGTTGCTGCTGGCCTGGAAGCCCCT |
| | | TTCCTGTACCTGTACGCCCTGGTCT |
| | | ACTTCCTGCAGTCTATCAACTTCGT |
| | | GCGGATCATCATGCGGCTGTGGCTG |
| | | TGCTGGAAGTGCAGAAGCAAGAACC |
| | | CACTGCTGTACGACGCCAATTACTT |
| | | CCTGTGTTGGCACACCAACTGCTAC |
| | | GACTACTGCATCCCCTACAACAGCG |
| | | TGACCAGCAGCATCGTGATCACCTC |
| | | TGGCGACGGAACAACCAGCCCTATC |
| | | AGCGAGCATGATTACCAGATCGGCG |
| | | GATATACAGAGAAGTGGGAGAGCGG |
| | | CGTGAAGGACTGCGTGGTGCTGCAC |
| | | AGCTACTTTACCTCCGATTACTACC |
| | | AACTGTATTCTACCCAGCTGAGCAC |
| | | CGACACCGGCGTGGAACACGTGACC |
| | | TTCTTCATCTACAACAAGATCGTGG |
| | | ACGAGCCTGAGGAACACGTGCAGAT |
| | | CCACACTATCGACGGCAGCTCTGGC |
| | | GTTGTGAACCCTGTGATGGAACCCA |
| | | TCTACGATGAGCCCACCACAACAAC |
| | | CTCCGTGCCCCTGTaaGTGTttacc |
| | | tgttaatgtagcatttgagctttgg |
| | | gctaagcgcaacattaaaccagtac |
| | | cagaggtgaaaatactcaataattt |
| | | gggtgtggacattgctgctaatact |
| | | gtgatctgggactacaaaagagatg |
| | | ctccagcacatatatctactattgg |
| | | tgtttgttctatgactgacatagcc |
| | | aagaaaccaactgaaacgatttgtg |
| | | caccactcactgtcttttttgatgg |
| | | tagagttgatggtcaagtagactta |
| | | tttagaaatgcccgtaatggtgttc |
| | | ttattacagaaggtagtgttaaagg |
| | | tttacaaccatctgtaggtcccaaa |
| | | caagctagtcttaatggagtcacat |
| | | taattggagaagccgtaaaaacaca |
| | | gttcaattattataagaaagttgat |
| | | ggtgttgtccaacaattacctgaaa |
| | | cttactttactcagagtagaaattt |
| | | acaagaatttaaacccaggagtcaa |
| | | atggaaattgatttcttagaattag |
| | | ctatggatgaattcattgaacggta |
| | | taaattagaaggctatgccttcgaa |
| | | catatcgtttatggagattttagtc |
| | | atagtcagttaggtggtGCGAaatt |
| | | gttgttgtt |
| CoVEG17 | 49 | ATTAAAGGTTTATACCTTCCCAGGT |
| expression | | AACAAACCAACCAACTTTCGATCTC |
| cassette | | TTGTAGATCTGTTCTCTAAACGAAC |
| | | TTTAAAATCTGTGTGGCTGTCACTC |
| | | GGCTGCATGCTTAGTGCACTCACGC |
| | | AGTATAATTAATAACTAATTACTGT |
| | | CGTTGACAGGACACGAGTAACTCGT |
| | | CTATCTTCTGCAGGCTGCTTACGGT |
| | | TTCGTCCGTGTTGCAGCCGATCATC |
| | | AGCACATCTAGGTTTCGTCCGGGTG |
| | | TGACCGAAAGGTAAATGGCCGACAG |
| | | CAACGGCACAATCACCGTGGAAGAG |
| | | CTGAAGAAACTGCTGGAACAGTGGA |
| | | ACCTGGTCATCGGCTTCCTGTTTCT |
| | | GACCTGGATCTGTCTGCTGCAGTTC |
| | | GCTTATGCCAATCGGAACAGATTCC |
| | | TGTACATCATCAAGCTGATCTTCCT |
| | | GTGGCTGCTGTGGCCTGTGACCCTG |
| | | GCTTGCTTCGTGCTGGCCGCTGTGT |
| | | ACCGGATCAACTGGATCACAGGCGG |
| | | AATCGCCATCGCCATGGCCTGCCTG |
| | | GTGGGCCTGATGTGGCTGAGCTACT |
| | | TCATCGCTTCTTTCAGACTGTTCGC |
| | | CAGAACCCGGAGCATGTGGTCCTTC |
| | | AACCCCGAGACAAACATCCTGCTGA |
| | | ACGTGCCTCTGCACGGCACCATCCT |
| | | GACAAGACCTCTGCTCGAGAGCGAG |
| | | CTGGTGATTGGCGCAGTGATTCTGA |
| | | GAGGCCATCTGAGGATCGCCGGACA |
| | | CCACCTGGGCAGATGCGACATCAAG |
| | | GACCTTCCAAAGGAAATCACCGTTG |
| | | CCACCAGCCGGACCCTGTCCTACTA |
| | | CAAACTGGGCGCCAGCCAAAGAGTG |
| | | GCCGGCGATAGCGGCTTTGCCGCCT |
| | | ACAGCAGATACCGCATCGGAAATTA |
| | | CAAGCTCAACACCGACCACAGCAGC |
| | | TCTTCTGATAACATCGCCCTGCTGG |
| | | TGCAGCGAAAACGGCGCggaagcgg |
| | | aggaagcggagctactaacttcagc |
| | | ctgctgaagcaggctggagatgtgg |
| | | aggagaaccctggacctATGAGCGA |
| | | CAACGGCCCTCAAAACCAGAGAAAT |
| | | GCCCCTCGGATCACATTTGGCGGAC |
| | | CTAGCGACAGCACCGGCAGCAACCA |
| | | GAATGGAGAAAGAAGCGGCGCCAGA |
| | | TCCAAGCAGCGGAGACCTCAGGGAC |
| | | TGCCCAACAACACCGCTAGCTGGTT |
| | | CACCGCCCTGACCCAACACGGCAAG |
| | | GAAGATCTGAAGTTCCCCAGAGGCC |
| | | AGGGCGTGCCTATCAACACAAACTC |
| | | TTCTCCCGACGACCAGATCGGATAC |
| | | TATAGACGGGCCACTCGGAGAATTC |
| | | GGGGCGGCGACGGAAAAATGAAGGA |
| | | CCTTTCTCCAAGATGGTACTTCTAC |
| | | TACCTCGGCACAGGCCCTGAGGCCG |
| | | GCCTGCCTTACGGCGCCAACAAGGA |
| | | TGGCATCATCTGGGTCGCCACCGAG |
| | | GGCGCCCTGAACACCCCTAAGGACC |
| | | ACATCGGCACAAGAAACCCCGCTAA |
| | | CAACGCCGCAATCGTGCTGCAGCTG |
| | | CCTCAGGGCACCACCCTGCCCAAGG |
| | | GCTTCTACGCCGAGGGCTCTAGAGG |
| | | TGGCTCCCAGGCTTCTAGCCGCTCC |
| | | TCCAGCCGCAGCAGAAACAGCAGCA |
| | | GGAACAGCACCCCCGGCAGCTCCCG |
| | | GGGCACCAGCCCCGCCAGAATGGCC |
| | | GGAAATGGCGGCGATGCCGCCCTGG |
| | | CCCTGCTCCTGCTGGACAGACTGAA |
| | | TCAGCTGGAAAGCAAGATGAGCGGC |
| | | AAAGGACAGCAGCAGCAAGGCCAGA |
| | | CCGTGACCAAGAAAAGCGCTGCTGA |
| | | AGCCTCCAAGAAACCTAGACAAAAG |
| | | CGGACCGCCACAAAGGCCTACAACG |
| | | TGACCCAAGCCTTTGGAAGAAGAGG |
| | | CCCCGAGCAGACACAGGGCAATTTC |
| | | GGCGACCAGGAGCTGATCCGGCAGG |
| | | GAACCGACTACAAGCACTGGCCTCA |
| | | GATCGCCCAGTTCGCCCCTAGCGCC |
| | | AGCGCCTTCTTCGGCATGAGCAGAA |
| | | TCGGCATGGAAGTGACCCCTTCTGG |
| | | CACCTGGCTGACCTACACCGGCGCT |
| | | ATCAAGCTGGACGATAAGGATCCTA |
| | | ACTTCAAGGACCAAGTGATCCTGCT |
| | | GAACAAGCATATCGACGCCTATAAG |
| | | ACCTTTCCACCTACAGAGCCTAAGA |
| | | AAGATAAGAAGAAGAAAGCCGACGA |
| | | GACACAGGCCCTGCCTCAGAGACAG |
| | | AAAAAGCAGCAGACAGTGACACTGC |
| | | TGCCAGCCGCTGACCTGGATGACTT |
| | | CAGCAAGCAGCTGCAGCAGAGCATG |
| | | TCTTCTGCTGATAGCACCCAGGCCC |
| | | GAAAACGGCGCggaagcggaggaag |
| | | cggagctactaacttcagcctgctg |
| | | aagcaggctggagatgtggaggaga |
| | | accctggacctATGTTCGTGTTCCT |
| | | GGTGCTGCTGCCTCTGGTCAGCTCC |
| | | CAGTGTGTGAACCTGACCACCAGAA |
| | | CCCAGCTGCCACCTGCTTATACAAA |
| | | CTCCTTCACTCGGGGGGTATACTAC |
| | | CCCGACAAGGTGTTCAGATCTAGCG |
| | | TGCTGCATTCTACACAAGACCTGTT |
| | | CCTGCCCTTCTTCAGCAACGTGACC |
| | | TGGTTCCACGCCATCCACGTGTCTG |
| | | GAACCAACGGAACCAAGAGATTCGA |
| | | CAACCCCGTGCTGCCTTTCAACGAC |
| | | GGCGTGTACTTCGCCAGCACCGAGA |
| | | AGTCCAACATCATCAGAGGATGGAT |
| | | TTTCGGCACCACACTGGACAGCAAA |
| | | ACCCAGAGCCTGCTGATCGTGAACA |
| | | ACGCCACCAACGTGGTGATCAAGGT |
| | | GTGCGAGTTCCAGTTCTGCAATGAT |
| | | CCCTTCCTGGGCGTGTACTACCACA |
| | | AGAACAACAAGTCTTGGATGGAAAG |
| | | CGAGTTCAGAGTGTATTCCAGCGCC |
| | | AACAATTGCACCTTCGAGTACGTGA |
| | | GCCAACCCTTTCTGATGGACCTTGA |
| | | AGGCAAGCAGGGCAACTTCAAAAAT |
| | | CTGCGAGAATTTGTGTTCAAGAACA |
| | | TCGACGGATACTTCAAGATCTACTC |
| | | TAAGCACACGCCAATCAACCTGGTG |
| | | AGAGATCTGCCCCAGGGCTTTAGCG |
| | | CTTTGGAACCTCTGGTGGACCTGCC |
| | | TATCGGAATCAACATCACCAGATTT |
| | | CAAACTCTCCTGGCCCTGCACAGAT |
| | | CTTATCTGACCCCTGGGGACAGTAG |
| | | TAGCGGCTGGACAGCCGGCGCCGCC |
| | | GCCTACTACGTGGGATACCTGCAGC |
| | | CTAGAACATTCCTGCTGAAGTACAA |
| | | TGAGAACGGAACAATCACAGACGCC |
| | | GTGGACTGCGCCCTGGATCCTTTGA |
| | | GCGAGACAAAGTGCACCCTGAAGTC |
| | | GTTCACCGTCGAAAAAGGCATCTAC |
| | | CAGACCAGCAACTTCCGCGTGCAGC |
| | | CTACGGAATCTATCGTGCGGTTCCC |
| | | CAACATCACCAACCTGTGCCCTTTC |
| | | GGCGAGGTGTTTAACGCTACAAGGT |
| | | TCGCCAGCGTGTATGCCTGGAACAG |
| | | AAAGAGAATCAGCAATTGCGTGGCC |
| | | GATTATAGCGTTCTGTACAACAGCG |
| | | CTTCCTTCAGCACCTTCAAGTGCTA |
| | | CGGCGTGTCTCCAACCAAGCTGAAC |
| | | GACCTCTGCTTCACCAATGTCTACG |
| | | CTGACTCTTTCGTGATTAGAGGCGA |
| | | TGAGGTTAGACAGATCGCACCTGGC |
| | | CAGACCGGCAAAATCGCTGACTACA |
| | | ACTACAAGCTGCCTGATGACTTCAC |
| | | AGGCTGTGTCATTGCCTGGAACTCA |
| | | AATAACCTGGACTCTAAAGTGGGCG |
| | | GCAACTACAACTACCTGTACCGGCT |
| | | GTTCCGGAAGAGCAATCTGAAACCT |
| | | TTTGAGCGGGACATCTCTACAGAGA |
| | | TCTACCAGGCCGGCAGCACACCCTG |
| | | CAACGGCGTTGAGGGCTTCAACTGC |
| | | TACTTCCCTCTGCAGAGCTACGGCT |
| | | TTCAGCCAACAAATGGAGTGGGCTA |
| | | CCAGCCGTACAGAGTGGTGGTGCTG |
| | | AGCTTCGAACTGCTGCATGCCCCAG |
| | | CCACAGTGTGTGGACCTAAGAAGTC |
| | | TACCAACCTGGTGAAGAACAAGTGC |
| | | GTGAACTTTAACTTTAACGGCCTGA |
| | | CCGGCACAGGCGTGCTGACCGAATC |
| | | CAACAAAAAGTTCCTGCCCTTCCAA |
| | | CAGTTCGGCAGAGACATCGCCGATA |
| | | CAACCGATGCCGTGCGGGACCCCCA |
| | | GACCTTAGAAATCCTAGATATCACC |
| | | CCGTGCAGCTTCGGCGGAGTCTCTG |
| | | TTATTACTCCTGGCACCAACACCAG |
| | | CAACCAAGTGGCTGTTCTGTACCAA |
| | | ggcGTGAACTGCACCGAAGTGCCTG |
| | | TGGCTATCCACGCCGATCAGCTGAC |
| | | CCCAACCTGGCGGGTGTATAGCACC |
| | | GGCTCTAACGTGTTCCAGACCCGGG |
| | | CTGGCTGCCTGATCGGCGCCGAACA |
| | | CGTCAACAACTCCTATGAATGTGAC |
| | | ATCCCCATCGGGGCTGGCATCTGCG |
| | | CCAGTTACCAGACACAGACAAATAG |
| | | CCCTGGCAGCGCCAGCAGCGTGGCC |
| | | TCCCAGAGTATCATTGCCTACACCA |
| | | TGAGCCTGGGCGCCGAGAACAGCGT |
| | | GGCCTATTCTAACAATAGCATCGCA |
| | | ATCCCTACCAACTTTACCATCTCTG |
| | | TGACAACCGAGATCCTGCCTGTGAG |
| | | CATGACCAAAACCAGCGTGGACTGC |
| | | ACGATGTACATCTGTGGCGACAGCA |
| | | CAGAATGCAGTAATCTGTTGCTGCA |
| | | GTACGGCAGCTTTTGCACCCAGTTG |
| | | AATAGAGCCCTGACCGGAATCGCCG |
| | | TAGAGCAGGACAAAAATACCCAGGA |
| | | GGTGTTCGCCCAGGTGAAACAGATC |
| | | TACAAGACACCTCCCATTAAGGACT |
| | | TCGGAGGTTTTAACTTCAGCCAGAT |
| | | CCTGCCCGACCCTTCCAAGCCTAGC |
| | | AAACGCTCCTTCATCGAGGACCTGC |
| | | TCTTCAACAAGGTGACACTGGCTGA |
| | | TGCCGGCTTCATCAAGCAGTACGGA |
| | | GATTGTCTGGGAGACATCGCCGCTA |
| | | GAGATCTGATCTGCGCCCAAAAGTT |
| | | CAACGGCCTGACCGTGCTGCCTCCT |
| | | CTGCTTACAGACGAGATGATCGCCC |
| | | AGTACACCAGCGCCCTGCTGGCTGG |
| | | CACCATCACAAGCGGCTGGACCTTC |
| | | GGAGCCGGAGCCGCTCTGCAAATCC |
| | | CCTTTGCCATGCAGATGGCCTACCG |
| | | GTTCAACGGCATCGGCGTGACACAG |
| | | AATGTGCTGTACGAGAACCAGAAGC |
| | | TGATCGCTAACCAGTTTAACAGCGC |
| | | TATCGGCAAGATCCAGGACTCGCTG |
| | | AGTAGCACCGCCTCTGCCCTGGGCA |
| | | AGCTGCAGGACGTCGTGAACCAGAA |
| | | CGCCCAAGCCCTGAACACACTGGTG |
| | | AAACAGCTGAGCAGCAACTTCGGCG |
| | | CCATCAGCTCTGTGCTGAACGATAT |
| | | CCTGAGCAGACTGGACCCTcccGAA |
| | | GCCGAGGTCCAGATCGACAGACTGA |
| | | TCACAGGAAGACTGCAGAGCCTGCA |
| | | AACGTACGTGACACAGCAGCTGATC |
| | | CGGGCAGCCGAAATCCGGGCCAGCG |
| | | CCAATCTGGCCGCTACCAAGATGAG |
| | | CGAGTGCGTGTTAGGCCAGAGCAAG |
| | | CGGGTGGATTTCTGCGGTAAGGGAT |
| | | ACCACCTGATGAGCTTTCCCCAGAG |
| | | CGCTCCTCACGGCGTGGTGTTTCTG |
| | | CACGTGACCTACGTTCCTGCCCAGG |
| | | AAAAGAACTTCACCACCGCCCCTGC |
| | | TATCTGCCACGATGGCAAGGCCCAC |
| | | TTCCCTAGAGAGGGCGTTTTCGTGT |
| | | CTAACGGCACACACTGGTTTGTGAC |
| | | CCAGAGAAACTTCTACGAGCCTCAG |
| | | ATCATCACCACAGACAACACCTTTG |
| | | TGAGCGGCAATTGCGACGTGGTGAT |
| | | CGGAATTGTTAATAATACCGTGTAC |
| | | GACCCTCTGCAGCCTGAGCTCGACA |
| | | GCTTCAAGGAAGAGCTGGACAAGTA |
| | | CTTCAAGAACCACACCTCCCCAGAT |
| | | GTGGACCTGGGCGATATTTCAGGCA |
| | | TCAACGCCTCCGTCGTGAATATCCA |
| | | GAAGGAGATCGACCGGCTCAACGAG |
| | | GTGGCCAAGAACCTTAACGAGAGCC |
| | | TGATCGACCTGCAGGAACTGGGCAA |
| | | ATATGAGCAGTACATCAAGTGGCCT |
| | | TGGTACATCTGGCTGGGCTTTATCG |
| | | CAGGCCTGATCGCTATCGTGATGGT |
| | | GACCATTATGCTGTGTTGTATGACC |
| | | AGCTGTTGTAGTTGTCTGAAGGGCT |
| | | GCTGTTCTTGCGGCAGCTGCTGCAA |
| | | GTTCGACGAAGACGACTCAGAGCCC |
| | | GTGCTGAAAGGCGTGAAGCTGCACT |
| | | ACACCCGAAAACGGCGCggaagcgg |
| | | aggaagcggagctactaacttcagc |
| | | ctgctgaagcaggctggagatgtgg |
| | | aggagaaccctggacctATGTATTC |
| | | TTTTGTGTCCGAGGAAACCGGCACA |
| | | CTGATCGTTAATAGCGTGCTGCTCT |
| | | TCCTGGCCTTCGTGGTGTTCCTGCT |
| | | GGTGACCCTGGCTATCCTGACCGCC |
| | | CTGAGACTGTGTGCCTACTGCTGCA |
| | | ACATCGTGAACGTGTCTCTGGTCAA |
| | | GCCTAGCTTCTACGTGTACAGCCGG |
| | | GTGAAGAACCTGAACAGCAGCAGAG |
| | | TGCCCGACCTGCTGGTGtaatcccc |
| | | cccccctaacgttactggccgaagc |
| | | cgcttggaataaggccggtgtgcgt |
| | | ttgtctatatgttattttccaccat |
| | | attgccgtcttttggcaatgtgagg |
| | | gcccggaaacctggccctgtcttct |
| | | tgacgagcattcctaggggtctttc |
| | | ccctctcgccaaaggaatgcaaggt |
| | | ctgttgaatgtcgtgaaggaagcag |
| | | ttcctctggaagcttcttgaagaca |
| | | aacaacgtctgtagcgaccctttgc |
| | | aggcagcggaaccccccacctggcg |
| | | acaggtgcctctgcggccaaaagcc |
| | | acgtgtataagatacacctgcaaag |
| | | gcggcacaaccccagtgccacgttg |
| | | tgagttggatagttgtggaaagagt |
| | | caaatggctctcctcaagcgtattc |
| | | aacaaggggctgaaggatgcccaga |
| | | aggtaccccattgtatgggatctga |
| | | tctggggcctcggtgcacatgcttt |
| | | acatgtgtttagtcgaggttaaaaa |
| | | aacgtctaggccccccgaaccacgg |
| | | ggacgtggttttcctttgaaaaaca |
| | | cgatgataatatggccacaaccatg |
| | | gaacaagagacttgcgcgcactctc |
| | | tcacttttgaggaatgcccaaaatg |
| | | ctctgctctacaataccgtaatgga |
| | | ttttacctgctaaagtatgatgaag |
| | | aatggtacccagaggagttattgac |
| | | tgatggagaggatgatgtctttgat |
| | | cccgaattagacatggaagtcgttt |
| | | tcgagttacagggaagcggagctac |
| | | taacttcagcctgctgaagcaggct |
| | | ggagatgtggaggagaaccctggac |
| | | ctATGGACCTGTTCATGAGAATCTT |
| | | CACCATCGGCACCGTGACACTGAAG |
| | | CAGGGCGAGATCAAGGATGCCACCC |
| | | CTAGCGACTTCGTGAGAGCCACCGC |
| | | CACAATTCCTATCCAGGCTAGCCTG |
| | | CCTTTTGGATGGCTGATCGTGGGCG |
| | | TCGCCCTGCTCGCCGTGTTCCAGAG |
| | | CGCCTCTAAGATCATTACACTGAAG |
| | | AAAAGATGGCAGCTGGCCCTCTCCA |
| | | AAGGCGTGCACTTCGTGTGTAATCT |
| | | GCTGCTGCTTTTTGTGACAGTGTAC |
| | | AGCCACCTGCTGCTGGTTGCTGCTG |
| | | GCCTGGAAGCCCCTTTCCTGTACCT |
| | | GTACGCCCTGGTCTACTTCCTGCAG |
| | | TCTATCAACTTCGTGCGGATCATCA |
| | | TGCGGCTGTGGCTGTGCTGGAAGTG |
| | | CAGAAGCAAGAACCCACTGCTGTAC |
| | | GACGCCAATTACTTCCTGTGTTGGC |
| | | ACACCAACTGCTACGACTACTGCAT |
| | | CCCCTACAACAGCGTGACCAGCAGC |
| | | ATCGTGATCACCTCTGGCGACGGAA |
| | | CAACCAGCCCTATCAGCGAGCATGA |
| | | TTACCAGATCGGCGGATATACAGAG |
| | | AAGTGGGAGAGCGGCGTGAAGGACT |
| | | GCGTGGTGCTGCACAGCTACTTTAC |
| | | CTCCGATTACTACCAACTGTATTCT |
| | | ACCCAGCTGAGCACCGACACCGGCG |
| | | TGGAACACGTGACCTTCTTCATCTA |
| | | CAACAAGATCGTGGACGAGCCTGAG |
| | | GAACACGTGCAGATCCACACTATCG |
| | | ACGGCAGCTCTGGCGTTGTGAACCC |
| | | TGTGATGGAACCCATCTACGATGAG |
| | | CCCACCACAACAACCTCCGTGCCCC |
| | | TGTaaGTGTttacctgttaatgtag |
| | | catttgagctttgggctaagcgcaa |
| | | cattaaaccagtaccagaggtgaaa |
| | | atactcaataatttgggtgtggaca |
| | | ttgctgctaatactgtgatctggga |
| | | ctacaaaagagatgctccagcacat |
| | | atatctactattggtgtttgttcta |
| | | tgactgacatagccaagaaaccaac |
| | | tgaaacgatttgtgcaccactcact |
| | | gtcttttttgatggtagagttgatg |
| | | gtcaagtagacttatttagaaatgc |
| | | ccgtaatggtgttcttattacagaa |
| | | ggtagtgttaaaggtttacaaccat |
| | | ctgtaggtcccaaacaagctagtct |
| | | taatggagtcacattaattggagaa |
| | | gccgtaaaaacacagttcaattatt |
| | | ataagaaagttgatggtgttgtcca |
| | | acaattacctgaaacttactttact |
| | | cagagtagaaatttacaagaattta |
| | | aacccaggagtcaaatggaaattga |
| | | tttcttagaattagctatggatgaa |
| | | ttcattgaacggtataaattagaag |
| | | gctatgccttcgaacatatcgttta |
| | | tggagattttagtcatagtcagtta |
| | | ggtggtGCGAaattgttgttgtt |
| Furin | 50 | CGAAAACGGCGC |
| cleavage | | |
| site | | |
| Viral | 34 | GTGTCaAAGcGCGAGGAACTGTTCA |
| packaging | | CCGGAGTttTGCCCATCCTGGTCGA |
| signal | | GCTGGACGGCGATGTGAACGGCCAC |
| (CoVEG8) | | AAGTTCAGCGTTTCTGGCGAGGGtg |
| | | agctttgggctaagcgcaacattaa |
| | | accagtaccagaggtgaaaatactc |
| | | aataatttgggtgtggacattgctg |
| | | ctaatactgtgatctgggactacaa |
| | | aagagatgctccagcacatatatct |
| | | actattggtgtttgttctatgactg |
| | | acatagccaagaaaccaactgaaac |
| | | gatttgtgcaccactcactgtcttt |
| | | tttgatggtagagttgatggtcaag |
| | | tagacttatttagaaatgcccgtaa |
| | | tggtgttcttattacagaaggtagt |
| | | gttaaaggtttacaaccatctgtag |
| | | gtcccaaacaagctagtcttaatgg |
| | | agtcacattaattggagaagccgta |
| | | aaaacacagttcaattattataaga |
| | | aagttgatggtgttgtccaacaatt |
| | | acctgaaacttactttactcagagt |
| | | agaaatttacaagaatttaaaccca |
| | | ggagtcaaatggaaattgatttctt |
| | | agaattagctatggatgaattcatt |
| | | gaacggtataaattagaaggctatg |
| | | ccttcgaacatatcgtttatggaga |
| | | ttttagtcata |
| Mutant S | 51 | MFVFLVLLPLVSSQCVNLTTRTQLP |
| protein | | PAYTNSFTRGVYYPDKVFRSSVLHS |
| | | TQDLFLPFFSNVTWFHAIHVSGTNG |
| | | TKRFDNPVLPFNDGVYFASTEKSNI |
| | | IRGWIFGTTLDSKTQSLLIVNNATN |
| | | VVIKVCBFQFCNDPFLGVYYHKNNK |
| | | SWMESEFRVYSSANNCTFEYVSQPF |
| | | LMDLEGKQGNFKNLREFVFKNIDGY |
| | | FKIYSKHTPINLVRDLPQGFSALEP |
| | | LVDLPIGINITRFQTLLALHRSYLT |
| | | PGDSSSGWTAGAAAYYVGYLQPRTF |
| | | LLKYNENGTITDAVDCALDPLSETK |
| | | CTLKSFTVEKGIYQTSNERVQPTES |
| | | IVRFPNITNLCPFGEVFNATRFASV |
| | | YAWNRKRISNCVADYSVLYNSASES |
| | | TFKCYGVSPTKLNDLCFTNVYADSF |
| | | VIRGDEVRQIAPGQTGKIADYNYKL |
| | | PDDFTGCVIAWNSNNLDSKVGGNYN |
| | | YLYRLFRKSNLKPFERDISTEIYQA |
| | | GSTPCNGVEGENCYFPLQSYGFQPT |
| | | NGVGYQPYRVVVLSFELLHAPATVC |
| | | GPKKSTNLVKNKCVNFNFNGLTGTG |
| | | VLTESNKKFLPFQQFGRDIADTTDA |
| | | VRDPQTLEILDITPCSFGGVSVIT |
| | | PGTNTSNQVAVLYQGVNCTEVPVAI |
| | | HADQLTPTWRVYSTGSNVFQTRAGC |
| | | LIGAEHVNNSYECDIPIGAGICASY |
| | | QTQTNSPGSASSVASQSIIAYTMSL |
| | | GAENSVAYSNNSIAIPTNFTISVTT |
| | | EILPVSMTKTSVDCTMYICGDSTEC |
| | | SNLLLQYGSFCTQLNRALTGIAVEQ |
| | | DKNTQEVFAQVKQIYKTPPIKDFGG |
| | | FNFSQILPDPSKPSKRSFIEDLLFN |
| | | KVTLADAGFIKQYGDCLGDIAARDL |
| | | ICAQKFNGLTVLPPLLTDEMIAQYT |
| | | SALLAGTITSGWTFGAGAALQIPFA |
| | | MQMAYRENGIGVTQNVLYENQKLIA |
| | | NQFNSAIGKIQDSLSSTASALGKLQ |
| | | DVVNQNAQALNTLVKQLSSNFGAIS |
| | | SVLNDILSRLDPPEAEVQIDRLITG |
| | | RLQSLQTYVTQQLIRAAEIRASANL |
| | | AATKMSECVLGQSKRVDFCGKGYHL |
| | | MSFPQSAPHGVVFLHVTYVPAQEKN |
| | | FTTAPAICHDGKAHFPREGVFVSNG |
| | | THWFVTQRNFYEPQUITTDNTFVSG |
| | | NCDVVIGIVNNTVYDPLQPELDSFK |
| | | EELDKYFKNHTSPDVDLGDISGINA |
| | | SVVNIQKEIDRINEVAKNLNESLID |
| | | LQELGKYEQYIKWPWYIWLGFIAGL |
| | | IAIVMVTIMLCCMTSCCSCLKGCCS |
| | | CGSCCKFDEDDSEPVLKGVKLHYT |
| Mutant S | 52 | ATGTTCGTGTTCCTGGTGCTGCTGC |
| gene | | CTCTGGTCAGCTCCCAGTGTGTGAA |
| (CoVEG9) | | CCTGACCACCAGAACCCAGCTGCCA |
| | | CCTGCTTATACAAACTCCTTCACTC |
| | | GGGGGGTATACTACCCCGACAAGGT |
| | | GTTCAGATCTAGCGTGCTGCATTCT |
| | | ACACAAGACCTGTTCCTGCCCTTCT |
| | | TCAGCAACGTGACCTGGTTCCACGC |
| | | CATCCACGTGTCTGGAACCAACGGA |
| | | ACCAAGAGATTCGACAACCCCGTGC |
| | | TGCCTTTCAACGACGGCGTGTACTT |
| | | CGCCAGCACCGAGAAGTCCAACATC |
| | | ATCAGAGGATGGATTTTCGGCACCA |
| | | CACTGGACAGCAAAACCCAGAGCCT |
| | | GCTGATCGTGAACAACGCCACCAAC |
| | | GTGGTGATCAAGGTGTGCGAGTTCC |
| | | AGTTCTGCAATGATCCCTTCCTGGG |
| | | CGTGTACTACCACAAGAACAACAAG |
| | | TCTTGGATGGAAAGCGAGTTCAGAG |
| | | TGTATTCCAGCGCCAACAATTGCAC |
| | | CTTCGAGTACGTGAGCCAACCCTTT |
| | | CTGATGGACCTTGAAGGCAAGCAGG |
| | | GCAACTTCAAAAATCTGCGAGAATT |
| | | TGTGTTCAAGAACATCGACGGATAC |
| | | TTCAAGATCTACTCTAAGCACACGC |
| | | CAATCAACCTGGTGAGAGATCTGCC |
| | | CCAGGGCTTTAGCGCTTTGGAACCT |
| | | CTGGTGGACCTGCCTATCGGAATCA |
| | | ACATCACCAGATTTCAAACTCTCCT |
| | | GGCCCTGCACAGATCTTATCTGACC |
| | | CCTGGGGACAGTAGTAGCGGCTGGA |
| | | CAGCCGGCGCCGCCGCCTACTACGT |
| | | GGGATACCTGCAGCCTAGAACATTC |
| | | CTGCTGAAGTACAATGAGAACGGAA |
| | | CAATCACAGACGCCGTGGACTGCGC |
| | | CCTGGATCCTTTGAGCGAGACAAAG |
| | | TGCACCCTGAAGTCGTTCACCGTCG |
| | | AAAAAGGCATCTACCAGACCAGCAA |
| | | CTTCCGCGTGCAGCCTACGGAATCT |
| | | ATCGTGCGGTTCCCCAACATCACCA |
| | | ACCTGTGCCCTTTCGGCGAGGTGTT |
| | | TAACGCTACAAGGTTCGCCAGCGTG |
| | | TATGCCTGGAACAGAAAGAGAATCA |
| | | GCAATTGCGTGGCCGATTATAGCGT |
| | | TCTGTACAACAGCGCTTCCTTCAGC |
| | | ACCTTCAAGTGCTACGGCGTGTCTC |
| | | CAACCAAGCTGAACGACCTCTGCTT |
| | | CACCAATGTCTACGCTGACTCTTTC |
| | | GTGATTAGAGGCGATGAGGTTAGAC |
| | | AGATCGCACCTGGCCAGACCGGCAA |
| | | AATCGCTGACTACAACTACAAGCTG |
| | | CCTGATGACTTCACAGGCTGTGTCA |
| | | TTGCCTGGAACTCAAATAACCTGGA |
| | | CTCTAAAGTGGGCGGCAACTACAAC |
| | | TACCTGTACCGGCTGTTCCGGAAGA |
| | | GCAATCTGAAACCTTTTGAGCGGGA |
| | | CATCTCTACAGAGATCTACCAGGCC |
| | | GGCAGCACACCCTGCAACGGCGTTG |
| | | AGGGCTTCAACTGCTACTTCCCTCT |
| | | GCAGAGCTACGGCTTTCAGCCAACA |
| | | AATGGAGTGGGCTACCAGCCGTACA |
| | | GAGTGGTGGTGCTGAGCTTCGAACT |
| | | GCTGCATGCCCCAGCCACAGTGTGT |
| | | GGACCTAAGAAGTCTACCAACCTGG |
| | | TGAAGAACAAGTGCGTGAACTTTAA |
| | | CTTTAACGGCCTGACCGGCACAGGC |
| | | GTGCTGACCGAATCCAACAAAAAGT |
| | | TCCTGCCCTTCCAACAGTTCGGCAG |
| | | AGACATCGCCGATACAACCGATGCC |
| | | GTGCGGGACCCCCAGACCTTAGAAA |
| | | TCCTAGATATCACCCCGTGCAGCTT |
| | | CGGCGGAGTCTCTGTTATTACTCCT |
| | | GGCACCAACACCAGCAACCAAGTGG |
| | | CTGTTCTGTACCAAggcGTGAACTG |
| | | CACCGAAGTGCCTGTGGCTATCCAC |
| | | GCCGATCAGCTGACCCCAACCTGGC |
| | | GGGTGTATAGCACCGGCTCTAACGT |
| | | GTTCCAGACCCGGGCTGGCTGCCTG |
| | | ATCGGCGCCGAACACGTCAACAACT |
| | | CCTATGAATGTGACATCCCCATCGG |
| | | GGCTGGCATCTGCGCCAGTTACCAG |
| | | ACACAGACAAATAGCCCTGGCAGCG |
| | | CCAGCAGCGTGGCCTCCCAGAGTAT |
| | | CATTGCCTACACCATGAGCCTGGGC |
| | | GCCGAGAACAGCGTGGCCTATTCTA |
| | | ACAATAGCATCGCAATCCCTACCAA |
| | | CTTTACCATCTCTGTGACAACCGAG |
| | | ATCCTGCCTGTGAGCATGACCAAAA |
| | | CCAGCGTGGACTGCACGATGTACAT |
| | | CTGTGGCGACAGCACAGAATGCAGT |
| | | AATCTGTTGCTGCAGTACGGCAGCT |
| | | TTTGCACCCAGTTGAATAGAGCCCT |
| | | GACCGGAATCGCCGTAGAGCAGGAC |
| | | AAAAATACCCAGGAGGTGTTCGCCC |
| | | AGGTGAAACAGATCTACAAGACACC |
| | | TCCCATTAAGGACTTCGGAGGTTTT |
| | | AACTTCAGCCAGATCCTGCCCGACC |
| | | CTTCCAAGCCTAGCAAACGCTCCTT |
| | | CATCGAGGACCTGCTCTTCAACAAG |
| | | GTGACACTGGCTGATGCCGGCTTCA |
| | | TCAAGCAGTACGGAGATTGTCTGGG |
| | | AGACATCGCCGCTAGAGATCTGATC |
| | | TGCGCCCAAAAGTTCAACGGCCTGA |
| | | CCGTGCTGCCTCCTCTGCTTACAGA |
| | | CGAGATGATCGCCCAGTACACCAGC |
| | | GCCCTGCTGGCTGGCACCATCACAA |
| | | GCGGCTGGACCTTCGGAGCCGGAGC |
| | | CGCTCTGCAAATCCCCTTTGCCATG |
| | | CAGATGGCCTACCGGTTCAACGGCA |
| | | TCGGCGTGACACAGAATGTGCTGTA |
| | | CGAGAACCAGAAGCTGATCGCTAAC |
| | | CAGTTTAACAGCGCTATCGGCAAGA |
| | | TCCAGGACTCGCTGAGTAGCACCGC |
| | | CTCTGCCCTGGGCAAGCTGCAGGAC |
| | | GTCGTGAACCAGAACGCCCAAGCCC |
| | | TGAACACACTGGTGAAACAGCTGAG |
| | | CAGCAACTTCGGCGCCATCAGCTCT |
| | | GTGCTGAACGATATCCTGAGCAGAC |
| | | TGGACCCTcccGAAGCCGAGGTCCA |
| | | GATCGACAGACTGATCACAGGAAGA |
| | | CTGCAGAGCCTGCAAACGTACGTGA |
| | | CACAGCAGCTGATCCGGGCAGCCGA |
| | | AATCCGGGCCAGCGCCAATCTGGCC |
| | | GCTACCAAGATGAGCGAGTGCGTGT |
| | | TAGGCCAGAGCAAGCGGGTGGATTT |
| | | CTGCGGTAAGGGATACCACCTGATG |
| | | AGCTTTCCCCAGAGCGCTCCTCACG |
| | | GCGTGGTGTTTCTGCACGTGACCTA |
| | | CGTTCCTGCCCAGGAAAAGAACTTC |
| | | ACCACCGCCCCTGCTATCTGCCACG |
| | | ATGGCAAGGCCCACTTCCCTAGAGA |
| | | GGGCGTTTTCGTGTCTAACGGCACA |
| | | CACTGGTTTGTGACCCAGAGAAACT |
| | | TCTACGAGCCTCAGATCATCACCAC |
| | | AGACAACACCTTTGTGAGCGGCAAT |
| | | TGCGACGTGGTGATCGGAATTGTTA |
| | | ATAATACCGTGTACGACCCTCTGCA |
| | | GCCTGAGCTCGACAGCTTCAAGGAA |
| | | GAGCTGGACAAGTACTTCAAGAACC |
| | | ACACCTCCCCAGATGTGGACCTGGG |
| | | CGATATTTCAGGCATCAACGCCTCC |
| | | GTCGTGAATATCCAGAAGGAGATCG |
| | | ACCGGCTCAACGAGGTGGCCAAGAA |
| | | CCTTAACGAGAGCCTGATCGACCTG |
| | | CAGGAACTGGGCAAATATGAGCAGT |
| | | ACATCAAGTGGCCTTGGTACATCTG |
| | | GCTGGGCTTTATCGCAGGCCTGATC |
| | | GCTATCGTGATGGTGACCATTATGC |
| | | TGTGTTGTATGACCAGCTGTTGTAG |
| | | TTGTCTGAAGGGCTGCTGTTCTTGC |
| | | GGCAGCTGCTGCAAGTTCGACGAAG |
| | | ACGACTCAGAGCCCGTGCTGAAAGG |
| | | CGTGAAGCTGCACTACACC |
| ORF3 gene | 54 | ATGGACCTGTTCATGAGAATCTTCA |
| (CoVEG14) | | CCATCGGCACCGTGACACTGAAGCA |
| | | GGGCGAGATCAAGGATGCCACCCCT |
| | | AGCGACTTCGTGAGAGCCACCGCCA |
| | | CAATTCCTATCCAGGCTAGCCTGCC |
| | | TTTTGGATGGCTGATCGTGGGCGTC |
| | | GCCCTGCTCGCCGTGTTCCAGAGCG |
| | | CCTCTAAGATCATTACACTGAAGAA |
| | | AAGATGGCAGCTGGCCCTCTCCAAA |
| | | GGCGTGCACTTCGTGTGTAATCTGC |
| | | TGCTGCTTTTTGTGACAGTGTACAG |
| | | CCACCTGCTGCTGGTTGCTGCTGGC |
| | | CTGGAAGCCCCTTTCCTGTACCTGT |
| | | ACGCCCTGGTCTACTTCCTGCAGTC |
| | | TATCAACTTCGTGCGGATCATCATG |
| | | CGGCTGTGGCTGTGCTGGAAGTGCA |
| | | GAAGCAAGAACCCACTGCTGTACGA |
| | | CGCCAATTACTTCCTGTGTTGGCAC |
| | | ACCAACTGCTACGACTACTGCATCC |
| | | CCTACAACAGCGTGACCAGCAGCAT |
| | | CGTGATCACCTCTGGCGACGGAACA |
| | | ACCAGCCCTATCAGCGAGCATGATT |
| | | ACCAGATCGGCGGATATACAGAGAA |
| | | GTGGGAGAGCGGCGTGAAGGACTGC |
| | | GTGGTGCTGCACAGCTACTTTACCT |
| | | CCGATTACTACCAACTGTATTCTAC |
| | | CCAGCTGAGCACCGACACCGGCGTG |
| | | GAACACGTGACCTTCTTCATCTACA |
| | | ACAAGATCGTGGACGAGCCTGAGGA |
| | | ACACGTGCAGATCCACACTATCGAC |
| | | GGCAGCTCTGGCGTTGTGAACCCTG |
| | | TGATGGAACCCATCTACGATGAGCC |
| | | CACCACAACAACCTCCGTGCCCCTG |
| | | Taa |
| Control S | 65 | ATGTTCGTGTTCCTGGTGCTGCTGC |
| only | | CTCTGGTCAGCTCCCAGTGTGTGAA |
| plasmid | | CCTGACCACCAGAACCCAGCTGCCA |
| expression | | CCTGCTTATACAAACTCCTTCACTC |
| cassette | | GGGGGGTATACTACCCCGACAAGGT |
| | | GTTCAGATCTAGCGTGCTGCATTCT |
| | | ACACAAGACCTGTTCCTGCCCTTCT |
| | | TCAGCAACGTGACCTGGTTCCACGC |
| | | CATCCACGTGTCTGGAACCAACGGA |
| | | ACCAAGAGATTCGACAACCCCGTGC |
| | | TGCCTTTCAACGACGGCGTGTACTT |
| | | CGCCAGCACCGAGAAGTCCAACATC |
| | | ATCAGAGGATGGATTTTCGGCACCA |
| | | CACTGGACAGCAAAACCCAGAGCCT |
| | | GCTGATCGTGAACAACGCCACCAAC |
| | | GTGGTGATCAAGGTGTGCGAGTTCC |
| | | AGTTCTGCAATGATCCCTTCCTGGG |
| | | CGTGTACTACCACAAGAACAACAAG |
| | | TCTTGGATGGAAAGCGAGTTCAGAG |
| | | TGTATTCCAGCGCCAACAATTGCAC |
| | | CTTCGAGTACGTGAGCCAACCCTTT |
| | | CTGATGGACCTTGAAGGCAAGCAGG |
| | | GCAACTTCAAAAATCTGCGAGAATT |
| | | TGTGTTCAAGAACATCGACGGATAC |
| | | TTCAAGATCTACTCTAAGCACACGC |
| | | CAATCAACCTGGTGAGAGATCTGCC |
| | | CCAGGGCTTTAGCGCTTTGGAACCT |
| | | CTGGTGGACCTGCCTATCGGAATCA |
| | | ACATCACCAGATTTCAAACTCTCCT |
| | | GGCCCTGCACAGATCTTATCTGACC |
| | | CCTGGGGACAGTAGTAGCGGCTGGA |
| | | CAGCCGGCGCCGCCGCCTACTACGT |
| | | GGGATACCTGCAGCCTAGAACATTC |
| | | CTGCTGAAGTACAATGAGAACGGAA |
| | | CAATCACAGACGCCGTGGACTGCGC |
| | | CCTGGATCCTTTGAGCGAGACAAAG |
| | | TGCACCCTGAAGTCGTTCACCGTCG |
| | | AAAAAGGCATCTACCAGACCAGCAA |
| | | CTTCCGCGTGCAGCCTACGGAATCT |
| | | ATCGTGCGGTTCCCCAACATCACCA |
| | | ACCTGTGCCCTTTCGGCGAGGTGTT |
| | | TAACGCTACAAGGTTCGCCAGCGTG |
| | | TATGCCTGGAACAGAAAGAGAATCA |
| | | GCAATTGCGTGGCCGATTATAGCGT |
| | | TCTGTACAACAGCGCTTCCTTCAGC |
| | | ACCTTCAAGTGCTACGGCGTGTCTC |
| | | CAACCAAGCTGAACGACCTCTGCTT |
| | | CACCAATGTCTACGCTGACTCTTTC |
| | | GTGATTAGAGGCGATGAGGTTAGAC |
| | | AGATCGCACCTGGCCAGACCGGCAA |
| | | AATCGCTGACTACAACTACAAGCTG |
| | | CCTGATGACTTCACAGGCTGTGTCA |
| | | TTGCCTGGAACTCAAATAACCTGGA |
| | | CTCTAAAGTGGGCGGCAACTACAAC |
| | | TACCTGTACCGGCTGTTCCGGAAGA |
| | | GCAATCTGAAACCTTTTGAGCGGGA |
| | | CATCTCTACAGAGATCTACCAGGCC |
| | | GGCAGCACACCCTGCAACGGCGTTG |
| | | AGGGCTTCAACTGCTACTTCCCTCT |
| | | GCAGAGCTACGGCTTTCAGCCAACA |
| | | AATGGAGTGGGCTACCAGCCGTACA |
| | | GAGTGGTGGTGCTGAGCTTCGAACT |
| | | GCTGCATGCCCCAGCCACAGTGTGT |
| | | GGACCTAAGAAGTCTACCAACCTGG |
| | | TGAAGAACAAGTGCGTGAACTTTAA |
| | | CTTTAACGGCCTGACCGGCACAGGC |
| | | GTGCTGACCGAATCCAACAAAAAGT |
| | | TCCTGCCCTTCCAACAGTTCGGCAG |
| | | AGACATCGCCGATACAACCGATGCC |
| | | GTGCGGGACCCCCAGACCTTAGAAA |
| | | TCCTAGATATCACCCCGTGCAGCTT |
| | | CGGCGGAGTCTCTGTTATTACTCCT |
| | | GGCACCAACACCAGCAACCAAGTGG |
| | | CTGTTCTGTACCAAggcGTGAACTG |
| | | CACCGAAGTGCCTGTGGCTATCCAC |
| | | GCCGATCAGCTGACCCCAACCTGGC |
| | | GGGTGTATAGCACCGGCTCTAACGT |
| | | GTTCCAGACCCGGGCTGGCTGCCTG |
| | | ATCGGCGCCGAACACGTCAACAACT |
| | | CCTATGAATGTGACATCCCCATCGG |
| | | GGCTGGCATCTGCGCCAGTTACCAG |
| | | ACACAGACAAATAGCCCTGGCAGCG |
| | | CCAGCAGCGTGGCCTCCCAGAGTAT |
| | | CATTGCCTACACCATGAGCCTGGGC |
| | | GCCGAGAACAGCGTGGCCTATTCTA |
| | | ACAATAGCATCGCAATCCCTACCAA |
| | | CTTTACCATCTCTGTGACAACCGAG |
| | | ATCCTGCCTGTGAGCATGACCAAAA |
| | | CCAGCGTGGACTGCACGATGTACAT |
| | | CTGTGGCGACAGCACAGAATGCAGT |
| | | AATCTGTTGCTGCAGTACGGCAGCT |
| | | TTTGCACCCAGTTGAATAGAGCCCT |
| | | GACCGGAATCGCCGTAGAGCAGGAC |
| | | AAAAATACCCAGGAGGTGTTCGCCC |
| | | AGGTGAAACAGATCTACAAGACACC |
| | | TCCCATTAAGGACTTCGGAGGTTTT |
| | | AACTTCAGCCAGATCCTGCCCGACC |
| | | CTTCCAAGCCTAGCAAACGCTCCTT |
| | | CATCGAGGACCTGCTCTTCAACAAG |
| | | GTGACACTGGCTGATGCCGGCTTCA |
| | | TCAAGCAGTACGGAGATTGTCTGGG |
| | | AGACATCGCCGCTAGAGATCTGATC |
| | | TGCGCCCAAAAGTTCAACGGCCTGA |
| | | CCGTGCTGCCTCCTCTGCTTACAGA |
| | | CGAGATGATCGCCCAGTACACCAGC |
| | | GCCCTGCTGGCTGGCACCATCACAA |
| | | GCGGCTGGACCTTCGGAGCCGGAGC |
| | | CGCTCTGCAAATCCCCTTTGCCATG |
| | | CAGATGGCCTACCGGTTCAACGGCA |
| | | TCGGCGTGACACAGAATGTGCTGTA |
| | | CGAGAACCAGAAGCTGATCGCTAAC |
| | | CAGTTTAACAGCGCTATCGGCAAGA |
| | | TCCAGGACTCGCTGAGTAGCACCGC |
| | | CTCTGCCCTGGGCAAGCTGCAGGAC |
| | | GTCGTGAACCAGAACGCCCAAGCCC |
| | | TGAACACACTGGTGAAACAGCTGAG |
| | | CAGCAACTTCGGCGCCATCAGCTCT |
| | | GTGCTGAACGATATCCTGAGCAGAC |
| | | TGGACCCTcccGAAGCCGAGGTCCA |
| | | GATCGACAGACTGATCACAGGAAGA |
| | | CTGCAGAGCCTGCAAACGTACGTGA |
| | | CACAGCAGCTGATCCGGGCAGCCGA |
| | | AATCCGGGCCAGCGCCAATCTGGCC |
| | | GCTACCAAGATGAGCGAGTGCGTGT |
| | | TAGGCCAGAGCAAGCGGGTGGATTT |
| | | CTGCGGTAAGGGATACCACCTGATG |
| | | AGCTTTCCCCAGAGCGCTCCTCACG |
| | | GCGTGGTGTTTCTGCACGTGACCTA |
| | | CGTTCCTGCCCAGGAAAAGAACTTC |
| | | ACCACCGCCCCTGCTATCTGCCACG |
| | | ATGGCAAGGCCCACTTCCCTAGAGA |
| | | GGGCGTTTTCGTGTCTAACGGCACA |
| | | CACTGGTTTGTGACCCAGAGAAACT |
| | | TCTACGAGCCTCAGATCATCACCAC |
| | | AGACAACACCTTTGTGAGCGGCAAT |
| | | TGCGACGTGGTGATCGGAATTGTTA |
| | | ATAATACCGTGTACGACCCTCTGCA |
| | | GCCTGAGCTCGACAGCTTCAAGGAA |
| | | GAGCTGGACAAGTACTTCAAGAACC |
| | | ACACCTCCCCAGATGTGGACCTGGG |
| | | CGATATTTCAGGCATCAACGCCTCC |
| | | GTCGTGAATATCCAGAAGGAGATCG |
| | | ACCGGCTCAACGAGGTGGCCAAGAA |
| | | CCTTAACGAGAGCCTGATCGACCTG |
| | | CAGGAACTGGGCAAATATGAGCAGT |
| | | ACATCAAGTGGCCTTGGTACATCTG |
| | | GCTGGGCTTTATCGCAGGCCTGATC |
| | | GCTATCGTGATGGTGACCATTATGC |
| | | TGTGTTGTATGACCAGCTGTTGTAG |
| | | TTGTCTGAAGGGCTGCTGTTCTTGC |
| | | GGCAGCTGCTGCAAGTTCGACGAAG |
| | | ACGACTCAGAGCCCGTGCTGAAAGG |
| | | CGTGAAGCTGCACTACACCTAAtcc |
| | | cccccccctaacgttactggccgaa |
| | | gccgcttggaataaggccggtgtgc |
| | | gtttgtctatatgttattttccacc |
| | | atattgccgtcttttggcaatgtga |
| | | gggcccggaaacctggccctgtctt |
| | | cttgacgagcattcctaggggtett |
| | | tcccctetegccaaaggaatgcaag |
| | | gtctgttgaatgtcgtgaaggaagc |
| | | agttcctctggaagcttcttgaaga |
| | | caaacaacgtctgtagcgacccttt |
| | | gcaggcageggaaccccccacctgg |
| | | cgacaggtgcctctgcggccaaaag |
| | | ccacgtgtataagatacacctgcaa |
| | | aggcggcacaaccccagtgccacgt |
| | | tgtgagttggatagttgtggaaaga |
| | | gtcaaatggctctcctcaagcgtat |
| | | tcaacaaggggctgaaggatgccca |
| | | gaaggtaccccattgtatgggatct |
| | | gatctggggcctcggtgcacatgct |
| | | ttacatgtgtttagtcgaggttaaa |
| | | aaaacgtctaggccccccgaaccac |
| | | ggggacgtggttttcctttgaaaaa |
| | | cacgatgataatatggccacaacca |
| | | tggaacaagagacttgcgcgcactc |
| | | tctcacttttgaggaatgcccaaaa |
| | | tgctctgctctacaataccgtaatg |
| | | gattttacctgctaaagtatgatga |
| | | agaatggtacccagaggagttattg |
| | | actgatggagaggatgatgtctttg |
| | | atcccgaattagacatggaagtcgt |
| | | tttcgagttacagtaa |

In some embodiments, the vector disclosed herein comprises the polynucleotide from any one of CoVEG 1-17 that encodes the SARS CoV2 Spike protein. In some embodiments, the vector disclosed herein comprises the polynucleotide from any one of CoVEG 1-17 that encodes the SARS CoV2 membrane protein. In some embodiments, the vector disclosed herein comprises the polynucleotide from any one of CoVEG 1-17 that encodes the SARS CoV2 envelope protein. In some embodiments, the vector disclosed herein comprises the polynucleotide from any one of CoVEG 1-17 that encodes the SARS CoV2 nucleocapsid protein. In some embodiments, the vector disclosed herein comprises the polynucleotide from any one of CoVEG 1-17 that encodes the EMCV L protein. In some embodiments, the vector disclosed herein comprises the polynucleotide from any one of CoVEG 1-17 that encodes the internal ribosome entry site (IRES). In some embodiments, the vector disclosed herein comprises the polynucleotide from any one of CoVEG 1-17 that encodes the viral packaging signal.

### ii) Polynucleotides

Polynucleotides of the present disclosure may include DNA, RNA, and DNA-RNA hybrid molecules. In some embodiments, polynucleotides are isolated from a natural source; prepared in vitro, using techniques e.g., PCR amplification or chemical synthesis; prepared in vivo, e.g., via recombinant DNA technology; or prepared or obtained by any appropriate method. In some embodiments, polynucleotides are of any shape (linear, circular, etc.) or topology (single-stranded, double-stranded, linear, circular, supercoiled, torsional, nicked, etc.). Polynucleotides may also comprise nucleic acid derivatives e.g., peptide nucleic acids (PNAS) and polypeptide-nucleic acid conjugates; nucleic acids having at least one chemically modified sugar residue, backbone, internucleotide linkage, base, nucleotide, nucleoside, or nucleotide analog or derivative; as well as nucleic acids having chemically modified 5′ or 3′ ends; and nucleic acids having two or more of such modifications. Not all linkages in a polynucleotide need to be identical.

A polynucleotide is said to “encode” a protein when it comprises a nucleic acid sequence that is capable of being transcribed and translated (e.g., DNA→RNA→protein) or translated (RNA→protein) in order to produce an amino acid sequence corresponding to the amino acid sequence of said protein. In vivo (e.g., within a eukaryotic cell) transcription and/or translation is performed by endogenous or exogenous enzymes. In some embodiments, transcription of the polynucleotides of the disclosure is performed by the endogenous polymerase II (polII) of the eukaryotic cell. In some embodiments, an exogenous RNA polymerase is provided on the same or a different vector. In some embodiments, viral polymerases may alternatively or additionally be used. In some embodiments, a viral promoter is used in combination with one or more viral polymerase. In some embodiments, the RNA polymerase is selected from a T3 RNA polymerase, a T5 RNA polymerase, a T7 RNA polymerase, an H8 RNA polymerase, EMCV RNA polymerase, HIV RNA polymerase, Influenza RNA polymerase, SP6 RNA polymerase, CMV RNA polymerase, T3 RNA polymerase, T1 RNA polymerase, SPO1 RNA polymerase, SP2 RNA polymerase, Phil5 RNA polymerase, and the like. Viral polymerases are RNA priming or capping polymerases. In some embodiments, IRES elements are used in conjunction with viral polymerases.

The polynucleotides disclosed herein may encode one or more antigens; and/or one or more enhancer proteins. In some embodiments, the polynucleotide encodes one antigen. In some embodiments, the polynucleotide encodes one enhancer protein. In some embodiments, the polynucleotide encodes more than one antigen; more than one enhancer protein, and/or one or more separating elements.

In some embodiments, the polynucleotide may encode a polypeptide that is not antigenic. In some embodiments, the polypeptide that is not antigenic may form a part of a VLP. Thus, the present disclosure provides vectors that comprise polynucleotides that encode one or more antigens, and/or polynucleotides that encode one or more non-antigenic polypeptides, and/or polynucleotides that encode one or more enhancer proteins. In some embodiments, the one or more antigens and the one or more non-antigenic polypeptides are capable of forming a virus like particle (VLP). In some embodiments, the one or more antigens may be derived from one or more proteins of a first virus, and the one or more non-antigenic polypeptides may be derived from one or more proteins of a second virus.

### iii) Separating Elements

In some embodiments, antigen(s) and enhancer protein(s) according to the present disclosure are encoded on the same vector. In some embodiments, antigen(s) and enhancer protein(s) according to the present disclosure are encoded on separate vectors. In some embodiments, if nucleic acid sequences encoding one or more antigens and one or more enhancer proteins are present in the same vector, the vector may comprise a separating element for separate expression of the proteins. In some embodiments, the vector is a bicistronic vector or a polycistronic vector. The separating element may be an internal ribosomal entry site (IRES) or 2A element. In some embodiments, a vector may comprise a nucleic acid encoding a 2A element, or a nucleic acid encoding an IRES.

In some embodiments, the first polynucleotide or the second polynucleotide, or both, are operatively linked to a polynucleotide encoding a 2A element. In some embodiments, the polynucleotide encoding the enhancer protein and/or the polynucleotide encoding the antigen are operatively linked to a polynucleotide encoding an a 2A element. Non-limiting examples of 2A elements include P2A, E2A, F2A, and T2A. In some embodiments, the amino acid sequence of the 2A peptide has at least 80% sequence identity (for instance, at least 85%, at least 90%, at least 95%, at least 96%, at least 97%, at least 98%, at least 99%, at least 99.5%, including any values and subranges that lie there between) to SEQ ID NO: 17. In some embodiments, the amino acid sequence of the 2A peptide is SEQ ID NO: 17.

In some embodiments, the nucleic acid sequence encoding the 2A peptide has at least 80% sequence identity (for instance, at least 85%, at least 90%, at least 95%, at least 96%, at least 97%, at least 98%, at least 99%, at least 99.5%, including any values and subranges that lie there between) to SEQ ID NO: 18 or 69. In some embodiments, the nucleic acid sequence encoding the 2A peptide is SEQ ID NO: 18 or 69.

In some embodiments, the first polynucleotide or the second polynucleotide, or both, are operatively linked to a polynucleotide encoding an internal ribosome entry site (IRES). In some embodiments, the polynucleotide encoding the enhancer protein and/or the polynucleotide encoding the antigen are operatively linked to a polynucleotide encoding an IRES. In some embodiments, the polynucleotide encoding the IRES has a nucleic acid sequence with at least 80% sequence identity (for instance, at least 85%, at least 90%, at least 95%, at least 96%, at least 97%, at least 98%, at least 99%, at least 99.5%, including any values and subranges that lie there between) to the nucleic acid sequence of SEQ ID NO: 24 or 67. In some embodiments, the polynucleotide encoding the IRES has a nucleic acid sequence of SEQ ID NO: 24 or 67.

In some embodiments, the antigen, and the enhancer protein are comprised in a single fusion protein. In some embodiments, the fusion protein may comprise a linking element. In some embodiments, the linking element may comprise a cleavage site (e.g. a furin, a cathepsin or an intein cleavage site) for enzymatic cleavage in cis or in trans. In other embodiments, the fusion protein or the linking element does not comprise a cleavage site and the expressed fusion protein comprises both the target protein and the enhancer protein. In some embodiments, the linking element is a 2A element.

### iv) Promoters

Vectors according to the present disclosure may comprise one or more promoters. The term “promoter” refers to a region or sequence located upstream or downstream from the start of transcription which is involved in recognition and binding of RNA polymerase and other proteins to initiate transcription. The polynucleotide(s) or vector(s) according to the present disclosure may comprise one or more promoters. The promoters may be any promoter known in the art. The promoter may be a forward promoter or a reverse promoter. In some embodiments, the promoter is a mammalian promoter. In some embodiments, one or more promoters are native promoters. In some embodiments, one or more promoters are non-native promoters. In some embodiments, one or more promoters are non-mammalian promoters. Non-limiting examples of RNA promoters for use in the disclosed compositions and methods include U1, human elongation factor-1 alpha (EF-1 alpha), cytomegalovirus (CMV), human ubiquitin, spleen focus-forming virus (SFFV), U6, H1, tRNA^{LyS}, tRNA^{Ser} and tRNA^{Arg}, CAG, PGK, TRE, UAS, UbC, SV40, T7, Sp6, lac, araBad, trp, and Ptac promoters.

The term “operatively linked” as used herein refers to elements or structures in a nucleic acid sequence that are linked by operative ability and not physical location. The elements or structures are capable of, or characterized by, accomplishing a desired operation. It is recognized by one of ordinary skill in the art that it is not necessary for elements or structures in a nucleic acid sequence to be in a tandem or adjacent order to be operatively linked.

In some embodiments, a promoter comprised by a vector according to the present disclosure is an inducible promoter.

In some embodiments, vectors according to the present disclosure may further comprise a polynucleotide sequence encoding a polymerase. In some embodiments, the polymerase is a viral polymerase. In some embodiments, the vectors disclosed herein comprises a polynucleotide sequence encoding a T7 RNA polymerase. In some embodiments, for example, a vector may comprise a T7 promoter configured for transcription of either or both of the polynucleotide encoding an antigen, and the second polynucleotide encoding the enhancer protein by a T7 RNA polymerase.

Antigens

In some embodiments, the expression or quality of the antigen is significantly improved by expression according to the disclosed methods, e.g., in conjunction with one or more enhancer proteins. In some embodiments, the antigen is derived from a single protein. In some embodiments, the antigen is derived from multiple proteins. In some embodiments, the antigen is a chimeric antigen comprising amino acid sequences from one or more proteins.

In some embodiments, the antigen is a viral antigen. The viral antigen may comprise the whole or part of an amino acid sequence derived from any viral protein, without limitation. In some embodiments, the viral antigen is the viral protein. In some embodiments, the amino acid sequence of the viral protein is the whole or part of a structural protein or multiple structural proteins of a virus. In some embodiments, the antigen or antigens assemble into VLPs and are released from the expressing cells.

In some embodiments, the viral antigen comprises the whole or an antigen fragment of any coronavirus protein, without limitation. In some embodiments, the coronavirus is a betacoronavirus. In some embodiments, the betacoronavirus is severe acute respiratory syndrome (SARS) virus. In some embodiments, the betacoronavirus is Middle East respiratory syndrome (MERS) virus, OC43, or HKU1. In some embodiments, the SARS virus is SARS-CoV-1. In some embodiments, the SARS virus is SARS-CoV-2.

In some embodiments, the viral antigen comprises the whole or an antigen fragment of any one or more of the following proteins: coronavirus spike protein, coronavirus M protein, coronavirus N protein, and coronavirus E protein.

In some embodiments, the coronavirus spike protein is selected from the group consisting of a SARS-Cov-2 spike protein, a Middle East respiratory syndrome (MERS) spike protein, and SARS-CoV spike protein. In some embodiments, the coronavirus M protein is selected from SARS-Cov-2 M protein, MERS M protein and SARS-CoV M protein. In some embodiments, the coronavirus N protein is selected from SARS-Cov-2 N protein, MERS N protein, and SARS-CoV N protein. In some embodiments, the coronavirus E protein is selected from SARS-Cov-2 E protein, MERS E protein, and SARS-CoV E protein.

In some embodiments, the polynucleotide encoding the coronavirus spike protein has a nucleic acid sequence with at least 70% sequence identity—for instance, at least 75%, at least 80%, at least 85%, at least 90%, at least 95%, at least 96%, at least 97%, at least 98%, at least 99%, at least 99.5%, including any values and subranges that lie there between—to the nucleic acid sequence of SEQ ID NO: 14 or 70. In some embodiments, the polynucleotide encoding the coronavirus spike protein has a nucleic acid sequence of SEQ ID NO: 14 or 70.

In some embodiments, the amino acid sequence of the coronavirus spike protein has at least 70% sequence identity—for instance, at least 75%, at least 80%, at least 85%, at least 90%, at least 95%, at least 96%, at least 97%, at least 98%, at least 99%, at least 99.5%, including any values and subranges that lie there between—to the amino acid sequence of SEQ ID NO: 13. In some embodiments, the amino acid sequence of the coronavirus spike protein is SEQ ID NO: 13.

In some embodiments, the SARS-Cov-2 spike protein is a mutant S protein (also denoted as “S (Mut)”) that comprises one or more amino acid mutations, as compared to SEQ ID NO: 13. In some embodiments, the mutant S protein is expressed at a higher level, as compared to the wild type S protein. In some embodiments, the mutant S protein is prefusion conformation-stabilized spike protein. In some embodiments, the mutation in the S protein stabilizes the trimeric state of the S protein. In some embodiments, the mutant S protein comprises one or more mutations in the internal endogenous proteolytic cleavage site of the S protein. In some embodiments, the mutant S protein comprises a deletion of the internal endogenous proteolytic cleavage site of the S protein. In some embodiments, the one or more mutations in the proteolytic cleavage site of the S protein inhibit the cleavage of the S protein during the assembly process. In some embodiments, a VLP comprising any one or more of the mutant S proteins disclosed herein is more immunogenic than a VLP comprising a wild type S protein, e.g., an S protein comprising an amino acid sequence of SEQ ID NO: 13.

In some embodiments, the mutant S protein comprises a modification (e.g. a substitution) of at least one amino acid residue selected from the group consisting of R682, R683, A684, R685, K986, and V987 in SEQ ID NO: 13. In some embodiments, the mutant S protein comprises at least one amino acid substitution selected from the group consisting of R682G, R683S, R685S, K986P, and V987P in SEQ ID NO: 13. In some embodiments, the mutation S protein comprises the amino acid substitutions, R682G, R683S, R685S, K986P, and V987P in SEQ ID NO: 13. In some embodiments, the mutant S protein comprises the following amino acid substitutions in an internal endogenous furin cleavage site: R682G, R683S, R685S. That is, in some embodiments, the mutant S protein comprises the following amino acids at an internal endogenous furin cleavage site: G at amino acid residue 682, S at amino acid residue 683, A at amino acid residue 684, and S at amino acid residue 685.

In some embodiments, the mutant S protein has at least 70% sequence identity—for instance, at least 75%, at least 80%, at least 85%, at least 90%, at least 95%, at least 96%, at least 97%, at least 98%, at least 99%, at least 99.5%, including any values and subranges that lie there between—to the amino acid sequence of SEQ ID NO: 51. In some embodiments, the amino acid sequence of the mutant S protein is SEQ ID NO: 51.

In some embodiments, the polynucleotide encoding the mutant S protein has a nucleic acid sequence with at least 70% sequence identity—for instance, at least 75%, at least 80%, at least 85%, at least 90%, at least 95%, at least 96%, at least 97%, at least 98%, at least 99%, at least 99.5%, including any values and subranges that lie there between—to the nucleic acid sequence of SEQ ID NO: 52. In some embodiments, the polynucleotide encoding the coronavirus spike protein has a nucleic acid sequence of SEQ ID NO: 52.

In some embodiments, the polynucleotide encoding the coronavirus M protein has a nucleic acid sequence with at least 80% sequence identity—for instance, at least 85%, at least 90%, at least 95%, at least 96%, at least 97%, at least 98%, at least 99%, at least 99.5%, including any values and subranges that lie there between—to the nucleic acid sequence of SEQ ID NO: 19 or 66. In some embodiments, the polynucleotide encoding the coronavirus M protein has a nucleic acid sequence of SEQ ID NO: 19 or 66.

In some embodiments, the amino acid sequence of the coronavirus M protein has at least 80% sequence identity—for instance, at least 85%, at least 90%, at least 95%, at least 96%, at least 97%, at least 98%, at least 99%, at least 99.5%, including any values and subranges that lie there between—to the amino acid sequence of SEQ ID NO: 33. In some embodiments, the amino acid sequence of the coronavirus M protein is SEQ ID NO: 33.

In some embodiments, the polynucleotide encoding the coronavirus N protein has a nucleic acid sequence with at least 80% sequence identity—for instance, at least 85%, at least 90%, at least 95%, at least 96%, at least 97%, at least 98%, at least 99%, at least 99.5%, including any values and subranges that lie there between—to the nucleic acid sequence of SEQ ID NO: 21 or 71. In some embodiments, the polynucleotide encoding the coronavirus N protein has a nucleic acid sequence of SEQ ID NO: 21 or 71.

In some embodiments, the amino acid sequence of the coronavirus N protein has at least 80% sequence identity—for instance, at least 85%, at least 90%, at least 95%, at least 96%, at least 97%, at least 98%, at least 99%, at least 99.5%, including any values and subranges that lie there between—to the amino acid sequence of SEQ ID NO: 20. In some embodiments, the amino acid sequence of the coronavirus N protein is SEQ ID NO: 20.

In some embodiments, the polynucleotide encoding the coronavirus E protein has a nucleic acid sequence with at least 80% sequence identity—for instance, at least 85%, at least 90%, at least 95%, at least 96%, at least 97%, at least 98%, at least 99%, at least 99.5%, including any values and subranges that lie there between—to the nucleic acid sequence of SEQ ID NO: 23 or 72. In some embodiments, the polynucleotide encoding the coronavirus E protein has a nucleic acid sequence of SEQ ID NO: 23 or 72.

In some embodiments, the amino acid sequence of the coronavirus E protein has at least 80% sequence identity—for instance, at least 85%, at least 90%, at least 95%, at least 96%, at least 97%, at least 98%, at least 99%, at least 99.5%, including any values and subranges that lie there between—to the amino acid sequence of SEQ ID NO: 22. In some embodiments, the amino acid sequence of the coronavirus E protein is SEQ ID NO: 22.

In some embodiments, the viral protein is derived from the any one of Groups I, II, III, IV, V, VI, or VII of viruses according to the Baltimore classification. In some embodiments, the viral protein is derived from an enveloped negative-sense, single stranded, segmented RNA virus (e.g. Influenza virus). In some embodiments, the viral protein is derived from an enveloped DNA virus (e.g. Hepatitis B virus). In some embodiments, the viral protein is derived from a non-enveloped DNA virus (e.g. Human Papillomavirus). In some embodiments, the viral protein is derived from a positive strand enveloped RNA virus (e.g. a coronavirus, e.g., SARS CoV2, and flaviviruses, e.g., West Nile virus). In some embodiments, the viral antigen comprises the whole or an antigen fragment of any protein derived from a virus selected from the group consisting of SARS-CoV-1, MERS-CoV, chikungunya virus, African Swine Fever virus, Dengue virus, Zika virus, Influenza virus (e.g., A, B, C), Human Immunodeficiency Virus (HIV), Ebola virus, Hepatitis virus (e.g., Hepatitis A, Hepatitis B, Hepatitis C, Hepatitis D, and Hepatitis E), herpes simplex virus type 1 (HSV-1), herpes simplex virus type 2 (HSV-2), West Nile virus, and Human Papillomavirus.

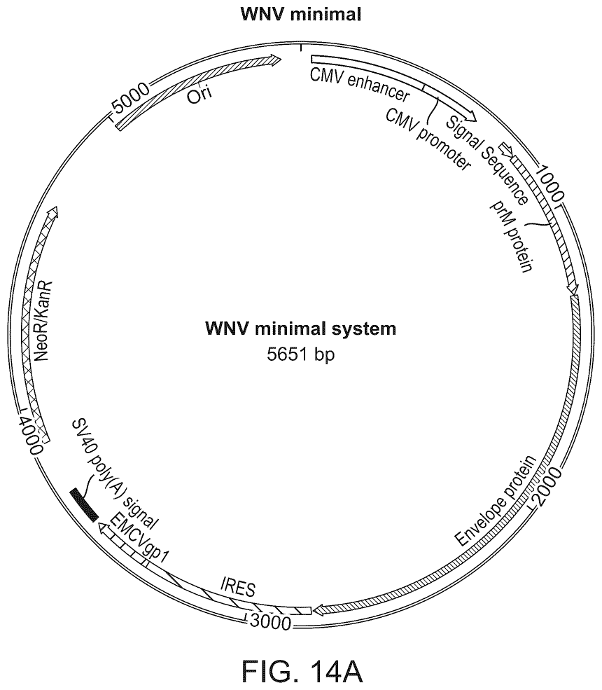

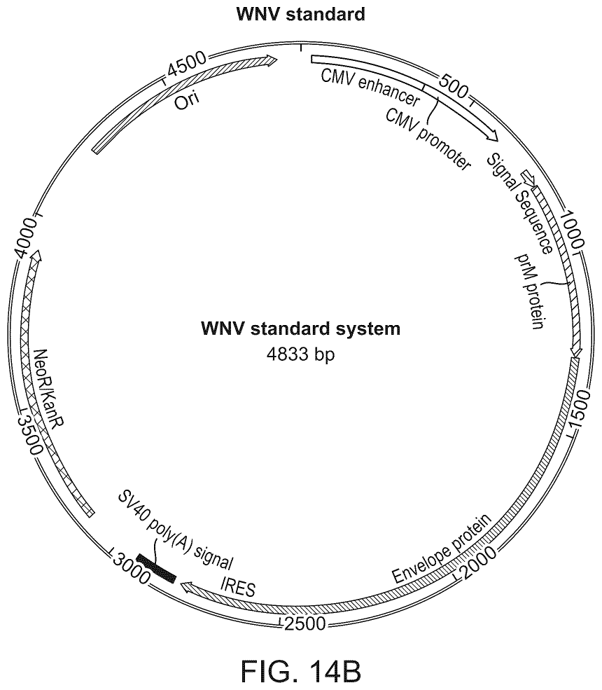

In some embodiments, the viral antigen comprises the whole or an antigen fragment of any protein derived from West Nile virus. In some embodiments, the West Nile viral protein is the precursor membrane (prM), the envelope glycoprotein (E), or a combination thereof. In some embodiments, the vector encoding one or more West Nile virus proteins, e.g., prM and/or E protein is West Nile Virus Minimal plasmid (WNV minimal plasmid), as depicted in FIG. 14A or West Nile Virus Standard plasmid (WNV standard plasmid), as depicted in FIG. 14B. In some embodiments, the vector encoding one or more West Nile virus proteins, e.g. prM and/or E protein comprises a nucleic acid sequence having at least 70% (for example, about 75%, about 80%, about 85%, about 90%, about 95%, about 96%, about 97%, about 98%, about 99%, about 99.5%, or about 100%) identity to SEQ ID NO: 55. In some embodiments, the vector encoding one or more West Nile virus proteins, e.g. prM and/or E protein comprises an expression cassette with a nucleic acid sequence having at least 70% (for example, about 75%, about 80%, about 85%, about 90%, about 95%, about 96%, about 97%, about 98%, about 99%, about 99.5%, or about 100%) identity to SEQ ID NO: 64.

In some embodiments, the polynucleotide encoding the West Nile virus E protein has a nucleic acid sequence with at least 80% sequence identity—for instance, at least 85%, at least 90%, at least 95%, at least 96%, at least 97%, at least 98%, at least 99%, at least 99.5%, including any values and subranges that lie there between—to the nucleic acid sequence of SEQ ID NO: 60. In some embodiments, the polynucleotide encoding the West Nile virus E protein has a nucleic acid sequence of SEQ ID NO: 60.

In some embodiments, the polynucleotide encoding the West Nile virus prM protein has a nucleic acid sequence with at least 80% sequence identity—for instance, at least 85%, at least 90%, at least 95%, at least 96%, at least 97%, at least 98%, at least 99%, at least 99.5%, including any values and subranges that lie there between—to the nucleic acid sequence of SEQ ID NO: 59. In some embodiments, the polynucleotide encoding the West Nile virus prM protein has a nucleic acid sequence of SEQ ID NO: 59.

The nucleic acid sequence of the vectors encoding West Nile viral antigens disclosed herein, and the genetic elements therein are listed in Table 3.

TABLE 3

| Name of | SEQ | |
|---|---|---|
| sequence | ID NO: | Sequence |
| WNV Minimal | 55 | GACTCTTCGCGATGTACGGGCCAGATATACGCGTTGA |
| plasmid | | CATTGATTATTGACTAGTTATTAATAGTAATCAATTA |
| sequence (FIG. | | CGGGGTCATTAGTTCATAGCCCATATATGGAGTTCCG |
| 14A) | | CGTTACATAACTTACGGTAAATGGCCCGCCTGGCTGA |
| | | CCGCCCAACGACCCCCGCCCATTGACGTCAATAATGA |
| | | CGTATGTTCCCATAGTAACGCCAATAGGGACTTTCCA |
| | | TTGACGTCAATGGGTGGACTATTTACGGTAAACTGCC |
| | | CACTTGGCAGTACATCAAGTGTATCATATGCCAAGTA |
| | | CGCCCCCTATTGACGTCAATGACGGTAAATGGCCCGC |
| | | CTGGCATTATGCCCAGTACATGACCTTATGGGACTTT |
| | | CCTACTTGGCAGTACATCTACGTATTAGTCATCGCTA |
| | | TTACCATGGTGATGCGGTTTTGGCAGTACATCAATGG |
| | | GCGTGGATAGCGGTTTGACTCACGGGGATTTCCAAGT |
| | | CTCCACCCCATTGACGTCAATGGGAGTTTGTTTTGGC |
| | | ACCAAAATCAACGGGACTTTCCAAAATGTCGTAACA |
| | | ACTCCGCCCCATTGACGCAAATGGGCGGTAGGCGTGT |
| | | ACGGTGGGAGGTCTATATAAGCAGAGCTggtttagtgaaccg |
| | | tcagatccgctagcgctaccggactcagatctcgagctcaagcttcgaattctgcagtcg |
| | | acggtaccgcgggcccgggatccaccggtcgccacATGGGCGGCAAGA |
| | | CAGGCATCGCCGTGATGATCGGCCTGATCGCCTCCGT |
| | | GGGCGCCGTGACCCTGAGCAACTTCCAGGGCAAGGT |
| | | GATGATGACAGTGAACGCCACAGATGTTACCGATGTT |
| | | ATCACAATCCCTACCGCCGCTGGAAAGAACTTGTGCA |
| | | TCGTACGGGCCATGGATGTGGGGTACATGTGCGACG |
| | | ACACCATCACCTACGAGTGCCCTGTGCTGAGCGCCGG |
| | | CAATGACCCCGAGGACATCGACTGCTGGTGCACCAA |
| | | GTCTGCCGTTTACGTGAGATATGGCAGGTGTACCAAA |
| | | ACCAGACACAGCAGGAGATCTCGGAGAAGCCTGACC |
| | | GTGCAAACACACGGCGAGTCCACCCTGGCCAACAAG |
| | | AAGGGCGCATGGATGGACAGCACCAAGGCCACTCGG |
| | | TACCTGGTGAAGACCGAGAGCTGGATCCTGAGAAAC |
| | | CCTGGATACGCCCTGGTGGCCGCCGTGATTGGCTGGA |
| | | TGCTGGGCTCTAACACCATGCAGAGAGTGGTGTTCGT |
| | | GGTGCTACTGCTCCTAGTGGCTCCTGCTTACAGCTTC |
| | | AACTGCCTGGGCATGTCTAACCGGGACTTCCTGGAAG |
| | | GCGTGTCCGGCGCTACATGGGTGGACCTGGTGCTCGA |
| | | GGGAGATAGCTGCGTGACCATCATGTCAAAGGACAA |
| | | GCCCACCATCGACGTGAAAATGATGAACATGGAAGC |
| | | TGCTAATCTGGCCGAGGTCAGATCTTACTGCTACCTG |
| | | GCCACAGTGAGTGATCTGAGCACAAAGGCCGCCTGC |
| | | CCCACCATGGGCGAGGCCCACAACGATAAGCGGGCC |
| | | GATCCTGCCTTCGTGTGTAGACAGGGCGTGGTGGACC |
| | | GGGGATGGGGCAACGGCTGCGGGCTGTTCGGCAAGG |
| | | GCAGCATCGATACCTGTGCCAAATTCGCCTGTAGCAC |
| | | CAAGGCCATCGGCCGGACCATTCTGAAAGAAAACAT |
| | | CAAGTACGAGGTGGCTATCTTCGTGCATGGCCCTACC |
| | | ACCGTCGAGAGCCACGGCAACTACTCCACACAGGTG |
| | | GGCGCCACACAGGCCGGCCGATTTTCTATCACACCTG |
| | | CCGCCCCCAGCTATACACTGAAACTGGGCGAGTACG |
| | | GCGAAGTGACAGTGGATTGCGAGCCTAGAAGCGGCA |
| | | TCGACACTAACGCCTACTACGTGATGACCGTGGGCAC |
| | | AAAAACCTTCCTGGTTCACAGAGAGTGGTTCATGGAC |
| | | CTGAACCTGCCTTGGTCCAGCGCCGGCAGCACCGTGT |
| | | GGCGCAATAGAGAGACACTGATGGAATTCGAGGAAC |
| | | CTCACGCCACCAAGCAGAGCGTGATCGCCCTCGGTAG |
| | | CCAGGAGGGCGCCCTGCACCAGGCCCTGGCTGGCGC |
| | | CATCCCCGTGGAATTCTCTAGCAACACCGTGAAACTG |
| | | ACCAGCGGCCACCTGAAGTGCAGAGTGAAGATGGAA |
| | | AAGCTCCAACTGAAGGGAACAACTTACGGCGTCTGC |
| | | AGCAAGGCTTTCAAGTTCCTGGGCACCCCTGCCGACA |
| | | CCGGACACGGAACCGTCGTGCTGGAACTGCAGTACA |
| | | CCGGCACAGACGGCCCATGTAAAGTGCCTATCAGCA |
| | | GCGTGGCCTCCCTGAACGACCTGACACCAGTGGGCA |
| | | GACTGGTGACCGTTAATCCTTTCGTCAGCGTGGCTAC |
| | | TGCCAATGCCAAGGTGCTGATCGAGCTGGAACCCCCC |
| | | TTCGGCGACTCTTATATCGTGGTGGGAAGAGGAGAAC |
| | | AACAGATCAACCACCACTGGCACAAGAGCGGTTCGT |
| | | CTATCGGAAAGGCTTTTACCACCACACTGAAGGGCGC |
| | | TCAGCGGCTGGCCGCCCTGGGCGACACAGCCTGGGA |
| | | CTTCGGCAGCGTGGGCGGAGTATTTACGTCCGTCGGC |
| | | AAGGCCGTCCATCAGGTGTTTGGAGGAGCCTTTCGGA |
| | | GCCTGTTCGGAGGCATGAGCTGGATCACCCAGGGCCT |
| | | GCTGGGCGCGCTGCTGCTGTGGATGGGAATTAACGCC |
| | | AGAGATAGAAGCATCGCCCTGACATTCCTGGCCGTGG |
| | | GCGGCGTGCTGCTGTTTCTGTCTGTGAACGTGCACGC |
| | | Gtaacccccccccctaacgttactggccgaagccgcttggaataaggccggtgtgcgtt |
| | | tgtctatatgttattttccaccatattgccgtcttttggcaatgtgagggcccggaaacctgg |
| | | ccctgtcttcttgacgagcattcctaggggtctttcccctctcgccaaaggaatgcaaggt |
| | | ctgttgaatgtcgtgaaggaagcagttcctctggaagcttcttgaagacaaacaacgtctg |
| | | tagcgaccctttgcaggcagcggaaccccccacctggcgacaggtgcctctgcggcca |
| | | aaagccacgtgtataagatacacctgcaaaggcggcacaaccccagtgccacgttgtg |
| | | agttggatagttgtggaaagagtcaaatggctctcctcaagcgtattcaacaaggggctg |
| | | aaggatgcccagaaggtaccccattgtatgggatctgatctggggcctcggtgcacatg |
| | | ctttacatgtgtttagtcgaggttaaaaaaacgtctaggccccccgaaccacggggacgt |
| | | ggttttcctttgaaaaacacgatgataatatggccacaaccatggaacaagagacttgcg |
| | | cgcactctctcacttttgaggaatgcccaaaatgctctgctctacaataccgtaatggatttt |
| | | acctgctaaagtatgatgaagaatggtacccagaggagttattgactgatggagaggatg |
| | | atgtctttgatcccgaattagacatggaagtcgttttcgagttacagtaaGCGAaattgtt |
| | | gttgttaacttgtttattgcagcttataatggttacaaataaagcaatagcatcacaaatttca |
| | | caaataaagcatttttttcactgcattctagttgtggtttgtccaaactcatcaatgtatcttaa |
| | | ggcgtCTTCTACTGGGCGGTTTTATGGACAGCAAGCGAA |
| | | CCGGAATTGCCAGCTGGGGCGCCCTCTGGTAAGGTTG |
| | | GGAAGCCCTGCAAAGTAAACTGGATGGCTTTCTCGCC |
| | | GCCAAGGATCTGATGGCGCAGGGGATCAAGCTCTGA |
| | | TCAAGAGACAGGATGAGGATCGTTTCGCATGATTGA |
| | | ACAAGATGGATTGCACGCAGGTTCTCCGGCCGCTTGG |
| | | GTGGAGAGGCTATTCGGCTATGACTGGGCACAACAG |
| | | ACAATCGGCTGCTCTGATGCCGCCGTGTTCCGGCTGT |
| | | CAGCGCAGGGGCGCCCGGTTCTTTTTGTCAAGACCGA |
| | | CCTGTCCGGTGCCCTGAATGAACTGCAAGACGAGGC |
| | | AGCGCGGCTATCGTGGCTGGCCACGACGGGCGTTCCT |
| | | TGCGCAGCTGTGCTCGACGTTGTCACTGAAGCGGGAA |
| | | GGGACTGGCTGCTATTGGGCGAAGTGCCGGGGCAGG |
| | | ATCTCCTGTCATCTCACCTTGCTCCTGCCGAGAAAGT |
| | | ATCCATCATGGCTGATGCAATGCGGCGGCTGCATACG |
| | | CTTGATCCGGCTACCTGCCCATTCGACCACCAAGCGA |
| | | AACATCGCATCGAGCGAGCACGTACTCGGATGGAAG |
| | | CCGGTCTTGTCGATCAGGATGATCTGGACGAAGAGCA |
| | | TCAGGGGCTCGCGCCAGCCGAACTGTTCGCCAGGCTC |
| | | AAGGCGAGCATGCCCGACGGCGAGGATCTCGTCGTG |
| | | ACCCATGGCGATGCCTGCTTGCCGAATATCATGGTGG |
| | | AAAATGGCCGCTTTTCTGGATTCATCGACTGTGGCCG |
| | | GCTGGGTGTGGCGGACCGCTATCAGGACATAGCGTTG |
| | | GCTACCCGTGATATTGCTGAAGAGCTTGGCGGCGAAT |
| | | GGGCTGACCGCTTCCTCGTGCTTTACGGTATCGCCGC |
| | | TCCCGATTCGCAGCGCATCGCCTTCTATCGCCTTCTTG |
| | | ACGAGTTCTTCTGAATTATTAACGCTTACAATTTCCTG |
| | | ATGCGGTATTTTCTCCTTACGCATCTGTGCGGTATTTC |
| | | ACACCGCATACAGGTGGCACTTTTCGGGGAAATGTGC |
| | | GCGGAACCCCTATTTGTTTATTTTTCTAAATACATTCA |
| | | AATATGTATCCGCTCATGAGACAATAACCCTGATAAA |
| | | TGCTTCAATAATAGCACGTGCTAAAACTTCATTTTTA |
| | | ATTTAAAAGGATCTAGGTGAAGATCCTTTTTGATAAT |
| | | CTCATGACCAAAATCCCTTAACGTGAGTTTTCGTTCC |
| | | ACTGAGCGTCAGACCCCGTAGAAAAGATCAAAGGAT |
| | | CTTCTTGAGATCCTTTTTTTCTGCGCGTAATCTGCTGC |
| | | TTGCAAACAAAAAAACCACCGCTACCAGCGGTGGTTT |
| | | GTTTGCCGGATCAAGAGCTACCAACTCTTTTTCCGAA |
| | | GGTAACTGGCTTCAGCAGAGCGCAGATACCAAATAC |
| | | TGTCCTTCTAGTGTAGCCGTAGTTAGGCCACCACTTC |
| | | AAGAACTCTGTAGCACCGCCTACATACCTCGCTCTGC |
| | | TAATCCTGTTACCAGTGGCTGCTGCCAGTGGCGATAA |
| | | GTCGTGTCTTACCGGGTTGGACTCAAGACGATAGTTA |
| | | CCGGATAAGGCGCAGCGGTCGGGCTGAACGGGGGGT |
| | | TCGTGCACACAGCCCAGCTTGGAGCGAACGACCTAC |
| | | ACCGAACTGAGATACCTACAGCGTGAGCTATGAGAA |
| | | AGCGCCACGCTTCCCGAAGGGAGAAAGGCGGACAGG |
| | | TATCCGGTAAGCGGCAGGGTCGGAACAGGAGAGCGC |
| | | ACGAGGGAGCTTCCAGGGGGAAACGCCTGGTATCTTT |
| | | ATAGTCCTGTCGGGTTTCGCCACCTCTGACTTGAGCG |
| | | TCGATTTTTGTGATGCTCGTCAGGGGGGCGGAGCCTA |
| | | TGGAAAAACGCCAGCAACGCGGCCTTTTTACGGTTCC |
| | | TGGGCTTTTGCTGGCCTTTTGCTCACATGTTCTT |
| Origin | 56 | TTGAGATCCTTTTTTTCTGCGCGTAATCTGCTGCTTGC |
| | | AAACAAAAAAACCACCGCTACCAGCGGTGGTTTGTTT |
| | | GCCGGATCAAGAGCTACCAACTCTTTTTCCGAAGGTA |
| | | ACTGGCTTCAGCAGAGCGCAGATACCAAATACTGTCC |
| | | TTCTAGTGTAGCCGTAGTTAGGCCACCACTTCAAGAA |
| | | CTCTGTAGCACCGCCTACATACCTCGCTCTGCTAATC |
| | | CTGTTACCAGTGGCTGCTGCCAGTGGCGATAA |
| CMV promoter | 57 | GACATTGATTATTGACTAGTTATTAATAGTAATCAAT |
| and enhancer | | TACGGGGTCATTAGTTCATAGCCCATATATGGAGTTC |
| sequence | | CGCGTTACATAACTTACGGTAAATGGCCCGCCTGGCT |
| | | GACCGCCCAACGACCCCCGCCCATTGACGTCAATAAT |
| | | GACGTATGTTCCCATAGTAACGCCAATAGGGACTTTC |
| | | CATTGACGTCAATGGGTGGACTATTTACGGTAAACTG |
| | | CCCACTTGGCAGTACATCAAGTGTATCATATGCCAAG |
| | | TACGCCCCCTATTGACGTCAATGACGGTAAATGGCCC |
| | | GCCTGGCATTATGCCCAGTACATGACCTTATGGGACT |
| | | TTCCTACTTGGCAGTACATCTACGTATTAGTCATCGCT |
| | | ATTACCATGGTGATGCGGTTTTGGCAGTACATCAATG |
| | | GGCGTGGATAGCGGTTTGACTCACGGGGATTTCCAAG |
| | | TCTCCACCCCATTGACGTCAATGGGAGTTTGTTTTGG |
| | | CACCAAAATCAACGGGACTTTCCAAAATGTCGTAACA |
| | | ACTCCGCCCCATTGACGCAAATGGGCGGTAGGCGTGT |
| | | ACGGTGGGAGGTCTATATAAGCAGAGCT |
| Signal sequence | 58 | GGCGGCAAGACAGGCATCGCCGTGATGATCGGCCTG |
| | | ATCGCCTCCGTGGGCGCC |
| prM gene | 59 | GTGACCCTGAGCAACTTCCAGGGCAAGGTGATGATG |
| | | ACAGTGAACGCCACAGATGTTACCGATGTTATCACAA |
| | | TCCCTACCGCCGCTGGAAAGAACTTGTGCATCGTACG |
| | | GGCCATGGATGTGGGGTACATGTGCGACGACACCAT |
| | | CACCTACGAGTGCCCTGTGCTGAGCGCCGGCAATGAC |
| | | CCCGAGGACATCGACTGCTGGTGCACCAAGTCTGCCG |
| | | TTTACGTGAGATATGGCAGGTGTACCAAAACCAGAC |
| | | ACAGCAGGAGATCTCGGAGAAGCCTGACCGTGCAAA |
| | | CACACGGCGAGTCCACCCTGGCCAACAAGAAGGGCG |
| | | CATGGATGGACAGCACCAAGGCCACTCGGTACCTGG |
| | | TGAAGACCGAGAGCTGGATCCTGAGAAACCCTGGAT |
| | | ACGCCCTGGTGGCCGCCGTGATTGGCTGGATGCTGGG |
| | | CTCTAACACCATGCAGAGAGTGGTGTTCGTGGTGCTA |
| | | CTGCTCCTAGTGGCTCCTGCTTACAGC |
| Envelope gene | 60 | TTCAACTGCCTGGGCATGTCTAACCGGGACTTCCTGG |
| | | AAGGCGTGTCCGGCGCTACATGGGTGGACCTGGTGCT |
| | | CGAGGGAGATAGCTGCGTGACCATCATGTCAAAGGA |
| | | CAAGCCCACCATCGACGTGAAAATGATGAACATGGA |
| | | AGCTGCTAATCTGGCCGAGGTCAGATCTTACTGCTAC |
| | | CTGGCCACAGTGAGTGATCTGAGCACAAAGGCCGCC |
| | | TGCCCCACCATGGGCGAGGCCCACAACGATAAGCGG |
| | | GCCGATCCTGCCTTCGTGTGTAGACAGGGCGTGGTGG |
| | | ACCGGGGATGGGGCAACGGCTGCGGGCTGTTCGGCA |
| | | AGGGCAGCATCGATACCTGTGCCAAATTCGCCTGTAG |
| | | CACCAAGGCCATCGGCCGGACCATTCTGAAAGAAAA |
| | | CATCAAGTACGAGGTGGCTATCTTCGTGCATGGCCCT |
| | | ACCACCGTCGAGAGCCACGGCAACTACTCCACACAG |
| | | GTGGGCGCCACACAGGCCGGCCGATTTTCTATCACAC |
| | | CTGCCGCCCCCAGCTATACACTGAAACTGGGCGAGTA |
| | | CGGCGAAGTGACAGTGGATTGCGAGCCTAGAAGCGG |
| | | CATCGACACTAACGCCTACTACGTGATGACCGTGGGC |
| | | ACAAAAACCTTCCTGGTTCACAGAGAGTGGTTCATGG |
| | | ACCTGAACCTGCCTTGGTCCAGCGCCGGCAGCACCGT |
| | | GTGGCGCAATAGAGAGACACTGATGGAATTCGAGGA |
| | | ACCTCACGCCACCAAGCAGAGCGTGATCGCCCTCGGT |
| | | AGCCAGGAGGGCGCCCTGCACCAGGCCCTGGCTGGC |
| | | GCCATCCCCGTGGAATTCTCTAGCAACACCGTGAAAC |
| | | TGACCAGCGGCCACCTGAAGTGCAGAGTGAAGATGG |
| | | AAAAGCTCCAACTGAAGGGAACAACTTACGGCGTCT |
| | | GCAGCAAGGCTTTCAAGTTCCTGGGCACCCCTGCCGA |
| | | CACCGGACACGGAACCGTCGTGCTGGAACTGCAGTA |
| | | CACCGGCACAGACGGCCCATGTAAAGTGCCTATCAG |
| | | CAGCGTGGCCTCCCTGAACGACCTGACACCAGTGGGC |
| | | AGACTGGTGACCGTTAATCCTTTCGTCAGCGTGGCTA |
| | | CTGCCAATGCCAAGGTGCTGATCGAGCTGGAACCCCC |
| | | CTTCGGCGACTCTTATATCGTGGTGGGAAGAGGAGAA |
| | | CAACAGATCAACCACCACTGGCACAAGAGCGGTTCG |
| | | TCTATCGGAAAGGCTTTTACCACCACACTGAAGGGCG |
| | | CTCAGCGGCTGGCCGCCCTGGGCGACACAGCCTGGG |
| | | ACTTCGGCAGCGTGGGCGGAGTATTTACGTCCGTCGG |
| | | CAAGGCCGTCCATCAGGTGTTTGGAGGAGCCTTTCGG |
| | | AGCCTGTTCGGAGGCATGAGCTGGATCACCCAGGGC |
| | | CTGCTGGGCGCGCTGCTGCTGTGGATGGGAATTAACG |
| | | CCAGAGATAGAAGCATCGCCCTGACATTCCTGGCCGT |
| | | GGGCGGCGTGCTGCTGTTTCTGTCTGTGAACGTGCAC |
| | | GCGtaa |
| IRES encoding | 61 | cccccccccctaacgttactggccgaagccgcttggaataaggccggtgtgcgtttgtct |
| sequence | | atatgttattttccaccatattgccgtcttttggcaatgtgagggcccggaaacctggccct |
| | | gtcttcttgacgagcattcctaggggtctttcccctctcgccaaaggaatgcaaggtctgtt |
| | | gaatgtcgtgaaggaagcagttcctctggaagcttcttgaagacaaacaacgtctgtagc |
| | | gaccctttgcaggcageggaaccccccacctggcgacaggtgcctctgcggccaaaa |
| | | gccacgtgtataagatacacctgcaaaggcggcacaaccccagtgccacgttgtgagtt |
| | | ggatagttgtggaaagagtcaaatggctctcctcaagcgtattcaacaaggggctgaag |
| | | gatgcccagaaggtaccccattgtatgggatctgatctggggcctcggtgcacatgcttta |
| | | catgtgtttagtcgaggttaaaaaaacgtctaggccccccgaaccacggggacgtggttt |
| | | tcctttgaaaaacacgatgataa |
| EMCV L | 62 | atggccacaaccatggaacaagagacttgcgcgcactctctcacttttgaggaatgccca |
| protein | | aaatgctctgctctacaataccgtaatggattttacctgctaaagtatgatgaagaatggta |
| encoding | | cccagaggagttattgactgatggagaggatgatgtctttgatcccgaattagacatggaa |
| sequence | | gtcgttttcgagttacagtaa |
| SV40 poly A | 63 | aacttgtttattgcagcttataatggttacaaataaagcaatagcatcacaaatttcacaaat |
| encoding | | aaagcatttttttcactgcattctagttgtggtttgtccaaactcatcaatgtatctta |
| sequence | | |
| Expression | 64 | ATGGGCGGCAAGACAGGCATCGCCGTGATGATCGGC |
| cassette of | | CTGATCGCCTCCGTGGGCGCCGTGACCCTGAGCAACT |
| WNV standard | | TCCAGGGCAAGGTGATGATGACAGTGAACGCCACAG |
| plasmid (FIG. | | ATGTTACCGATGTTATCACAATCCCTACCGCCGCTGG |
| 11B) | | AAAGAACTTGTGCATCGTACGGGCCATGGATGTGGG |
| | | GTACATGTGCGACGACACCATCACCTACGAGTGCCCT |
| | | GTGCTGAGCGCCGGCAATGACCCCGAGGACATCGAC |
| | | TGCTGGTGCACCAAGTCTGCCGTTTACGTGAGATATG |
| | | GCAGGTGTACCAAAACCAGACACAGCAGGAGATCTC |
| | | GGAGAAGCCTGACCGTGCAAACACACGGCGAGTCCA |
| | | CCCTGGCCAACAAGAAGGGCGCATGGATGGACAGCA |
| | | CCAAGGCCACTCGGTACCTGGTGAAGACCGAGAGCT |
| | | GGATCCTGAGAAACCCTGGATACGCCCTGGTGGCCGC |
| | | CGTGATTGGCTGGATGCTGGGCTCTAACACCATGCAG |
| | | AGAGTGGTGTTCGTGGTGCTACTGCTCCTAGTGGCTC |
| | | CTGCTTACAGCTTCAACTGCCTGGGCATGTCTAACCG |
| | | GGACTTCCTGGAAGGCGTGTCCGGCGCTACATGGGTG |
| | | GACCTGGTGCTCGAGGGAGATAGCTGCGTGACCATC |
| | | ATGTCAAAGGACAAGCCCACCATCGACGTGAAAATG |
| | | ATGAACATGGAAGCTGCTAATCTGGCCGAGGTCAGA |
| | | TCTTACTGCTACCTGGCCACAGTGAGTGATCTGAGCA |
| | | CAAAGGCCGCCTGCCCCACCATGGGCGAGGCCCACA |
| | | ACGATAAGCGGGCCGATCCTGCCTTCGTGTGTAGACA |
| | | GGGCGTGGTGGACCGGGGATGGGGCAACGGCTGCGG |
| | | GCTGTTCGGCAAGGGCAGCATCGATACCTGTGCCAAA |
| | | TTCGCCTGTAGCACCAAGGCCATCGGCCGGACCATTC |
| | | TGAAAGAAAACATCAAGTACGAGGTGGCTATCTTCGT |
| | | GCATGGCCCTACCACCGTCGAGAGCCACGGCAACTA |
| | | CTCCACACAGGTGGGCGCCACACAGGCCGGCCGATTT |
| | | TCTATCACACCTGCCGCCCCCAGCTATACACTGAAAC |
| | | TGGGCGAGTACGGCGAAGTGACAGTGGATTGCGAGC |
| | | CTAGAAGCGGCATCGACACTAACGCCTACTACGTGAT |
| | | GACCGTGGGCACAAAAACCTTCCTGGTTCACAGAGA |
| | | GTGGTTCATGGACCTGAACCTGCCTTGGTCCAGCGCC |
| | | GGCAGCACCGTGTGGCGCAATAGAGAGACACTGATG |
| | | GAATTCGAGGAACCTCACGCCACCAAGCAGAGCGTG |
| | | ATCGCCCTCGGTAGCCAGGAGGGCGCCCTGCACCAG |
| | | GCCCTGGCTGGCGCCATCCCCGTGGAATTCTCTAGCA |
| | | ACACCGTGAAACTGACCAGCGGCCACCTGAAGTGCA |
| | | GAGTGAAGATGGAAAAGCTCCAACTGAAGGGAACAA |
| | | CTTACGGCGTCTGCAGCAAGGCTTTCAAGTTCCTGGG |
| | | CACCCCTGCCGACACCGGACACGGAACCGTCGTGCTG |
| | | GAACTGCAGTACACCGGCACAGACGGCCCATGTAAA |
| | | GTGCCTATCAGCAGCGTGGCCTCCCTGAACGACCTGA |
| | | CACCAGTGGGCAGACTGGTGACCGTTAATCCTTTCGT |
| | | CAGCGTGGCTACTGCCAATGCCAAGGTGCTGATCGAG |
| | | CTGGAACCCCCCTTCGGCGACTCTTATATCGTGGTGG |
| | | GAAGAGGAGAACAACAGATCAACCACCACTGGCACA |
| | | AGAGCGGTTCGTCTATCGGAAAGGCTTTTACCACCAC |
| | | ACTGAAGGGCGCTCAGCGGCTGGCCGCCCTGGGCGA |
| | | CACAGCCTGGGACTTCGGCAGCGTGGGCGGAGTATTT |
| | | ACGTCCGTCGGCAAGGCCGTCCATCAGGTGTTTGGAG |
| | | GAGCCTTTCGGAGCCTGTTCGGAGGCATGAGCTGGAT |
| | | CACCCAGGGCCTGCTGGGCGCGCTGCTGCTGTGGATG |
| | | GGAATTAACGCCAGAGATAGAAGCATCGCCCTGACA |
| | | TTCCTGGCCGTGGGCGGCGTGCTGCTGTTTCTGTCTGT |
| | | GAACGTGCACGCGtaaGCGAaattgttgttgttaacttgtttattgcagctta |
| | | taatggttacaaataaagcaatagcatcacaaatttcacaaataaagcatttttttcactgcat |
| | | tctagttgtggtttgtccaaactcatcaatgtatctta |

In some embodiments, the viral antigen comprises the whole or an antigen fragment of any protein derived from the Influenza virus. The strain of the Influenza virus is not limited, and may be any strain that is currently known or later discovered, e.g., for example, H1N1, H3N2, or an Influenza B strain. In some embodiments, the Influenza viral protein is the HA protein, NA protein, M1 protein, M2 protein, or any combination thereof. In some embodiments, the viral antigen comprises the whole or an antigen fragment of any protein derived from the Hepatitis B virus. In some embodiments, the Hepatatis B viral protein is the sAg (S protein), sAg (M protein), sAg (L protein), preS1, preS2, cAg (core antigen), or any combination thereof. In some embodiments, the viral antigen comprises the whole or an antigen fragment of any protein derived from the Human Papilloma virus. In some embodiments, Human Papilloma viral protein is the L1 protein of HPV 6, L1 protein of HPV 11, L1 protein of HPV 16, L1 protein of HPV 18, or any combination thereof.

In some embodiments, the viral antigen comprises the whole or an antigen fragment of any one or more of the proteins derived from each of the viruses listed below in Table 4. For instance, in some embodiments, the viral antigen may comprise the whole or an antigen fragment of any protein derived from the avian Influenza virus (H5N3). Table 4

TABLE 4

| Virus | Viral protein |
|---|---|
| Avian influenza | HA, NA and M1 |
| (H5N3) | |
| BFDV | VP1 |
| BTV | VP3 and VP7 |
| Ebola | VP40 and glycoprotein |
| Enterovirus 71 | P1 and 3CD |
| GHPV | VP1 and VP2 |
| HBV | sAg (S protein) |
| HBV | sAg (S protein) and VSPalphaS |
| HBV | sAg (M protein) |
| HBV | Core antigen |
| HBV | Surface and core antigens |
| HBV | sAg (S protein), preS1 and preS2 |
| HCV | Core protein, E1 and E2 |
| HDV | HBsAg and L-HDAg |
| HEV | Capsid protein |
| HIV | Pr55gag |
| HIV | Pr160gag-pol |
| HIV | Gag protein |
| HIV | Pr55gag and RT |
| HIV | Pr55gag and TN |
| HPV11 | L1 protein |
| HPV16 | L1 protein |
| IBDV | VP2 and VP3 |
| Influenza A | M1 and ESAT6-HA |
| Influenza A | HA (H1N1) and M1 (H3N2) |
| Influenza A | HA (H1N1) and M1 (H3N2) |
| Influenza A | HA (H3N2) and M1 (H1N1) |
| Influenza A H1N1 | HA and M1 |
| Influenza A H3N2 | HA and M1 |
| IPCV | Coat protein |
| JC polyomavirus | VP1 |
| Marburg | VP40 and glycoprotein |
| MS2 | Coat protein |
| NDV | HN, F, NP and MP |
| No | Capsid protein |
| No | VP1 |
| Nv | Capsid protein |
| PhMV | Coat protein |
| PhMV | Coat protein, CPV epitopes and F |
| | protein (CDV) |
| Polyomavirus | VP1 |
| PPV | VP2 |
| RHDV | VP60 |
| Rotavirus | VP2, VP6 and VP7 |
| Rotavirus | VP2 and VP6 |
| SARS | SP, EP and MP |
| SIV | Pr55gag and envelope protein |
| SV40 | VP1 |
| SVDV | P1 and 3CD |

Enhancer Proteins

Without being bound by any theory, it is thought that the co-expression of the enhancer proteins with an antigen, may improve one or more aspects of antigen expression, including but not limited to yield, quality, folding, posttranslational modification, activity, localization, and downstream activity, or may reduce one or more of misfolding, altered activity, incorrect posttranslational modifications, and/or toxicity.

In some embodiments, the enhancer protein is a picornavirus leader (L) protein, or a functional variant thereof. In some embodiments, the picornavirus leader (L) protein is capable of blocking the nuclear pore, thereby inhibiting nucleocytoplasmic transport (“NCT”). As used herein, the term “functional variant” refers to a protein that is homologous to the picornavirus leader (L) protein and/or shares substantial sequence similarity to the picornavirus leader (L) protein (e.g., more than 30%, 40%, 50%, 60%, 70%, 80%, 85% 90%, 95%, or 99% sequence identity). In some embodiments, the functional variant shares one or more functional characteristics of the picornavirus leader (L) protein. For example, in some embodiments, a functional variant of the picornavirus leader (L) protein retains the ability to inhibit NCT.

In some embodiments, the picornavirus leader (L) protein is an L protein from the Cardiovirus, Hepatovirus, or Aphthovirus genera. For example, the enhancer protein may be from Bovine rhinitis A virus, Bovine rhinitis B virus, Equine rhinitis A virus, Foot-and-mouth disease virus, Hepatovirus A, Hepatovirus B, Marmota himalayana hepatovirus, Phopivirus, Cardiovirus A, Cardiovirus B, Theiler's Murine encephalomyelitis virus (TMEV), Vilyuisk human encephalomyelitis virus (VHEV), Theiler-like rat virus (TRV), or Saffold virus (SAF-V).

In some embodiments, the picornavirus leader (L) protein is the L protein of Theiler's virus or a functional variant thereof. In some embodiments, the L protein shares at least 90% identity to SEQ ID NO: 1. In some embodiments, the enhancer protein may comprise or consist of SEQ ID NO: 1. In some embodiments, the enhancer protein may share at least 70%, at least 75%, at least 80%, at least 85%, at least 90%, at least 95%, at least 98%, at least 99% or 100% identity to SEQ ID NO: 1.

In some embodiments, the picornavirus leader (L) protein is the L protein of Encephalomyocarditis virus (EMCV) or a functional variant thereof. In some embodiments, the L protein may share at least 90% identity to SEQ ID NO: 2. In some embodiments, the enhancer protein may comprise or consist of SEQ ID NO: 2. In some embodiments, the enhancer protein may share at least 70%, at least 75%, at least 80%, at least 85%, at least 90%, at least 95%, at least 98%, at least 99% or 100% identity to SEQ ID NO: 2.

In some embodiments, the nucleic acid sequence encoding the enhancer protein may comprise or consist of SEQ ID NO: 68. In some embodiments, the nucleic acid sequence encoding the enhancer protein may share at least 70%, at least 75%, at least 80%, at least 85%, at least 90%, at least 95%, at least 98%, at least 99% or 100% identity to SEQ ID NO: 68.

In some embodiments, the picornavirus leader (L) protein is selected from the group consisting of the L protein of poliovirus, the L protein of HRV16, the L protein of mengo virus, and the L protein of Saffold virus 2 or a functional variant thereof.

In some embodiments, the picornavirus leader (L) protein is selected from the proteins listed in Table 5 or functional variants thereof. The polynucleotide encoding the picornavirus leader (L) protein may encode an amino acid sequence at least 70%, at least 75%, at least 80%, at least 85%, at least 90%, at least 95%, at least 98%, at least 99% or 100% identical to an amino acid sequence listed in Table 2. The amino acid sequence of the picornavirus leader (L) protein may be at least 70%, at least 75%, at least 80%, at least 85%, at least 90%, at least 95%, at least 98%, at least 99% or 100% identical to an amino acid sequence listed in Table 2. The amino acid sequence of the picornavirus leader (L) protein may be at least 70%, at least 75%, at least 80%, at least 85%, at least 90%, at least 95%, at least 98%, at least 99% or 100% identical to the amino acid sequence of SEQ ID NO: 1, 2, 3, 4, 5, 6, or 12. In some embodiments, an enhancer protein may have an amino acid sequence comprising, or consisting of, one of the amino acid sequences listed in Table 2. In some embodiments, an enhancer protein may have an amino acid sequence comprising or consisting of the amino acid sequence of SEQ ID NO: 1, 2, 3, 4, 5, 6, or 12.

TABLE 5
Illustrative enhancer proteins

| Nuclear pore | | | |
|---|---|---|---|
| blocking viral protein | Origin | Family | Amino acid sequence |
| Leader protein | Theiler's virus | Picornaviridae | MACKHGYPDVCPICTAVDAT |
| | | | PGFEYLLMADGEWYPTDLLC |
| | | | VDLDDDVFWPSDTSNQSQTM |
| | | | DWTDVPLIRDIVMEPQ |
| | | | (SEQ ID NO: 12) |
| Leader protein | Theiler's-like | Picornaviridae | MACKHGYPLMCPLCTALDK |
| | virus | | TSDGLFTLLFDNEWYPTDLLT |
| | | | VDLEDEVFYPDDPHMEWTDL |
| | | | PLIQDIEMEPQ |
| | | | (SEQ ID NO: 1) |
| Leader protein | EMCV | Picornaviridae | MATTMEQETCAHSLTFEECP |
| | | | KCSALQYRNGFYLLKYDEEW |
| | | | YPEELLTDGEDDVFDPELDM |
| | | | EVVFELQ |
| | | | (SEQ ID NO: 2) |
| Leader protein | Poliovirus | Picornaviridae | NYHLATQDDLQNAVNVMWS |
| | (Enterovirus C) | | RDLLVTESRAQGTDSIARCNC |
| | | | NAGVYYCESRRKYYPVSFVG |
| | | | PTFQYMEANNYYPARYQSH |
| | | | MLIGHGFASPGDCGGILRCHH |
| | | | GVIGIITAGGEGLVAFSDIRDL |
| | | | YAYEE |
| | | | (SEQ ID NO: 3) |
| Leader protein | Equine rhinitis | Picornaviridae | MVTMAGNMICNVFAGLATEI |
| | B virus 1 | | CSPKQGPLLDNELPLPLELAE |
| | | | FPNKDNNCWVAALSHYYTL |
| | | | CDVTNHVTKVTPTTSGIRYYL |
| | | | TAWQSILQTDLENGYYPAAF |
| | | | AVETGLCHGPFPMQQHGYVR |
| | | | NATSHPYNFCLCSEPVPGEDY |
| | | | WHAVVKVDLSRTEARVDKW |
| | | | LCIDDDRMYLSGPPTRVKLAS |
| | | | SYKIPTWIESLAQFCLQLHPV |
| | | | QHRRTLANSLRNEQCR |
| | | | (SEQ ID NO: 4) |
| Leader protein | Mengo virus | Picornaviridae | MATTMEQEICAHSMTFEECP |
| | (Cardiovirus) | | KCSALQYRNGFYLLKYDEEW |
| | | | YPEESLTDGEDDVFDPDLDM |
| | | | EVVFETQ |
| | | | (SEQ ID NO: 5) |
| Leader protein | Saffold virus 2 | Picornaviridae | MACKHGYPFLCPLCTAIDTT |
| | (Cardiovirus) | | HDGSFTLLIDNEWYPTDLLTV |
| | | | DLDDDVFHPDDSVMEWTDL |
| | | | PLIQDVVMEPQ |
| | | | (SEQ ID NO: 6) |

The antigens, enhancer proteins, and/or fusion proteins, or the polynucleotides encoding such, may be modified to comprise one or more markers, labels, or tags. For example, in some embodiments, a protein of the present disclosure may be labeled with any label that will allow its detection, e.g., a radiolabel, a fluorescent agent, biotin, a peptide tag, an enzyme fragment, or the like. The proteins may comprise an affinity tag, e.g., a His-tag, a GST-tag, a Strep-tag, a biotin-tag, an immunoglobulin binding domain, e.g., an IgG binding domain, a calmodulin binding peptide, and the like. In some embodiments, polynucleotides of the present disclosure comprise a selectable marker, e.g., an antibiotic resistance marker.

Viral Packaging Signal

In some embodiments, the vectors disclosed herein comprise a polynucleotide sequence encoding a viral packaging signal (interchangeably referred to herein as “viral packaging sequence” or packaging signal” or “psi sequence”). In some embodiments, the polynucleotide sequence encoding a viral packaging signal is a DNA polynucleotide, an RNA polynucleotide, or a combination thereof. In some embodiments, the viral packaging signal is an RNA polynucleotide. In some embodiments, the vectors comprise more than one copy of the polynucleotide sequence encoding a viral packaging signal, for example, 2, 3, 4 or 5 copies of the polynucleotide sequence.

The viral packaging signal may be derived from any virus. In some embodiments, the viral packaging signal is derived from the same virus as the antigens that are expressed from the vector. In some embodiments, the viral packaging signal is derived from a different virus as the antigens that are expressed from the vector. In some embodiments, the viral packaging signal is derived from a virus selected from the group consisting of SARS-CoV-2, SARS-CoV-1, MERS-CoV, chikungunya virus, African Swine Fever virus, Dengue virus, Zika virus, Influenza virus (e.g., A, B, C), Human Immunodeficiency Virus (HIV), Ebola virus, Hepatitis virus (e.g., Hepatitis A, Hepatitis B, Hepatitis C, Hepatitis D, and Hepatitis E), herpes simplex virus type 1 (HSV-1), herpes simplex virus type 2 (HSV-2), West Nile virus, and Human Papillomavirus.

In some embodiments, the polynucleotide encoding the viral packaging element has at least about 70% identity (for example, about 75%, about 80%, about 85%, about 90%, about 95%, about 96%, about 97%, about 98%, about 99%, about 99.5%, or about 100%), including all values and subranges that lie therebetween, to the polynucleotide of SEQ ID NO: 34.

The location of the polynucleotide encoding the viral packaging signal on the vector is not limited. In some embodiments, the location of the polynucleotide encoding the viral packaging signal on the vector may be 5′ to all the nucleic acid sequences encoding the viral antigens. In some embodiments, the location of the polynucleotide encoding the viral packaging signal on the vector may be 3′ to all the nucleic acid sequences encoding the viral antigens. In some embodiments, the location of one copy of the polynucleotide encoding the viral packaging signal on the vector is 5′ to all the nucleic acid sequences encoding the viral antigens, and the location of the other copy of the polynucleotide encoding the viral antigen is 3′ to all the nucleic acid sequences encoding the viral antigens. Further, the size of the viral packaging signal is not limited and may be in the range of about 50 bps to about 3 kb, for example, about 100 bps, about 200 bps, about 300 bps, about 400 bps, about 500 bps, about 550 bps, about 600 bps, about 650 bps, about 700 bps, about 800, bps, about 900 bps, about 1 kb, about 2 kb, or about 3 kb, including all values and subranges that lie therebetween. In some embodiments, the size of the viral packaging signal is about 600 to about 700 bps, for example, about 650 bps. In some embodiments, the size of the viral packaging signal is about 661 bps.

The disclosure further provides vectors, comprising an expression cassette, said expression cassette comprising a promoter linked to a target gene, wherein the vector comprises a polynucleotide encoding any one of the viral packaging elements disclosed herein.

Order of the Genetic Elements in the Expression Cassette

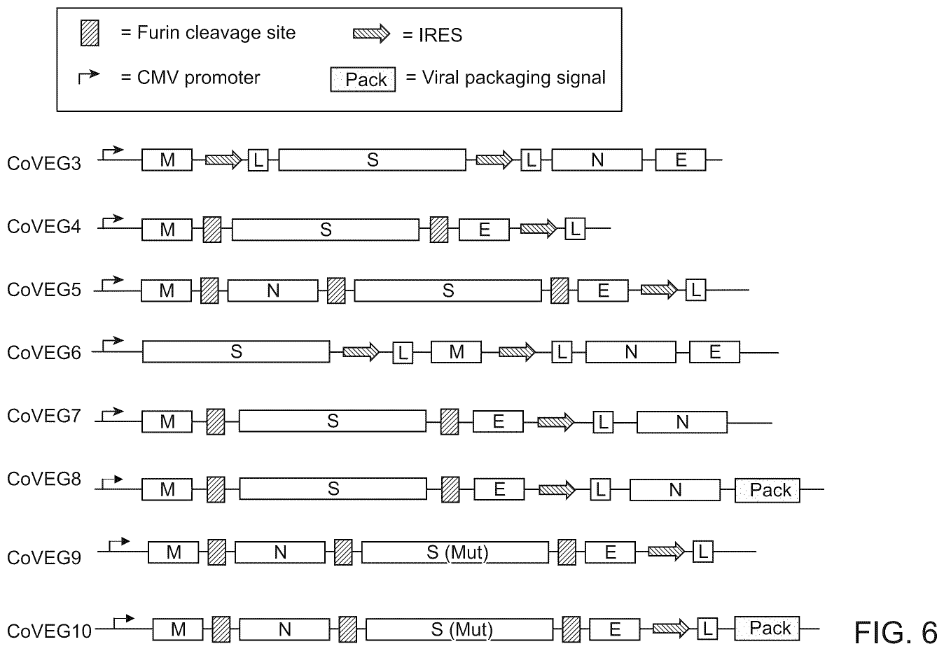

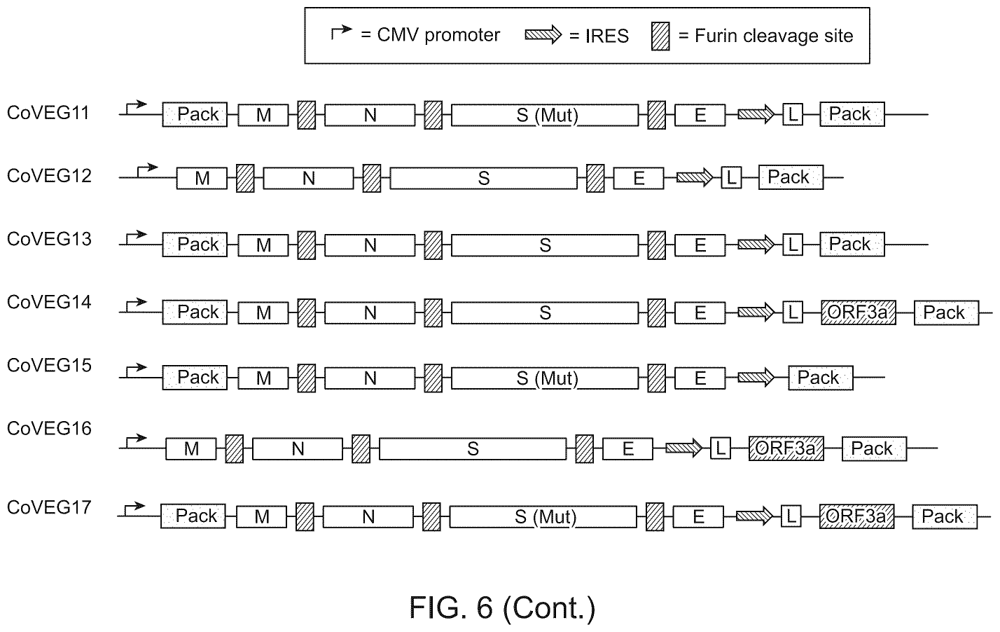

In the vectors disclosed herein, the polynucleotides sequences encoding one or more viral antigens, and the polynucleotide sequence encoding the enhancer, and/or one or more regulatory elements (e.g., a polynucleotide encoding the IRES sequence, the CMV protein, a polynucleotide encoding the viral packaging signal, and a polynucleotide encoding the proteolytic cleavage site) may be ordered in any possible combination. For instance, the order of elements in the expression cassette may be as depicted for any one of the plasmids CoVEG 3-17 in FIG. 6. Without being bound by a theory, it is thought that the order of elements in the expression cassette might be related to the expression of antigens encoded by the vector, and/or formation of VLPs. Furthermore, it is thought that when the expression cassette comprises the genes in the following order from 5′ to 3′—M, N, S, and E—it might result in higher protein expression and more stable VLP formation.

In some embodiments, the vector comprises an expression cassette, comprising the elements in the same 5′ to 3′ order as CoVEG3. In some embodiments, the vector comprises an expression cassette, comprising the following elements in the 5′ to 3′ order: a promoter, a polynucleotide encoding an M protein wherein the M protein comprises SEQ ID NO: 33 or an amino acid sequence at least 95% identical thereto, a polynucleotide encoding IRES wherein the IRES sequence comprises SEQ ID NO: 24 or a polynucleotide sequence at least 95% identical thereto, a first polynucleotide encoding an enhancer L protein wherein the L protein comprises SEQ ID NO: 2 or an amino acid sequence at least 95% identical thereto, a polynucleotide encoding an S protein wherein the S protein comprises SEQ ID NO: 13 or an amino acid sequence at least 95% identical thereto, a second polynucleotide encoding IRES wherein the IRES sequence comprises SEQ ID NO: 24 or a polynucleotide sequence at least 95% identical thereto, a second polynucleotide encoding an enhancer L protein wherein the L protein comprises SEQ ID NO: 2 or an amino acid sequence at least 95% identical thereto, a polynucleotide encoding an N protein wherein the N protein comprises SEQ ID NO: 20 or an amino acid sequence at least 95% identical thereto, and a polynucleotide encoding an E protein wherein the E protein comprises SEQ ID NO: 22 or an amino acid sequence at least 95% identical thereto.

In some embodiments, the vector comprises an expression cassette, comprising the elements in the same 5′ to 3′ order as CoVEG4. In some embodiments, the vector comprises an expression cassette, comprising the following elements in the 5′ to 3′ order: a promoter, a polynucleotide encoding an M protein wherein the M protein comprises SEQ ID NO: 33 or an amino acid sequence at least 95% identical thereto, a polynucleotide encoding a proteolytic cleavage site, a polynucleotide encoding an S protein wherein the S protein comprises SEQ ID NO: 13 or an amino acid sequence at least 95% identical thereto, a polynucleotide encoding a proteolytic cleavage site, a polynucleotide encoding a E protein wherein the E protein comprises SEQ ID NO: 22 or an amino acid sequence at least 95% identical thereto, a polynucleotide encoding IRES wherein the IRES sequence comprises SEQ ID NO: 24 or a polynucleotide sequence at least 95% identical thereto, a polynucleotide encoding an enhancer L protein wherein the L protein comprises SEQ ID NO: 2 or an amino acid sequence at least 95% identical thereto.

In some embodiments, the vector comprises an expression cassette, comprising the elements in the same 5′ to 3′ order as CoVEG5. In some embodiments, the vector comprises an expression cassette, comprising the following elements in the 5′ to 3′ order: a promoter, a polynucleotide encoding an M protein wherein the M protein comprises SEQ ID NO: 33 or an amino acid sequence at least 95% identical thereto, a polynucleotide encoding a proteolytic cleavage site, a polynucleotide encoding an N protein, wherein the N protein comprises SEQ ID NO: 20 or an amino acid sequence at least 95% identical thereto, a polynucleotide encoding a proteolytic cleavage site, a polynucleotide encoding an S protein wherein the S protein comprises SEQ ID NO: 13 or an amino acid sequence at least 95% identical thereto, a polynucleotide encoding a proteolytic cleavage site, a polynucleotide encoding a E protein wherein the E protein comprises SEQ ID NO: 22 or an amino acid sequence at least 95% identical thereto, a polynucleotide encoding IRES wherein the IRES sequence comprises SEQ ID NO: 24 or a polynucleotide sequence at least 95% identical thereto, and a polynucleotide encoding an enhancer L protein wherein the L protein comprises SEQ ID NO: 2 or an amino acid sequence at least 95% identical thereto.

In some embodiments, the vector comprises an expression cassette, comprising the elements in the same 5′ to 3′ order as CoVEG6. In some embodiments, the vector comprises an expression cassette, comprising the following elements in the 5′ to 3′ order: a promoter, a polynucleotide encoding an S protein wherein the S protein comprises SEQ ID NO: 13 or an amino acid sequence at least 95% identical thereto, a polynucleotide encoding IRES wherein the IRES sequence comprises SEQ ID NO: 24 or a polynucleotide sequence at least 95% identical thereto, a polynucleotide encoding an enhancer L protein wherein the L protein comprises SEQ ID NO: 2 or an amino acid sequence at least 95% identical thereto, a polynucleotide encoding an M protein wherein the M protein comprises SEQ ID NO: 33 or an amino acid sequence at least 95% identical thereto, a polynucleotide encoding IRES wherein the IRES sequence comprises SEQ ID NO: 24 or a polynucleotide sequence at least 95% identical thereto, a polynucleotide encoding an enhancer L protein wherein the L protein comprises SEQ ID NO: 2 or an amino acid sequence at least 95% identical thereto, a polynucleotide encoding an N protein, wherein the N protein comprises SEQ ID NO: 20 or an amino acid sequence at least 95% identical thereto, and a polynucleotide encoding a E protein wherein the E protein comprises SEQ ID NO: 22 or an amino acid sequence at least 95% identical thereto.

In some embodiments, the vector comprises an expression cassette, comprising the elements in the same 5′ to 3′ order as CoVEG7. In some embodiments, the vector comprises an expression cassette, comprising the following elements in the 5′ to 3′ order: a promoter, a polynucleotide encoding an M protein wherein the M protein comprises SEQ ID NO: 33 or an amino acid sequence at least 95% identical thereto, a polynucleotide encoding a proteolytic cleavage site, a polynucleotide encoding an S protein wherein the S protein comprises SEQ ID NO: 13 or an amino acid sequence at least 95% identical thereto, a polynucleotide encoding a proteolytic cleavage site, a polynucleotide encoding a E protein wherein the E protein comprises SEQ ID NO: 22 or an amino acid sequence at least 95% identical thereto, a polynucleotide encoding IRES wherein the IRES sequence comprises SEQ ID NO: 24 or a polynucleotide sequence at least 95% identical thereto, a polynucleotide encoding an enhancer L protein wherein the L protein comprises SEQ ID NO: 2 or an amino acid sequence at least 95% identical thereto, a polynucleotide encoding an N protein, wherein the N protein comprises SEQ ID NO: 20 or an amino acid sequence at least 95% identical thereto.

In some embodiments, the vector comprises an expression cassette, comprising the elements in the same 5′ to 3′ order as CoVEG8. In some embodiments, the vector comprises an expression cassette, comprising the following elements in the 5′ to 3′ order: a promoter, a polynucleotide encoding an M protein wherein the M protein comprises SEQ ID NO: 33 or an amino acid sequence at least 95% identical thereto, a polynucleotide encoding a proteolytic cleavage site, a polynucleotide encoding an S protein wherein the S protein comprises SEQ ID NO: 13 or an amino acid sequence at least 95% identical thereto, a polynucleotide encoding a proteolytic cleavage site, a polynucleotide encoding a E protein wherein the E protein comprises SEQ ID NO: 22 or an amino acid sequence at least 95% identical thereto, a polynucleotide encoding IRES wherein the IRES sequence comprises SEQ ID NO: 24 or a polynucleotide sequence at least 95% identical thereto, a polynucleotide encoding an enhancer L protein wherein the L protein comprises SEQ ID NO: 2 or an amino acid sequence at least 95% identical thereto, a polynucleotide encoding an N protein, wherein the N protein comprises SEQ ID NO: 20 or an amino acid sequence at least 95% identical thereto, and a polynucleotide encoding a viral packaging signal wherein the viral packaging signal comprises SEQ ID NO: 34 or a polynucleotide sequence at least 9% identical thereto.

In some embodiments, the vector comprises an expression cassette, comprising the elements in the same 5′ to 3′ order as CoVEG9. In some embodiments, the vector comprises an expression cassette, comprising the following elements in the 5′ to 3′ order: a promoter, a polynucleotide encoding an M protein wherein the M protein comprises SEQ ID NO: 33 or an amino acid sequence at least 95% identical thereto, a polynucleotide encoding a proteolytic cleavage site, a polynucleotide encoding an N protein, wherein the N protein comprises SEQ ID NO: 20 or an amino acid sequence at least 95% identical thereto, a polynucleotide encoding a proteolytic cleavage site, wherein the S protein comprises SEQ ID NO: 51 or SEQ ID NO: 52 or an amino acid sequence at least 95% identical thereto, a polynucleotide encoding a proteolytic cleavage site, a polynucleotide encoding a E protein wherein the E protein comprises SEQ ID NO: 22 or an amino acid sequence at least 95% identical thereto, a polynucleotide encoding IRES wherein the IRES sequence comprises SEQ ID NO: 24 or a polynucleotide sequence at least 95% identical thereto, a polynucleotide encoding an enhancer L protein wherein the L protein comprises SEQ ID NO: 2 or an amino acid sequence at least 95% identical thereto.

In some embodiments, the vector comprises an expression cassette, comprising the elements in the same 5′ to 3′ order as CoVEG10. In some embodiments, the vector comprises an expression cassette, comprising the following elements in the 5′ to 3′ order: a promoter, a polynucleotide encoding an M protein wherein the M protein comprises SEQ ID NO: 33 or an amino acid sequence at least 95% identical thereto, a polynucleotide encoding a proteolytic cleavage site, a polynucleotide encoding an N protein, wherein the N protein comprises SEQ ID NO: 20 or an amino acid sequence at least 95% identical thereto, a polynucleotide encoding a proteolytic cleavage site, a polynucleotide encoding a mutated S protein wherein the S protein comprises SEQ ID NO: 51 or SEQ ID NO: 52 or an amino acid sequence at least 95% identical thereto, a polynucleotide encoding a proteolytic cleavage site, a polynucleotide encoding a E protein wherein the E protein comprises SEQ ID NO: 22 or an amino acid sequence at least 95% identical thereto, a polynucleotide encoding IRES wherein the IRES sequence comprises SEQ ID NO: 24 or a polynucleotide sequence at least 95% identical thereto, a polynucleotide encoding an enhancer L protein wherein the L protein comprises SEQ ID NO: 2 or an amino acid sequence at least 95% identical thereto, and a polynucleotide encoding a viral packaging wherein the viral packaging signal comprises SEQ ID NO: 34 or a polynucleotide sequence at least 9% identical thereto.

In some embodiments, the vector comprises an expression cassette, comprising the elements in the same 5′ to 3′ order as CoVEG1 1. In some embodiments, the vector comprises an expression cassette, comprising the following elements in the 5′ to 3′ order: a promoter, a first polynucleotide encoding a viral packaging wherein the viral packaging signal comprises SEQ ID NO: 34 or a polynucleotide sequence at least 9% identical thereto, a polynucleotide encoding an M protein wherein the M protein comprises SEQ ID NO: 33 or an amino acid sequence at least 95% identical thereto, a polynucleotide encoding a proteolytic cleavage site, a polynucleotide encoding an N protein, wherein the N protein comprises SEQ ID NO: 20 or an amino acid sequence at least 95% identical thereto, a polynucleotide encoding a proteolytic cleavage site, a polynucleotide encoding a mutated S protein wherein the S protein comprises SEQ ID NO: 51 or SEQ ID NO: 52 or an amino acid sequence at least 95% identical thereto, a polynucleotide encoding a proteolytic cleavage site, a polynucleotide encoding a E protein wherein the E protein comprises SEQ ID NO: 22 or an amino acid sequence at least 95% identical thereto, a polynucleotide encoding IRES wherein the IRES sequence comprises SEQ ID NO: 24 or a polynucleotide sequence at least 95% identical thereto, a polynucleotide encoding an enhancer L protein wherein the L protein comprises SEQ ID NO: 2 or an amino acid sequence at least 95% identical thereto, and a second polynucleotide encoding a viral packaging signal wherein the viral packaging signal comprises SEQ ID NO: 34 or a polynucleotide sequence at least 9% identical thereto.

In some embodiments, the vector comprises an expression cassette, comprising the elements in the same 5′ to 3′ order as CoVEG12. In some embodiments, the vector comprises an expression cassette, comprising the following elements in the 5′ to 3′ order: a promoter, a polynucleotide encoding an M protein wherein the M protein comprises SEQ ID NO: 33 or an amino acid sequence at least 95% identical thereto, a polynucleotide encoding a proteolytic cleavage site, a polynucleotide encoding an N protein, wherein the N protein comprises SEQ ID NO: 20 or an amino acid sequence at least 95% identical thereto, a polynucleotide encoding a proteolytic cleavage site, a polynucleotide encoding a S protein wherein the S protein comprises SEQ ID NO: 13 or an amino acid sequence at least 95% identical thereto, a polynucleotide encoding a proteolytic cleavage site, a polynucleotide encoding a E protein wherein the E protein comprises SEQ ID NO: 22 or an amino acid sequence at least 95% identical thereto, a polynucleotide encoding IRES wherein the IRES sequence comprises SEQ ID NO: 24 or a polynucleotide sequence at least 95% identical thereto, a polynucleotide encoding an enhancer L protein wherein the L protein comprises SEQ ID NO: 2 or an amino acid sequence at least 95% identical thereto, and a polynucleotide encoding a viral packaging signal wherein the viral packaging signal comprises SEQ ID NO: 34 or a polynucleotide sequence at least 9% identical thereto.

In some embodiments, the vector comprises an expression cassette, comprising the elements in the same 5′ to 3′ order as CoVEG13. In some embodiments, the vector comprises an expression cassette, comprising the following elements in the 5′ to 3′ order: a promoter, a first polynucleotide encoding a viral packaging signal (wherein the viral packaging signal comprises SEQ ID NO: 34 or a polynucleotide sequence at least 9% identical thereto, a polynucleotide encoding an M protein wherein the M protein comprises SEQ ID NO: 33 or an amino acid sequence at least 95% identical thereto, a polynucleotide encoding a proteolytic cleavage site, a polynucleotide encoding an N protein, wherein the N protein comprises SEQ ID NO: 20 or an amino acid sequence at least 95% identical thereto, a polynucleotide encoding a proteolytic cleavage site, a polynucleotide encoding a S protein wherein the S protein comprises SEQ ID NO: 13 or an amino acid sequence at least 95% identical thereto, a polynucleotide encoding a proteolytic cleavage site, a polynucleotide encoding a E protein wherein the E protein comprises SEQ ID NO: 22 or an amino acid sequence at least 95% identical thereto, a polynucleotide encoding IRES wherein the IRES sequence comprises SEQ ID NO: 24 or a polynucleotide sequence at least 95% identical thereto, a polynucleotide encoding an enhancer L protein wherein the L protein comprises SEQ ID NO: 2 or an amino acid sequence at least 95% identical thereto, and a second polynucleotide encoding a viral packaging signal wherein the viral packaging signal comprises SEQ ID NO: 34 or a polynucleotide sequence at least 9% identical thereto.

In some embodiments, the vector comprises an expression cassette, comprising the elements in the same 5′ to 3′ order as CoVEG14. In some embodiments, the vector comprises an expression cassette, comprising the following elements in the 5′ to 3′ order: a promoter, a first polynucleotide encoding a viral packaging wherein the viral packaging signal comprises SEQ ID NO: 34 or a polynucleotide sequence at least 9% identical thereto, a polynucleotide encoding an M protein wherein the M protein comprises SEQ ID NO: 33 or an amino acid sequence at least 95% identical thereto, a polynucleotide encoding a proteolytic cleavage site, a polynucleotide encoding an N protein, wherein the N protein comprises SEQ ID NO: 20 or an amino acid sequence at least 95% identical thereto, a polynucleotide encoding a proteolytic cleavage site, a polynucleotide encoding a S protein wherein the S protein comprises SEQ ID NO: 13 or an amino acid sequence at least 95% identical thereto, a polynucleotide encoding a proteolytic cleavage site, a polynucleotide encoding a E protein wherein the E protein comprises SEQ ID NO: 22 or an amino acid sequence at least 95% identical thereto, a polynucleotide encoding IRES wherein the IRES sequence comprises SEQ ID NO: 24 or a polynucleotide sequence at least 95% identical thereto, a polynucleotide encoding an enhancer L protein wherein the L protein comprises SEQ ID NO: 2 or an amino acid sequence at least 95% identical thereto, a polynucleotide encoding ORF3a wherein the ORF3a encodes SEQ ID NO: 53, or an amino acid sequence with at least 95% identity thereto, and a second polynucleotide encoding a viral packaging signal wherein the viral packaging signal comprises SEQ ID NO: 34 or a polynucleotide sequence at least 9% identical thereto.

In some embodiments, the vector comprises an expression cassette, comprising the elements in the same 5′ to 3′ order as CoVEG15. In some embodiments, the vector comprises an expression cassette, comprising the following elements in the 5′ to 3′ order: a promoter, a first polynucleotide encoding a viral packaging signal wherein the viral packaging signal comprises SEQ ID NO: 34 or a polynucleotide sequence at least 95% identical thereto, a polynucleotide encoding an M protein wherein the M protein comprises SEQ ID NO: 33 or an amino acid sequence at least 95% identical thereto, a polynucleotide encoding a proteolytic cleavage site, a polynucleotide encoding an N protein wherein the N protein comprises SEQ ID NO: 20 or an amino acid sequence at least 95% identical thereto, a polynucleotide encoding a proteolytic cleavage site, a polynucleotide encoding a mutated S wherein the S protein comprises SEQ ID NO: 51 or SEQ ID NO: 52 or an amino acid sequence at least 95% identical thereto, a polynucleotide encoding a proteolytic cleavage site, a polynucleotide encoding a E protein wherein the E protein comprises SEQ ID NO: 22 or an amino acid sequence at least 95% identical thereto, a polynucleotide encoding IRES wherein the IRES sequence comprises SEQ ID NO: 24 or a polynucleotide sequence at least 95% identical thereto, and a second polynucleotide encoding a viral packaging signal (wherein the viral packaging signal comprises SEQ ID NO: 34 or a polynucleotide sequence at least 9% identical thereto.

In some embodiments, the vector comprises an expression cassette, comprising the elements in the same 5′ to 3′ order as CoVEG16. In some embodiments, the vector comprises an expression cassette, comprising the following elements in the 5′ to 3′ order: a promoter, a polynucleotide encoding an M protein wherein the M protein comprises SEQ ID NO: 33 or an amino acid sequence at least 95% identical thereto, a polynucleotide encoding a proteolytic cleavage site, a polynucleotide encoding an N protein, wherein the N protein comprises SEQ ID NO: 20 or an amino acid sequence at least 95% identical thereto, a polynucleotide encoding a proteolytic cleavage site, a polynucleotide encoding a S protein wherein the S protein comprises SEQ ID NO: 13 or an amino acid sequence at least 95% identical thereto, a polynucleotide encoding a proteolytic cleavage site, a polynucleotide encoding a E protein wherein the E protein comprises SEQ ID NO: 22 or an amino acid sequence at least 95% identical thereto, a polynucleotide encoding IRES wherein the IRES sequence comprises SEQ ID NO: 24 or a polynucleotide sequence at least 95% identical thereto, a polynucleotide encoding an enhancer L protein wherein the L protein comprises SEQ ID NO: 2 or an amino acid sequence at least 95% identical thereto, a polynucleotide encoding ORF3a encodes SEQ ID NO: 53, or an amino acid sequence with at least 95% identity thereto, and a polynucleotide encoding a viral packaging signal, wherein the viral packaging signal comprises SEQ ID NO: 34 or a polynucleotide sequence at least 95% identical thereto.

In some embodiments, the vector comprises an expression cassette, comprising the elements in the same 5′ to 3′ order as CoVEG17. In some embodiments, the vector comprises an expression cassette, comprising the following elements in the 5′ to 3′ order: a promoter, a first polynucleotide encoding a viral packaging signal wherein the viral packaging signal comprises SEQ ID NO: 34 or a polynucleotide sequence at least 9% identical thereto, a polynucleotide encoding an M protein wherein the M protein comprises SEQ ID NO: 33 or an amino acid sequence at least 95% identical thereto, a polynucleotide encoding a proteolytic cleavage site, a polynucleotide encoding an N protein, wherein the N protein comprises SEQ ID NO: 20 or an amino acid sequence at least 95% identical thereto, a polynucleotide encoding a proteolytic cleavage site, a polynucleotide encoding a mutated S wherein the S protein comprises SEQ ID NO: 51 or SEQ ID NO: 52 or an amino acid sequence at least 95% identical thereto, a polynucleotide encoding a proteolytic cleavage site, a polynucleotide encoding a E protein wherein the E protein comprises SEQ ID NO: 22 or an amino acid sequence at least 95% identical thereto, a polynucleotide encoding IRES wherein the IRES sequence comprises SEQ ID NO: 24 or a polynucleotide sequence at least 95% identical thereto, a polynucleotide encoding an enhancer L protein wherein the L protein comprises SEQ ID NO: 2 or an amino acid sequence at least 95% identical thereto, a polynucleotide encoding ORF3a wherein the ORF3a protein comprises SEQ ID NO: 53 and a second polynucleotide encoding a viral packaging signal wherein the viral packaging signal comprises SEQ ID NO: 34 or a polynucleotide signal with at least 95% identity thereto.

Expression of Antigens and VLPs in Cells

The disclosure provides methods of expressing an antigen in a eukaryotic cell, comprising contacting the cell with any one of the vectors disclosed herein. In some embodiments, the vector is contacted with the cell in vitro, ex vivo or in vivo. In some embodiments, the vector is contacted with the cell (in vivo) in a subject.

In some embodiments, the expression of one or more antigens results in the formation of a virus like particle (VLP). In some embodiments, a VLP is immunogenic. In some embodiments, a VLP is capable of eliciting an immune response in a subject. In some embodiments, the VLP is enveloped. In some embodiments, the VLP is non-enveloped. The number of antigens present in a VLP is not limited. In some embodiments, a VLP comprises one antigen, two antigens, three antigens, four antigens, five antigens, six antigens, seven antigens, eight antigens, nine antigens, ten antigens, or a higher number of antigens. In some embodiments, the VLP comprises three antigens. In some embodiments, the VLP comprises four antigens. In some embodiments, the structural proteins that form a VLP and the immunogenic viral antigens that are a part of the VLP are derived from the same virus (i.e., a native VLP). In some embodiments, the structural viral proteins that form a VLP are derived from one virus and the immunogenic viral antigens that get incorporated to that said VLP are derived from another virus (i.e., a chimeric VLP). In some embodiments, the viral proteins are mutated to enhance VLP assembly, VLP secretion and/or loading of the immunogenic antigen or antigens to the said VLP.

In some embodiments, the vector comprises a DNA polynucleotide encoding a viral packaging signal, such that contacting the cell with the vector results in expression of the viral packaging signal. In some embodiments, the VLPs encapsidate the viral packaging signal. In some embodiments, the expression of the viral packaging signal increases or promotes the formation of VLPs. In some embodiments, a greater number of VLPs are formed in the presence of a viral packaging signal, as compared to in the absence of a viral packaging signal. In some embodiments, contacting the cell with any one of disclosed vectors encoding the viral packaging signal results in the expression of a greater number of VLPs, as compared to a control vector lacking the DNA polynucleotide encoding the viral packaging signal. In some embodiments, contacting the cell with any one of disclosed vectors encoding the viral packaging signal results in the packaging of the viral packaging signal within the VLPs, which in turn leads to enhanced immune response due to an improved adjuvating characteristics or other mechanisms. In some embodiments, the packaging signals and proteins are derived from the same virus from which the VLP is formed (i.e., native packaging). In some embodiments, the packaging signals and proteins are derived from another virus with a known packaging mechanism (i.e., chimeric packaging).

In some embodiments, the expression cassette comprises a polynucleotide sequence encoding a first antigen, a second antigen, a third antigen, a fourth antigen, or a combination thereof. In some embodiments, the expression cassette comprises a polynucleotide sequence encoding a first antigen, a second antigen, and a third antigen. In some embodiments, the expression cassette comprises a polynucleotide sequence encoding a first antigen, a second antigen, a third antigen, and a fourth antigen.

In some embodiments, the first antigen is a coronavirus spike protein, the second antigen is a coronavirus membrane (M) protein, and the third antigen is a coronavirus envelope (E) protein. In some embodiments, wherein the first antigen is a coronavirus spike protein, the second antigen is a coronavirus membrane (M) protein, the third antigen is a coronavirus envelope (E) protein and the fourth antigen is a coronavirus nucleocapsid (N) protein.

In some embodiments, the vector causes: (i) expression of the antigen at a higher expression level; and/or (ii) expression of the antigen for a longer period of time; and/or (iii) expression of the antigen with better protein quality, as compared to a vector lacking the enhancer protein. In some embodiments, the vector causes: (i) expression of a virus like particle (VLP) comprising the antigen at a higher expression level; and/or (ii) expression of a VLP comprising the antigen for a longer period of time; and/or (iii) expression of a VLP comprising the antigen with better protein quality, than a vector lacking the enhancer protein. As used herein, “protein quality” might refer to without limitation, protein folding, posttranslational modification, functional activity, localization, and downstream activity. Thus, in some embodiments, the antigen which is co-expressed with an enhancer protein using any of the methods or vectors or compositions disclosed herein may have improved protein folding, improved posttranslational modification, improved functional activity, improved localization, and improved downstream activity, as compared to the antigen which is not co-expressed with an enhancer protein.

As used herein, the terms “transfection,” “transduction,” and “transformation” refer to the process of introducing nucleic acids into cells (e.g., eukaryotic cells). The vectors disclosed herein may be introduced into a cell (e.g., a eukaryotic cell) using any method known in the art. For example, the vector can be introduced into a cell using chemical, physical, biological, or viral means. Methods of introducing a vector into a cell include, but are not limited to, the use of calcium phosphate, dendrimers, cationic polymers, lipofection, fugene, cell-penetrating peptides, peptide dendrimers, electroporation, cell squeezing, sonoporation, optical transfection, protoplast fusion, impalefection, hydrodynamic delivery, gene gun, magnetofection, particle bombardment, nucleofection, viral transduction, injection, transformation, transfection, direct uptake, projectile bombardment, and liposomes. Other non-limiting examples of methods include viral transfection, direct uptake, projectile bombardment, direct injection with or without electroporation/sonoporation while using or not using cationic polymers, lipids, lipid formulations, and jet-gene devices. Antigens and enhancer proteins can be stably or transiently expressed in cells using expression vectors. Techniques of expression in eukaryotic cells are well known to those in the art. (See Current Protocols in Human Genetics: Chapter 12 “Vector Therapy” & Chapter 13 “Delivery Systems for Gene Therapy”).

In some embodiments, vectors can be introduced into a host cell by insertion into the genome using standard methods to produce stable cell lines, optionally through the use of lentiviral transfection, baculovirus gene transfer into mammalian cells (BacMam), retroviral transfection, CRISPR/Cas9, and/or transposons. In some embodiments, polynucleotides or vectors can be introduced into a host cell for transient transfection. In some embodiments, transient transfection may be effected through the use of viral vectors, helper lipids, e.g., PEI, Lipofectamine, and/or Fectamine 293. The genetic elements can be encoded as DNA on e.g. a vector or as RNA from e.g. PCR. The genetic elements can be separated in different or combined on the same vector.

The host cell used to express the antigen and enhancer protein is not limited, and may include a prokaryotic host (e.g., E. coli) or a eukaryotic host (e.g., Saccharomyces cerevisiae, insect cells, e.g., Sf21 cells, or mammalian cell lines and primary cells, e.g., NIH 3T3, HeLa, COS cells). Such cells are available from a wide range of sources (e.g., the American Type Culture Collection, Rockland, Md.; also, see, e.g., Ausubel et al., supra). Non limiting examples of insect cells are, Spodoptera frugiperda (S1) cells, e.g. Sf9, Sf21, Trichoplusia ni cells, e.g. High Five cells, and Drosophila S2 cells. Examples of fungi (including yeast) host cells are S. cerevisiae, Kluyveromyces lactis (K lactis), species of Candida including C. albicans and C. glabrata, Aspergillus nidulans, Schizosaccharomyces pombe (S. pombe), Pichia pastoris, and Yarrowia lipolytica. Examples of mammalian cells are COS cells, baby hamster kidney cells, mouse L cells, LNCaP cells, Chinese hamster ovary (CHO) cells, human embryonic kidney (HEK) cells, African green monkey cells, CV1 cells, HeLa cells, MDCK cells, Vero and Hep-2 cells. Xenopus laevis oocytes, or other cells of amphibian origin, may also be used. Prokaryotic host cells include bacterial cells, for example, E. coli, B. subtilis, and mycobacteria.

Vaccine Compositions

The disclosure provides vaccine compositions comprising any one of the vectors disclosed herein, and at least one pharmaceutically acceptable carrier, excipient, and/or vehicle, for example, solvents, buffers, solutions, dispersion media, coatings, antibacterial and antifungal agents, isotonic and absorption delaying agents. As used herein, the term “pharmaceutically acceptable” means being approved by a regulatory agency of the Federal or a state government or listed in the U.S. Pharmacopeia, European Pharmacopeia or other generally recognized pharmacopeia for use in mammals, and more particularly in humans. In some embodiments, the pharmaceutically acceptable carrier, excipient, and/or vehicle may comprise saline, buffered saline, dextrose, water, glycerol, sterile isotonic aqueous buffer, and combinations thereof. In some embodiments, the pharmaceutically acceptable carrier, excipient, and/or vehicle comprises phosphate buffered saline, sterile saline, lactose, sucrose, calcium phosphate, dextran, agar, pectin, peanut oil, sesame oil, pharmaceutical grades of mannitol, lactose, starch, magnesium stearate, sodium saccharine, cellulose, magnesium carbonate, polyol (e.g., glycerol, propylene glycol, and liquid polyethylene glycol, and the like) or suitable mixtures thereof. In some embodiments, the compositions disclosed herein further comprise minor amounts of emulsifying or wetting agents, or pH buffering agents.

In some embodiments, the composition is in a solid form, e.g. a lyophilized powder suitable for reconstitution, a liquid solution, suspension, emulsion, tablet, pill, capsule, sustained release formulation, or powder. In some embodiments, delivery vehicles e.g. liposomes, nanocapsules, nanoparticles, microparticles, microspheres, lipid particles, vesicles, polymers, peptides, and the like, may be used for the introduction of the vectors and vaccine compositions disclosed herein into suitable host cells. In some embodiments, the vectors and vaccine compositions disclosed herein may be formulated for delivery either encapsulated in a lipid particle, a liposome, a vesicle, a nanosphere, or a nanoparticle or the like.

In some embodiments, the compositions disclosed herein comprise other conventional pharmaceutical ingredients, e.g. preservatives, or chemical stabilizers, e.g. chlorobutanol, potassium sorbate, sorbic acid, sulfur dioxide, propyl gallate, the parabens, ethyl vanillin, glycerin, phenol, parachlorophenol or albumin. In some embodiments, the compositions disclosed herein comprise antibacterial and antifungal agents, e.g., parabens, chlorobutanol, phenol, sorbic acid or thimerosal; isotonic agents, e.g., sugars or sodium chloride and/or agents delaying absorption, e.g., aluminum monostearate and gelatin.

In some embodiments, the vaccine composition comprises an adjuvant. As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses.

In some embodiments, the adjuvant is alum. In some embodiments, the adjuvant is monophosphoryl lipid A (MPL). In some embodiments, other adjuvants may be used in addition or as an alternative. The inclusion of any adjuvant described in Vogel et al., “A Compendium of Vaccine Adjuvants and Excipients (2nd Edition),” herein incorporated by reference in its entirety for all purposes, is envisioned within the scope of this disclosure. Other adjuvants include complete Freund's adjuvant (a non-specific stimulator of the immune response containing killed Mycobacterium tuberculosis), incomplete Freund's adjuvants and aluminum hydroxide adjuvant, GMCSP, BCG, MDP compounds, e.g. thur-MDP and nor-MDP, CGP (MTP-PE), lipid A, and monophosphoryl lipid A (MPL), MF-59, RIBI, which contains three components extracted from bacteria, MPL, trehalose dimycolate (TDM) and cell wall skeleton (CWS) in a 2% squalene/Tween® 80 emulsion. In some embodiments, the adjuvant may be a paucilamellar lipid vesicle; for example, Novasomes®. Novasomes® are paucilamellar nonphospholipid vesicles ranging from about 100 nm to about 500 nm. They comprise Brij 72, cholesterol, oleic acid and squalene. Novasomes have been shown to be an effective adjuvant (see, U.S. Pat. Nos. 5,629,021, 6,387,373, and 4,911,928). In some embodiments, the compositions may be free of added adjuvant. Alum-free compositions that induce robust immune responses are especially useful in adults about 60 and older.

Methods of Eliciting an Immune Response in a Subject

The disclosure further provides methods of eliciting an immune response in a subject, comprising administering an effective amount of any one of the vaccine compositions disclosed herein to the subject. In some embodiments, tissue at an administration site of the subject expresses the antigen and/or a VLP comprising the antigen. In some embodiments, tissue at an administration site of the subject: (i) expresses the antigen and/or a VLP comprising the antigen at a higher expression level; and/or (ii) expresses the antigen and/or a VLP comprising the antigen for a longer period of time; and/or (iii) expresses the antigen and/or a VLP comprising the antigen with better protein quality, as compared to when a vector lacking the enhancer protein is administered.

In some embodiments, the method elicits an antibody response in the subject. In some embodiments, the antibody response is a neutralizing antibody response. In some embodiments, the method elicits a cellular immune response. In some embodiments, the method elicits a prophylactic, protective and/or therapeutic immune response in the subject.

In some embodiments, the vector comprises a DNA polynucleotide encoding a viral packaging signal, such that the tissue at an administration site of the subject expresses the viral packaging signal. In some embodiments, the VLPs encapsidate the viral packaging signal. In some embodiments, the VLPs encapsidate a polynucleotide comprising the viral packaging signal. In some embodiments, the VLPs encapsidate a polynucleotide consisting of the viral packaging signal. In some embodiments, the VLPs encapsidating the viral packaging signal are more immunogenic than control VLPs comprising the antigen but lacking the viral packaging signal. Without being bound by a theory, it is thought that a greater number of VLPs may be formed in the presence of a viral packaging signal, as compared to in the absence of the viral packaging signal. Thus, in some embodiments, the disclosed vectors encoding a viral packaging signal promote the formation of a greater number of VLPs, as compared to a control vector which does not encode the viral packaging signal. Without being bound by a theory, it is also thought that the RNA viral packaging signals may act as an adjuvant by acting as an agonist of Toll-like Receptors (TLRs).

Methods of administering any one of the compositions or vectors disclosed herein include, but are not limited to, parenteral administration (e.g., intradermal, intramuscular, intravenous and subcutaneous), epidural, and mucosal (e.g., intranasal and oral or pulmonary routes or by suppositories), subdermal, and intraperitoneal. In some embodiments, compositions of the present invention are administered intramuscularly, intravenously, subcutaneously, transdermally or intradermally. The compositions or vectors may be administered by any convenient route, for example by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and may be administered together with other biologically active agents. In some embodiments, the administration is intradermal administration. In some embodiments, the administration is intramuscular administration. In some embodiments, the administration is subcutaneous administration. In some embodiments, the administration is intranasal administration. In some embodiments, the compositions or vectors disclosed herein are administered by injection. In some embodiments, the injection is performed using a needle, a syringe, a microneedle, or a needle-less injection device. In some embodiments, the compositions or vectors disclosed herein are administered intranasally, either by drops, large particle aerosol (greater than about 10 microns), or spray into the upper respiratory tract or small particle aerosol (less than 10 microns) or spray into the lower respiratory tract. In some embodiments, the injection is followed by electroporation.

The vectors or vaccine compositions disclosed herein may be administered on a single dose schedule or a multiple dose schedule. Multiple doses may be used in a primary immunization schedule or in a booster immunization schedule. In a multiple dose schedule the various doses may be given by the same or different routes e.g., a parenteral prime and mucosal boost, a mucosal prime and parenteral boost, etc. In some aspects, a follow-on boost dose is administered within a time period of about 1 hour to about several years (for example, about 12 hours, about 1 day, about 2 days, about 5 days, about 1 week, about 2 weeks, about 3 weeks, about 4 weeks, about 5 weeks, about 6 weeks, about 1 month, about 6 months, about 1 year, about 2 years, including all values and subranges that lie there between) after the prior dose.

Immunogenic Effects

In some embodiments, inclusion of the enhancer protein in a polynucleotide encoding one or more viral antigen proteins increases functional viral-like particle (VLP) production relative to a polynucleotide without an enhancer protein. In some embodiments, inclusion of the enhancer protein increases functional VLP production by about 10%, about 20%, about 30%, about 40%, about 50%, about 60%, about 70%, about 80%, about 90%, about 100%, about 125%, about 150%, about 175%, about 200%, about 250%, about 300%, about 350%, about 400%, about 500%, or about 1000% relative to a vector without an enhancer protein. Functional VLP production as used herein may be measured by method known in the art, including but not limited to: the level of protein aggregation, the titer of neutralizing antibodies in vivo, induced Th1 response, the amount of VLPs over time relative to VLP half-life, and/or cell death associated with mis-folded VLPs.

In some embodiments, inclusion of the enhancer protein in a polynucleotide encoding one or more viral antigen proteins increases the duration or the amount of neutralizing antibodies in a subject relative to a vaccine composition without an enhancer protein. In some embodiments inclusion of the enhancer protein increases the duration or the amount of neutralizing antibodies in a subject by about 2-fold, about 3-fold, about 4-fold, about 5-fold, about 6-fold, about 7-fold, about 8-fold, about 9-fold, or about 10-fold relative to a vaccine composition without an enhancer protein.

In some embodiments, inclusion of the enhancer protein increases Th1 cellular response relative to a vaccine composition without an enhancer protein. In some embodiments, inclusion of the enhancer protein increases Th1 response by about 25%, about 50%, about 100%, about 200%, about 300%, about 400%, about 500%, or about 1000% relative to a vaccine composition without an enhancer protein.

Kits and Articles of Manufacture

The disclosure provides kits comprising any one or more of the vectors disclosed herein. The disclosure further provides kits comprising any one or more of the polynucleotides disclosed herein. The disclosure also provides kits comprising any one or more of the vaccine compositions disclosed herein. The kits disclosed herein are useful for performing, or aiding in the performance of, the disclosed methods. In some embodiments, the kits comprise a pharmaceutically acceptable carrier. In some embodiments, the kits comprise instructions for proper use and safety information of the product or formulation. In some embodiments, the kits comprise dosage information based on the application and method of administration as determined by a doctor.

The present application also provides articles of manufacture comprising any one of the vaccine compositions or kits described herein. Examples of an article of manufacture include vials (e.g. sealed sterile vials).

In some embodiments, the kits comprise one or more containers or vials filled with one or more of the ingredients of the vaccine compositions disclosed herein. In some embodiments, the kit comprises two containers, one containing the vector, or polynucleotide, or vaccine composition disclosed herein, and the other containing an adjuvant. In some embodiments, the kits further comprise a notice reflecting approval by a governmental agency for manufacture, use or sale for human administration.

The inventions are further illustrated by the following additional examples that should not be construed as limiting. Those of skill in the art, in light of the present disclosure, would be able to appreciate that many changes can be made to the specific embodiments which are disclosed and still obtain a like or similar result without departing from the spirit and scope of the inventions.

### Examples

### Example 1: Construction of polynucleotides encoding SARS-CoV-2 and L protein

CoVEG1 and CoVEG2 plasmids encode SARS-CoV-2 and the L enhancer protein.

Plasmid CoVEG1 comprises polynucleotides encoding viral proteins of full-length S protein (SEQ ID NO: 14), M protein (SEQ ID NO: 19), and E protein (SEQ ID NO: 23) of SARS-CoV-2. Plasmid CoVEG2 comprises polynucleotides encoding viral proteins of full-length S protein, M protein, N protein (SEQ ID NO: 21) and E protein of SARS-CoV-2. The backbone of CoVEG1 and CoVEG2 plasmids is shown in FIG. 1. In addition, the CoVEG1 and CoVEG2 plasmids also comprise a polynucleotide encoding the L protein from EMCV (SEQ ID NO: 16).

The nucleic acid sequence of the complete insert in CoVEG2 is represented by SEQ ID NO: 30. See Table 1. The expression of this construct gives rise to three polypeptides: the SARS-CoV-2 Spike protein having amino acid sequence of SEQ ID NO: 13, CoVEG2 polypeptide 1 having amino acid sequence of SEQ ID NO: 25, and CoVEG2 polypeptide 2 having amino acid sequence of SEQ ID NO: 26. The nucleic acid sequence of the insert in CoVEG1 is represented by SEQ ID NO: 31. The expression of this construct gives rise to two polypeptides: the SARS-CoV-2 Spike protein having amino acid sequence of SEQ ID NO: 13, and CoVEG1 polypeptide having amino acid sequence of SEQ ID NO: 32. See Table 1.

The plasmid backbone (based on the design principles of the pVaxl plasmid) and insert for both the plasmids were generated using gene synthesis and do not contain any animal or human source material. The plasmid backbone consists of a Kanamycin resistance gene, the ColE1 origin of replication, the Human cytomegalovirus immediate-early promoter and Simian virus (SV40) Poly A signal. Polynucleotides encoding viral proteins were cloned in between the CMV promoter and the SV40 PolyA signal. After gene synthesis and plasmid preparation, the plasmid was transformed into E. coli for cloning and then screened using kanamycin. A representative colony was selected, and its plasmid sequence verified and used as source plasmid for further development. After transcription, the viral proteins were expressed from a single polycistronic mRNA.

Without being bound by any particular theory, it is thought that when co-expressed, the S, E and M proteins assemble into VLPs, and are secreted by expressing cells; and that the VLP secretion is significantly increased when N protein is also expressed together with the S, E and M proteins.

### Example 2: Expression of SARS-CoV-2 S, E, M, and N Proteins in Eukaryotic Cells Observed by Immunofluorescence

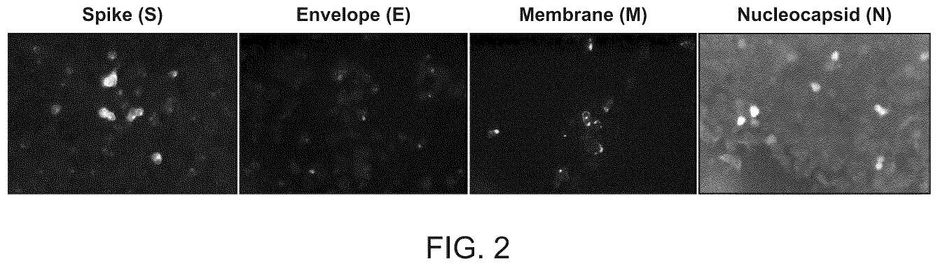

HEK 293 (eukaryotic) cells were transfected with the pCoVEG2 plasmid. Twenty-four hours later, cells were fixed, permeabilized and analyzed by immunocytochemistry using commercial Alexa Fluor 568 fluorescently labelled secondary antibodies for detection. FIG. 2 shows the expression of S, M, N and E proteins in HEK 293 cells, demonstrating that pCoVEG2 disclosed herein expresses the viral antigens in cells.

### Example 3: Expression of SARS-CoV-2 S, E, M, and N Proteins in Eukaryotic Cells Observed by SDS-PAGE and Western Blot

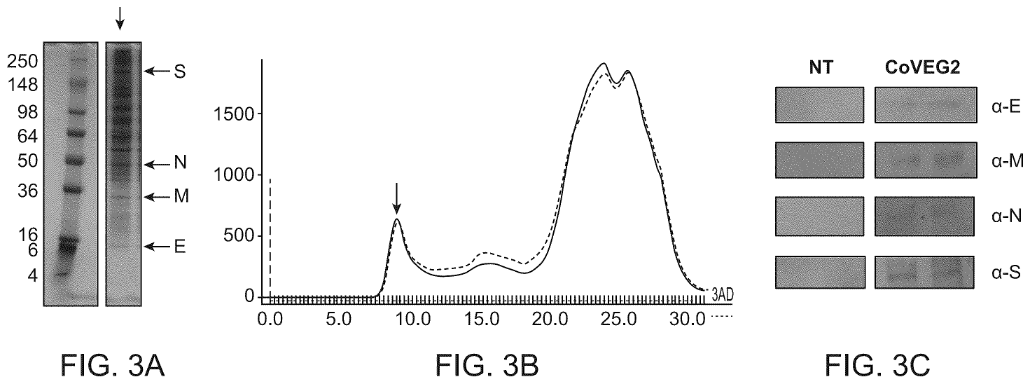

HEK 293 cells were transfected with the pCoVEG2 plasmid and incubated for 96 hours. Thereafter, cell culture supernatant was harvested and concentrated. The concentrate was run over Superose 6 GL resin packed in the Tricorn 10/300 column using PBS as eluant. The void fraction, which contains secreted VLPs, was analyzed by sodium dodecyl sulfate poly-acrylamide gel electrophoresis (SDS-PAGE) and/or western blotting using monoclonal antibodies against S, N, M or E to demonstrate the presence of S, N, M and E proteins. See FIG. 3.

These data demonstrate that the DNA vector CoVEG2 disclosed herein expresses all SARS-CoV-2 viral structural proteins (S, M, E, and N proteins) in HEK 293 cells and that secreted VLPs components can be detected in cell culture supernatants. These results suggest that CoVEG1 and CoVEG2 plasmids could potentially be used as highly effective DNA vaccines against SARS-CoV-2.

### Example 4: Immunogenicity of Polynucleotide Vaccine

To determine the immunogenicity of CoVEG1 and CoVEG2, these plasmids are injected intradermally into 6 weeks old BALB/c mice in 2 week intervals, for a total of 3 injections at Day 1, 15, and 29. The elicited humoral immune response [the titer of anti-S antibody using a respective enzyme linked immunosorbent assay (ELISA)] as well as cellular immune response [the presence of antigen reactive T cells using a respective IFN-γ and IL-4 enzyme-linked immune absorbent Spot (Dual color ELISpot) assay] is measured. To measure the neutralizing versus total antibodies, in vitro viral neutralization assays are performed. For this, isolated serum from day 43 is diluted and incubated with SARS-CoV-2 life virus before adding to VERO cells. Virus isolation is determined by the absence of successful infection of the cells compared to the native virus.

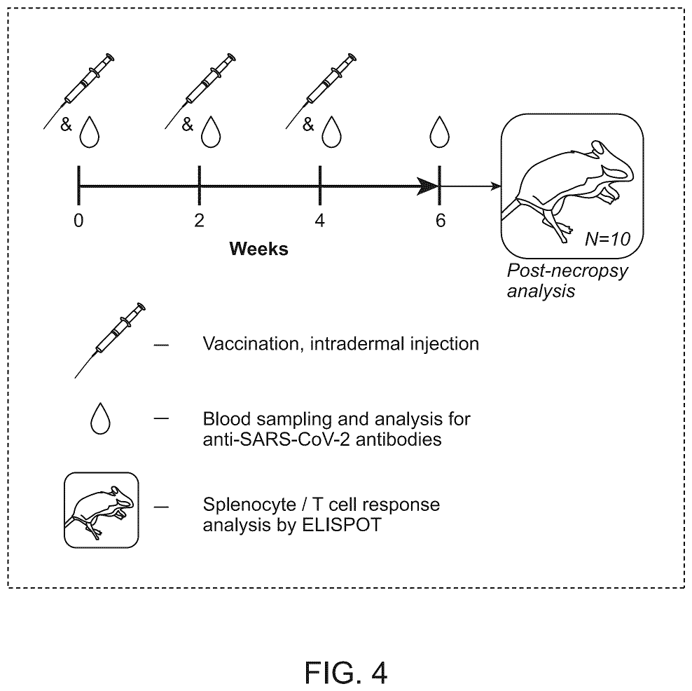

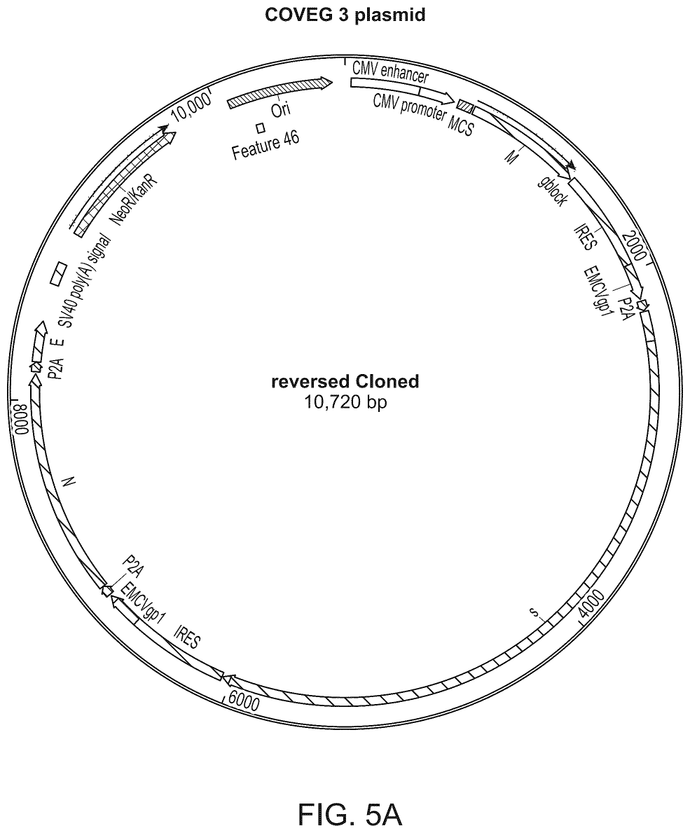

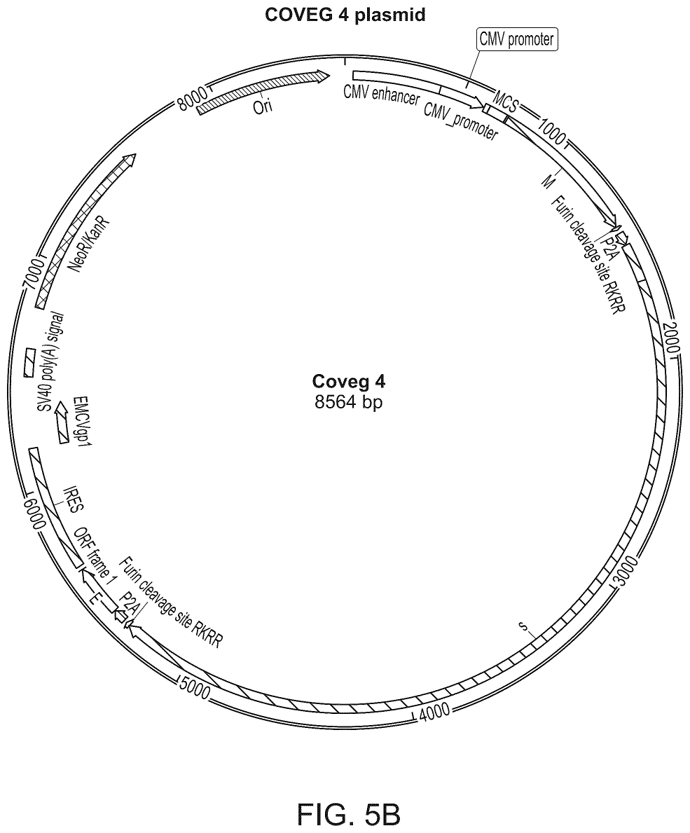

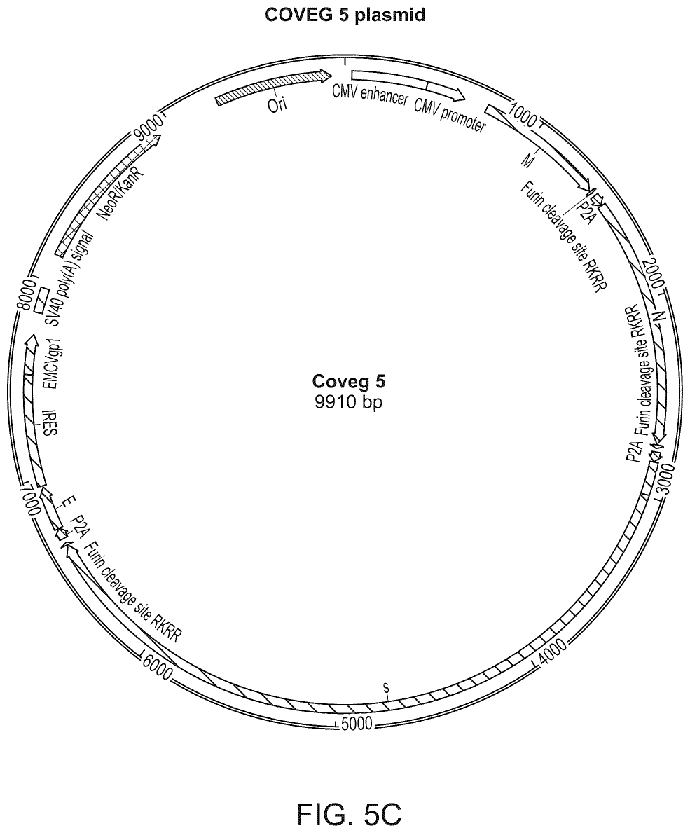

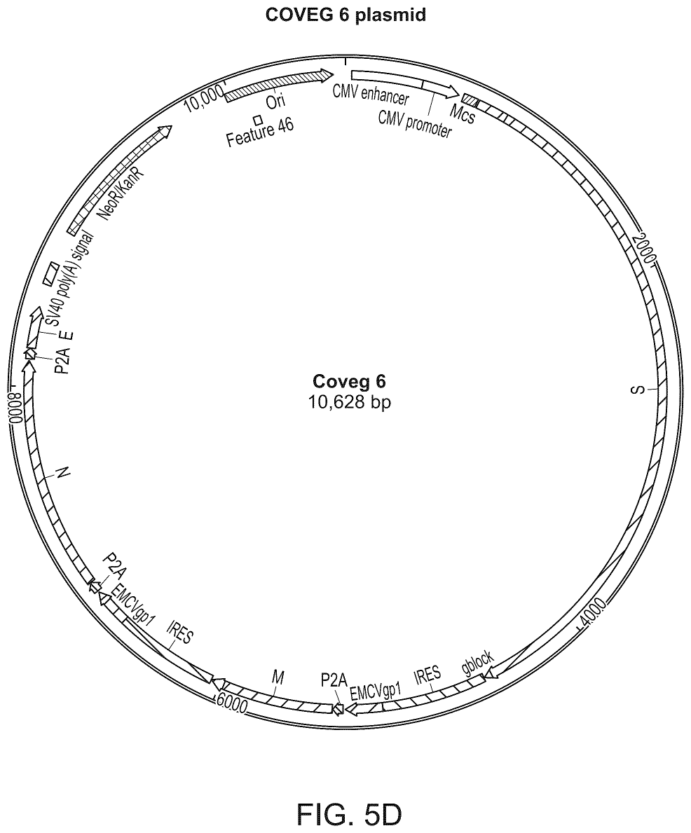

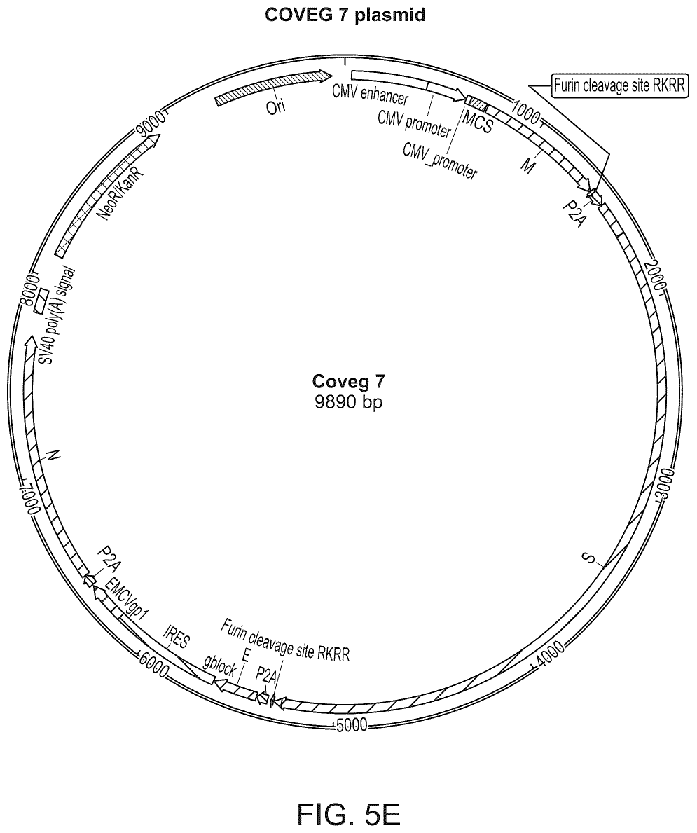

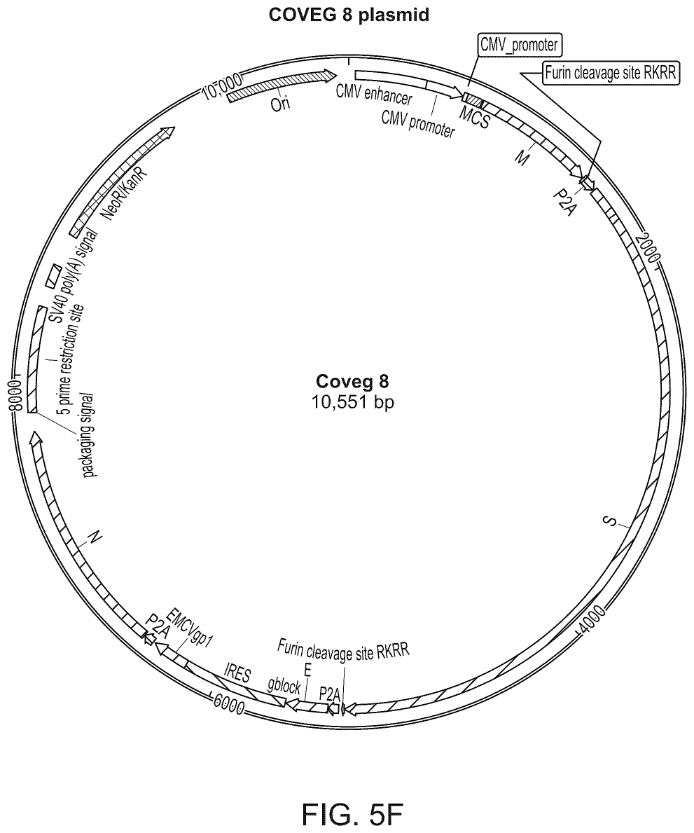

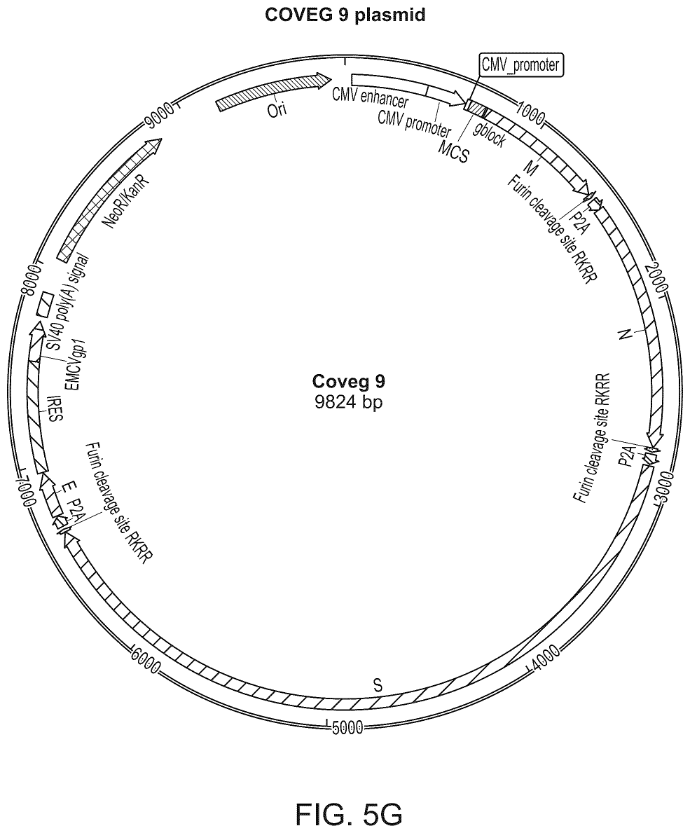

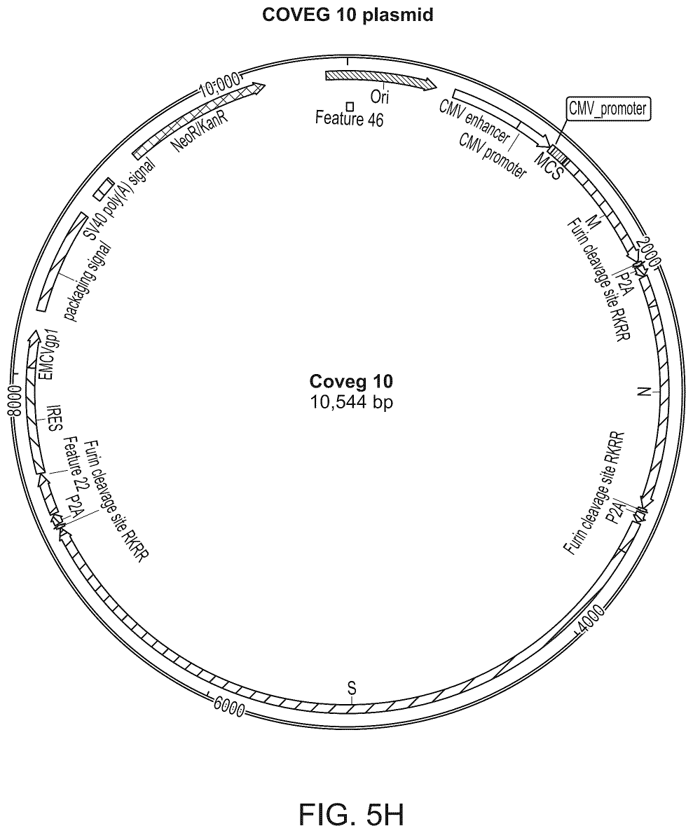

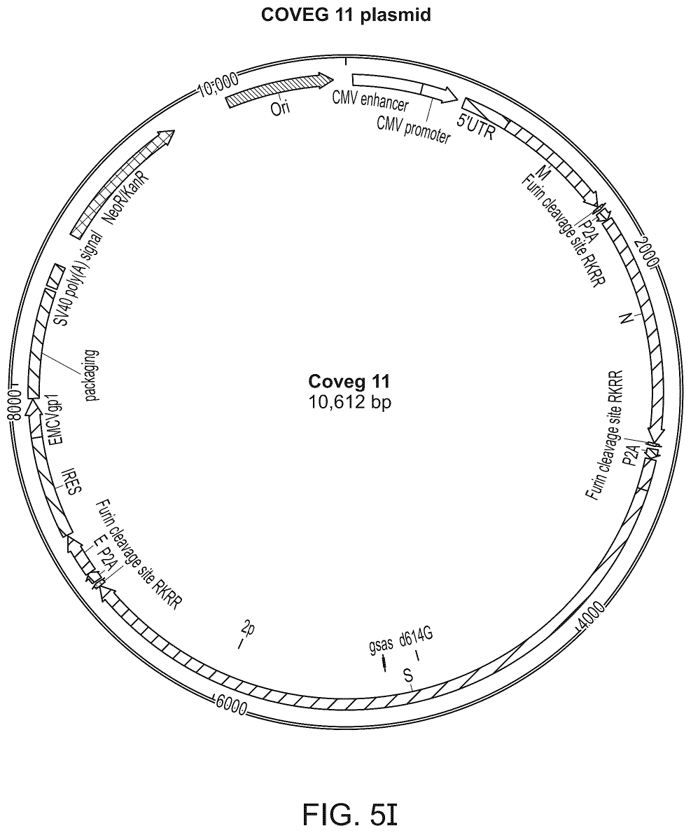

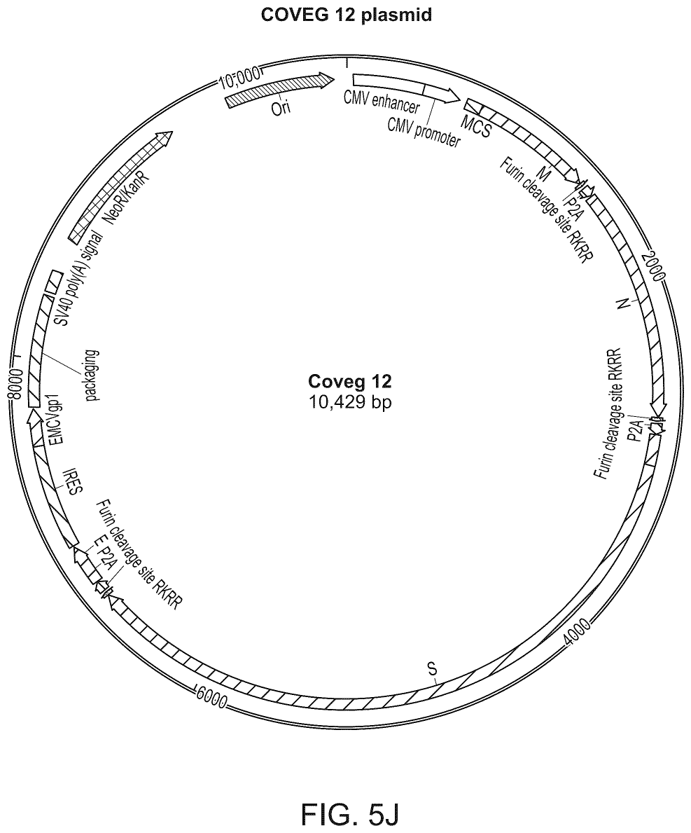

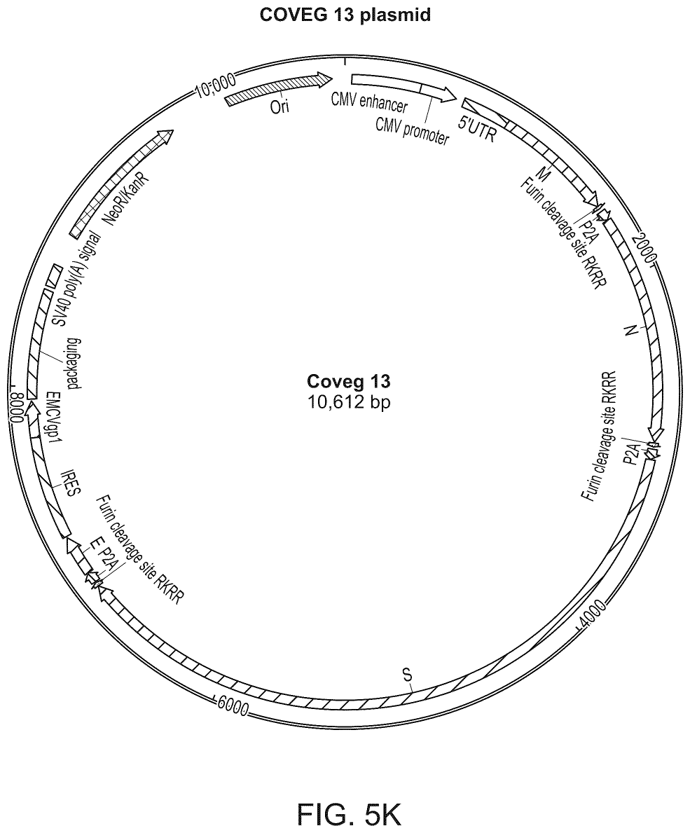

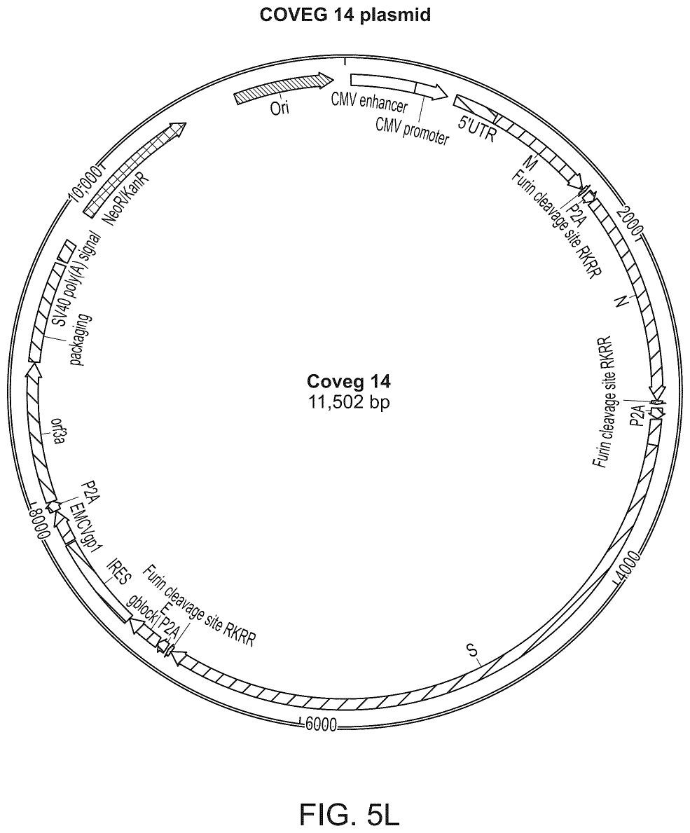

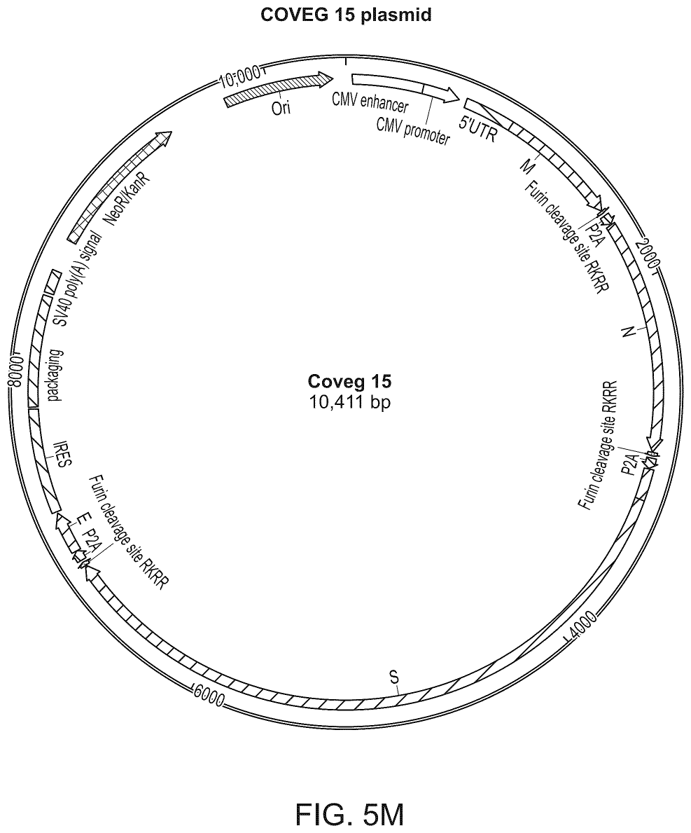

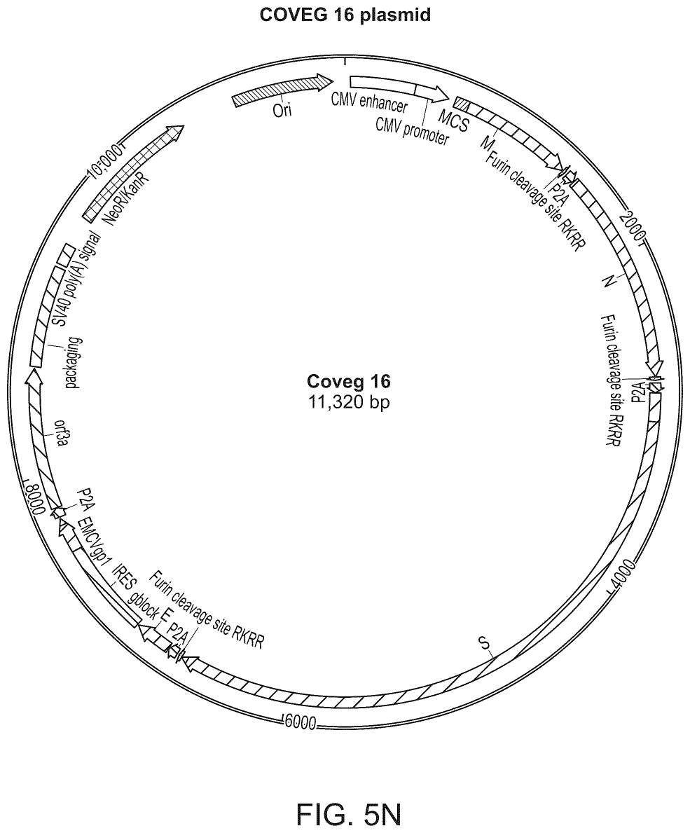

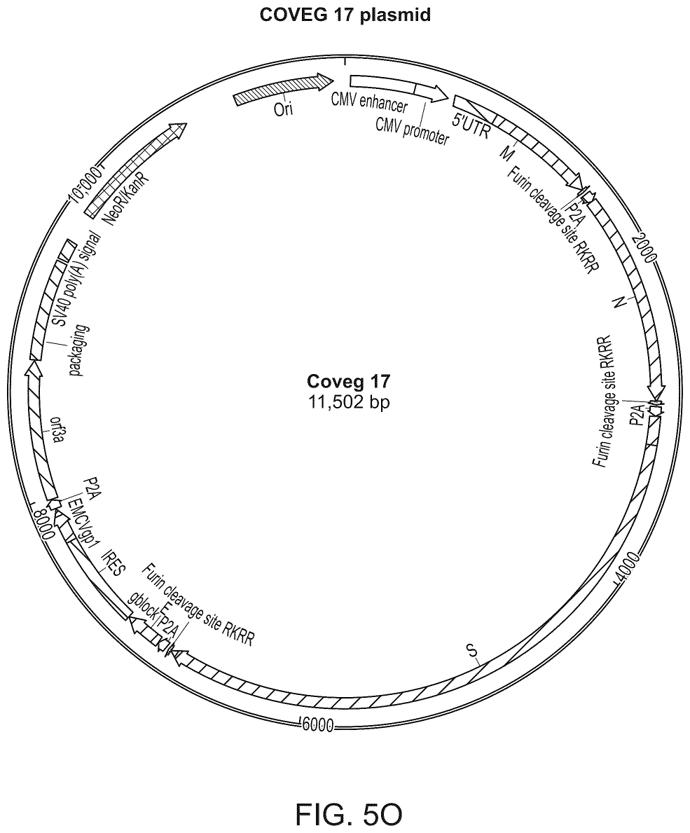

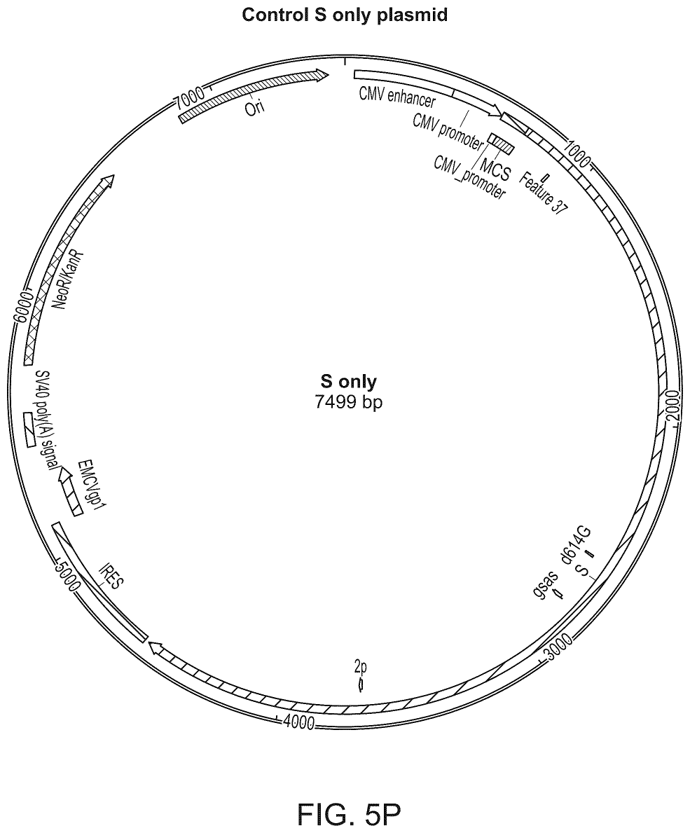

Anti-SARS-CoV-2 antibody analysis comprising anti-S protein antibody ELISA assay is performed based on commercially available materials. Alternatively, in-house developed cell-based and VLP-based ELISA assays is used. For ELISpot analysis, spleen is collected and T cells are isolated. ELISpot assessment is performed by priming the T cells with Miltenyi Biotec Peptivator SARS-CoV-2 peptide pools to activate SARS-CoV-2 reactive T cells. In addition, the toxicokinetic and pharmacodynamic characteristics of the plasmids are determined. See FIG. 4.

Female BALB/c mice (6-8 weeks of age) weighing 15 to 25 grams are randomly assigned to 4 groups with each group containing 10 animals. Mice are dosed intradermally with either the vehicle—PBS, a reference item EG-BB, which encodes the enhancer protein(s) under the control a CMV promoter, and two doses of CoVEG1 and CoVEG2 at 1 and 25 μg. Mice are evaluated twice daily for mortality and moribundity. Clinical observations and body weights are collected weekly starting Week-1 and thereafter at least every 2 weeks during the study period.

Dosed mice are bled at pre-defined timepoints before dosing and serum are separated by centrifugation. The obtained serum samples are then analyzed for antibodies against the full length recombinant S protein (S1+S2) using a quantitative ELISA, as shown below in Table 3.

TABLE 3
Anti-Vaccine Antibody sample collection

| | Time Points | | |
|---|---|---|---|
| Group Nos. | Day 15^{a} | Day 29^{a} | Day 43 |
| 1-4 | X | X | X |

| | |
|---|---|
| Method/Comments: | Jugular venipuncture (Day 15 and 29) or from |
| | the abdominal aorta under isoflurane anesthesia |
| | (Day 43 and at unscheduled euthanasia) |
| Target Volume ^{b} (mL): | 0.12 mL (Day 15 and 29) or as much as possible |
| | (Day 43 or unscheduled euthanasia) |
| Anticoagulant: | None, in SST |
| Special Requirements: | None |
| Processing: | Serum |

X = Sample collected; SST = serum separator tube
^{a}Sample collected before dosing.
^{b} Additional blood samples obtained (e.g., due to sample quality) if permissible sampling frequency and blood volume are not exceeded.

For the Day 43 time point, the resultant serum is split into 2 approximately equal aliquots; the first aliquot will be used for anti-vaccine antibody (AVA) analysis and the second aliquot kept for testing for neutralizing antibodies. The aliquots are frozen immediately over dry ice or in a freezer set to maintain −80° C.

At the end of the study, all animals are euthanized. Spleens are collected using cell culture clean procedures for IFN-γ and IL-4 evaluation by ELISpot.

For evaluation of T-cell mediated toxicity, a quantitative assessment is performed using ELISpot assay. Splenocytes from harvested spleens are stimulated with Miltenyi Biotec's SARS-CoV-2 PepTivator Peptide Pools which covers the sequence of 5, M and N SARS-CoV-2 proteins. Splenocytes are tested at 2 concentrations of 3 different SARS-CoV-2 peptide pools in addition to a negative (medium) and positive control (Phorbol Myristate Acetate/Ionomycin).

### Example 5: Immunogenicity of Polynucleotide Vaccine in Humans

To assess the safety, reactogenicity and immunogenicity of CoVEG1 and CoVEG2, an open-label, multi-center, dose-ranging study is conducted in males and non-pregnant females, starting at 18 years of age, inclusive, who are in good health and meet all eligibility criteria. Approximately 45 subjects are enrolled into one of three cohorts (1, 25, and 200 μg). Subjects receive an intradermal injection (100 μl) of CoVEG1 and CoVEG2 on Days 1 and 29 and are followed through 12 months post second vaccination (Day 394). Follow-up visits occur in 1, 2 and 4 weeks post each vaccination (Days 8, 15, 29, 36 and 57), as well as 3, 6- and 12-months post second vaccination (Days 119, 209 and 394).

The safety and reactogenicity of 2-dose vaccination schedule of CoVEG1 and CoVEG2 administered as intradermal injection, given 28 days apart, across 2 dosages in healthy adults is evaluated based on the percentage of Participants with Adverse Events (AEs), percentage of Participants with Administration (Injection) Site Reactions, and percentage of Participants with Adverse Events of Special Interest (AESIs).

To evaluate immunogenicity, the following parameters are assessed following a 2-dose vaccination schedule of CoVEG1 and CoVEG2, at Day 15, Day 29 (before the second dose) and at Day 57:
(a) IgG, IgM and/or IgA ELISA to the S antibody by a validated ELISA method;
(b) Immune memory indications are assessed by immune phenotyping PBMCs by flow cytometry and validated ELISpot assay focusing on CD49b+T-bet+resting memory Th cell precursors and CXCR4+S1P1+ memory plasma cell precursors cells on Day 15, Day 29, Day 57, Day 180, Day 270 and/or Day 394. The measurements include: Geometric mean fold rise (GMFR) in IgG, IgM and/or IgA titer from baseline (Day 1 to Day 394); Geometric mean titer (GMT) of antibody (Day 15, Day 29, Day 57, Day 180, Day 270 and Day 394); and Percentage of subjects who seroconverted (Day 1 to Days 15, 29, 57, 180, 270 and 394). Seroconversion is defined as 4-fold change in antibody titer from baseline; and
(c) IFN-γ response as a measure of CD8 T cell response and phenotype of memory immune cells will be measured in PBMC isolated from subjects by a validated ELISpot assays. PBMC will be isolated on Days 15, 29, 57, 180, 270 and 394.

### Example 6: Design of Further Polynucleotides for Expression of SARS-CoV-2 S, E, M, and N Proteins and Mutants with and without the Enhancer L Protein

Plasmids CoVEG 3-17 comprise expression cassettes encoding different viral proteins in the order indicated in FIG. 6. The plasmid backbone (based on the design principles of the pVaxl plasmid) and insert for the plasmids were generated using gene synthesis. The plasmid backbone consists of a Kanamycin resistance gene, the ColE1 origin of replication, the Human cytomegalovirus immediate-early promoter and Simian virus (SV40) Poly A signal. Polynucleotides encoding viral proteins were cloned in between the CMV promoter and the SV40 PolyA signal. After gene synthesis and plasmid preparation, the plasmid was transformed into E. coli for cloning and then screened using kanamycin. A representative colony was selected, and its plasmid sequence verified and used as source plasmid for further development. After transcription, the viral proteins were expressed from a single polycistronic mRNA.

### Example 7: Expression of SARS-CoV-2 S and N Proteins Observed by Immunofluorescence

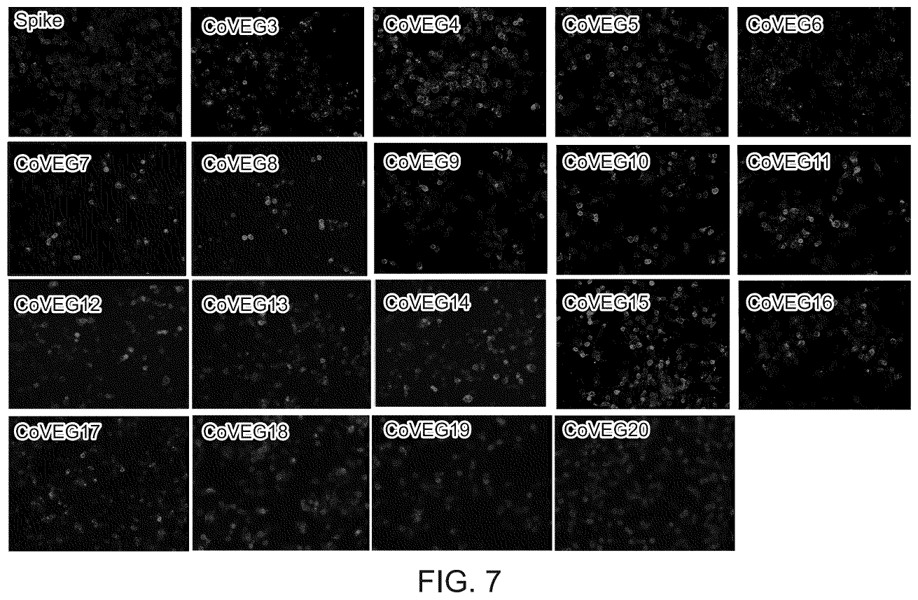

HEK293T cells were seeded at 40,000 cells/well in a 24 well plate 24h prior to transfection. Cells were transfected with the pCoVEG 3-20 plasmids using PEI complexes following manufacturers description. Media was changed 12 hours after transfection and cells were incubated at 37° C., 5% CO₂ for 48 h. Cell media was removed, and cells were fixed with 10% neutral buffered formalin for 10 minutes following permeabilization with 0.2% Triton X-100 in PBS for 10 min. Unspecific binding was blocked by adding EZ block (SCYTEK) before immunostaining was performed. Stain with an anti-spike (S) protein antibody that binds to the receptor binding domain (RBD)—also referred to herein as “anti-RBD”—was added at a dilution of 1:500 and incubated for 1 hour at room temperature. The stain was removed, cells were washed and secondary antibody (Alexa Fluor 568 fluorescently labelled secondary anti-Rabbit, 1:1000 dilution) was added. The stain was incubated for 1 hour at room temperature before removal of the stain and washing. Cells were imaged using a EVOS cell imaging system. FIG. 7 shows the expression of the S protein in HEK293 cells, demonstrating that all tested CoVEG plasmids were capable of expressing the spike protein.

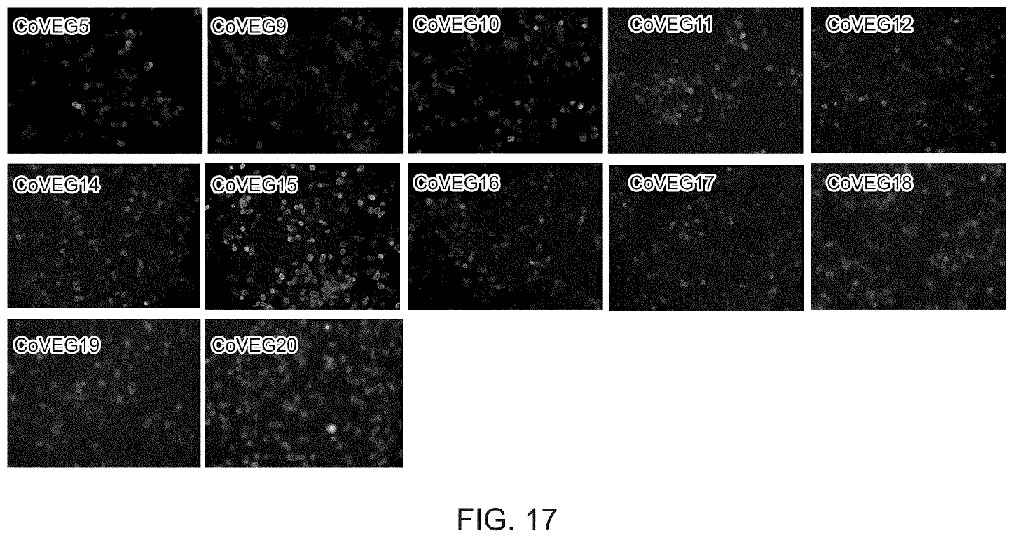

Additionally, to check for the expression of VLPs, cells were analyzed for expression of the nucleocapsid (N) protein using immunofluorescence staining. For this experiment, HEK293T cells were seeded at 40,000 cells/well in a 24 well plate 24 hours prior to transfection. Cells were transfected with the pCoVEG 5, 9-12, and 14-20 plasmids using PEI complexes following the manufacturers description. The media was changed 12 hours after transfection and cells were incubated at 37° C., 5% CO₂ for 48 hours. The cell media was removed, and cells were fixed with 10% neutral buffered formalin for 10 minutes following permeabilization with 0.2% Triton X-100 in PBS for 10 minutes. Unspecific binding was blocked by adding EZ block (SCYTEK) before immunostaining. Stain with anti-nucleocapsid (N) protein antibody was added at a dilution of 1:1000 and incubated for 1 hour at room temperature. The stain was removed, cells were washed and secondary antibody (Alexa Fluor 488 fluorescently labelled secondary anti-mouse, 1:1000 dilution) was added. The secondary antibody was incubated for 1 hour at room temperature before removal of the stain and washing. Cells were imaged using a EVOS cell imaging system. FIG. 17 shows the expression of the N protein in HEK293 cells, demonstrating that all tested CoVEG plasmids were capable of expressing the nucleocapsid protein.

### Example 8: L Protein Required for Detectable SARS-CoV-2 VLP Formation

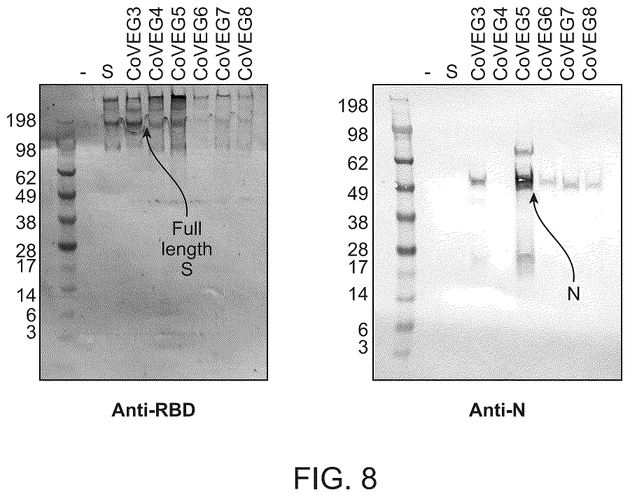

To isolate intact viral-like particles (VLPs), 4×10⁶ HEK293 cells were transfected with the pCoVEG 3-20 plasmids in a 150 mm dish using PEI complexes following manufacturers description. The media was changed 12 hours after transfection and cells were incubated at 37° C., 5% CO₂ for 72 hours. VLP containing supernatants were harvested, spun down (1,500×g, 15 min) and concentrated using an Amicon centrifugal filter unit (100 kDa cut off). Concentrate was spun down (4,500×g, 15 minutes) to remove precipitate and VLPs were pelleted (100,000×g, 1.5 hours) through a 20% sucrose cushion. VLPs were resuspended in PBS and analyzed by western blot, as shown in FIG. 8. FIG. 8 shows that CoVEG 3-8 plasmids were capable of expressing the S protein and CoVEG 3 and 5-8 plasmids were capable of expressing the N protein. These data demonstrate that the DNA vectors CoVEG 3, and 5-8 disclosed herein express SARS-CoV-2 viral structural proteins necessary for the formation of VLPs in HEK 293 cells and that secreted VLPs components can be detected in cell culture supernatants.

FIG. 11 shows the expression of S protein and N protein from CoVEG 5, 9, 11, 16, 10, and 15 plasmids. The results showed that expressing the mutant S protein (from CoVEG 9, 11, 10, and 15) increased the amount of Spike protein expressed and presented on VLPs. Expression of ORF3 protein (from CoVEG 16) appeared to decrease the amount of S and N proteins in the VLPs. Absence of the enhancer L protein upon expression of the CoVEG 15 plasmid resulted in a similar amount of S protein, but a far greater amount of N protein. Without being bound by a theory, it is thought that in the absence of the enhancer L protein, a higher amount of N protein is expressed resulting in unbalanced VLP formation.

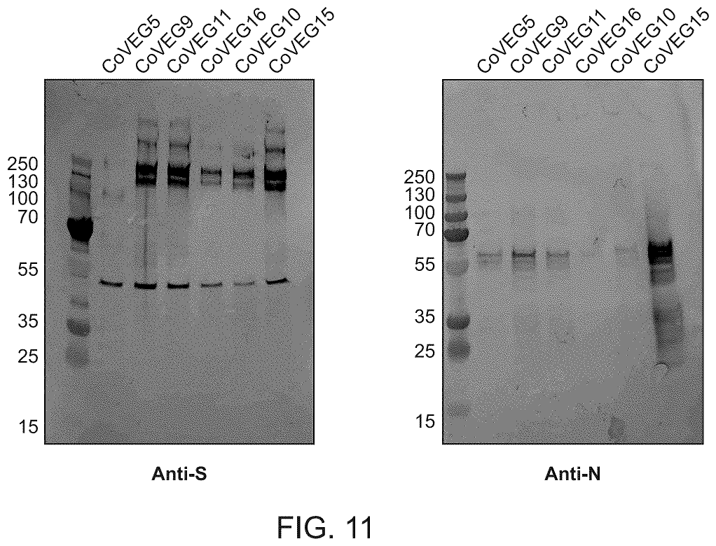

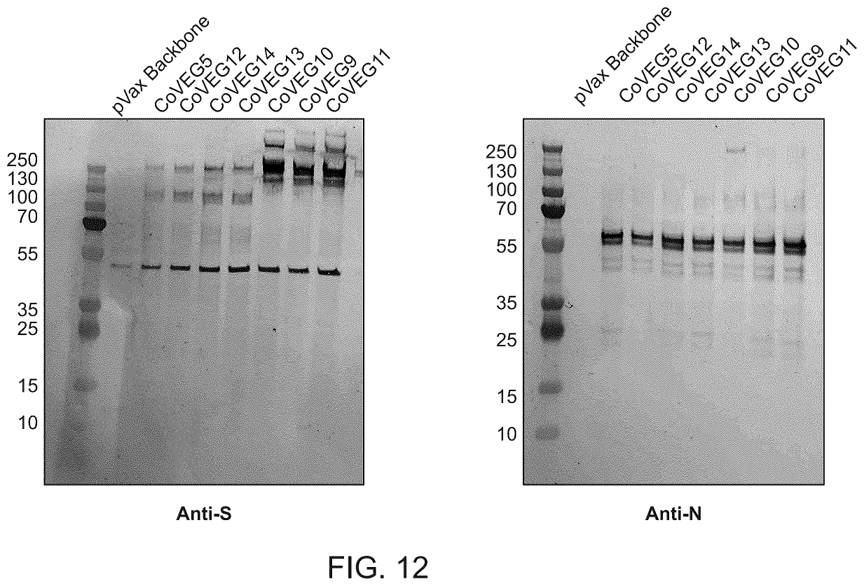

FIG. 12 shows the expression of S protein and N protein from CoVEG 5, 12, 14, 13, 10, 9 and 11 plasmids. These data demonstrate that expressing the mutant S protein (from CoVEG 9, 10, and 11 plasmids) increased the amount of Spike protein expressed and presented on VLPs, as compared to the comparator plasmids that expressed wild type S protein, while the amount of N protein appeared constant across plasmids.

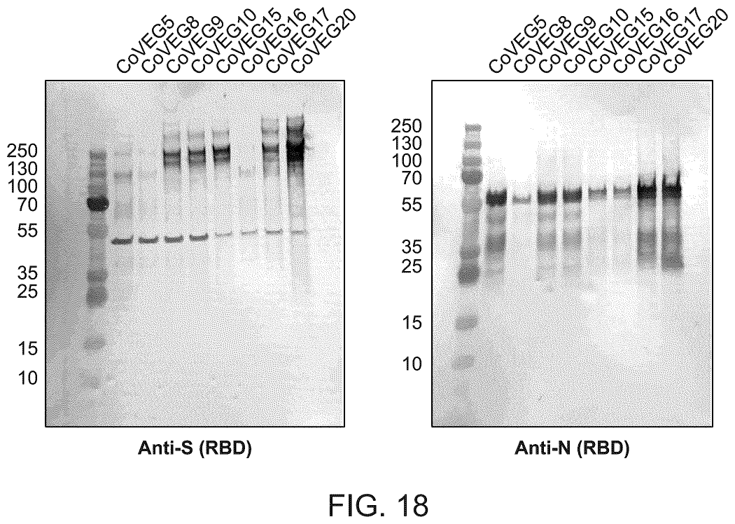

FIG. 18 shows the expression of S protein and N protein from CoVEG 5, 8, 9, 10, 15, 16, 17 and 20 plasmids. Notably, the presence of the enhancer L protein resulted in different S and N protein expression ratios, e.g. as shown by the CoVEG 10 (L protein) versus the CoVEG 20 (no L protein) S and N western blotting in FIG. 18.

The presence of secreted VLPs were also confirmed by ELISA. For this experiment, 24,000 HEK293 cells were transfected with pCoVEG 5 and 9-14 plasmids or plasmids containing either the Spike protein or the Nucleocapsid protein as controls. Experiments were performed in a 24 well plate using PEI complexes following the manufacturer's descriptions. The media was changed 12 hours after transfection and cells were incubated at 37° C. and 5% CO₂ for 72 hours. VLP containing supernatants were harvested, spun down (1,500×g, 15 min) and 75 μl of the cleared supernatant was used to coat ELISA plates over night at 4° C. After incubation, the plates were washed twice with 0.05% Twen-20 in PBS and wells were blocked using EZ block (2 hours at 37° C.). The plates were washed twice with 0.05% Tween-20 in PBS. To detect VLPs in the coating material, anti-RBD (Sino Biological, mouse anti-RBD SARS-CoV-2 (2019-nCoV) Spike Neutralizing Antibody, Mouse Mab, 40592-MM57 SARS-CoV-2, 1:500 dilution in EZ Block) or anti-N(Novus Biologicals, Mouse anti-SARS-CoV-2 Nucleocapsid, Clone: B3449M, N2787B09, 1:1000 dilution in EZ Block) were added to the wells. Antibodies were incubated for 1 hour at room temperature before washing three times with 0.05% Tween-20 in PBS and adding 75 μl of secondary antibody (Goat-Anti-mouse, HRP-conjugate, 1:2,000 dilution, Southern Biotech, Goat anti-Mouse IgG(H+L), horseradish peroxidase (HRP), Polyclonal, OB103405) and incubating for 1 hour at room temperature. Wells were thoroughly washed (5x with 0.05% Tween-20 in PBS), and binding was developed using 75 μl 3,3′,5,5′-tetramethylbenzidine (TMB) substrate (Surmodisc Inc TMB One Component HRP Microwell Substrate). The reaction was carried out for 30 minutes with 75 μl Stop Solution (Surmodisc Inc 450 NM LIQ STOP REAGENT) and Absorbance was measured at 450 nm.

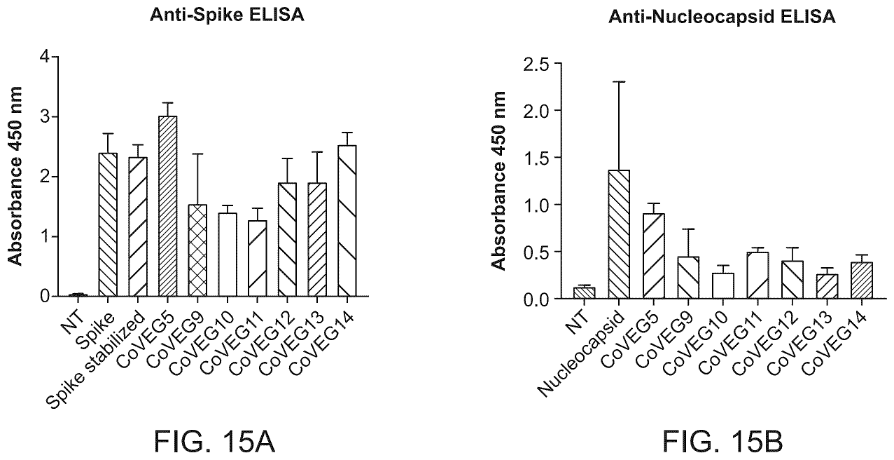

FIG. 15 shows ELISA results from VLP secretion of CoVEG 5 and 9-14 plasmids compared with single protein S and N expressing vectors. Both Spike and Nucleocapsid proteins secreted from HEK293 cells. However, while the S protein demonstrated high ELISA VLP signal relative to single protein expression, the N protein demonstrated a notably lower VLP signal relative to single protein expression. It may be that N signal in VLPs is lower than the S signal in VLPs because the N protein is on the interior of the VLP and not accessible to the antibody.

To further ensure that the expressed structural proteins from SARS-CoV2 were forming intact VLP, 20×10⁶ HEK293 cells were transfected with the pCoVEG 10 plasmid in 5-150 mm dishes using PEI complexes following manufacturers description. The media was changed 12 hours after transfection and cells were incubated at 37° C., 5% CO₂ for 72 hours. VLP containing supernatants were harvested, spun down (1,500×g, 15 minutes) and concentrated using an Amicon centrifugal filter unit (100 kDa cut off). Concentrate was spun down (4,500×g, 15 min) to remove precipitate and VLPs were pelleted (100,000×g, 1.5 h) through a 20% sucrose cushion. VLPs were resuspended in PBS and used for co-Immunoprecipitation (co-IP). For the co-IP resuspended VLPs were incubated with anti-S RBD antibody (Sino Biological, mouse anti-RBD SARS-CoV-2 (2019-nCoV) Spike Neutralizing Antibody, Mouse Mab, 40592-MM57 SARS-CoV-2) for 60 minutes before adding 100 μl washed Protein A/G agarose resin (Thermo Fisher scientific). Resin was incubated for 120 minutes before eluting with 0.1M glycine pH 2. Eluates were immediately neutralized by adding 5 times volume of 1M Tris pH 8.0. Fractions were analyzed for the presence of N protein by western blot using anti-N antibody (Novus Biologicals, Mouse anti-SARS-CoV-2 Nucleocapsid, Clone: B3449M, N2787B09).

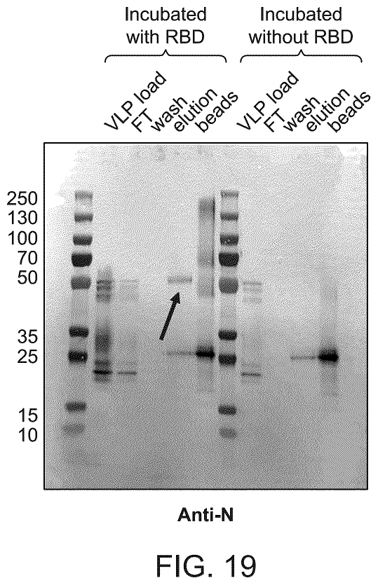

FIG. 19 shows the western blot result of the co-IP experiments of the CoVEG 10 plasmid. The N-protein is packed in intact VLPs as demonstrated by the presence of an N-signal in the elution fraction after incubation with RBD (left side, arrow). This signal, compared with the absence of signal in the washing fraction, indicates the N protein is retained within the particles. As a control for the possibility of non-specific N protein binding to the outside of particles, the co-IP was run in parallel without the anti-RBD antibody (right side). The N protein signal was not detectable in the elution fraction, demonstrating that the N protein did not bind the resin non-specifically.

To visualize the secreted VLPs, 20×10⁶ HEK293 cells were transfected with the pCoVEG 10 and 20 plasmids in 5×150 mm dishes using PEI complexes and following the manufacturer's description. The media was changed 12 hours after transfection and the cells were incubated at 37° C., 5% CO₂ for 72 hours. VLP containing supernatants were harvested, spun down (1,500×g, 15 minutes) and concentrated using an Amicon 100 kDa centrifugal filter unit. The concentrate was spun down (4,500×g, 15 minutes) to remove precipitate and VLPs were pelleted (100,000×g, 1.5 hours) through a 20% sucrose cushion. VLPs were resuspended in PBS, flash frozen, and stored at −80° C. until used for transmission electron microcopy (TEM).

For the TEM experiments, VLPs were ultracentrifuged for 2 hours at 25000 g on a 20% sucrose cushion using a TLS-55 (Optima TLX Ultracentrifuge, Beckman). 10 μl were put on a microscopy copper grid (Sigma Aldrich) and fixed with 2% (v/v) paraformaldehyde for 5 minutes. Samples were then negatively stained with 5 mL of phosphotungstic acid (Sigma Aldrich). The grid was examined with a Hitachi HT7700 TEM operating at 100 KeV.

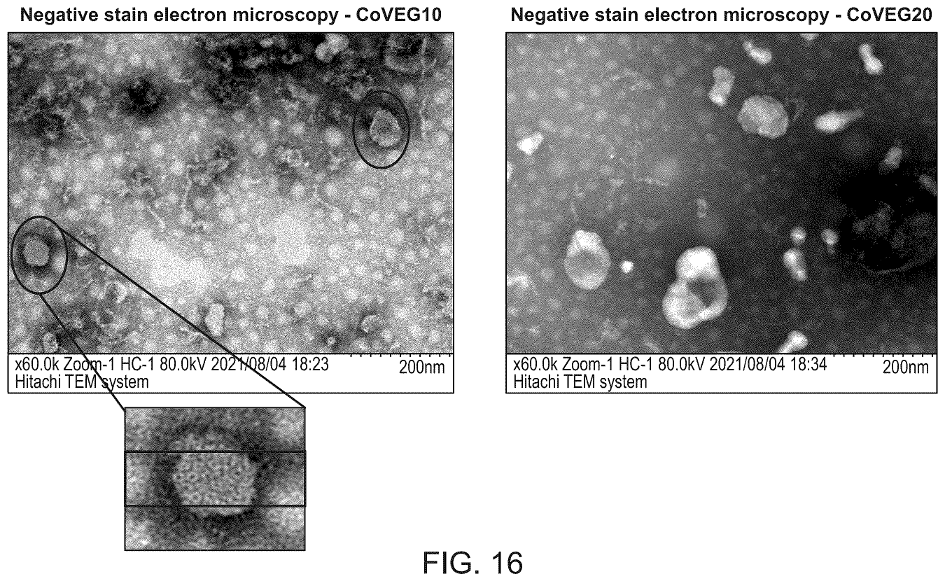

FIG. 16 shows the presence of VLPs in the isolated material for CoVEG 10. The presence of a larger particle with a clear Spike trimerization surface could be observed for CoVEG10 (see zoom inset). CoVEG 20 which is identical to CoVEG10 apart from the presence of the L regulatory protein, failed to generate recognizable VLPs.

Intact and immunogenic VLPs are highly dependent on the ratio of all VLP forming proteins. Herein it was demonstrated that the L protein controlled expression of all VLP forming proteins and the correct formation of the VLPs.

### Example 9: L Protein Increased Neutralizing Antibodies In Vivo to SARS—CoV-2 Proteins and was Required for Th1 Response

To determine the immunogenicity of plasmids CoVEG 3-8, the plasmids were diluted to 1 mg/ml in PBS and 50 μl was injected intramuscularly into 6 week old BALB/c mice in 2 week intervals, for a total of 2 injections at day 1 and 15. Blood was collected on days 14, day 28, day 42 and day 56, and the serum was isolated and snap frozen in the presence of an anti-coagulant.

To determine the immunogenicity of the plasmids CoVEG 5, 8 and 9-14 as well the as S-only plasmid, the plasmids were diluted to 2 mg/ml or 0.5 mg/ml in PBS and 50 μl was injected intramuscularly (2 mg/ml) or intradermally (0.5 mg/ml) into 6 week old BALB/c mice in 2 week intervals, for a total of 2 injections at day 1 and 15. Blood was collected on day 14, day 28, day 42 and day 56 and the serum was isolated and snap frozen in the presence of an anti-coagulant.

To determine immunogenicity of plasmids CoVEG 9, 10 and 20, as well as the S only plasmid with and without the enhancer protein, the plasmids were diluted to 2 mg/ml or 0.2 mg/ml in PBS and 50 μl was injected intramuscularly into 6 week old C57BL/6 mice in 2 week intervals, for a total of 1-3 injections at day 1, 15 and 29. Blood was collected on day 0, day 14, day 28, day 42, day 56 and day 70 and the serum was isolated and snap frozen in the presence of an anti-coagulant. To measure the binding antibody concentration, i.e., the elicited humoral immune response, enzyme linked immunosorbent assays (ELISA) were performed using purified SARS-CoV-2 Spike RBD protein (Creative Diagnostics@ DAGC149 Recombinant SARS-CoV-2 Spike Protein Receptor Binding Domain [His]) as a coating material. For this experiment, high-binding 96-well plates were coated with 75 μl of a 2 μg/ml SARS-CoV-2-Spike RBD solution, and plates were incubated over-night at 4° C. After incubation, plates were washed twice with 0.05% Tween-20 in PBS, and wells were blocked using EZ block (2 hours at 37° C.). Plates were washed twice with 0.05% Tween-20 in PBS. Serum was collected from the mice after 56 days and added to the wells (1:500 dilution for binding antibody detection, 1:100-1:7812500 for Endpoint Titer measurement). Serum was incubated for 1 hour at room temperature before washing thrice with 0.05% Tween-20 in PBS and adding 75 μl of secondary antibody (Goat-Anti-rabbit, HRP-conjugate, 1:4,000 dilution) and incubating for 1 hour at room temperature. Wells were thoroughly washed (5x with 0.05% Tween-20 in PBS) and binding was developed using 75 μl 3,3′,5,5′-tetramethylbenzidine (TMB) substrate (Surmodisc Inc TMB One Component HRP Microwell Substrate). The reaction was carried out for 30 minutes before stopping with 75 μl Stop Solution (Surmodisc Inc 450 NM LIQ STOP REAGENT). The Absorbance was measured at 450 nm.

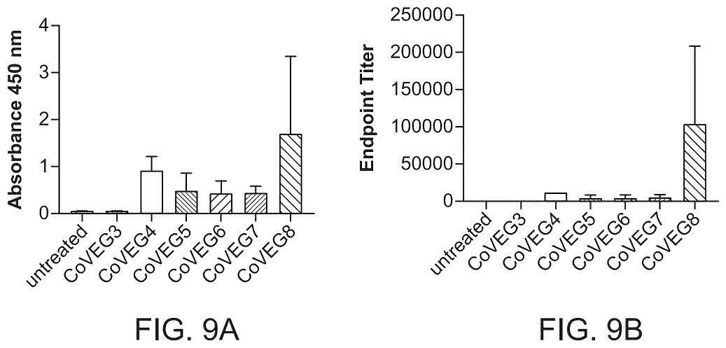

FIG. 9A shows the total binding antibody measured using ELISA, and FIG. 9B shows the measured endpoint titers. These results demonstrate that CoVEG 3-8 plasmids are capable of eliciting a strong immune response when injected into mice and thus, producing anti-SARS CoV2 antibodies. CoVEG 8 was identified as being particularly superior in being able to induce a strong immune response in these mice. These results demonstrate that the plasmids disclosed herein are highly effective DNA vaccines against SARS-CoV-2.

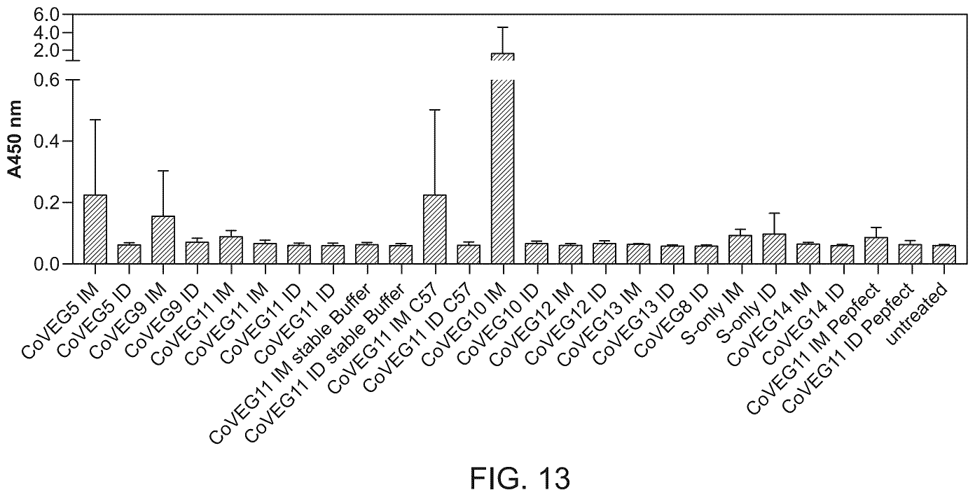

FIG. 13 shows ELISA results from injection of CoVEG 5, 9, 11, 10, 12, 13, 8 and 14 plasmids, either intramuscularly (IM) or intradermally (ID). Remarkably, the intramuscular injection of CoVEG10 induced a much higher immune response as compared to the injection of the Spike protein alone. Intramuscular injections of CoVEG5, CoVEG9, and CoVEG11 also induced a higher immune response as compared to the injection of the S protein alone. These results further demonstrate that the plasmids disclosed herein are highly effective DNA vaccines against SARS CoV2 that perform better than vaccines that express the Spike protein alone.

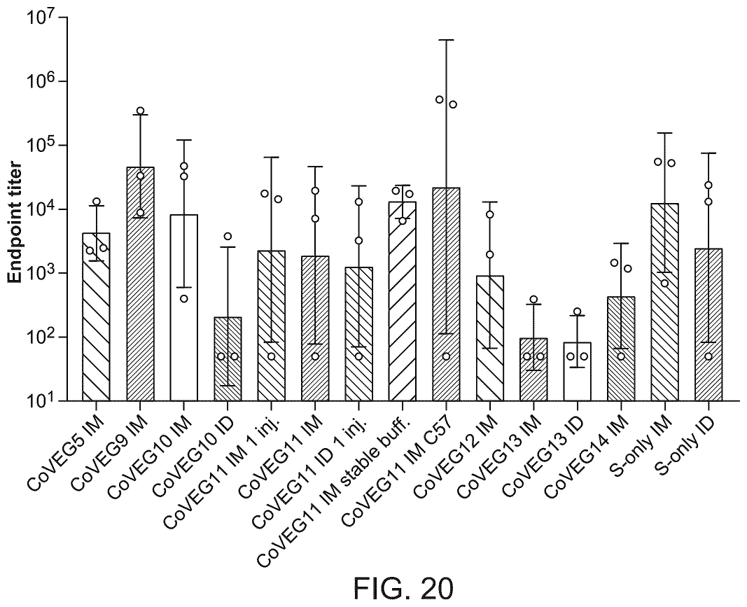

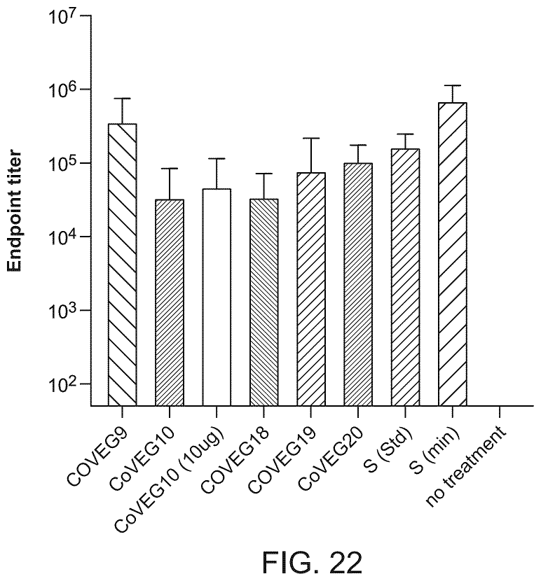

FIGS. 20 and 22 show antibody binding titers from CoVEG 5 and 9-14, 18, 19, and 20 plasmids, as well as S only, on day 42 after IM or ID injection. Interestingly, all of the tested CoVEGs were capable of inducing an immune response, with CoVEG 9 the most efficacious. Notably, the VLP forming CoVEG 9 performed better than the spike protein alone.

Additionally, the cellular immune response was measured using the presence of antigen reactive T cells using IFN-γ and IL-4 enzyme-linked immune absorbent Spot (ELISpot) assays. For ELISpot analysis, spleen was collected and T cells were isolated. ELISpot assessment was performed by priming the T cells with Miltenyi Biotec Peptivator SARS-CoV-2 peptide pools to activate SARS-CoV-2 reactive T cells. For the analysis, Mouse IL-4 Single color ELISPOT and Mouse INF-γ Single color ELISPOT (Immunospot, Cellular Technology Limited) were used according to manufacturer's instructions. In short, 96 well PVDF membrane plates were coated with IL-4 or INF-γ capture antibody and incubated over night at 4° C. After washing, 150,000 splenocytes in 100 μl CTL test medium, seeded on pre-coated plates, and incubated for 15 minutes at 37° C. and 4% CO₂. Cells were activated with either PMA/Ionophore (as a positive control) or 0.6 μl of reconstituted Miltenyi Biotec Peptivator SARS-CoV-2 peptide pools (S, N or M) per well. Reactions were incubated for 24 hours before developing and counting using an ImmunoSpot analyzer (CTL).

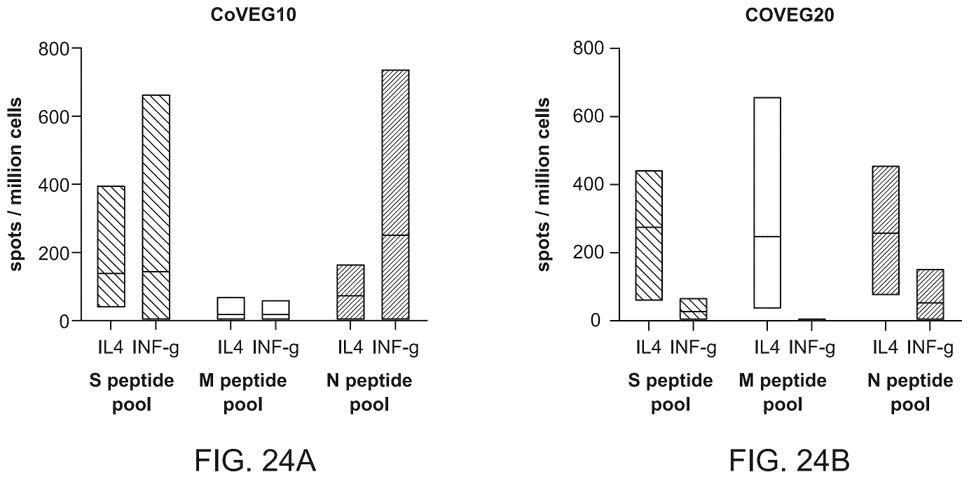

FIG. 24 shows the result of the T-cell analysis of CoVEG10 and CoVEG20. Importantly, to generate a successful vaccine against respiratory viruses e.g., SARS-CoV-2, a Th1 preference (INF-γ) or at least a balanced Th1/Th2 respond is needed. Surprisingly, the addition of the L regulatory protein in CoVEG10 influenced T-cellular immune response in favor of the INF-γ (Th1) response. Notably, the absence of the regulatory protein in CoVEG20 reversed the cellular response to IL4 (Th2). Th1 responses are necessary to generate a long-lasting immune response to a virus.

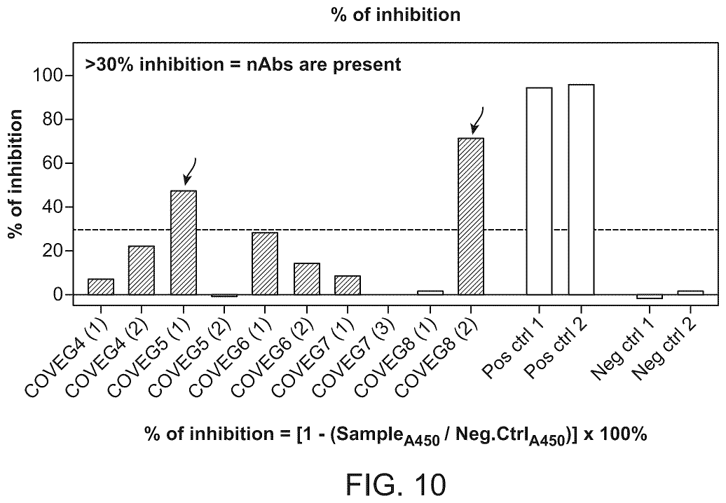

To test whether the serum has neutralizing antibodies against SARS CoV2 that bind to the Spike protein, in vitro viral neutralization assays using the cPass™ neutralization assay (GenScript) were performed according to manufacturer's instructions. The cPass™ allows the detection of total neutralizing antibodies in a sample by mimicking the interaction between the virus and the host cell in vitro. In the assay, if neutralizing antibodies are present in the sample being tested, then the binding of the receptor binding domain (RBD) to host cell membrane receptor, ACE2 is inhibited. However, if neutralizing antibodies are absent in the sample, then the RBD is able to bind to ACE2. FIG. 10 shows the percent (%) inhibition of RBD binding to the ACE2 receptor. If the inhibition of RBD binding to ACE2 is more than 30% (red dotted line), then the sample is identified as having neutralizing antibodies. As shown in FIG. 10, serum samples obtained from mice injected with COVEG5 and COVEG8 show a high percentage of inhibition, and therefore, generated neutralizing antibodies. These results further underline that the disclosed plasmids are highly effective DNA vaccines against SARS-CoV-2.

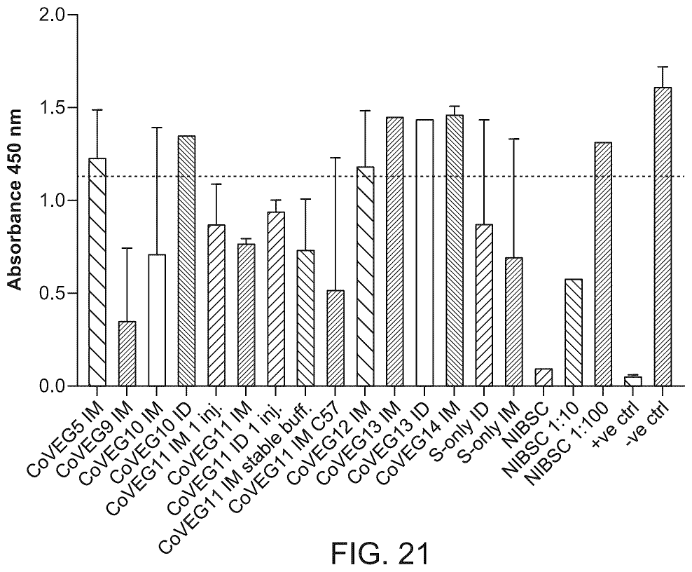

FIG. 21 shows the neutralizing antibodies of the samples shown in FIG. 20. These data indicate that not all generated antibodies possess neutralizing capacity. A lower signal in this assay confirmed neutralization as defined by a signal less than 70% of the negative control (dashed line). Signals lower than 30% of the negative control were confirmed to be strongly neutralizing. The tested CoVEGs performed differently depending on the design and the injection site. Surprisingly, ID injection did not induce strong neutralizing antibodies, whereas IM injections did induce strong neutralizing antibodies. Additionally, CoVEG13 and 14 did not meet the criteria for neutralization. However, CoVEG9 showed the highest neutralization of all tested constructs including the S spike protein only constructs, again demonstrating that the VLP approach provides a more potent vaccine than the sub-unit S spike only vaccines.

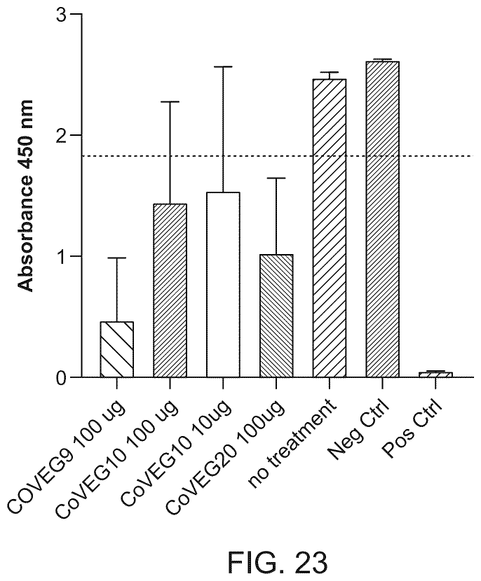

FIG. 23 shows neutralizing antibodies of CoVEG 9, 10 and 20 plasmids, in which plasmid 20 is the control for the absence of the enhancer L protein. Although CoVEG20 showed neutralizing potential, Th2 overhang as demonstrated in T-cell analysis, it is not a viable option for a vaccine.

Further, the neutralization capacity with and without the enhancer protein over time was analyzed by cPass™. SARS-CoV-2 Surrogate Virus Neutralization Test (sVNT) Kit. For this, serum samples from immunized animals (immunized with CoVEG9, Spike+ enhancer protein L and Spike without enhancer) were collected on day 42 and day 70 and cPass™ was performed as described above.

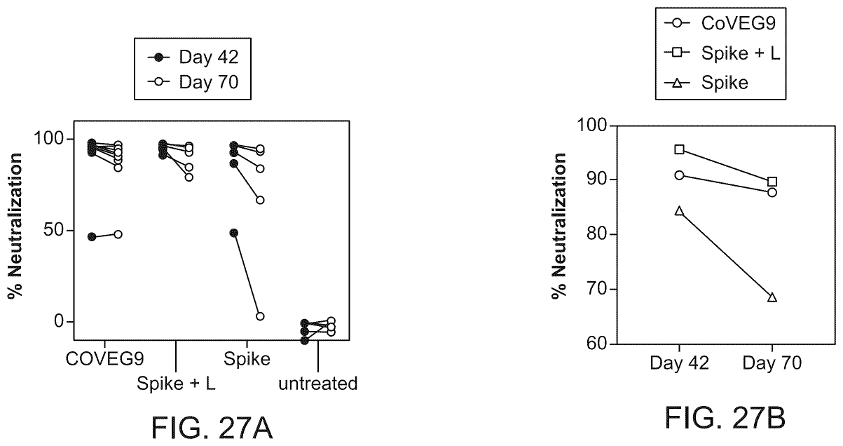

FIG. 27 A shows the individual values of the analyzed serum samples and FIG. 27 B shows the median of the same data as summary. Interestingly, the addition of the enhancer protein clearly showed a benefit of neutralizing antibodies over the time. Firstly, both constructs with the enhancer protein L (CoVEG9 and Spike+L) showed higher median neutralization values (FIG. 27B B, CoVEG9 circles, Spike+L squares). Secondly, the level of neutralizing antibodies remained high, even after 70 days post injection compared to the construct without the enhancer protein (FIG. 27B Spike triangles).

This further demonstrated the advantage of the addition of the enhancer protein to the vaccine candidates.

### Example 10: L Protein Increased Functional West Nile Virus (WNV) VLP Production when Co-Expressed with WNV E and M Proteins

A plasmid encoding the precursor membrane protein (prM), the envelope glycoprotein (E) of NY99 strain of WNV and an enhancer protein was constructed as described in Example 6 (see FIG. 14A, SEQ ID NO: 55). Also, a control plasmid encoding just the precursor membrane protein (prM), and the envelope glycoprotein (E) of NY99 strain of WNV was constructed (FIG. 11B). HEK293T cells were cultured in DMEM supplemented with 10% FBS at 37° C. at 5% CO₂. On Day 1, the cells were seeded on 24-well plates at 20,000 cells per well and grown overnight. On Day 2, cells seeded to the 24-well plates were transiently transfected with a plasmid encoding the precursor membrane protein (prM), the envelope glycoprotein (E) of NY99 strain of WNV and an enhancer protein. A control plasmid was used in all experiments, which encodes just the precursor membrane protein (prM) and the envelope glycoprotein (E) of NY99 strain of WNV, and not the enhancer protein.

Each well of a 24-well plate was transfected using plasmid/PEI complexes, which were formed using 0.5 ug of the corresponding plasmid and 1 ug of PEI in 50 μl Opti-MEM. The complexes were formed by incubating plasmid/PEI mixture at room temperature for 30 min. Cell medium in 24-well plates was replaced by fresh Opti-MEM and complexes were added to the wells. On Day 3, the complexes were removed from transfected cells and replaced with fresh Opti-MEM.

On Day 4, cell culture supernatants were collected, removed from cell debris by centrifugation at 500×g for 5 minutes and saved for downstream analysis by ELISA. Cells were fixed using 250 μl of 10% neutral buffered formalin (10 minutes at room temperature), and permeabilized using 0.2% Triton-X 100 (10 minutes at room temperature) and washed.

Fluorescence microscopy was used to visualize protein expression in cells as followed. Cells were stained using mouse anti-WNV_E and rabbit anti-WNV_M primary antibodies (1:500 dilution in PBS, 1 h at room temperature), washed, developed with goat anti-mouse Alexa Fluor 488 secondary antibodies (1:1000 dilution in PBS, 1 h at room temperature), washed, and imaged using fluorescence microscopy.

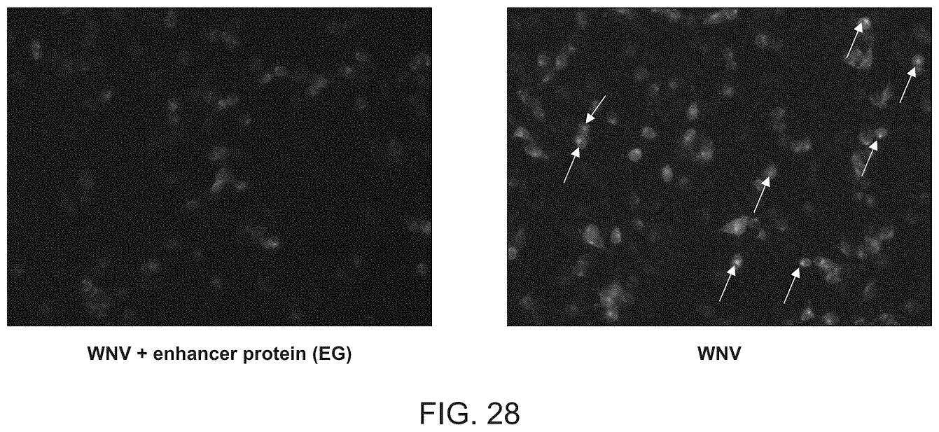

The results of the immunostaining experiments are shown in FIG. 28. As observed in other experiments described herein, the quantity of the WNV+enhancer protein (EG) was lower compared to the quantity of the WNV without it. However, the quality seemed to be higher when the enhancer protein was present as observed by the formation of nuclei (FIG. 28, right, as indicated by arrows). These data demonstrate the higher quality of proteins expressed with the compositions and methods provided herein.

ELISA assays were used to demonstrate the secretion of expressed antigens. For this, supernatant from transfected cells were collected on days 4 (48 hours after transfection), 5 (72 hours), 6 (96 hours), 7 (120 hours) and 8 (144 hours). High-binding 96-well plates were coated with the cell culture supernatants using 75 μl of cell culture supernatant per well and incubated at +4° C. overnight. The next day, the coated wells were washed using PBST buffer and blocked using 200 μl of EZ Block™ reagent (Scytek Laboratories) per well for 2 h at +37° C. The wells were washed 3 times with PBST and incubated with a primary antibody (mouse anti-WNV_E, diluted 1:1000 in EZ Block, 75 μl per well) for 1 hour at room temperature. The wells were then washed 3 times with PBST and incubated with the goat anti-mouse HRP secondary antibody diluted 1:1000 in EZ Block reagent, 75 μl per well, for 1 hour at room temperature. The wells were then washed 5 times using PBST and 75 μl of TMB substrate was added to each well and incubated 30 minutes at room temperature, followed by the addition of 75 μl of Stop Solution, and absorbance measured at 450 nm using a plate reader. Additionally, to demonstrate that the VLP secretion was not caused by cell death and the unspecific release of intracellular protein, cells were imaged every day and ELISA results were compared to the images.

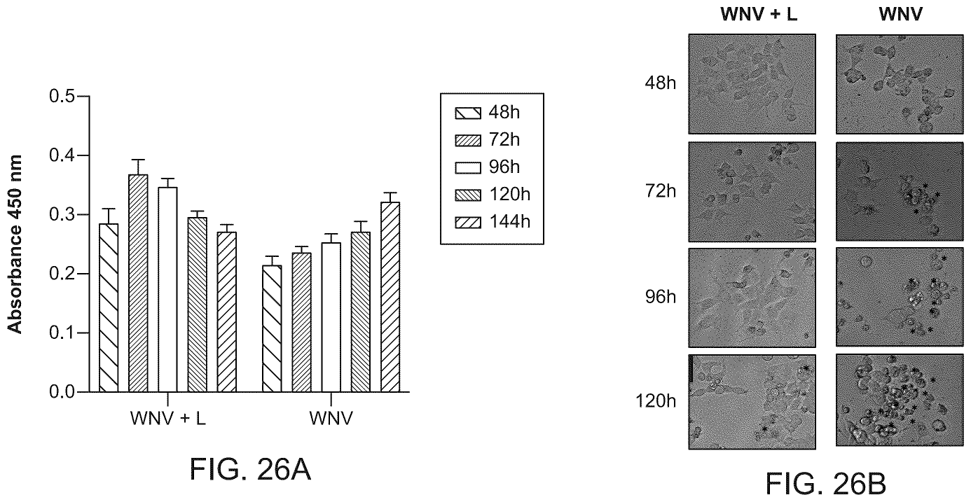

FIG. 26 A illustrates the secretion of VLPs from transfected cells over time as measured by ELISA. Whereas the WNV construct with the enhancer protein showed the highest secretion 72 hours after transfection as was expected for intact VLPs and healthy cells, the WNV construct without the enhancer protein showed a steady increase in secreted material over time. The latter was more consistent with increased cell death and unspecific release of protein material that most likely are not fully formed VLPs. The results were confirmed by analysis of cell images taken at the time of harvest from the supernatant. Whereas the WNV with the enhancer protein showed little to no cell death (as indicated by stars) over the time of the experiment, WNV without the enhancer protein showed visible cell death (as indicated by stars), after 72 hours this became increasingly more pronounced over the time of the experiment. The cell images are further proof that the released material from the WNV without the enhancer protein was most likely due to the release of protein from cell death rather from controlled secretion of VLPs.

This example further demonstrates that the methods and compositions of the disclosure improve the quality of produced antigen.

### Example 11: L Protein Increased Total West Nile Virus (WNV) VLP Production when Co-Expressed with WNV— E and M Proteins

To isolate intact VLPs, 4×10⁶ HEK293 cells in a 150 mm dish were transfected with a plasmid encoding the precursor membrane protein (prM) and the envelope glycoprotein (E) of NY99 strain of WNV, and an enhancer protein. A control plasmid was used in all experiments, which encodes just the precursor membrane protein (prM) and the envelope glycoprotein (E) of NY99 strain of WNV, and not the enhancer protein. The transfections were conducted using PEI complexes following the manufacturers description using 40 μg plasmid and 80 μg PEI per 150 mm dish. Media was changed 12 hours after transfection and cells were incubated at 37° C., 5% CO₂ for 72 hours. VLP containing supernatants were harvested, spun down (1,500×g, 15 minutes) and concentrated using an Amicon Ultra centrifugal filter unit (100 kDa cut off). Concentrate was spun down (4,500×g, 15 minutes) to remove precipitate and VLPs were pelleted through a 20% sucrose cushion at 100,000×g for 1.5 hours. VLPs were resuspended in PBS and analyzed by ELISA.

ELISAs were performed as described above, and as known in the art, for instance, as described in Cold Spring Harb Protoc; doi:10.1101/pdb.prot093708. Briefly, high-binding 96-well plates were coated using VLPs resuspended in PBS in serial dilutions from 1:20 to 1:72,000 to visualize the difference of expression quantity between the constructs with and without the enhancer protein and incubated overnight at 4° C. Plates were washed and blocked with EZ block (Scytek Laboratories) for 2 hours at 37° C. Anti-West Nile Virus Antibody, clone E16, was diluted in EZ block (1:5,000) and plates were incubated for 1 hour at RT. Wells were washed and goat anti-mouse HRP labeled detection antibody (Southern Biotech) was added for detection, followed by washes and the incubation with TMB substrate and the stop solution. Signal was read out as absorbance at 450 nm using a plate reader.

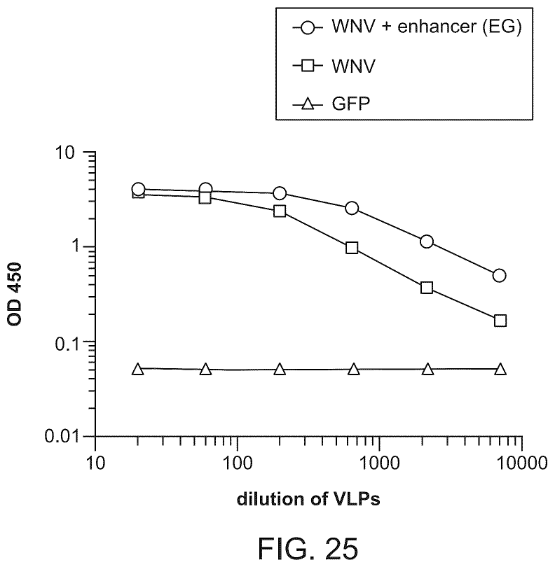

FIG. 25 demonstrates the difference between the total expression and secretion of West Nile virus constructs with and without the enhancer protein. As shown, the addition of the enhancer protein (circles) led to a higher expression of West Nile virus particles compared to the construct lacking the enhancer protein (squares). This demonstrates that the compositions and methods of the disclosure are beneficial for WNV VLP vaccine production.

### Example 12: Immunogenicity of West Nile Virus Proteins Co-Expressed with the L Enhancer Protein in Mice

The ability to evoke immune responses in vivo upon vaccination with a plasmid encoding the precursor membrane (prM), the envelope glycoprotein (E) of WNV, and the enhancer protein, is evaluated using BALB/c mice as follows. 6-week-old female BALB/c mice are randomized into groups based on body weight. Mice are dosed with the plasmids using intradermal or intramuscular injections on Day 1 and Day 21. Mouse serum samples are collected on Day 1 (pre-vaccination), on Day 21 (prior to boost) and on 42. On day 42, mice are sacrificed and splenocytes are isolated.

The elicited humoral immune response is measured by evaluating the titer of anti-M and anti-E antibodies a respective enzyme linked immunosorbent assay (ELISA). Additionally, cellular immune response is measured by evaluating the presence of antigen reactive T cells using a respective IFN-γ and IL-4 enzyme-linked immune absorbent Spot (Dual color ELISpot) assay.

ELISAs are performed as described here, and as known in the art, for instance, as described in Cold Spring Harb Protoc; doi:10.1101/pdb.prot093708. Briefly, high-binding 96-well plates are coated using recombinant prM and E proteins (Abcam) at 2 μg/ml concentration and blocked. Serum samples are serially diluted in EZ Block reagent and added to pre-coated wells, washed and detected using goat anti-mouse HRP labeled detection antibody, followed by washes and the incubation with TMB substrate and the stop solution. Endpoint titer is defined as the reciprocal maximal antibody dilution at which the ELISA signal (absorbance at 450 nm) is above 3 standard deviations of background signal.

Dual color ELISpot assay is conducted as described here, and as known in the art, for instance, as described in Cold Spring Harb Protoc 2010 doi:10.1101/pdb.prot5369. Briefly, splenocytes are isolated on Day 42, stimulated with respective prM or E peptide arrays (Biodefense and Emerging Infections Research Resources Repository) and added to the pre-prepared ELISpot microplates. Negative (medium) and positive controls (Phorbol Myristate Acetate/Ionomycin) are included in the assay. The number of antigen-reactive IFN-gamma and IL-4 secreting T cells are counted using an ELISpot reader.

Finally, the presence of WNV neutralizing antibodies in mouse sera isolated at different time points (see above) is evaluated as described herein, and as described in The Journal of Infectious Diseases, Volume 196, Issue 12, 15 Dec. 2007, Pages 1732-1740, and Virology, Volume 346, Issue 1, 1 Mar. 2006, Pages 53-65. Briefly, WNV reporter-virus particles (RVPs) are generated in HEK293T cells by transiently transfecting WNV prM and E proteins (to form virus-like particles also known as subviral particles), complemented with transiently transfected reporter-replicon (luciferase) and transiently transfected capsid protein. Isolated RVPs are incubated with mouse serum samples at different serial dilutions and added to pre-plated PHK-21 cells and incubated for 2 days, after which the reporter gene activity is measured using a microplate reader. The reduction in the reporter gene activity reflects the level of WNV neutralizing antibodies in mouse sera.

### Example 13: Expression and Immunogenicity of Additional Polynucleotide Constructs

Construction of plasmids encoding viral proteins derived from other viruses, e.g., Influenza viral proteins (e.g., HA, NA, M1, M2, or any combination thereof), Hepatitis B viral proteins (e.g., sAg (S protein), sAg (M protein), sAg (L protein), preS1, preS2, cAg (core antigen), or any combination thereof), Human Papillomavirus (e.g., L1 protein of HPV 6, L1 protein of HPV 11, L1 protein of HPV 16, L1 protein of HPV 18, or any combination thereof) is performed using the methods described in Example 6. Expression of these proteins in different combinations in HEK293T cells and isolation of the VLPs is performed using methods described in Examples 7, 8, 10 and 11. Finally, the immunogenicity of the plasmids encoding these proteins is tested using the methods described in Example 9 and 12.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### NUMBERED EMBODIMENTS

Embodiment 1. A vector for use as a vaccine, comprising an expression cassette comprising a polynucleotide encoding a viral protein and a polynucleotide encoding an enhancer protein, wherein the enhancer protein is a picornavirus leader (L) protein or a functional variant thereof.

Embodiment 2. The vector of embodiment 1, wherein the amino acid sequence of the enhancer protein has at least 95% identity to SEQ ID NO: 1, or at least 95% identity to SEQ ID NO: 2.

Embodiment 3. The vector of embodiment 1 or embodiment 2, wherein the amino acid sequence of the enhancer protein is SEQ ID NO: 1, or SEQ ID NO: 2.

Embodiment 4. The vector of any one of embodiments 1-3, wherein the polynucleotide encoding the enhancer protein is operatively linked to a polynucleotide encoding an internal ribosome entry site (IRES).

Embodiment 5. The vector of embodiment 4, wherein the polynucleotide encoding the IRES is SEQ ID NO: 24.

Embodiment 6. The vector of any one of embodiments 1-5, wherein the viral protein is a viral antigen.

Embodiment 6.1 The vector of any one of embodiments 1-6, wherein the viral protein is derived from a virus selected from the group consisting of coronavirus, influenza virus, Hepatitis B virus, Human Papilloma virus (HPV), West Nile virus, and Human Immunodeficiency Virus (HIV) virus.

Embodiment 6.2 The vector of embodiment 6.1, wherein the viral protein is derived from a coronavirus.

Embodiment 7. The vector of any one of embodiments 1-6.2, wherein the coronavirus is a betacoronavirus.

Embodiment 8. The vector of embodiment 7, wherein the betacoronavirus is severe acute respiratory syndrome (SARS) virus.

Embodiment 9. The vector of embodiment 8, wherein the SARS virus is a SARS-CoV-2 virus.

Embodiment 10. The vector of embodiment 7, wherein the betacoronavirus is Middle East respiratory syndrome (MERS) virus.

Embodiment 11. The vector of any one of embodiments 1-10, wherein the coronavirus protein is a coronavirus spike protein.

Embodiment 12. The vector of embodiment 11, wherein the spike protein shares at least 70% identity, at least 75%, at least 80%, at least 85%, at least 90%, at least 95%, at least 96%, at least 97%, at least 98%, at least 99%, at least 99.5% or 100% identity to SEQ ID NO: 13.

Embodiment 13. The vector of embodiment 12, wherein the spike protein is SEQ ID NO: 13.

Embodiment 13.1 The vector of embodiment 11, wherein the spike protein is a mutant spike protein.

Embodiment 13.2 The vector of embodiment 13.1, wherein the mutant spike protein comprises the amino acid substitutions, R682G, R683S, R685S, K986P, and V987P, in SEQ ID NO: 13.

Embodiment 13.3 The vector of embodiment 13.1, wherein the mutant spike protein comprises an amino acid sequence of SEQ ID NO: 51.

Embodiment 14. The vector of any one of embodiments 1-13.3, wherein the coronavirus protein is a coronavirus membrane (M) protein.

Embodiment 15. The vector of embodiment 14, wherein the M protein shares at least 80%, at least 85%, at least 90%, at least 95%, at least 96%, at least 97%, at least 98%, at least 99%, at least 99.5% or 100% identity to SEQ ID NO: 33.

Embodiment 16. The vector of embodiment 14 or embodiment 15, wherein the M protein is SEQ ID NO: 33.

Embodiment 17. The vector of any one of embodiments 1-16, wherein the coronavirus protein is a coronavirus envelope (E) protein.

Embodiment 18. The vector of embodiment 17, wherein the E protein shares at least 80%, at least 85%, at least 90%, at least 95%, at least 96%, at least 97%, at least 98%, at least 99%, at least 99.5% or 100% identity to SEQ ID NO: 22.

Embodiment 19. The vector of embodiment 17 or embodiment 18, wherein the E protein is SEQ ID NO: 22.

Embodiment 20. The vector of any one of embodiments 1-19, wherein the coronavirus protein is a coronavirus nucleocapsid (N) protein.

Embodiment 21. The vector of embodiment 20, wherein the N protein shares at least 80%, at least 85%, at least 90%, at least 95%, at least 96%, at least 97%, at least 98%, at least 99%, at least 99.5% or 100% identity to SEQ ID NO: 20.

Embodiment 22. The vector of embodiment 20 or embodiment 21, wherein the N protein is SEQ ID NO: 20.

Embodiment 23. The vector of any one of embodiments 1-22, wherein the coronavirus protein forms a virus-like particle (VLP).

Embodiment 23.1 The vector of embodiment 6.1, wherein the viral protein is derived from West Nile virus.

Embodiment 23.2 The vector of embodiment 23.1, wherein the viral protein is precursor membrane protein (preM), envelope glycoprotein (E), or a combination thereof.

Embodiment 24. A vector for use as a vaccine, comprising an expression cassette comprising a polynucleotide, wherein the polynucleotide comprises a nucleic acid sequence having at least 70% identity, at least 75%, at least 80%, at least 85%, at least 90%, at least 95%, at least 96%, at least 97%, at least 98%, at least 99%, at least 99.5% or 100% identity to the nucleic acid sequence of SEQ ID NO: 30.

Embodiment 25. The vector of embodiment 24, wherein the polynucleotide comprises the nucleic acid sequence of SEQ ID NO: 30.

Embodiment 26. A vector for use as a vaccine, comprising an expression cassette comprising a polynucleotide, wherein the polynucleotide comprises a nucleic acid sequence having at least 70% identity, at least 75%, at least 80%, at least 85%, at least 90%, at least 95%, at least 96%, at least 97%, at least 98%, at least 99%, at least 99.5% or 100% identity to the nucleic acid sequence of SEQ ID NO: 31.

Embodiment 27. The vector of embodiment 26, wherein the polynucleotide comprises the nucleic acid sequence of SEQ ID NO: 31.

Embodiment 27.1 A vector for use as a vaccine, comprising a nucleic acid sequence having at least 70% identity, at least 75%, at least 80%, at least 85%, at least 90%, at least 95%, at least 96%, at least 97%, at least 98%, at least 99%, at least 99.5% or 100% identity to a nucleic acid sequence selected from the group consisting of SEQ ID NO: 35-49, and 55.

Embodiment 28. The vector of any one of embodiments 1-27.1, wherein the vector is a naked polynucleotide.

Embodiment 29. The vector of any one of embodiments 1-28, wherein the vector is a deoxyribonucleic acid (DNA) polynucleotide.

Embodiment 30. The vector of any one of embodiments 1-28, wherein the vector is a ribonucleic acid (RNA) polynucleotide.

Embodiment 31. The vector of any one of embodiments 1-30, wherein the vector comprises a plasmid.

Embodiment 32. The vector of any one of embodiments 1-30, wherein the vector comprises linear DNA.

Embodiment 33. The vector of any one of embodiments 1-32, wherein the expression cassette comprises a promoter operatively linked to each of the polynucleotide sequences of the expression cassette.

Embodiment 33.1 The vector of any one of embodiments 1-33, wherein the vector comprises a DNA polynucleotide, said DNA polynucleotide encoding a viral packaging signal.

Embodiment 33.2 The vector of embodiment 33.1, wherein the viral packaging signal is a RNA polynucleotide.

Embodiment 33.3 The vector of embodiment 33.2, wherein the viral packaging signal is derived from a coronavirus.

Embodiment 34. A vaccine composition, comprising the vector of any one of embodiments 1 to 33.4 and a pharmaceutically acceptable carrier.

Embodiment 35. The vaccine composition of embodiment 34, wherein the vaccine composition comprises an adjuvant.

Embodiment 36. The vaccine composition of embodiment 35, wherein the adjuvant is alum.

Embodiment 37. The vaccine composition of embodiment 35, wherein the adjuvant is monophosphoryl lipid A (MPL).

Embodiment 38. A method of expressing a viral antigen in a eukaryotic cell, comprising contacting the cell with the vector of any one of embodiments 1 to 33.4.

Embodiment 39. The method of embodiment 38, wherein contacting the cell with the vector results in: (i) expression of the antigen at a higher expression level; and/or (ii) expression of the antigen for a longer period of time; and/or (iii) expression of the antigen with better protein quality, than a vector lacking the enhancer protein.

Embodiment 40. The method of embodiment 38 or embodiment 39, wherein contacting the cell with the vector results in: (i) expression of a virus like particle (VLP) comprising the antigen at a higher expression level; and/or (ii) expression of a VLP comprising the antigen for a longer period of time; and/or (iii) expression of a VLP comprising the antigen with better protein quality, than a vector lacking the enhancer protein.

Embodiment 40.1 The method of embodiment 40, wherein the vector comprises a DNA polynucleotide encoding a viral packaging signal, wherein contacting the cell with the vector results in expression of the viral packaging signal, and wherein the VLPs encapsidate the viral packaging signal.

Embodiment 40.2 The method of embodiment 40.1, wherein the vector results in the formation of a greater number of VLPs, as compared to a control vector lacking the DNA polynucleotide encoding the viral packaging signal.

Embodiment 41. A method of eliciting an immune response in a subject, comprising administering an effective amount of the vaccine composition of any one of embodiments 34 to 37 to the subject.

Embodiment 42. The method of embodiment 41, wherein tissue at an administration site of the subject expresses the antigen and/or a VLP comprising the antigen.

Embodiment 43. The method of embodiment 42, wherein tissue at an administration site of the subject: (i) expresses the antigen and/or a VLP comprising the antigen at a higher expression level; and/or (ii) expresses the antigen and/or a VLP comprising the antigen for a longer period of time; and/or (iii) expresses the antigen and/or a VLP comprising the antigen with better protein quality, than when a vector lacking the enhancer protein is administered.

Embodiment 43.1 The method of any one of embodiments 41-43, wherein the vector comprises a DNA polynucleotide encoding a viral packaging signal, wherein tissue at an administration site of the subject expresses the viral packaging signal, and wherein the VLPs encapsidate the viral packaging signal.

Embodiment 43.2 The method of embodiment 43 or 43.1, wherein the vector results in the expression of a greater number of VLPs, as compared to a control vector lacking the DNA polynucleotide encoding the viral packaging signal.

Embodiment 43.3 The method of embodiment 43-43.2, wherein the VLPs encapsidating the viral packaging signal are more immunogenic than control VLPs comprising the antigen but lacking the viral packaging signal.

Embodiment 44. The method of any one of embodiments 41 to 43, wherein the method elicits an antibody response in the subject.

Embodiment 45. The method of embodiment 44, wherein the antibody response is a neutralizing antibody response.

Embodiment 46. The method of any one of embodiments 41 to 43, wherein the method elicits a cellular immune response.

Embodiment 47. The method of any one of embodiments 41 to 46, wherein the method elicits a prophylactic, protective and/or therapeutic immune response in the subject.

Embodiment 48. The method of any one of embodiments 41 to 47, wherein the administration is intradermal administration, intramuscular administration, subcutaneous administration, or intranasal administration.

Embodiment 49. A polynucleotide comprising an expression cassette comprising a polynucleotide encoding a coronavirus protein and a polynucleotide encoding an enhancer protein, wherein the enhancer protein is a picornavirus leader (L) protein or a functional variant thereof.

Embodiment 50. The polynucleotide of embodiment 49, wherein the amino acid sequence of the enhancer protein has at least 95% identity to SEQ ID NO: 1, or at least 95% identity to SEQ ID NO: 2.

Embodiment 51. The polynucleotide of embodiment 49 or embodiment 50, wherein the amino acid sequence of the enhancer protein is SEQ ID NO: 1, or SEQ ID NO: 2.

Embodiment 52. The polynucleotide of any one of embodiments 49-51, wherein the polynucleotide encoding the enhancer protein is operatively linked to a polynucleotide encoding an internal ribosome entry site (IRES).

Embodiment 53. The polynucleotide of embodiment 52, wherein the polynucleotide encoding the IRES is SEQ ID NO: 24.

Embodiment 54. The polynucleotide of any one of embodiments 49-53, wherein the coronavirus protein is a coronavirus antigen.

Embodiment 55. The polynucleotide of any one of embodiments 49-54, wherein the coronavirus is a betacoronavirus.

Embodiment 56. The polynucleotide of embodiment 55, wherein the betacoronavirus is severe acute respiratory syndrome (SARS) virus.

Embodiment 57. The polynucleotide of embodiment 56, wherein the SARS virus is a SARS-CoV-2 virus.

Embodiment 58. The polynucleotide of embodiment 55, wherein the betacoronavirus is Middle East respiratory syndrome (MERS) virus.

Embodiment 59. The polynucleotide of any one of embodiments 49-58, wherein the coronavirus protein is a coronavirus spike protein.

Embodiment 60. The polynucleotide of embodiment 59, wherein the spike protein shares at least 70% identity, at least 75%, at least 80%, at least 85%, at least 90%, at least 95%, at least 96%, at least 97%, at least 98%, at least 99%, at least 99.5% or 100% identity to SEQ ID NO: 13.

Embodiment 61. The polynucleotide of embodiment 59 or embodiment 60, wherein the spike protein is SEQ ID NO: 13.

Embodiment 62. The polynucleotide of any one of embodiments 49-61, wherein the coronavirus protein is a coronavirus membrane (M) protein.

Embodiment 63. The polynucleotide of embodiment 62, wherein the M protein shares at least 80%, at least 85%, at least 90%, at least 95%, at least 96%, at least 97%, at least 98%, at least 99%, at least 99.5% or 100% identity to SEQ ID NO: 33.

Embodiment 64. The polynucleotide of embodiment 62 or embodiment 63, wherein the M protein is SEQ ID NO: 33.

Embodiment 65. The polynucleotide of any one of embodiments 49-64, wherein the coronavirus protein is a coronavirus envelope (E) protein.

Embodiment 66. The polynucleotide of embodiment 65, wherein the E protein shares at least 80%, at least 85%, at least 90%, at least 95%, at least 96%, at least 97%, at least 98%, at least 99%, at least 99.5% or 100% identity to SEQ ID NO: 22.

Embodiment 67. The polynucleotide of embodiment 65 or embodiment 66, wherein the E protein is SEQ ID NO: 22.

Embodiment 68. The polynucleotide of any one of embodiments 49-67, wherein the coronavirus protein is a coronavirus nucleocapsid (N) protein.

Embodiment 69. The polynucleotide of embodiment 68, wherein the N protein shares at least 80%, at least 85%, at least 90%, at least 95%, at least 96%, at least 97%, at least 98%, at least 99%, at least 99.5% or 100% identity to SEQ ID NO: 20.

Embodiment 70. The polynucleotide of embodiment 68 or embodiment 69, wherein the N protein is SEQ ID NO: 20.

Embodiment 71. The polynucleotide of any one of embodiments 49-70, wherein the coronavirus protein forms a virus-like particle (VLP).

Embodiment 72. A polynucleotide comprising a nucleic acid sequence having at least 70% identity, at least 75%, at least 80%, at least 85%, at least 90%, at least 95%, at least 96%, at least 97%, at least 98%, at least 99%, at least 99.5% or 100% identity to the nucleic acid sequence of SEQ ID NO: 30.

Embodiment 73. The polynucleotide of embodiment 72, wherein the polynucleotide comprises a nucleic acid sequence of SEQ ID NO: 30.

Embodiment 74. A polynucleotide comprising a nucleic acid sequence having at least 70% identity, at least 75%, at least 80%, at least 85%, at least 90%, at least 95%, at least 96%, at least 97%, at least 98%, at least 99%, at least 99.5% or 100% identity to the nucleic acid sequence of SEQ ID NO: 31.

Embodiment 75. The polynucleotide of embodiment 74, wherein the polynucleotide comprises a nucleic acid sequence of SEQ ID NO: 31.

Embodiment 76. The polynucleotide of any one of embodiments 49-75, wherein the polynucleotide is a naked polynucleotide.

Embodiment 77. The polynucleotide of any one of embodiments 49-76, wherein the polynucleotide is a deoxyribonucleic acid (DNA) polynucleotide.

Embodiment 78. The polynucleotide of any one of embodiments 49-76, wherein the polynucleotide is a ribonucleic acid (RNA) polynucleotide.

Embodiment 79. The polynucleotide of any one of embodiments 49-71 and 76-78, wherein the expression cassette comprises a promoter operatively linked to each of the polynucleotide sequences of the expression cassette.

Embodiment 80. A kit comprising a vector, wherein the vector comprises an expression cassette comprising a polynucleotide encoding a coronavirus protein and a polynucleotide encoding an enhancer protein, wherein the enhancer protein is a picornavirus leader (L) protein or a functional variant thereof.

Embodiment 81. The kit of embodiment 80, wherein the amino acid sequence of the enhancer protein has at least 95% identity to SEQ ID NO: 1, or at least 95% identity to SEQ ID NO: 2.

Embodiment 82. The kit of embodiment 80 or embodiment 81, wherein the polynucleotide encoding the enhancer protein is operatively linked to a polynucleotide encoding an internal ribosome entry site (IRES).

Embodiment 83. The kit of embodiment 82, wherein the polynucleotide encoding the IRES is SEQ ID NO: 24.

Embodiment 84. The kit of any one of embodiments 80-83, wherein the coronavirus protein is a coronavirus antigen.

Embodiment 85. The kit of any one of embodiments 80-84, wherein the coronavirus is a betacoronavirus.

Embodiment 86. The kit of embodiment 85, wherein the betacoronavirus is severe acute respiratory syndrome (SARS) virus.

Embodiment 87. The kit of embodiment 86, wherein the SARS virus is a SARS-CoV-2 virus.

Embodiment 88. The kit of embodiment 85, wherein the betacoronavirus is Middle East respiratory syndrome (MERS) virus.

Embodiment 89. The kit of any one of embodiments 80-88, wherein the coronavirus protein is a coronavirus spike protein.

Embodiment 90. The kit of embodiment 89, wherein the spike protein shares at least 70% identity, at least 75%, at least 80%, at least 85%, at least 90%, at least 95%, at least 96%, at least 97%, at least 98%, at least 99%, at least 99.5% or 100% identity to SEQ ID NO: 13.

Embodiment 91. The kit of embodiment 90, wherein the spike protein is SEQ ID NO: 13.

Embodiment 92. The kit of any one of embodiments 80-91, wherein the coronavirus protein is a coronavirus membrane (M) protein.

Embodiment 93. The kit of embodiment 92, wherein the M protein shares at least 80%, at least 85%, at least 90%, at least 95%, at least 96%, at least 97%, at least 98%, at least 99%, at least 99.5% or 100% identity to SEQ ID NO: 33.

Embodiment 94. The kit of embodiment 92 or embodiment 93, wherein the M protein is SEQ ID NO: 33.

Embodiment 95. The kit of any one of embodiments 80-94, wherein the coronavirus protein is a coronavirus envelope (E) protein.

Embodiment 96. The kit of embodiment 95, wherein the E protein shares at least 80%, at least 85%, at least 90%, at least 95%, at least 96%, at least 97%, at least 98%, at least 99%, at least 99.5% or 100% identity to SEQ ID NO: 22.

Embodiment 97. The kit of embodiment 95 or embodiment 96, wherein the E protein is SEQ ID NO: 22.

Embodiment 98. The kit of any one of embodiments 80-97, wherein the coronavirus protein is a coronavirus nucleocapsid (N) protein.

Embodiment 99. The kit of embodiment 98, wherein the N protein shares at least 80%, at least 85%, at least 90%, at least 95%, at least 96%, at least 97%, at least 98%, at least 99%, at least 99.5% or 100% identity to SEQ ID NO: 20.

Embodiment 100. The kit of embodiment 98 or embodiment 99, wherein the N protein is SEQ ID NO: 20.

Embodiment 101. The kit of embodiment 80, wherein the expression cassette comprises a polynucleotide, comprising a nucleic acid sequence having at least 70% identity, at least 75%, at least 80%, at least 85%, at least 90%, at least 95%, at least 96%, at least 97%, at least 98%, at least 99%, at least 99.5% or 100% identity to the nucleic acid sequence of SEQ ID NO: 30.

Embodiment 102. The kit of embodiment 80, wherein the expression cassette comprises a polynucleotide, comprising a nucleic acid sequence having at least 70% identity, at least 75%, at least 80%, at least 85%, at least 90%, at least 95%, at least 96%, at least 97%, at least 98%, at least 99%, at least 99.5% or 100% identity to the nucleic acid sequence of SEQ ID NO: 31.

Embodiment 103. The kit of any one of embodiments 80-102, wherein the kit comprises a pharmaceutically acceptable carrier.

Embodiment 104. A vector, comprising an expression cassette, said expression cassette comprising a promoter linked to a target gene, wherein the vector comprises a nucleic acid sequence encoding a viral packaging element.

Embodiment 105. The vector of embodiment 104, wherein the viral packaging element is a RNA polynucleotide.

Embodiment 106. The vector of embodiment 104 or 105, wherein the viral packaging element is derived from a coronavirus.

Embodiment 107. The vector of embodiment 106, wherein the viral packaging element is derived from SARS-CoV2.

Embodiment 108. The vector of any one of embodiments 104-107, wherein the nucleic acid sequence encoding the viral packaging element has at least about 70% identity to the nucleic acid sequence of SEQ ID NO: 34.

Embodiment 109. The method of expressing a target protein in a eukaryotic cell, comprising contacting the cell with the vector of any one of embodiments 104-108.

Embodiment 110. The method of embodiment 109, wherein contacting the cell with the vector results in the formation of virus-like particles (VLPs) comprising the target protein.

Embodiment 111. The method of embodiment 110, wherein contacting the cell with the vector results in the formation of a greater number of virus-like particles (VLPs) comprising the target protein, as compared to a control vector comprising the expression cassette but lacking the nucleic acid sequence encoding the viral packaging element.

Embodiment 112. The vector of any one of embodiments 33.1-33.3, or the method of any one of embodiments 40.1, 40.2, 43.1-43.3, wherein the nucleic acid sequence encoding the viral packaging element has at least about 70% identity to the nucleic acid sequence of SEQ ID NO: 34.

Embodiment 113. A vector for use as a vaccine, comprising an expression cassette, comprising the following elements in the 5′ to 3′ order: a promoter, a polynucleotide encoding SEQ ID NO: 33 (M protein), a polynucleotide encoding a first proteolytic cleavage site, a polynucleotide encoding SEQ ID NO: 20 (N protein), a polynucleotide encoding a second proteolytic cleavage site, a polynucleotide encoding SEQ ID NO: 13 (S protein), a polynucleotide encoding a third proteolytic cleavage site, a polynucleotide encoding SEQ ID NO: 22 (E protein), polynucleotide encoding SEQ ID NO: 24 (IRES), and a polynucleotide encoding SEQ ID NO: 2 (enhancer L protein).

Embodiment 114. A vector for use as a vaccine, comprising an expression cassette, comprising the following elements in the 5′ to 3′ order: a promoter, a polynucleotide encoding SEQ ID NO: 33 (M protein), a polynucleotide encoding a first proteolytic cleavage site, a polynucleotide encoding SEQ ID NO: 13 (S protein), a polynucleotide encoding a second proteolytic cleavage site, a polynucleotide encoding SEQ ID NO: 22 (E protein), polynucleotide encoding SEQ ID NO: 24 (IRES), a polynucleotide encoding SEQ ID NO: 2 (enhancer L protein), a polynucleotide encoding SEQ ID NO: 20 (N protein), and a polynucleotide encoding SEQ ID NO: 34 (viral packaging signal).

Embodiment 115. A vector for use as a vaccine, comprising an expression cassette, comprising the following elements in the 5′ to 3′ order: a promoter, a polynucleotide encoding an M protein wherein the M protein comprises SEQ ID NO: 33 or an amino acid sequence at least 95% identical thereto, a polynucleotide encoding a proteolytic cleavage site, a polynucleotide encoding an N protein, wherein the N protein comprises SEQ ID NO: 20 or an amino acid sequence at least 95% identical thereto, a polynucleotide encoding a proteolytic cleavage site, a polynucleotide encoding a mutated S protein wherein the S protein comprises SEQ ID NO: 51 or SEQ ID NO: 52 or an amino acid sequence at least 95% identical thereto, a polynucleotide encoding a proteolytic cleavage site, a polynucleotide encoding a E protein wherein the E protein comprises SEQ ID NO: 22 or an amino acid sequence at least 95% identical thereto, a polynucleotide encoding IRES wherein the IRES sequence comprises SEQ ID NO: 24 or a polynucleotide sequence at least 95% identical thereto, and a polynucleotide encoding an enhancer L protein wherein the L protein comprises SEQ ID NO: 2 or an amino acid sequence at least 95% identical thereto.

Embodiment 116. A vector for use as a vaccine, comprising an expression cassette, comprising the following elements in the 5′ to 3′ order: a promoter, a first polynucleotide encoding a viral packaging signal wherein the viral packaging signal comprises SEQ ID NO: 34 or a polynucleotide sequence at least 9% identical thereto, a polynucleotide encoding an M protein wherein the M protein comprises SEQ ID NO: 33 or an amino acid sequence at least 95% identical thereto, a polynucleotide encoding a proteolytic cleavage site, a polynucleotide encoding an N protein, wherein the N protein comprises SEQ ID NO: 20 or an amino acid sequence at least 95% identical thereto, a polynucleotide encoding a proteolytic cleavage site, a polynucleotide encoding a mutated S protein wherein the S protein comprises SEQ ID NO: 51 or SEQ ID NO: 52 or an amino acid sequence at least 95% identical thereto, a polynucleotide encoding a proteolytic cleavage site, a polynucleotide encoding a E protein wherein the E protein comprises SEQ ID NO: 22 or an amino acid sequence at least 95% identical thereto, a polynucleotide encoding IRES wherein the IRES sequence comprises SEQ ID NO: 24 or a polynucleotide sequence at least 95% identical thereto, a polynucleotide encoding an enhancer L protein wherein the L protein comprises SEQ ID NO: 2 or an amino acid sequence at least 95% identical thereto, and a second polynucleotide encoding a viral packaging signal, wherein the viral packaging signal comprises SEQ ID NO: 34 or a polynucleotide sequence at least 9% identical thereto.

1. A polynucleotide encoding: a viral antigen protein and a nucleocytoplasmic transport (NCT) inhibitor protein.

2. The polynucleotide of claim 1, wherein the polynucleotide encodes an NCT inhibitor protein with at least 70% sequence identity to SEQ ID NO: 2.

3. The polynucleotide of claim 1, wherein the polynucleotide encodes the NCT inhibitor protein of SEQ ID NO: 2.

4-5. (canceled)

6. The polynucleotide of claim 1, wherein the viral antigen protein is a viral structural protein.

7. The polynucleotide of claim 1, wherein the viral antigen protein is derived from a coronavirus.

8-19. (canceled)

20. The polynucleotide of claim 6, wherein the viral structural protein forms a virus-like particle (VLP).

21. The polynucleotide of claim 1, wherein the viral antigen protein is derived from a West Nile virus protein.

22-26. (canceled)

27. The polynucleotide of claim 1, wherein the viral antigen protein is derived from an Influenza viral protein.

28. The polynucleotide of claim 27, wherein the viral antigen protein is one or more of HA, NA, M1, and M2.

29. The polynucleotide of claim 1, wherein the viral antigen protein is derived from a Hepatitis B viral protein.

30. (canceled)

31. The polynucleotide of claim 1, wherein the polynucleotide encodes a viral packaging signal.

32. (canceled)

33. A vector comprising the polynucleotide of claim 1.

34. A vaccine composition, comprising the polynucleotide of claim 1, and a pharmaceutically acceptable carrier and/or an adjuvant.

35. (canceled)

36. A method of expressing a viral antigen protein in a eukaryotic cell comprising contacting a cell with the vector of claim 33.

37-38. (canceled)

39. A method of eliciting an immune response in a subject in need thereof, comprising administering an effective amount of the vaccine composition of claim 34, to the subject.

40-45. (canceled)

46. The method of claim 39, wherein the administration is intradermal administration, intramuscular administration, subcutaneous administration, or intranasal administration.

47. A kit comprising the vaccine composition of claim 34, one or more vials, and instructions for use thereof.

48-59. (canceled)
